SEQUENCE LISTING (758 sequences)

SEQ ID NO: 1 - DNA, 70 bp, Artificial Sequence
  Synthetic polynucleotide sequence
ctgcagatcgaccccaattttggctccaaggaagattttgacagtctcttgcaatcggctaaaaaaaaga

SEQ ID NO: 2 - DNA, 70 bp, Artificial Sequence
  Synthetic polynucleotide sequence
aggtgccttggcagtgtccagcatctaaaaaataggtttggagatggttatacaatagttgtacgaatag

SEQ ID NO: 3 - DNA, 70 bp, Artificial Sequence
  Synthetic polynucleotide sequence
attttccccaactacaacctgggccacgggctcatggagatggcctacaacgagtacatcaacgagtact

SEQ ID NO: 4 - DNA, 70 bp, Artificial Sequence
  Synthetic polynucleotide sequence
ctttatgcccggcttcgaggtgtaccagcagaagaaatcgaaaaggttgcaaactggagtattaagagcc

SEQ ID NO: 5 - DNA, 70 bp, Artificial Sequence
  Synthetic polynucleotide sequence
gtcccccaccctctactggcttggcaactttctctgggacatgtgtaactacttggtgccagcatgcatc

SEQ ID NO: 6 - DNA, 70 bp, Artificial Sequence
  Synthetic polynucleotide sequence
tcgagcttttgggaaagaaatgactgaaatcgagaaatatgccagcaaagtggaccatgtaatgcagtta

SEQ ID NO: 7 - DNA, 70 bp, Artificial Sequence
  Synthetic polynucleotide sequence
ccacgcagatgacttcatccaggaaatggagcatggaatatacacagatgtaggggagaagggaagccag

SEQ ID NO: 8 - DNA, 70 bp, Artificial Sequence
  Synthetic polynucleotide sequence
gatgcagtgaagcagcagaagtgcaacatattctctttgattttcttatttctgggaattatttcttttt

SEQ ID NO: 9 - DNA, 70 bp, Artificial Sequence
  Synthetic polynucleotide sequence
tggtgttcaatgtcatccccacgctggccgacatcatcattggcatcatctacttcagcatgttcttcaa

SEQ ID NO: 10 - DNA, 70 bp, Artificial Sequence
  Synthetic polynucleotide sequence
tgaaactgtgaagtattttaataatgaaagatatgaagcacagagatatgatggattcttgaagacgtat

SEQ ID NO: 11 - DNA, 70 bp, Artificial Sequence
  Synthetic polynucleotide sequence
acgtagggagtttcatgactgagtcccagaatctcagcacccacctgcttatcctctatggtgtccaggg

SEQ ID NO: 12 - DNA, 70 bp, Artificial Sequence
  Synthetic polynucleotide sequence
atctatgtcttcagccacctggaccgcagcctcctggaggacatccgccacttcaacatctttgactcgg

SEQ ID NO: 13 - DNA, 70 bp, Artificial Sequence
  Synthetic polynucleotide sequence
tcctcagcatcttccttttcatgtgtaaccatgtgtccgcgctggcttccaactattggctcagcctctg

SEQ ID NO: 14 - DNA, 70 bp, Artificial Sequence
  Synthetic polynucleotide sequence
aggaagacgaagaactagtgaaaggacaaaaactaattaagaaggaattcatagaaactggaaaggtgaa

SEQ ID NO: 15 - DNA, 70 bp, Artificial Sequence
  Synthetic polynucleotide sequence
catcgacctggagactgacaacctcatccaggctaccatccgcacccagtttgatacctgcactgtcctg

SEQ ID NO: 16 - DNA, 70 bp, Artificial Sequence
  Synthetic polynucleotide sequence
ggaactctgaggagtaatattttatttgggaagaaatatgaaaaggaacgatatgaaaaagtcataaagg

SEQ ID NO: 17 - DNA, 70 bp, Artificial Sequence
  Synthetic polynucleotide sequence
aaataccacacacacttgctacagttcgatggggagggcggctggaagttcgagaagctggactcagctg

SEQ ID NO: 18 - DNA, 70 bp, Artificial Sequence
  Synthetic polynucleotide sequence
atatgttttgggtctttgatgaagtaaaaagaggcatttataagagaactgctgtcattcaagaatctga

SEQ ID NO: 19 - DNA, 70 bp, Artificial Sequence
  Synthetic polynucleotide sequence
tctccagcggttcctgcagatactgaaggttttgtttccttcttggtcatcacaaaatgccttgatgttc

SEQ ID NO: 20 - DNA, 70 bp, Artificial Sequence
  Synthetic polynucleotide sequence
cagagtgtcatcttatttccttggaaaactgttatctgatttattacccatgaggatgttaccaagtatt

SEQ ID NO: 21 - DNA, 70 bp, Artificial Sequence
  Synthetic polynucleotide sequence
aggaatcgttactctttcagatttcctttttttctggggaactgtatttttaataacaacaacattggtt

SEQ ID NO: 22 - DNA, 70 bp, Artificial Sequence
  Synthetic polynucleotide sequence
aaaacccctcttcattcttgctgatctgtactcatctttcagtctctgcctctttagcaaccagagccta

SEQ ID NO: 23 - DNA, 70 bp, Artificial Sequence
  Synthetic polynucleotide sequence
tctcttaaaacacaaatacagtttcctggtgggatgtgcctcaatctcagatgtcatagctcaggttgtt

SEQ ID NO: 24 - DNA, 70 bp, Artificial Sequence
  Synthetic polynucleotide sequence
atattatgtacacggggacagttgactgctggaggaagattgcaaaagacgaaggagccaaggccttctt

SEQ ID NO: 25 - DNA, 70 bp, Artificial Sequence
  Synthetic polynucleotide sequence
acatcatgtacacaggcacgcttgactgctggcggaagattgctcgtgatgaaggaggcaaagctttttt

SEQ ID NO: 26 - DNA, 70 bp, Artificial Sequence
  Synthetic polynucleotide sequence
atgttcaactttaactggaatgataggaggactaattgcttccactttgactggattgatccttaagcag

SEQ ID NO: 27 - DNA, 70 bp, Artificial Sequence
  Synthetic polynucleotide sequence
atatcccttttaactgggattgtgaatttatccgactgcattttgggcataaccggaagaagcatcttaa

SEQ ID NO: 28 - DNA, 70 bp, Artificial Sequence
  Synthetic polynucleotide sequence
tgtgcaactacattttggaaaagaaagaaaaagacacctgacatatgcggaatttactcagtttttattg

SEQ ID NO: 29 - DNA, 70 bp, Artificial Sequence
  Synthetic polynucleotide sequence
ggggtcggcgtcatcatcatccttacgggggtgcccattttctttctgggagtgttctggagaagcaaac

SEQ ID NO: 30 - DNA, 70 bp, Artificial Sequence
  Synthetic polynucleotide sequence
tgagttcctgtccctgtatgagacagagaggctgatccaggagctggccaagtgcaagattgacacacac

SEQ ID NO: 31 - DNA, 70 bp, Artificial Sequence
  Synthetic polynucleotide sequence
actatatgaattggacttatgtcttctacttttttggtactattggaatattttggtttcttttgtggat

SEQ ID NO: 32 - DNA, 70 bp, Artificial Sequence
  Synthetic polynucleotide sequence
cctcctcaaacactatcgggggcccgcaggggatgccacggtcgcctctgagaaggaatcagtcatgtaa

SEQ ID NO: 33 - DNA, 70 bp, Artificial Sequence
  Synthetic polynucleotide sequence
tgaggaaaatgagagacagaaaccccaaggtggcagagattcctttcaactctaccaacaagtaccagct

SEQ ID NO: 34 - DNA, 70 bp, Artificial Sequence
  Synthetic polynucleotide sequence
gctttgccttcgactgtgatgacgtgaacttcaccacggacaacctctgctttgtgccgctcatgtccat

SEQ ID NO: 35 - DNA, 70 bp, Artificial Sequence
  Synthetic polynucleotide sequence
accagcatgttcagaagctcaacaagttcttggagccttacaacgactctatccaagcccaaaagaatga

SEQ ID NO: 36 - DNA, 70 bp, Artificial Sequence
  Synthetic polynucleotide sequence
aataattggattaaagcctgaaggagtgccaaggatagattgtgtttcaaagaatgaagatataccaaat

SEQ ID NO: 37 - DNA, 70 bp, Artificial Sequence
  Synthetic polynucleotide sequence
ggagctggacgactgcagcaagtttgtgcagtacgagacggacctgaccttcgtgggctgcgtaggcatg

SEQ ID NO: 38 - DNA, 70 bp, Artificial Sequence
  Synthetic polynucleotide sequence
agacacagatccgcgtcgtgaaggcgttccgtagctctctctatgaaggtttagaaaagcctgaatctcg

SEQ ID NO: 39 - DNA, 70 bp, Artificial Sequence
  Synthetic polynucleotide sequence
ctcctgttggatggtgaggtcactccatatgccaatacaaacaacaatgcggtggattgcaaccaagtgc

SEQ ID NO: 40 - DNA, 70 bp, Artificial Sequence
  Synthetic polynucleotide sequence
atttgttggaaatattgtttacacatatgttgttgttactgtttgtctgaaagctggtttggagaccaca

SEQ ID NO: 41 - DNA, 70 bp, Artificial Sequence
  Synthetic polynucleotide sequence
tgataaaaagggcaaaaagggcaaaaaagacagggacatggatgaactgaagaaagaagtttctatggat

SEQ ID NO: 42 - DNA, 70 bp, Artificial Sequence
  Synthetic polynucleotide sequence
cggtgagaacattgggtacagtgagaaagaccgttttcagggacgttttgatgtaaaaattaaattttaa

SEQ ID NO: 43 - DNA, 70 bp, Artificial Sequence
  Synthetic polynucleotide sequence
cctcctcagcagaagattccgctgtgggggcaataagaagcgcaggcaaatcaatgaagatgagccgtaa

SEQ ID NO: 44 - DNA, 70 bp, Artificial Sequence
  Synthetic polynucleotide sequence
atcgagatcgagcattttgtggacatcatcgcgggcctggccattctcttcggtgccacattttttattg

SEQ ID NO: 45 - DNA, 70 bp, Artificial Sequence
  Synthetic polynucleotide sequence
ctcctgctgaaccggatcaccccttatcgagagaaaatctacatgacactctccgcttatatcgagatgg

SEQ ID NO: 46 - DNA, 70 bp, Artificial Sequence
  Synthetic polynucleotide sequence
gggcacgaaagcttctctctgtcccttggcttctgtgtggtcccagaagatatgcctcctccagtttcaa

SEQ ID NO: 47 - DNA, 70 bp, Artificial Sequence
  Synthetic polynucleotide sequence
aaagcttgaagcgcttattcgtagaacgtttagaagatgaaactcttttaattaatatgatctgtggggt

SEQ ID NO: 48 - DNA, 70 bp, Artificial Sequence
  Synthetic polynucleotide sequence
ccccctcttccagggcattggtctggcatctgtggtcatcgagtcatatttgaatgtctactacatcatc

SEQ ID NO: 49 - DNA, 70 bp, Artificial Sequence
  Synthetic polynucleotide sequence
gattccaggaggatttatctgtcaaaaatttgcagccaacagagttttcggctttgctattgtggcaaca

SEQ ID NO: 50 - DNA, 70 bp, Artificial Sequence
  Synthetic polynucleotide sequence
attgtacgagatcctaaaatcttgctactagatgaagccacttctgccttagacacagaaagtgaaaaga

SEQ ID NO: 51 - DNA, 70 bp, Artificial Sequence
  Synthetic polynucleotide sequence
tgtgagtgtgaaaataagttttgaagatgaaccaagaaagaaatacgtggatgctgaaacttcattatag

SEQ ID NO: 52 - DNA, 70 bp, Artificial Sequence
  Synthetic polynucleotide sequence
cccacttgtgtgcaagataactggaaaatgtctttctgtgtgctccgaggagaatgcaaaggctggtgga

SEQ ID NO: 53 - DNA, 70 bp, Artificial Sequence
  Synthetic polynucleotide sequence
cactggccccggaagacttagagatgtttgtgctggactttgaggatggtgacctgtgggagtccatcag

SEQ ID NO: 54 - DNA, 70 bp, Artificial Sequence
  Synthetic polynucleotide sequence
ccccgacatattcgcagtgatcataattctcatcttgacaggacttttaactcttggtgtgaaagagtcg

SEQ ID NO: 55 - DNA, 70 bp, Artificial Sequence
  Synthetic polynucleotide sequence
aatgaattacactggtcttgcagaatatcccgatttttttgctgtgtgccttatattacttctagcaggt

SEQ ID NO: 56 - DNA, 70 bp, Artificial Sequence
  Synthetic polynucleotide sequence
agacttatttcagatccccatggttcccctgattccagccctgagcatcgtcctcaacatctgcctcatg

SEQ ID NO: 57 - DNA, 70 bp, Artificial Sequence
  Synthetic polynucleotide sequence
ttcaagaaggttatctcaagaaactggcttggaaataagtgaagaaattaacgaagaagacttaaaggag

SEQ ID NO: 58 - DNA, 70 bp, Artificial Sequence
  Synthetic polynucleotide sequence
ttaaaacacttgccatggttacatcattcttaaccaacatttgcatctcctatctagccaagtatctatt

SEQ ID NO: 59 - DNA, 70 bp, Artificial Sequence
  Synthetic polynucleotide sequence
gctttatgcactgttttaatgttaaatcggacactcagcaaattacagtgggtttcagtttttatgctgt

SEQ ID NO: 60 - DNA, 70 bp, Artificial Sequence
  Synthetic polynucleotide sequence
ctttcaggttctgcccatcattgtctttttcagctgtgtcatatccgttctctaccacgtgggcctcatg

SEQ ID NO: 61 - DNA, 70 bp, Artificial Sequence
  Synthetic polynucleotide sequence
tcttcaatttgtctttgggatcttggtcatcagaactgatcttggatatactgtatttcagtggctggga

SEQ ID NO: 62 - DNA, 70 bp, Artificial Sequence
  Synthetic polynucleotide sequence
caacgatgaacttatcctgacgtttggcgccgatgacgtggtctgcaccagaatttatgtccgggaatga

SEQ ID NO: 63 - DNA, 70 bp, Artificial Sequence
  Synthetic polynucleotide sequence
tgtaagagcctggtgaaatgggagagtgagaataaaatggtctgtgagcagaagctcctgaagggagagg

SEQ ID NO: 64 - DNA, 70 bp, Artificial Sequence
  Synthetic polynucleotide sequence
agaagggccccctcatcgcgcccgggcccgacggggccccggccaagggcgacggccccgtgggcctggg

SEQ ID NO: 65 - DNA, 70 bp, Artificial Sequence
  Synthetic polynucleotide sequence
gtacctctactgtgtgctgtttatattaagcggccttttattttacttcctgtttgtccactacaagttt

SEQ ID NO: 66 - DNA, 70 bp, Artificial Sequence
  Synthetic polynucleotide sequence
tatgagccaccagcagaaaaagagcaaaaggtcctcatccaatcagtcagttgcacaggaacagaagata

SEQ ID NO: 67 - DNA, 70 bp, Artificial Sequence
  Synthetic polynucleotide sequence
gggccgggtctgcctggatgtggccatagtgtttgtcatctatgatgaagtggtgaagctgctcaacaaa

SEQ ID NO: 68 - DNA, 70 bp, Artificial Sequence
  Synthetic polynucleotide sequence
gtaagtcacaagtcagcattcgctacaattccatgcctgtcccaggaccaaatggaaccatccttatgga

SEQ ID NO: 69 - DNA, 70 bp, Artificial Sequence
  Synthetic polynucleotide sequence
atttcatcttctcagatacagcggtgcttctgtttgatttctggagtgtccacagtcctgctggcatggc

SEQ ID NO: 70 - DNA, 70 bp, Artificial Sequence
  Synthetic polynucleotide sequence
ggtgtctggaagatctgccccatactgaaaggtgtgggcttcacggtcatcctcatctcactgtatgtcg

SEQ ID NO: 71 - DNA, 70 bp, Artificial Sequence
  Synthetic polynucleotide sequence
accgggtacctctctgacaacatcttcactcactttgtcgccagctttattgcaggtggatgtgccacgt

SEQ ID NO: 72 - DNA, 70 bp, Artificial Sequence
  Synthetic polynucleotide sequence
ccctctctactacataaacaaagaatgctttaaatctgctttatacaaacaaactgtcaacccaatctta

SEQ ID NO: 73 - DNA, 70 bp, Artificial Sequence
  Synthetic polynucleotide sequence
cttcgtccagatcggaaagggtgatgtgtccaatctagatcccaagttctcatttgaaggcaccaaactg

SEQ ID NO: 74 - DNA, 70 bp, Artificial Sequence
  Synthetic polynucleotide sequence
ggggagagagacccagagatgtgagagagagtcagagacagagacagagagagagagagagagacacaga

SEQ ID NO: 75 - DNA, 70 bp, Artificial Sequence
  Synthetic polynucleotide sequence
gacatgaactgaagaacagagatgttgaaatgggtaactcagtgattgaagagaatgaaatgaagaaacc

SEQ ID NO: 76 - DNA, 70 bp, Artificial Sequence
  Synthetic polynucleotide sequence
aaattggtctgcgcgctgtcgtgtattatttctgtaccactctcattgctgttattctaggtattgtgct

SEQ ID NO: 77 - DNA, 70 bp, Artificial Sequence
  Synthetic polynucleotide sequence
gatcgagaccatccccacagctgatgccttcatggacctgatcagaaatatgtttccaccaaaccttgtg

SEQ ID NO: 78 - DNA, 70 bp, Artificial Sequence
  Synthetic polynucleotide sequence
catccggaacatgttcccagccaacctagtagaagccacattcaaacagtaccgcaccaagaccacccca

SEQ ID NO: 79 - DNA, 70 bp, Artificial Sequence
  Synthetic polynucleotide sequence
cgagaccaagctgctggtggtggacagggaaactgacgagttcttcaagaaatgcagagtgatcccatct

SEQ ID NO: 80 - DNA, 70 bp, Artificial Sequence
  Synthetic polynucleotide sequence
ctgaaaccatcgaactgactggctttgatgataagatactagaagaagatcacaaagggagaaaaagaac

SEQ ID NO: 81 - DNA, 70 bp, Artificial Sequence
  Synthetic polynucleotide sequence
tttttgctgctgtgcctgacgggcgtcaccttcgccttcctcttcgtcggcgtggtcttcctgggcacgc

SEQ ID NO: 82 - DNA, 70 bp, Artificial Sequence
  Synthetic polynucleotide sequence
cctggtgaaggtgcagctggatgctctgcccttctttgtcatcaccatgatcaagatcgtgctcattaat

SEQ ID NO: 83 - DNA, 70 bp, Artificial Sequence
  Synthetic polynucleotide sequence
aaaatgtgtggctgcttccataaatgtcatcccaggttgggagagactggagattacagacctgctactg

SEQ ID NO: 84 - DNA, 70 bp, Artificial Sequence
  Synthetic polynucleotide sequence
aagcaccttcccagttgtcctgacaagggcttcacagataaactgttctacatctacacatccggcacca

SEQ ID NO: 85 - DNA, 70 bp, Artificial Sequence
  Synthetic polynucleotide sequence
cctgcccaccccaggggaggcccaggatgctgatttgaaggatgtaaatgtgattccagccaccgcctga

SEQ ID NO: 86 - DNA, 70 bp, Artificial Sequence
  Synthetic polynucleotide sequence
cttttgtgaatgctttttcaatttttggaagcattgcactttattttggcatcatgtttgactttcatag

SEQ ID NO: 87 - DNA, 70 bp, Artificial Sequence
  Synthetic polynucleotide sequence
caagcagttccagttatactccgtgtacttcctgatcctgtccatcatctacttcttgggggccatgctg

SEQ ID NO: 88 - DNA, 70 bp, Artificial Sequence
  Synthetic polynucleotide sequence
aactgaattgaaacagctgaatttacacaaagatactgagccaaaacccctggagggaactcatctaatg

SEQ ID NO: 89 - DNA, 70 bp, Artificial Sequence
  Synthetic polynucleotide sequence
cctgggctactccagcggcttctctcttagctgcatggtgttcttcctaattgcagtcatctacaaaaag

SEQ ID NO: 90 - DNA, 70 bp, Artificial Sequence
  Synthetic polynucleotide sequence
ctattatcgcactgtgatcttctcagccatgtttgggggctacagcctgtattacttcaatcgcaagacc

SEQ ID NO: 91 - DNA, 70 bp, Artificial Sequence
  Synthetic polynucleotide sequence
gatcggtctctctaacatcactcaggggggtatttatgtcttcaaactctttgactactactctgccagt

SEQ ID NO: 92 - DNA, 70 bp, Artificial Sequence
  Synthetic polynucleotide sequence
ctttctcttctccctgataaagtacactccgctgacctacaacaagaagtacacgtacccgtggtggggc

SEQ ID NO: 93 - DNA, 70 bp, Artificial Sequence
  Synthetic polynucleotide sequence
cgtggtgttttttatttgctgtggaattcctgtttttttcctggagacagctctggggcagttcacaagt

SEQ ID NO: 94 - DNA, 70 bp, Artificial Sequence
  Synthetic polynucleotide sequence
caaagcaaaacaaagcttgaaaagactcagaggatatgatgatgtcaccaaagatattaatgaaatgaga

SEQ ID NO: 95 - DNA, 70 bp, Artificial Sequence
  Synthetic polynucleotide sequence
atttaggagcctacgtttttattatcttcaccggcttcctcattaccttcttggcttttaccttcttcaa

SEQ ID NO: 96 - DNA, 70 bp, Artificial Sequence
  Synthetic polynucleotide sequence
ctctcttttagagcaggaggtgaaacccagcacagaacttgagtatttagggccagatgagaacgactga

SEQ ID NO: 97 - DNA, 70 bp, Artificial Sequence
  Synthetic polynucleotide sequence
agagatcaaccagattttcaccaagatgaataaggtgtctgaagtgtacccggaaaaggaggaactgaaa

SEQ ID NO: 98 - DNA, 70 bp, Artificial Sequence
  Synthetic polynucleotide sequence
tttataaccactctgtatattataagcaaagttttttcatattgtatctttttctgttgagcactttatg

SEQ ID NO: 99 - DNA, 70 bp, Artificial Sequence
  Synthetic polynucleotide sequence
caaagcatacccaccagaagagaaaatcgactcagctgtcactgatggtaagataaatggaaggccttaa

SEQ ID NO: 100 - DNA, 70 bp, Artificial Sequence
  Synthetic polynucleotide sequence
agaaaaattgaacgagaaataaagtgtagtccttctgaaagccccttaatggaaaaaaagaatagcttga

SEQ ID NO: 101 - DNA, 70 bp, Artificial Sequence
  Synthetic polynucleotide sequence
atctccactgttgtctggtttcatgtctggcctgctgtttgtactcatcagaattttcatcttaaaaaag

SEQ ID NO: 102 - DNA, 70 bp, Artificial Sequence
  Synthetic polynucleotide sequence
tcatcttctttattctagttttcactgtgatccagtaccagccgatcacctacaaccactaccagtaccc

SEQ ID NO: 103 - DNA, 70 bp, Artificial Sequence
  Synthetic polynucleotide sequence
aagataaaaccaaacttttattagattcctgtgttatcagtgaccatcccaaaatacagatcaagaactc

SEQ ID NO: 104 - DNA, 70 bp, Artificial Sequence
  Synthetic polynucleotide sequence
tcctcaactaccgaaacatctggaaaaatctgcttatcctgggcttcaccaactttatcgcccatgccat

SEQ ID NO: 105 - DNA, 70 bp, Artificial Sequence
  Synthetic polynucleotide sequence
tcgagtgcatgaagatattgaaatgaccaagactcaatccatttatgatgacatgaagaaccacagggaa

SEQ ID NO: 106 - DNA, 70 bp, Artificial Sequence
  Synthetic polynucleotide sequence
tacgtcttcatcatcttcactgtgctcctggttctgttcttcatcttcacctacttcaaagttcctgaga

SEQ ID NO: 107 - DNA, 70 bp, Artificial Sequence
  Synthetic polynucleotide sequence
ctctcaagcagtggctttctacaacatacgaacttatgaagcagttttttcacctcaactaaaaaaaaat

SEQ ID NO: 108 - DNA, 70 bp, Artificial Sequence
  Synthetic polynucleotide sequence
caacttatgcacctattggagaagtgcataaagatgataaaccagtgaattgtcaccagtatgatggtct

SEQ ID NO: 109 - DNA, 70 bp, Artificial Sequence
  Synthetic polynucleotide sequence
agcgaagtggagagcgccagagtaatggtgaaggcattggagtctttcagcaatcttctaaacaaagtct

SEQ ID NO: 110 - DNA, 70 bp, Artificial Sequence
  Synthetic polynucleotide sequence
cgctggggatgttctttggttttacctccgtcattgtggcaggagtcctggagatggagcgcttacacta

SEQ ID NO: 111 - DNA, 70 bp, Artificial Sequence
  Synthetic polynucleotide sequence
aaagaaggataaaggcaaagataatgttgaaagtgctcaagcatctgaagtcaaacctctgagaagctga

SEQ ID NO: 112 - DNA, 70 bp, Artificial Sequence
  Synthetic polynucleotide sequence
ggttgatcatcactccagggacatttaaagagcgtattattaaaagtattaccccagaaacaccaacaga

SEQ ID NO: 113 - DNA, 70 bp, Artificial Sequence
  Synthetic polynucleotide sequence
ctacctgtcccaggagagaaacagaagggcatcaatcataacaaagtatcaagcctacgatgtctcctaa

SEQ ID NO: 114 - DNA, 70 bp, Artificial Sequence
  Synthetic polynucleotide sequence
cagatcagcaagtgagagaaattcttctggtgatgctgattcagctcatatggagtatccctatggagag

SEQ ID NO: 115 - DNA, 70 bp, Artificial Sequence
  Synthetic polynucleotide sequence
cacagaacttacctgcctcttcatgcagttctccatcgtgccatacctgtctcggaaactgggcctggat

SEQ ID NO: 116 - DNA, 70 bp, Artificial Sequence
  Synthetic polynucleotide sequence
ttaccactctgggccattttagtctcttatttctgtgaatactggcttttttataccattatggcgtaca

SEQ ID NO: 117 - DNA, 70 bp, Artificial Sequence
  Synthetic polynucleotide sequence
actttgaacaatatgcgtgtgtatgggaccattttcctgaccttcatgaccctggtggtgtttgtggggg

SEQ ID NO: 118 - DNA, 70 bp, Artificial Sequence
  Synthetic polynucleotide sequence
gcatgacagtgatatatcagcatatacttacgagcgcactttgatgatggaacaaaggtcccagatgctt

SEQ ID NO: 119 - DNA, 70 bp, Artificial Sequence
  Synthetic polynucleotide sequence
gctttgcttttgtgtcctgactgagcttgatgtagccgaagaaaaaaaggatgagaggaaaactgactag

SEQ ID NO: 120 - DNA, 70 bp, Artificial Sequence
  Synthetic polynucleotide sequence
catcttggccagtgatgctgttgctgtgacttttgcagatcagatatttggaatatttaactggataatt

SEQ ID NO: 121 - DNA, 70 bp, Artificial Sequence
  Synthetic polynucleotide sequence
tttggtccttattattcttctgtttattagcattatcttgacttttaagggttacttgattagctgtgtt

SEQ ID NO: 122 - DNA, 70 bp, Artificial Sequence
  Synthetic polynucleotide sequence
tgcctgtctatttcctgggtgtttactggcaacacaagcccaagtgtttcagtgacttcattgagctgct

SEQ ID NO: 123 - DNA, 70 bp, Artificial Sequence
  Synthetic polynucleotide sequence
tattaatttatgccatgaagaaaaaatatcaagagaaagatatcaatgcatcagaaaatggaagtgtcat

SEQ ID NO: 124 - DNA, 70 bp, Artificial Sequence
  Synthetic polynucleotide sequence
tattagttctctcacctatttgccaagaatcaaagaatatctggatcaactacctgattttccctacaaa

SEQ ID NO: 125 - DNA, 70 bp, Artificial Sequence
  Synthetic polynucleotide sequence
cgtcctaattatttcaggtatctatctcttcattggcatgggcatcaattatcgacttttggcaaaagaa

SEQ ID NO: 126 - DNA, 70 bp, Artificial Sequence
  Synthetic polynucleotide sequence
agttaataagtatttagatttctccctttttaagcatagaggatttctgatatatctgtctggaaatgtc

SEQ ID NO: 127 - DNA, 70 bp, Artificial Sequence
  Synthetic polynucleotide sequence
cgtcatcactggcttctcctacgccttccccaaggccgtcagtgtcttcttcaaggagctcatacaggag

SEQ ID NO: 128 - DNA, 70 bp, Artificial Sequence
  Synthetic polynucleotide sequence
tgacatttctctctttagaaatcctttcttctacatatttacttggtcttttctcctcagtcagttagca

SEQ ID NO: 129 - DNA, 70 bp, Artificial Sequence
  Synthetic polynucleotide sequence
ctttagctatgttttctacatgtccagcttcttcctcatctcagctgccctcttcatgggtggcagcttc

SEQ ID NO: 130 - DNA, 70 bp, Artificial Sequence
  Synthetic polynucleotide sequence
ttttgctactgaattctggggtctaatgtcatgcagcatattttttgggtttatggttggaacaatagga

SEQ ID NO: 131 - DNA, 70 bp, Artificial Sequence
  Synthetic polynucleotide sequence
tccctatgtacacctgatgaagtatgtggaggaggagttctcagaaatcaaggagacctgggtgctcttg

SEQ ID NO: 132 - DNA, 70 bp, Artificial Sequence
  Synthetic polynucleotide sequence
aatcttgggacaggaataattatatccttcatctatggttggcaactaacactgttactcttagcaattg

SEQ ID NO: 133 - DNA, 70 bp, Artificial Sequence
  Synthetic polynucleotide sequence
agtggttcacagaagaagtcacaagacaagggtcctaaaacaggatcagtaaagaaggaaaaagcagtaa

SEQ ID NO: 134 - DNA, 70 bp, Artificial Sequence
  Synthetic polynucleotide sequence
ttaaaattttcaaagagtcaattttggagatcttggatgaggaagagttgctagaagagctctgtgtatc

SEQ ID NO: 135 - DNA, 70 bp, Artificial Sequence
  Synthetic polynucleotide sequence
cttccaagggattgggagattgattttacttctcggatttctctactttttcgtgtgctccctggatatt

SEQ ID NO: 136 - DNA, 70 bp, Artificial Sequence
  Synthetic polynucleotide sequence
cctgcctcatcctcctagtcaagatgctcaactccctgctcaagggccaagtggccaaggtcatccagaa

SEQ ID NO: 137 - DNA, 70 bp, Artificial Sequence
  Synthetic polynucleotide sequence
agaaataaaagaaattttacggttctggctcacaaagggtgttgatggttttagtttggatgctgttaaa

SEQ ID NO: 138 - DNA, 70 bp, Artificial Sequence
  Synthetic polynucleotide sequence
aaccagagtacgctccatggtgagtgactttgctgttttcctcactatcttcacaatggtgattattgat

SEQ ID NO: 139 - DNA, 70 bp, Artificial Sequence
  Synthetic polynucleotide sequence
tcaagccaaacttcatcactacctacaagtgcgagtgtgtcgcccctgacacagtgaatacaaccgtgtt

SEQ ID NO: 140 - DNA, 70 bp, Artificial Sequence
  Synthetic polynucleotide sequence
tcaacaccatagcccccatcatttccaacttcttcctctgctcctatgccctcatcaacttcagctgctt

SEQ ID NO: 141 - DNA, 70 bp, Artificial Sequence
  Synthetic polynucleotide sequence
ttactttgtattcctgataatcagtgtgatgttagaagatcgaatcatatcctgtcctgtatctgtctga

SEQ ID NO: 142 - DNA, 70 bp, Artificial Sequence
  Synthetic polynucleotide sequence
ttcagcagtattcaacatcctctttgttattggcatgtgtgctctgttttctagagaaatcttaaacctg

SEQ ID NO: 143 - DNA, 70 bp, Artificial Sequence
  Synthetic polynucleotide sequence
ttatctacattgtcatcatgaaatataacgcttgcatacatcagtgctttgagaggaggacaaaaggtgc

SEQ ID NO: 144 - DNA, 70 bp, Artificial Sequence
  Synthetic polynucleotide sequence
agataaagaaatagagcaattaatagaattagctaactaccaagtcctaagtcagcagcaaaaaagtaga

SEQ ID NO: 145 - DNA, 70 bp, Artificial Sequence
  Synthetic polynucleotide sequence
ctgtggccactacggtcaccaccactggaaggacaagctcaaccggtttaataagaaatatgtgaagaag

SEQ ID NO: 146 - DNA, 70 bp, Artificial Sequence
  Synthetic polynucleotide sequence
aaataaggaaggtcacgtccagtgaaactgatgaaattcgagaactcttatcaagaaatctctatcaaat

SEQ ID NO: 147 - DNA, 70 bp, Artificial Sequence
  Synthetic polynucleotide sequence
ttcctcttcctgctgcctcctattgtgttggactcaggctatttcatgcctagcaggctgttctttgaca

SEQ ID NO: 148 - DNA, 70 bp, Artificial Sequence
  Synthetic polynucleotide sequence
tcttacttaatttgggtagaagaagtaagattggatcaaattttcaacacatgatgatgtttgctggcct

SEQ ID NO: 149 - DNA, 70 bp, Artificial Sequence
  Synthetic polynucleotide sequence
atcaacctcatggactttaaccctgacccgaggagccgctatacattctggacttttgtggtgggtggca

SEQ ID NO: 150 - DNA, 70 bp, Artificial Sequence
  Synthetic polynucleotide sequence
gataaaatgaaagaagatgaaccatggcgaataacagataatgagcttgaactttataagaccaagacat

SEQ ID NO: 151 - DNA, 70 bp, Artificial Sequence
  Synthetic polynucleotide sequence
tgattgaaccatatcgtctccatgaaagctgcaaagatttaacaactgctgagaaattaaaaagagaaac

SEQ ID NO: 152 - DNA, 70 bp, Artificial Sequence
  Synthetic polynucleotide sequence
tctgttcaaaatgctccagatagaccttgaagatcatttagccaggagagggcaaatatgtggcccttct

SEQ ID NO: 153 - DNA, 70 bp, Artificial Sequence
  Synthetic polynucleotide sequence
aatactgactctgatcccaagtggatatttagcagggatatttggagcaaaaaaaatgcttggtgctggt

SEQ ID NO: 154 - DNA, 70 bp, Artificial Sequence
  Synthetic polynucleotide sequence
agggatttatatcaatgtcttagatattgctccaaggtattccagttttctcatgggagcatcaagagga

SEQ ID NO: 155 - DNA, 70 bp, Artificial Sequence
  Synthetic polynucleotide sequence
ctatgagaaattcaagactcccaaggataaaacaaaaatgatctacacagctgccacaactgaagaaaca

SEQ ID NO: 156 - DNA, 70 bp, Artificial Sequence
  Synthetic polynucleotide sequence
tacatctctggttgctctcgttgttcctgtttccattggaatgtttgttaatcacaaatggccccaaaaa

SEQ ID NO: 157 - DNA, 70 bp, Artificial Sequence
  Synthetic polynucleotide sequence
aaaatttggccttcaacagttggtggttgccaagatatcggatgtgtacaatatgagtgtgtggtctcta

SEQ ID NO: 158 - DNA, 70 bp, Artificial Sequence
  Synthetic polynucleotide sequence
agctgtgacaatgatgccgctgcccacattttggtccattcttttttttattatgcttctcttgcttgga

SEQ ID NO: 159 - DNA, 70 bp, Artificial Sequence
  Synthetic polynucleotide sequence
acagcaacaagagcaccagaagtatatggtcccactgcaggcctcagcacaagagaagaatggactctga

SEQ ID NO: 160 - DNA, 70 bp, Artificial Sequence
  Synthetic polynucleotide sequence
cgtggagagaaagagtgccccaaccagtcttcaggaggaagagatgcccatgaagcaggtccagaactaa

SEQ ID NO: 161 - DNA, 70 bp, Artificial Sequence
  Synthetic polynucleotide sequence
ggtttgctaccggttttgcctactatagtttggctatgggtgtggaagaatttggagtcaacctctacat

SEQ ID NO: 162 - DNA, 70 bp, Artificial Sequence
  Synthetic polynucleotide sequence
ttttgggataagcctaacctgcctcaccatctacaaggctgaactctttccaacgccagtgcggatgaca

SEQ ID NO: 163 - DNA, 70 bp, Artificial Sequence
  Synthetic polynucleotide sequence
ccttacttgagtatcttctctattttttatcttttctcatgacttgtgaaaattcttcagttgttggaat

SEQ ID NO: 164 - DNA, 70 bp, Artificial Sequence
  Synthetic polynucleotide sequence
ctacttgggcttatctatagctttaagattcccagcacttgttttatatattgttttcatttttgctatg

SEQ ID NO: 165 - DNA, 70 bp, Artificial Sequence
  Synthetic polynucleotide sequence
gtgcctgaggaaaaactataaacgctacatcaaaaaccacgagggcgggctgagcaccagtgagttcttt

SEQ ID NO: 166 - DNA, 70 bp, Artificial Sequence
  Synthetic polynucleotide sequence
ttcaacttgtcagagaaagcccctccttctggttttcatattcgttgtaattttctttacattcctcagc

SEQ ID NO: 167 - DNA, 70 bp, Artificial Sequence
  Synthetic polynucleotide sequence
atcacaaaaaagaaacactgaagcaataaagataatggaccacatcgctcaaaagaatgggaagttgcct

SEQ ID NO: 168 - DNA, 70 bp, Artificial Sequence
  Synthetic polynucleotide sequence
aagaccaagaaaaaataaagaaaagatgatttacctccaagttcagaaactagacattccattgaactaa

SEQ ID NO: 169 - DNA, 70 bp, Artificial Sequence
  Synthetic polynucleotide sequence
atctgggaaaaaaacaagagactcaatggagacagaagaaaatcccaaggttctaataactgcattctga

SEQ ID NO: 170 - DNA, 70 bp, Artificial Sequence
  Synthetic polynucleotide sequence
gctaagagtcaaaggaatgaaacacagaaaaactccaagtcacacaaggatgttaaaagatggtcaagaa

SEQ ID NO: 171 - DNA, 70 bp, Artificial Sequence
  Synthetic polynucleotide sequence
cctgctgtgtgttctcaacttcctgtctcccttctacttttttgcccatgtcttcatggtcctagatgag

SEQ ID NO: 172 - DNA, 70 bp, Artificial Sequence
  Synthetic polynucleotide sequence
catcttcggctttgggacagccttcatgaacagctttcacctgtatttgttctttcgctttggcatctcg

SEQ ID NO: 173 - DNA, 70 bp, Artificial Sequence
  Synthetic polynucleotide sequence
tgttgtgaaaaatgaaggaataacggccttatattctggactgaaacctactatgattcgagcattccct

SEQ ID NO: 174 - DNA, 70 bp, Artificial Sequence
  Synthetic polynucleotide sequence
aaactaataaaaagtggacaattaagagcaacaaagaatggcatcataagtgatgctgtttcaacaaata

SEQ ID NO: 175 - DNA, 70 bp, Artificial Sequence
  Synthetic polynucleotide sequence
acaattctctgttgatagagtccatcaaatcctttcagaaaacaccacactattccaaactgcacctgaa

SEQ ID NO: 176 - DNA, 70 bp, Artificial Sequence
  Synthetic polynucleotide sequence
ttggagcctggtggctaggcctgctcatttcttcagctttattggttctcacctctttcccctttttttt

SEQ ID NO: 177 - DNA, 70 bp, Artificial Sequence
  Synthetic polynucleotide sequence
gtttggcttttatgaagtctttaaagtcttgtatagcaatatgcttggagaggagaatacttatctctgg

SEQ ID NO: 178 - DNA, 70 bp, Artificial Sequence
  Synthetic polynucleotide sequence
cttttaagggagtcctaaggcgacagaacatgggtcaacaccttgatgtaaaacttgttcctagttcatc

SEQ ID NO: 179 - DNA, 70 bp, Artificial Sequence
  Synthetic polynucleotide sequence
ctatttctacacttttaatagcctcaaagcactctgggtcaaaggtcaacattctaccactggaaaagat

SEQ ID NO: 180 - DNA, 70 bp, Artificial Sequence
  Synthetic polynucleotide sequence
tcgaagacccattggtaagatgacaataactgagcaaaagtatgaaggagaatatagatatgttaattct

SEQ ID NO: 181 - DNA, 70 bp, Artificial Sequence
  Synthetic polynucleotide sequence
tctcttcttcttcatgcttctgactctcggcctagatagccagtttgcctttctggagaccattgtgaca

SEQ ID NO: 182 - DNA, 70 bp, Artificial Sequence
  Synthetic polynucleotide sequence
attgctggccgggctgcagcccatctacagcctctatacgtccttcttcgccaacctcatctacttcctc

SEQ ID NO: 183 - DNA, 70 bp, Artificial Sequence
  Synthetic polynucleotide sequence
ggccatgttattcatggaggaattgttctgccacttatttattttgttttcacacgaaaaaacccattca

SEQ ID NO: 184 - DNA, 70 bp, Artificial Sequence
  Synthetic polynucleotide sequence
tcctcgactggaagacggtgaaccagaagatgccgtggaatatcgtgttattgctgggtggtggctatgc

SEQ ID NO: 185 - DNA, 70 bp, Artificial Sequence
  Synthetic polynucleotide sequence
ctcttggaccccagaccacaccggaagaaatgagccagaagtgagcgctgggaacagggtggagtctccg

SEQ ID NO: 186 - DNA, 70 bp, Artificial Sequence
  Synthetic polynucleotide sequence
gtattcagatttcatcctttgttagctcactttataatttgtattttttttctgtatagaactaaatata

SEQ ID NO: 187 - DNA, 70 bp, Artificial Sequence
  Synthetic polynucleotide sequence
ggagaacatccaagaaggccctaaggagaccattgaaatagaaacacaagttcctgagaagaaaaaagga

SEQ ID NO: 188 - DNA, 70 bp, Artificial Sequence
  Synthetic polynucleotide sequence
aagtggcttggctgttgctggattcgagtggaatgcgctcttcgtggtgctgctactgggctggctgttt

SEQ ID NO: 189 - DNA, 70 bp, Artificial Sequence
  Synthetic polynucleotide sequence
ggtggatgtgttgaagaactatgagatcatcttctacctggccggctctgaggtggccctggctggggtc

SEQ ID NO: 190 - DNA, 70 bp, Artificial Sequence
  Synthetic polynucleotide sequence
tttaccaagatcccaaagtagagaggattctaattcttcctctgagaaatccaagtttattatagatgat

SEQ ID NO: 191 - DNA, 70 bp, Artificial Sequence
  Synthetic polynucleotide sequence
cttcgtcctcaccaagtccttcctgccagtggtgagcaccttcggcctccaggtgcctttcttcttcttc

SEQ ID NO: 192 - DNA, 70 bp, Artificial Sequence
  Synthetic polynucleotide sequence
agagccttcatttttattttgacatttctgctgtatgcaagttttcacttatctcgaaagcctatcagca

SEQ ID NO: 193 - DNA, 70 bp, Artificial Sequence
  Synthetic polynucleotide sequence
gaaagacttggaactttcagacaccgaggagccccccaactatgatgaggagatgagtggggggatcgag

SEQ ID NO: 194 - DNA, 70 bp, Artificial Sequence
  Synthetic polynucleotide sequence
aatactaaatattggactttttgctgtgtgttcacttggaattttcatgtttgtttatttctccttatga

SEQ ID NO: 195 - DNA, 70 bp, Artificial Sequence
  Synthetic polynucleotide sequence
tcagcggctctctcagcactatatcctctgcttttaattcattggcaactgttacgatggaagacctgat

SEQ ID NO: 196 - DNA, 70 bp, Artificial Sequence
  Synthetic polynucleotide sequence
catggctgctaaatgccactgtggaggagaacatcatctttgagagtcccttcaacaaacaacggtacaa

SEQ ID NO: 197 - DNA, 70 bp, Artificial Sequence
  Synthetic polynucleotide sequence
ctattaaatgctacagtagaagaaaatattacttttggaagtccttttaacaaacagaggtacaaagctg

SEQ ID NO: 198 - DNA, 70 bp, Artificial Sequence
  Synthetic polynucleotide sequence
tctgagcaagaagaagaagaccaaaagggaacagttgtcagagaagaggatccaagaagaatatgaaaaa

SEQ ID NO: 199 - DNA, 70 bp, Artificial Sequence
  Synthetic polynucleotide sequence
tccacctgccaaagaagaaaaaatggctattctcatggatcacaactgccctattaaaacaaaaatgtac

SEQ ID NO: 200 - DNA, 70 bp, Artificial Sequence
  Synthetic polynucleotide sequence
caagagctacgatttccccattgggatgggcatagtaaaaagaattacctttctgaaatacattcctatc

SEQ ID NO: 201 - DNA, 70 bp, Artificial Sequence
  Synthetic polynucleotide sequence
actggattctacaagatggctcagccgataccttcactcgaaacttaactctcatgtccattctcaccat

SEQ ID NO: 202 - DNA, 70 bp, Artificial Sequence
  Synthetic polynucleotide sequence
cttaaacttcacaccagaaccttctgacattttctcctgcattgtgactcacgaaattgaccgctacaca

SEQ ID NO: 203 - DNA, 70 bp, Artificial Sequence
  Synthetic polynucleotide sequence
cccttgagaacagcgtaggccttttcctgtctgcctttcttctgcttgggctcttcaaggcactgggctg

SEQ ID NO: 204 - DNA, 70 bp, Artificial Sequence
  Synthetic polynucleotide sequence
gcagttggttatataaaaatatcctggtcaacttggggagaaatgacattatctctcttttctctcctga

SEQ ID NO: 205 - DNA, 70 bp, Artificial Sequence
  Synthetic polynucleotide sequence
ggagtacacgctgagcttcctcacacaccagcgcttccagttcagtagcctacagcaggggaagatgttt

SEQ ID NO: 206 - DNA, 70 bp, Artificial Sequence
  Synthetic polynucleotide sequence
tgacctcatcaaggatgccctcctgaaagccaacctcatgacagatgacctcccttgccacttcatttct

SEQ ID NO: 207 - DNA, 70 bp, Artificial Sequence
  Synthetic polynucleotide sequence
gggcctgtggaaaggaactttgcccaacatcatgaggaatgctatcgtcaactgtgctgaggtggtgacc

SEQ ID NO: 208 - DNA, 70 bp, Artificial Sequence
  Synthetic polynucleotide sequence
ggaagtatatttggattaatgggtgtatacatttatgatggagaactggtatcaaagaatggattttttc

SEQ ID NO: 209 - DNA, 70 bp, Artificial Sequence
  Synthetic polynucleotide sequence
aagctcgcagtgccctctctcatctacaccttgcagaataacctccagtatgttgccatctctaacctac

SEQ ID NO: 210 - DNA, 70 bp, Artificial Sequence
  Synthetic polynucleotide sequence
atgtacaaacccaagaaagttgttgggatagaagaacacacagtcggctatggagagctactcttgctat

SEQ ID NO: 211 - DNA, 70 bp, Artificial Sequence
  Synthetic polynucleotide sequence
gagagcgaagacgtgaagatcggggtgctgtttgcttccaaggctatcctgcagctgctagtgaacccct

SEQ ID NO: 212 - DNA, 70 bp, Artificial Sequence
  Synthetic polynucleotide sequence
ggctttgttatcatgtttctctccacagttatgtttgctttttctgggacctatactctactctttgtgg

SEQ ID NO: 213 - DNA, 70 bp, Artificial Sequence
  Synthetic polynucleotide sequence
tcctgagtcaaggacaatgtgtgtaccggggaaaagtctgcaatcttgtgccatatttgagggatttggg

SEQ ID NO: 214 - DNA, 70 bp, Artificial Sequence
  Synthetic polynucleotide sequence
ccctatggagccttctggcttgcctccgctttctgcatcttcagtgtccttttcactttgttctgtgtcc

SEQ ID NO: 215 - DNA, 70 bp, Artificial Sequence
  Synthetic polynucleotide sequence
aatgtcagagaaaataaccagaacattacaaataatactggaagttgtaccagaagaagataagttatga

SEQ ID NO: 216 - DNA, 70 bp, Artificial Sequence
  Synthetic polynucleotide sequence
tgatggagcagatcacactggcttacaaggagcagtcagggccgtcacctctggaggaaacaagggctct

SEQ ID NO: 217 - DNA, 70 bp, Artificial Sequence
  Synthetic polynucleotide sequence
tgaaaacatccctgctgttgtgatagagattaaaaacatgccaaacaaacaacctgaatcatctttgtga

SEQ ID NO: 218 - DNA, 70 bp, Artificial Sequence
  Synthetic polynucleotide sequence
tgcctccatcctcatctacttcaagcctcaatacaaggcagccgaccccatcagcaccttcctcttctcc

SEQ ID NO: 219 - DNA, 70 bp, Artificial Sequence
  Synthetic polynucleotide sequence
tccaaagcaaaccatttattattgaacacatttggcatgtatagatgtactattcagcttcagagttaca

SEQ ID NO: 220 - DNA, 70 bp, Artificial Sequence
  Synthetic polynucleotide sequence
tttgtgaaaaactacaagaatcccaatctgaccatttccttcactgctgaacgaagtattgaagatgaac

SEQ ID NO: 221 - DNA, 70 bp, Artificial Sequence
  Synthetic polynucleotide sequence
gattcagcctccaggttcatggcctatcacaagcccctgaaaaactcacaggattacacagaagctctgc

SEQ ID NO: 222 - DNA, 70 bp, Artificial Sequence
  Synthetic polynucleotide sequence
aaaagacaagacctatagctacctgaataaactaccagtgaaaagcgaatatccctctataaaactggtg

SEQ ID NO: 223 - DNA, 70 bp, Artificial Sequence
  Synthetic polynucleotide sequence
tttttggagtcgtaggagttacatctgctaacatgcttattttcattcttccttcatctctttatttaaa

SEQ ID NO: 224 - DNA, 70 bp, Artificial Sequence
  Synthetic polynucleotide sequence
tatctttggttttattggtgcatctgcagcttctatgttgatttttattcttccttctgccttctatatc

SEQ ID NO: 225 - DNA, 70 bp, Artificial Sequence
  Synthetic polynucleotide sequence
tatctttcaagcaatcaaaggttttcgcaattctccagtgggagtaaaccacagactacgagggagtttg

SEQ ID NO: 226 - DNA, 70 bp, Artificial Sequence
  Synthetic polynucleotide sequence
catcctgttggccctcattgagggcgttggcatcctcctcactcgctacacagcccagcagttccgaaat

SEQ ID NO: 227 - DNA, 70 bp, Artificial Sequence
  Synthetic polynucleotide sequence
attgaaggaaacccagaacatggcctggtccaaaccaagaaatgtacagattttgaatatggtgactagg

SEQ ID NO: 228 - DNA, 70 bp, Artificial Sequence
  Synthetic polynucleotide sequence
gctccgaaaccggttcatcggcctccagttcttcttcaaaacaggttctgtgatctgcttcgccttagtt

SEQ ID NO: 229 - DNA, 70 bp, Artificial Sequence
  Synthetic polynucleotide sequence
caagaaaaagaaaaagaaaaaattttctgttgatgctgttagtgatgacgatgttctgaaggagaaatca

SEQ ID NO: 230 - DNA, 70 bp, Artificial Sequence
  Synthetic polynucleotide sequence
gtcaagttctacatcgacgtcaagaagttccccatctccattggcatcatcgtgttcagctacacgtctc

SEQ ID NO: 231 - DNA, 70 bp, Artificial Sequence
  Synthetic polynucleotide sequence
agcatatcatcagctatttgttgggacagaaagaattcgagctccagagattattttccagccatctctc

SEQ ID NO: 232 - DNA, 70 bp, Artificial Sequence
  Synthetic polynucleotide sequence
tggtgaatttctaactcttgtgctacttggtatcgtccaaaatccaaatatagtcaacagtgtagtggct

SEQ ID NO: 233 - DNA, 70 bp, Artificial Sequence
  Synthetic polynucleotide sequence
aacttataaaatgcctctcgggaacctcaccatcgcggtctcaggagataaaatcctcagtgtcatggag

SEQ ID NO: 234 - DNA, 70 bp, Artificial Sequence
  Synthetic polynucleotide sequence
agtatacaaaagatgggccttgcatctacaataatctagaatttggaattgaccttgacacacgagtggc

SEQ ID NO: 235 - DNA, 70 bp, Artificial Sequence
  Synthetic polynucleotide sequence
gaccaccaagtttttcatccccctcactaccttcctcctgtacaactttgctgacctatgtggccggcag

SEQ ID NO: 236 - DNA, 70 bp, Artificial Sequence
  Synthetic polynucleotide sequence
ttgctgtctttcttacaattctgtgtatggttttaattgactatgccattgggatcccatctccaaaact

SEQ ID NO: 237 - DNA, 70 bp, Artificial Sequence
  Synthetic polynucleotide sequence
tgaggagcaacaacgttgaggactgcaagatgatgcaggtgagctcaggagataagatggaagatgcaac

SEQ ID NO: 238 - DNA, 70 bp, Artificial Sequence
  Synthetic polynucleotide sequence
caatggagctcctatctctcctatgtcaggtatggctttgagggtgtgatcctgacgatctatggcatgg

SEQ ID NO: 239 - DNA, 70 bp, Artificial Sequence
  Synthetic polynucleotide sequence
gccaacatcatgaatgagaagtgtgtggtcgagaatgctgagaaaatcctagggtaccttaacaccaacg

SEQ ID NO: 240 - DNA, 70 bp, Artificial Sequence
  Synthetic polynucleotide sequence
ttcctgcactggctgatgagtgtgtacgtcgtcgagctgctcaggtctttcttttatgtcacggagacca

SEQ ID NO: 241 - DNA, 70 bp, Artificial Sequence
  Synthetic polynucleotide sequence
agaaaagcatgttttttcatgcaaaacccagcagagaaataaagaagtcatcaagcgtgaatccagtatt

SEQ ID NO: 242 - DNA, 70 bp, Artificial Sequence
  Synthetic polynucleotide sequence
attcatcgtctacacagaggccattaaaaacatggaggtgtcccagctgtggtcggtgctctacttcttc

SEQ ID NO: 243 - DNA, 70 bp, Artificial Sequence
  Synthetic polynucleotide sequence
tcgaaggtggagagagccaacgcctgcaactcggtgatccgccagctaatgaagaaggaattcaccctgg

SEQ ID NO: 244 - DNA, 70 bp, Artificial Sequence
  Synthetic polynucleotide sequence
aaattcatatataagaaaaaaagagattccattcagttcagagcagaagtggatggcggtgaaatgcagt

SEQ ID NO: 245 - DNA, 70 bp, Artificial Sequence
  Synthetic polynucleotide sequence
ttgttcagataaaactggaacactgacgaagaatgaaatgactgttactcacatatttacttcagatggt

SEQ ID NO: 246 - DNA, 70 bp, Artificial Sequence
  Synthetic polynucleotide sequence
gtaaagtcaccaatattgaaggcttgctacataagaataactggaatatagaggacaataatattaaaaa

SEQ ID NO: 247 - DNA, 70 bp, Artificial Sequence
  Synthetic polynucleotide sequence
caactttataattatagtgttgtaaaatatttttgtctgtctcttttgttaaatcatccaaccccctcta

SEQ ID NO: 248 - DNA, 70 bp, Artificial Sequence
  Synthetic polynucleotide sequence
attaccaagaggcaaaaagcaccaacatcatgtccagcttcaataagatgatccctcagcaagctctcgt

SEQ ID NO: 249 - DNA, 70 bp, Artificial Sequence
  Synthetic polynucleotide sequence
ctgtgtgtctaacatagaaaggaatctgcagaaagaagctggtgttctctccgtgttggttgccttgatg

SEQ ID NO: 250 - DNA, 70 bp, Artificial Sequence
  Synthetic polynucleotide sequence
agaagaaagccaacgcacccaaaaaggagaagtctgtccttcaggggaagctcacaaagctagccgtgca

SEQ ID NO: 251 - DNA, 70 bp, Artificial Sequence
  Synthetic polynucleotide sequence
tgaatgctttccagagtggaagttccattcagggggctctaaggcggcaaccctccatcgccagccagca

SEQ ID NO: 252 - DNA, 70 bp, Artificial Sequence
  Synthetic polynucleotide sequence
tctggtaattcgaggaggagagaagatgcaaattaatgtacaagaggtggtgttgggagacctggtggaa

SEQ ID NO: 253 - DNA, 70 bp, Artificial Sequence
  Synthetic polynucleotide sequence
tctcccaacaaaaataacaatgctgttgacagtggaattcaccttacaatagaaatgaacaagtctgcta

SEQ ID NO: 254 - DNA, 70 bp, Artificial Sequence
  Synthetic polynucleotide sequence
tcttcaacctgtacaccatcctcaccgtcatgctccagttctttgtgcacttcctgagccttgtctacct

SEQ ID NO: 255 - DNA, 70 bp, Artificial Sequence
  Synthetic polynucleotide sequence
gctatttcaaataatagaacaattttgcccatggtttccagaacaaggaactttagatctaaaagattgg

SEQ ID NO: 256 - DNA, 70 bp, Artificial Sequence
  Synthetic polynucleotide sequence
ggtaacatatcagatgaaatggccaaaactgcacagtggaaggcactttccatgaatactgagaatgcca

SEQ ID NO: 257 - DNA, 70 bp, Artificial Sequence
  Synthetic polynucleotide sequence
ggaagatgctgaaccaaacttcgatgaggatggccaggatgagtacaatgagctgcacatgccagtgtga

SEQ ID NO: 258 - DNA, 70 bp, Artificial Sequence
  Synthetic polynucleotide sequence
ctaccacgagatcggccgctccatctccaccctcatgtcagacaagcaattccacgaggcagcctacctg

SEQ ID NO: 259 - DNA, 70 bp, Artificial Sequence
  Synthetic polynucleotide sequence
gctgatcaagatcttccaggaccacccactacagaagacttataactacaacgtgttgatggtgcccaaa

SEQ ID NO: 260 - DNA, 70 bp, Artificial Sequence
  Synthetic polynucleotide sequence
aagaaaactgatcagtgattttgccattatcttgtccattctcatcttttgtgtaatagatgccctagta

SEQ ID NO: 261 - DNA, 70 bp, Artificial Sequence
  Synthetic polynucleotide sequence
attagtcttgtttttttctctgaaaaactctgctactgaatttgatgtctcattgcctgaggtattttag

SEQ ID NO: 262 - DNA, 70 bp, Artificial Sequence
  Synthetic polynucleotide sequence
cctcggggggctccgtggtttgttcggcttgccctggctcacagctgccactgtgcgccacgtcactcac

SEQ ID NO: 263 - DNA, 70 bp, Artificial Sequence
  Synthetic polynucleotide sequence
acttgaaaatattttacgtcttggagaacaagaatctaagcagaatggaataggcattaatccatactgt

SEQ ID NO: 264 - DNA, 70 bp, Artificial Sequence
  Synthetic polynucleotide sequence
gaaactgagaaacttagtataatgattgaagaatgtggaggcttagacaaaattgaagctctacaaaacc

SEQ ID NO: 265 - DNA, 70 bp, Artificial Sequence
  Synthetic polynucleotide sequence
ttgctgagtagcccaaaggaatctatcaaaaaggaagcatgttggacgatatctaatattacagctggaa

SEQ ID NO: 266 - DNA, 70 bp, Artificial Sequence
  Synthetic polynucleotide sequence
gtttggagaaaattgaagttttacagcaacatgaaaatgaagacatatataaattagcatttgaaatcat

SEQ ID NO: 267 - DNA, 70 bp, Artificial Sequence
  Synthetic polynucleotide sequence
ctatgagaagacaacgaaatgaagttgtagttgaattaaggaagaataaaagagatgaacatctcttaaa

SEQ ID NO: 268 - DNA, 70 bp, Artificial Sequence
  Synthetic polynucleotide sequence
gaatgaagagctataagaacaatgctctaaaccctgaagaaatgagacgaagaagagaggaagagggcat

SEQ ID NO: 269 - DNA, 70 bp, Artificial Sequence
  Synthetic polynucleotide sequence
aaataaaataaataatattgacaatatgtaccgaaatttccaaatggaagtgctatctggagagcagaac

SEQ ID NO: 270 - DNA, 70 bp, Artificial Sequence
  Synthetic polynucleotide sequence
cttcaggggctggaggaagaataaatctgagataagaaccaatgcagaagatagggctcgagctgtaatt

SEQ ID NO: 271 - DNA, 70 bp, Artificial Sequence
  Synthetic polynucleotide sequence
tggacatgaaataaatgagaggaaagagaaaacaaaaccagttccaggatacaataatgatacagggaaa

SEQ ID NO: 272 - DNA, 70 bp, Artificial Sequence
  Synthetic polynucleotide sequence
gaagctagaagcattttttggctttctcatcactattatggccctcacatttggatatgagtatgttaca

SEQ ID NO: 273 - DNA, 70 bp, Artificial Sequence
  Synthetic polynucleotide sequence
acgggctgcggaagctggaagctttttttggactccttataaccattatggccttgacctttggctatga

SEQ ID NO: 274 - DNA, 70 bp, Artificial Sequence
  Synthetic polynucleotide sequence
aggaaagaaggtggacccaaatttgtaaacggtgtgcagcaatatatatcaattcgttctgagataatcg

SEQ ID NO: 275 - DNA, 70 bp, Artificial Sequence
  Synthetic polynucleotide sequence
tcccacaggatgcctacttcatcaccttcatgctgctctttgccgtttctaatggctacctggtgtccct

SEQ ID NO: 276 - DNA, 70 bp, Artificial Sequence
  Synthetic polynucleotide sequence
cagatttttttccatcttttacttggctattaatgctggaagtttgctttccacaatcatcacacccatg

SEQ ID NO: 277 - DNA, 70 bp, Artificial Sequence
  Synthetic polynucleotide sequence
gcccacttctcggaaaacccaatcctctacctgctcctgcctcttatctttgtcaacaagtgttcgtttg

SEQ ID NO: 278 - DNA, 70 bp, Artificial Sequence
  Synthetic polynucleotide sequence
agaagggtcctctcatcgtgtccgggcccgatggtgccccgtccaagggcgatggccctgcgggcctggg

SEQ ID NO: 279 - DNA, 70 bp, Artificial Sequence
  Synthetic polynucleotide sequence
caatgtttgatgattattctgctacactgcctctgctaattgtagtcattttggagaatattgctgtatg

SEQ ID NO: 280 - DNA, 70 bp, Artificial Sequence
  Synthetic polynucleotide sequence
ctgcctttccctgacacttttggggtctgcctgggagaggaggggagaaagcaccatgagtgctcactaa

SEQ ID NO: 281 - DNA, 70 bp, Artificial Sequence
  Synthetic polynucleotide sequence
gcctgggcctcccacacctgcactgcccacacactcatacagctctcactccccacgtgctccacgcctc

SEQ ID NO: 282 - DNA, 70 bp, Artificial Sequence
  Synthetic polynucleotide sequence
tgccgcaactcagatatttttttccttgggggctggatttggagtattgattgcatttgccagttacaac

SEQ ID NO: 283 - DNA, 70 bp, Artificial Sequence
  Synthetic polynucleotide sequence
aaggtgatttacttcacagctttgttcccttacctggtcctgaccatctttctcatcagagggctgaccc

SEQ ID NO: 284 - DNA, 70 bp, Artificial Sequence
  Synthetic polynucleotide sequence
tttaattgcagctatactggagctagttggaatcatctggatttatgcaatgtactctttgttttcttta

SEQ ID NO: 285 - DNA, 70 bp, Artificial Sequence
  Synthetic polynucleotide sequence
taacgtatttcttgagaaaaataaaatgtactctagagaatggaatcgtgatgtccaatacagaggcaga

SEQ ID NO: 286 - DNA, 70 bp, Artificial Sequence
  Synthetic polynucleotide sequence
aatccatgatgctgttttgcatattttgatgaagaaagattacagtacttcaaagtttaatcccagtcag

SEQ ID NO: 287 - DNA, 70 bp, Artificial Sequence
  Synthetic polynucleotide sequence
catcatcatgctgattggctcttttattctcatggggtttgcatttaacgaagttggaggttatgagagc

SEQ ID NO: 288 - DNA, 70 bp, Artificial Sequence
  Synthetic polynucleotide sequence
tggagacatgtctgcaacatcaatgctgtccttttgctggccatcaacatcttcctctggggctattttg

SEQ ID NO: 289 - DNA, 70 bp, Artificial Sequence
  Synthetic polynucleotide sequence
cggctggttaaaagatgcactaaacctgatagaaaagaattccagaagattgccatggcaacagcaatag

SEQ ID NO: 290 - DNA, 70 bp, Artificial Sequence
  Synthetic polynucleotide sequence
gcccctgaatatctactttggatttattcctgaaataatcttcatgacctctttgtttggctatttggtt

SEQ ID NO: 291 - DNA, 70 bp, Artificial Sequence
  Synthetic polynucleotide sequence
tcctatttggaacttggccacaaatcgcctcacttttctaaactctttcaaaatgaaaatgtccgtgatt

SEQ ID NO: 292 - DNA, 70 bp, Artificial Sequence
  Synthetic polynucleotide sequence
cagcatcctcatccacttcatcaacatgttcctcttctcccacagccccagcaacaggctgctctacccc

SEQ ID NO: 293 - DNA, 70 bp, Artificial Sequence
  Synthetic polynucleotide sequence
tgacctgcatgctgtttttccttgtttcggtcatctacaagaagttccaacttggctgtgctataggcca

SEQ ID NO: 294 - DNA, 70 bp, Artificial Sequence
  Synthetic polynucleotide sequence
ccaggaaccctggatccagtttgccaccatccgagacaacatcctctttgggaagacatttgatgcacag

SEQ ID NO: 295 - DNA, 70 bp, Artificial Sequence
  Synthetic polynucleotide sequence
atttctggtattcctaattcctttccttcattttatcatttttctttttactcttcgatgtctggaatgg

SEQ ID NO: 296 - DNA, 70 bp, Artificial Sequence
  Synthetic polynucleotide sequence
atcctgccactgtttctcctctacgctgggtttcaggtggcttgcggccaatgtggagctcctggggaat

SEQ ID NO: 297 - DNA, 70 bp, Artificial Sequence
  Synthetic polynucleotide sequence
gagcgagtatctggtgagtcacagggtggtgggcaatgcgggggcaggagcggctccaggtggccagagc

SEQ ID NO: 298 - DNA, 70 bp, Artificial Sequence
  Synthetic polynucleotide sequence
aaatgaagaagaagttaaaaagatgtgtatgtataaatatccaggaatgaagaaaaaaatgggagaattt

SEQ ID NO: 299 - DNA, 70 bp, Artificial Sequence
  Synthetic polynucleotide sequence
aaaaatgattggcatttgtccacagttagatatacactttgatgttttgacagtagaagaaaatttatca

SEQ ID NO: 300 - DNA, 70 bp, Artificial Sequence
  Synthetic polynucleotide sequence
agagttctataggttaaagaaaatacaagagaagaaaaagattctaaaggaaaaatctgagaaggacttg

SEQ ID NO: 301 - DNA, 70 bp, Artificial Sequence
  Synthetic polynucleotide sequence
acaagattgatgctgctctcttctggatgcccaatggaaagacctacttcttccgtggaaacaagtacta

SEQ ID NO: 302 - DNA, 70 bp, Artificial Sequence
  Synthetic polynucleotide sequence
gacatttgaagaagttccctacagtgaattagaaaatggcaaacgtgatgtggatataaccattattttt

SEQ ID NO: 303 - DNA, 70 bp, Artificial Sequence
  Synthetic polynucleotide sequence
attgatgctgtttttgaagaatttgggttcttttatttctttactggatcttcacagttggagtttgacc

SEQ ID NO: 304 - DNA, 70 bp, Artificial Sequence
  Synthetic polynucleotide sequence
ctgaggaaaacactggaaaaacctacttctttgttgctaacaaatactggaggtatgatgaatataaacg

SEQ ID NO: 305 - DNA, 70 bp, Artificial Sequence
  Synthetic polynucleotide sequence
atgaattttatttctctattctggccatcccttccaactggtatacaggctgcttatgaagattttgaca

SEQ ID NO: 306 - DNA, 70 bp, Artificial Sequence
  Synthetic polynucleotide sequence
agcaacttgggagccaacaactatgatgactacaggatggactggcttgtgcctgccacctgtgaaccca

SEQ ID NO: 307 - DNA, 70 bp, Artificial Sequence
  Synthetic polynucleotide sequence
caagatcgtcttctttaaaggagacaggtactgggtgttcaaggacaataacgtagaggaaggatacccg

SEQ ID NO: 308 - DNA, 70 bp, Artificial Sequence
  Synthetic polynucleotide sequence
cacctacttcttcaagggcgcccactactggcgcttccccaagaacagcatcaagaccgagccggacgcc

SEQ ID NO: 309 - DNA, 70 bp, Artificial Sequence
  Synthetic polynucleotide sequence
aatactacagctacgacgaaaggaaaaggaaaatggaaaaagactatccaaagaatactgaagaagaatt

SEQ ID NO: 310 - DNA, 70 bp, Artificial Sequence
  Synthetic polynucleotide sequence
ggtgtggcgctacattaatttcaagatgtctcctggcttccccaagaagctgaatagggtagaacctaac

SEQ ID NO: 311 - DNA, 70 bp, Artificial Sequence
  Synthetic polynucleotide sequence
acccaaagtaactacaacaaaaaagacaattactaccactgagattatgaacaaacctgaagaaacagct

SEQ ID NO: 312 - DNA, 70 bp, Artificial Sequence
  Synthetic polynucleotide sequence
ccaaatgctgatcatgacaattttagaaattgttttctttgcccacaatgaatacctggttagtgaaata

SEQ ID NO: 313 - DNA, 70 bp, Artificial Sequence
  Synthetic polynucleotide sequence
ccattcctttgactttcaaggtttcaacggggactggaccgcaagaacacagggaaagttccagatttat

SEQ ID NO: 314 - DNA, 70 bp, Artificial Sequence
  Synthetic polynucleotide sequence
acctcatgtggctaaatattttgatgaccaagttttctggaagtttcctcatttggctgttggattttaa

SEQ ID NO: 315 - DNA, 70 bp, Artificial Sequence
  Synthetic polynucleotide sequence
ccttgaatcaaaattcaaagtccaagacacatgtggagtccacaacctccatgggatgccgggggtcctg

SEQ ID NO: 316 - DNA, 70 bp, Artificial Sequence
  Synthetic polynucleotide sequence
cttgtccctcctttcccctaagcagaaaactggccctgccctgcccctgccccatttcctcctcatctga

SEQ ID NO: 317 - DNA, 70 bp, Artificial Sequence
  Synthetic polynucleotide sequence
gcaaattatataacagcattgatttgtaatttgtgacaacgtttggttaatacaccagtgaaccttaaaa

SEQ ID NO: 318 - DNA, 70 bp, Artificial Sequence
  Synthetic polynucleotide sequence
tgcatgacttcagctcctaaatgttgattccaagtgcctgttctgtatggtgttgttcctttatatggga

SEQ ID NO: 319 - DNA, 70 bp, Artificial Sequence
  Synthetic polynucleotide sequence
ttttttttttttttttttttcacgagacaaggaggggggttataatgagctcaaagcctgtgccggtctg

SEQ ID NO: 320 - DNA, 70 bp, Artificial Sequence
  Synthetic polynucleotide sequence
ttttatttaagtgaataatttaaagtcttctcctcccccactgcccctgcagtaaagtgctttggccccc

SEQ ID NO: 321 - DNA, 70 bp, Artificial Sequence
  Synthetic polynucleotide sequence
atccttgaagtcactcaagaaacaaggtgctttcctttcgattgtcacgtatcaaaccacacaaaaagcc

SEQ ID NO: 322 - DNA, 70 bp, Artificial Sequence
  Synthetic polynucleotide sequence
agaatctttctccaagaggatgacagtgctgacctggaaggcagagaggaccagtccatcttcccctgca

SEQ ID NO: 323 - DNA, 70 bp, Artificial Sequence
  Synthetic polynucleotide sequence
catctctgatgccaaattgacagcacaaagtgaagaaaaacttgtatatattttgcctttggaaaggaca

SEQ ID NO: 324 - DNA, 70 bp, Artificial Sequence
  Synthetic polynucleotide sequence
gtagtgttcaggaaattctttttccactattttttttattttggttaatattaattagcatgatgcatcc

SEQ ID NO: 325 - DNA, 70 bp, Artificial Sequence
  Synthetic polynucleotide sequence
gttaacaaatacataaattttaaaattattcttcctctcaaacataggggtgatagcaaacctgtgataa

SEQ ID NO: 326 - DNA, 70 bp, Artificial Sequence
  Synthetic polynucleotide sequence
tgggaaaaggacaaaaatacctcctccagcatcgtttggctcacggaaaagtcctccacgccgtgctttg

SEQ ID NO: 327 - DNA, 70 bp, Artificial Sequence
  Synthetic polynucleotide sequence
tcataaagagttcaagcactacgagaagcttaaagaacacgagagaaggcgttatctggagtcactggga

SEQ ID NO: 328 - DNA, 70 bp, Artificial Sequence
  Synthetic polynucleotide sequence
atcatattcttcaaatttagactcatcttctattctcttatcttcattcaatgttattaattatactctc

SEQ ID NO: 329 - DNA, 70 bp, Artificial Sequence
  Synthetic polynucleotide sequence
aaccagtcataactgcaagctgtttacacaaggaatattatgagacaaagaaaagttgcttttcaacaag

SEQ ID NO: 330 - DNA, 70 bp, Artificial Sequence
  Synthetic polynucleotide sequence
tgcataaagaatatgatgacaagaaagattttcttctttcaagaaaagtaaagaaagtggcaactaaata

SEQ ID NO: 331 - DNA, 70 bp, Artificial Sequence
  Synthetic polynucleotide sequence
gcctaggtaacagagcaaaactctgtctctgaaaaaaaaaaaaatcaaccttgaaaattcaaatattgat

SEQ ID NO: 332 - DNA, 70 bp, Artificial Sequence
  Synthetic polynucleotide sequence
gggtactgggaccctggtccacgcagctgacccctcttgaggacactgcggttacgtactgggggggctt

SEQ ID NO: 333 - DNA, 70 bp, Artificial Sequence
  Synthetic polynucleotide sequence
catcgcgctttccttggctttttctttctgggtgagggtcagtttcatgatgcttgctcggcaaacttgt

SEQ ID NO: 334 - DNA, 70 bp, Artificial Sequence
  Synthetic polynucleotide sequence
cctccaaaaattacggtggaccggtacggagtcgaggaaagcacaagagctgagtggaaccaagtgaacg

SEQ ID NO: 335 - DNA, 70 bp, Artificial Sequence
  Synthetic polynucleotide sequence
tcctgtaactttttaaattttggaccaagaaagaaacaccgaacaaatcctatgagaacactaataatgg

SEQ ID NO: 336 - DNA, 70 bp, Artificial Sequence
  Synthetic polynucleotide sequence
cttaaggctgagtttagagctttccactcatactcttccttcctctcccacatttcttgatctccacccc

SEQ ID NO: 337 - DNA, 70 bp, Artificial Sequence
  Synthetic polynucleotide sequence
tattacgtcgttggtaagtctagtttatagttaataatctattgctgctacaatttatctgattatcttg

SEQ ID NO: 338 - DNA, 70 bp, Artificial Sequence
  Synthetic polynucleotide sequence
ctctaatttctatgattgctttttagaagggaaaagatacaatgcttccaccttaataattcgttgtaaa

SEQ ID NO: 339 - DNA, 70 bp, Artificial Sequence
  Synthetic polynucleotide sequence
tagctttgtgtcaaatgactagatcatctgcttcctttgggaatttctaacagatagtaagtaaatagcg

SEQ ID NO: 340 - DNA, 70 bp, Artificial Sequence
  Synthetic polynucleotide sequence
ccagaagaatattcacgcacataacagcaccagcattttaacaaaagcaagactgtagctcaaatctatt

SEQ ID NO: 341 - DNA, 70 bp, Artificial Sequence
  Synthetic polynucleotide sequence
taatgataagctcaaatgatgtggcgactctgctgcatcccattaagatcgcaggcacgatagaaaatat

SEQ ID NO: 342 - DNA, 70 bp, Artificial Sequence
  Synthetic polynucleotide sequence
tttcacacagagaaggtttttttttttgtgctgcctccagttcttttttcatggtggatttcaaaatctc

SEQ ID NO: 343 - DNA, 70 bp, Artificial Sequence
  Synthetic polynucleotide sequence
ttcatcatcagcatttaattcctccttccaaaacaattaacaaaaaagcccacaaaaaaggagccaatca

SEQ ID NO: 344 - DNA, 70 bp, Artificial Sequence
  Synthetic polynucleotide sequence
cacactttaattccagtctaaaattaaagtcttcagtctccacattccctactttccaaattcagctttc

SEQ ID NO: 345 - DNA, 70 bp, Artificial Sequence
  Synthetic polynucleotide sequence
aacagggcatcatcctagacgacccactatcagcatttcaaccgagttatgtactttatacagtcccatc

SEQ ID NO: 346 - DNA, 70 bp, Artificial Sequence
  Synthetic polynucleotide sequence
gtgaaccagattagccaaacggtaagtgtccttttcgtaatgctccagttataaaacagatatgataggt

SEQ ID NO: 347 - DNA, 70 bp, Artificial Sequence
  Synthetic polynucleotide sequence
cgtgcaccccatcctcatgctgtctcatgtgctcatgaccgaaacccggcattcttgtgactcacaaggg

SEQ ID NO: 348 - DNA, 70 bp, Artificial Sequence
  Synthetic polynucleotide sequence
ttaaaatttggtcacttttgtgtagcaatcatttttatgccttttccttgagatttttttttctcaaaaa

SEQ ID NO: 349 - DNA, 70 bp, Artificial Sequence
  Synthetic polynucleotide sequence
aagcattctctttgtgaattgctccgcatacccaacatatgtaaaagaatctgtttcctgtcctttgtga

SEQ ID NO: 350 - DNA, 70 bp, Artificial Sequence
  Synthetic polynucleotide sequence
tttaaaattttcttacaaaatttattaaaaagacatacaaacaacatatgggagatgagaataagattcc

SEQ ID NO: 351 - DNA, 70 bp, Artificial Sequence
  Synthetic polynucleotide sequence
cttcttttctggagcctgtttttgggcactaattcttcttggtggatgactcataaacaaatgttgtttt

SEQ ID NO: 352 - DNA, 70 bp, Artificial Sequence
  Synthetic polynucleotide sequence
tttcagtggaagctgtctgttctcactggcttggcttggatggctgatgccatggagatgatgatcctca

SEQ ID NO: 353 - DNA, 70 bp, Artificial Sequence
  Synthetic polynucleotide sequence
ttacagttttgaatgtaaatctatttataaccccacctgctcttcccctacccaattattttgagtcaca

SEQ ID NO: 354 - DNA, 70 bp, Artificial Sequence
  Synthetic polynucleotide sequence
tttggagtgtaaaccggtttaatgcagaggacctgggggtcccccccacagggctggtggaggaagggca

SEQ ID NO: 355 - DNA, 70 bp, Artificial Sequence
  Synthetic polynucleotide sequence
tttcggttaaccggcgttgggccggcggaattcaagtgctgatgatgaacctgcgtggccttcggtgtcc

SEQ ID NO: 356 - DNA, 70 bp, Artificial Sequence
  Synthetic polynucleotide sequence
ctctcctaagcagacagagaatgtccaggaaataaaatatttaaaaaaccattgaaaacatccaacaaac

SEQ ID NO: 357 - DNA, 70 bp, Artificial Sequence
  Synthetic polynucleotide sequence
ataaacgctcttgcaatgaagagtaacagagtgagcacatttcttccaacacagataaacagcaggaggc

SEQ ID NO: 358 - DNA, 70 bp, Artificial Sequence
  Synthetic polynucleotide sequence
gctgaggtgagcatgttaaaacgttgatattgctgaagaatcagagtttgcaaaaagactacaacaagaa

SEQ ID NO: 359 - DNA, 70 bp, Artificial Sequence
  Synthetic polynucleotide sequence
gcctcccacacagaaacgcaaaacataaaaaccaaaaccagacaacgaaaacatattcaaagcagcagcc

SEQ ID NO: 360 - DNA, 70 bp, Artificial Sequence
  Synthetic polynucleotide sequence
tcccccaggaagccctaaaagtacagtgggactgttcgggaaaaacttggaacccccaggacctacataa

SEQ ID NO: 361 - DNA, 70 bp, Artificial Sequence
  Synthetic polynucleotide sequence
ttggcgttttattaaaaatggaaaaagttgttttagtgagcactcatggtgctttctcccctcctccccc

SEQ ID NO: 362 - DNA, 70 bp, Artificial Sequence
  Synthetic polynucleotide sequence
ctgctgtcagctccttctcattcagggacctaggaaaccaggatccccaggtgatttaaaaaaaaaaaaa

SEQ ID NO: 363 - DNA, 70 bp, Artificial Sequence
  Synthetic polynucleotide sequence
cttttaggactaccttgatttattccgtccctgggctggtggggccgaggagtgtcggaggagagccagt

SEQ ID NO: 364 - DNA, 70 bp, Artificial Sequence
  Synthetic polynucleotide sequence
agagggcaccccaagcaaaacatacacacaaaacaatcaccaaaggcccacaaacggatgacaaagccaa

SEQ ID NO: 365 - DNA, 70 bp, Artificial Sequence
  Synthetic polynucleotide sequence
tcatcctcctgttctggaagccactgagatacaaggcctggaaccccaaatacgagctgttcccctcgcg

SEQ ID NO: 366 - DNA, 70 bp, Artificial Sequence
  Synthetic polynucleotide sequence
aaccagagcactcgagtgttttacacaagctgacattgatgagatgaaggatatcctatgtctttgaatg

SEQ ID NO: 367 - DNA, 70 bp, Artificial Sequence
  Synthetic polynucleotide sequence
tcatggccacagacgggacaaaccaaggctttattgtgaatgggagagtgtgggaagaacctgttttatt

SEQ ID NO: 368 - DNA, 70 bp, Artificial Sequence
  Synthetic polynucleotide sequence
ttcagatttacaaacctgacaaaatatatctgtaatgtgcctggatcaaagtacctttcttgaataggta

SEQ ID NO: 369 - DNA, 70 bp, Artificial Sequence
  Synthetic polynucleotide sequence
atgatgaagattacagtccaagcgggatatccacagtggccaggacaaccaggcatactacaggaattac

SEQ ID NO: 370 - DNA, 70 bp, Artificial Sequence
  Synthetic polynucleotide sequence
aggttcaccaaacaggtagcaccatcaaaaagaacatggattaaattggggggccaaccatgtctagggg

SEQ ID NO: 371 - DNA, 70 bp, Artificial Sequence
  Synthetic polynucleotide sequence
tcattcataaatggggcatcccttatgacaacagcgttatagtccgacgggagtaaataaaaacagctgt

SEQ ID NO: 372 - DNA, 70 bp, Artificial Sequence
  Synthetic polynucleotide sequence
attgatagttatgacccaatcatgtatcggcggcaagtctcatattgtttccaacaaccgacgttatttg

SEQ ID NO: 373 - DNA, 70 bp, Artificial Sequence
  Synthetic polynucleotide sequence
ttaatgggcacattttactttgcatttgcttggaagtgagttaagcgtttttttttccctaagaaaatcg

SEQ ID NO: 374 - DNA, 70 bp, Artificial Sequence
  Synthetic polynucleotide sequence
ttgtttcatatttgtggggtaacgtctccatgaaaggatgtatgttgggagctttggctggactcacaat

SEQ ID NO: 375 - DNA, 70 bp, Artificial Sequence
  Synthetic polynucleotide sequence
ttcttatataactgcagaaagataaatatcactttgtttgttcctgtaggttttctttagtgtaatccat

SEQ ID NO: 376 - DNA, 70 bp, Artificial Sequence
  Synthetic polynucleotide sequence
ccatagctagcaccataccccaagaccaggatgaaataagccagggaaaggagacacatcttattccctt

SEQ ID NO: 377 - DNA, 70 bp, Artificial Sequence
  Synthetic polynucleotide sequence
aacaacatcatcagcatcttcaagttcatcgagccactggcggagatcgcggtgcaggacacgtacttca

SEQ ID NO: 378 - DNA, 70 bp, Artificial Sequence
  Synthetic polynucleotide sequence
attgctaaagttggcttcttcgccggtatcctgttacctgcatttattttgatcgcattagcgccgattt

SEQ ID NO: 379 - DNA, 70 bp, Artificial Sequence
  Synthetic polynucleotide sequence
ggtatgaatgtcagtttttgtttacaatttctcaccacccacaccaaagtcctaccactgcctgtaccta

SEQ ID NO: 380 - DNA, 70 bp, Artificial Sequence
  Synthetic polynucleotide sequence
gacattgcagtgacataagcgacattggcaaagagatacacaaatgtgaccagtgggatggagatgaaga

SEQ ID NO: 381 - DNA, 70 bp, Artificial Sequence
  Synthetic polynucleotide sequence
ttaaagttggtaagcagctagacatcatttagaagcagacgggttaaaatagacaagaaatagcaaagac

SEQ ID NO: 382 - DNA, 70 bp, Artificial Sequence
  Synthetic polynucleotide sequence
aaggcttttatttgtggttcaagcctcattcagcataaaagaattcacacaggtgagaaaccctatgaat

SEQ ID NO: 383 - DNA, 70 bp, Artificial Sequence
  Synthetic polynucleotide sequence
tgtccttgcagaatatccaggatttctttgctttcgggcctcgtgttgctgctcacctggattggtgggt

SEQ ID NO: 384 - DNA, 70 bp, Artificial Sequence
  Synthetic polynucleotide sequence
tatgtattttggatatgggatcccacacagcttggagaacaatcaacagccaccagcgtcaagctcccaa

SEQ ID NO: 385 - DNA, 70 bp, Artificial Sequence
  Synthetic polynucleotide sequence
cggtccttgaaggtgtcgcagcggttggactttccgattgccggtattcccgttcaccagttacgtggat

SEQ ID NO: 386 - DNA, 70 bp, Artificial Sequence
  Synthetic polynucleotide sequence
tttcttcaacacagccagcatgtctatcagcggcatccagcatccaaatcacaagtgacaaagaaccttc

SEQ ID NO: 387 - DNA, 70 bp, Artificial Sequence
  Synthetic polynucleotide sequence
ccgtaacgtgctgtctcacatacccgtcccggttctcggggcgctggtcatctatgcggcaatccgtatg

SEQ ID NO: 388 - DNA, 70 bp, Artificial Sequence
  Synthetic polynucleotide sequence
actgactggggtgcactgagaacaggcaagactttaaattcccataaaatgtctgacttcactgaaactt

SEQ ID NO: 389 - DNA, 70 bp, Artificial Sequence
  Synthetic polynucleotide sequence
tatgtttatttttaaaagtaacttactatctatcttgtctttttcgtatcagaaaaggtgctgttaggaa

SEQ ID NO: 390 - DNA, 70 bp, Artificial Sequence
  Synthetic polynucleotide sequence
gtcccgagctgcctgtccaccgctccttccacagcactgctctgtgctgcttgcatcactgtggtttccc

SEQ ID NO: 391 - DNA, 70 bp, Artificial Sequence
  Synthetic polynucleotide sequence
aagagtttaataatcacaatgccattgggcaaagagatgaaggaatcctcaagcttcacatctgtctgct

SEQ ID NO: 392 - DNA, 70 bp, Artificial Sequence
  Synthetic polynucleotide sequence
ttttggagccacgggtggacctgatacagtggcccgagcccatggacagaaaggagactcaagtctgccc

SEQ ID NO: 393 - DNA, 70 bp, Artificial Sequence
  Synthetic polynucleotide sequence
acaaataatctctgcttaaaagtgctctagggccatggattcatgtaaaaatgccgcaggggggactgaa

SEQ ID NO: 394 - DNA, 70 bp, Artificial Sequence
  Synthetic polynucleotide sequence
caggagtgagtcccaacatttccccaggccagtacagatacagatcctgcacctgcactgagtgtcaacc

SEQ ID NO: 395 - DNA, 70 bp, Artificial Sequence
  Synthetic polynucleotide sequence
tacaggcatttgccaccgcacccagctaatttttttttttgtactttcagtagagacagggctccaccat

SEQ ID NO: 396 - DNA, 70 bp, Artificial Sequence
  Synthetic polynucleotide sequence
caggacttttgatccctaacaaatgtgcgcagagtgacctttaaaaccaagtacaattaagctaatacct

SEQ ID NO: 397 - DNA, 70 bp, Artificial Sequence
  Synthetic polynucleotide sequence
gaggagagatcctctgtagtccctcaaccgatttatccctgggggcatgctctgtttttccaatgttgaa

SEQ ID NO: 398 - DNA, 70 bp, Artificial Sequence
  Synthetic polynucleotide sequence
agctttgatttagaatcttaactgctattacacgtctcttctgtctaccttcaaataggtcatctaatac

SEQ ID NO: 399 - DNA, 70 bp, Artificial Sequence
  Synthetic polynucleotide sequence
ggtcagtagaacctattttcagactcaaaaaccatcttcagaaagaaaaggcccagggaaggaatgtatg

SEQ ID NO: 400 - DNA, 70 bp, Artificial Sequence
  Synthetic polynucleotide sequence
ccgacatcctcaaggtgctgacgaagccgctcacacacctcatcgtgcagttggactccgacatgatcat

SEQ ID NO: 401 - DNA, 70 bp, Artificial Sequence
  Synthetic polynucleotide sequence
ggggttatttctcaggcctaggtattgatgttaccttaattctttagtctggaagaacttgtttggggtg

SEQ ID NO: 402 - DNA, 70 bp, Artificial Sequence
  Synthetic polynucleotide sequence
taatccatagagacacatggaaacaactacatgaagagtcctcagcgcataagggagtgttatcagtaac

SEQ ID NO: 403 - DNA, 70 bp, Artificial Sequence
  Synthetic polynucleotide sequence
ccttcgcgcctggctcacgcccatctcgaccaacaaccgcggccttgcactacagaaagaccccaacccc

SEQ ID NO: 404 - DNA, 70 bp, Artificial Sequence
  Synthetic polynucleotide sequence
acatattcagcttggttctctttggaagtatactttggattcctggggtgtatcatttatgatgcgagaa

SEQ ID NO: 405 - DNA, 70 bp, Artificial Sequence
  Synthetic polynucleotide sequence
tgaaagaggggaaatcagacagagtaattctcttcagcacccagtttatagatgaggctgacattctggc

SEQ ID NO: 406 - DNA, 70 bp, Artificial Sequence
  Synthetic polynucleotide sequence
tgaggaagggctcatgccccttatttatgggaaccatttcattctaacagaataaaccgagaaggaaacc

SEQ ID NO: 407 - DNA, 70 bp, Artificial Sequence
  Synthetic polynucleotide sequence
atctgtaccccaagcttccctttctttaacttggtggtgtactttcgttttcttttcctaacagcacctt

SEQ ID NO: 408 - DNA, 70 bp, Artificial Sequence
  Synthetic polynucleotide sequence
agaactatcatcttggattgaataattcggctcatattcgtcttcattttcaccaccattaattaatggc

SEQ ID NO: 409 - DNA, 70 bp, Artificial Sequence
  Synthetic polynucleotide sequence
ggtctccttcctctgttctcctgttcatccacaagtgaaccctgctgatgtggactccgaaactgtgatg

SEQ ID NO: 410 - DNA, 70 bp, Artificial Sequence
  Synthetic polynucleotide sequence
tgactgttacaggattttttattggtggaccttctaatatgattagttctgctatttctgcggacttggg

SEQ ID NO: 411 - DNA, 70 bp, Artificial Sequence
  Synthetic polynucleotide sequence
taatgaatatatgtatatacacatacctgcccagatcgagatgtagaatatttccagcactccagaaggc

SEQ ID NO: 412 - DNA, 70 bp, Artificial Sequence
  Synthetic polynucleotide sequence
gatgtaggtgaggctcatgtcaaacacgatggaggtccctgggtacttgtgatgcaggtagtccacgtcc

SEQ ID NO: 413 - DNA, 70 bp, Artificial Sequence
  Synthetic polynucleotide sequence
gcttgtttctccctcctgttcccgccaattgtgtgacaggatggacacggccgcctgccttcctccctgg

SEQ ID NO: 414 - DNA, 70 bp, Artificial Sequence
  Synthetic polynucleotide sequence
cgatggcgttgtggtgagcacaggaaggccattatagtcccaatccttgaggcaatggttgaagtccggc

SEQ ID NO: 415 - DNA, 70 bp, Artificial Sequence
  Synthetic polynucleotide sequence
ttgagaaaaaaaaatctcaaggaaaaggcataaaaatgattgctacacaaaagtgaccaaattttaagaa

SEQ ID NO: 416 - DNA, 70 bp, Artificial Sequence
  Synthetic polynucleotide sequence
ttgtcacttctcatactctcctatcttaactgactacctacttggtgcatccattcatttcacggatgac

SEQ ID NO: 417 - DNA, 70 bp, Artificial Sequence
  Synthetic polynucleotide sequence
tgcagcacgtggtgctggccggcctgacgccctcctctgcattctcagcattaatgctgctgccggagac

SEQ ID NO: 418 - DNA, 70 bp, Artificial Sequence
  Synthetic polynucleotide sequence
tcttctcggctttcgccagcgatctgctgcatctctacctcggcctgggcctcctcgctggctttggttg

SEQ ID NO: 419 - DNA, 70 bp, Artificial Sequence
  Synthetic polynucleotide sequence
ctggcccaagataaaaaattgattagctattttagtctgcttcccattttaagttttaaaatctgatatg

SEQ ID NO: 420 - DNA, 70 bp, Artificial Sequence
  Synthetic polynucleotide sequence
tgctcccaatatctactttctgtttttttcctatggcattgttgtaggtcttggatgtggtttattatac

SEQ ID NO: 421 - DNA, 70 bp, Artificial Sequence
  Synthetic polynucleotide sequence
atttcaaaactgagaaattcatgggaatgatgtattttgtggaatcaagaacaaaattatagtgcgataa

SEQ ID NO: 422 - DNA, 70 bp, Artificial Sequence
  Synthetic polynucleotide sequence
aagtggtattccaactgcccacactgcataagctgtcaccttgaagatggcaaaattgaaaatttttttt

SEQ ID NO: 423 - DNA, 70 bp, Artificial Sequence
  Synthetic polynucleotide sequence
gcttggctctttgctgagtcacttacagcaatattagtttgcagattatccatatacaaactctttttaa

SEQ ID NO: 424 - DNA, 70 bp, Artificial Sequence
  Synthetic polynucleotide sequence
gatagaatacaagcctgaggcaggttgcctggtgtactccttgagcgcaccgatcacgaacgggccgata

SEQ ID NO: 425 - DNA, 70 bp, Artificial Sequence
  Synthetic polynucleotide sequence
gactccaacacactagcagcggaactaataaagacaacattcgtgggtagcgccatttcgaggagataac

SEQ ID NO: 426 - DNA, 70 bp, Artificial Sequence
  Synthetic polynucleotide sequence
atgctcagcatcttctactttgccattccggtgggcagtggtctgggctacattgcaggctccaaagtga

SEQ ID NO: 427 - DNA, 70 bp, Artificial Sequence
  Synthetic polynucleotide sequence
ttttttttttttatgagttcaatatttttatttctttacaatgatttcagaagagattacaaagagatta

SEQ ID NO: 428 - DNA, 70 bp, Artificial Sequence
  Synthetic polynucleotide sequence
agaaaatgaactaatatatgatgctattatcaaaaggaaaaaaagaaaaagaaacatctttcatggtctt

SEQ ID NO: 429 - DNA, 70 bp, Artificial Sequence
  Synthetic polynucleotide sequence
ggcagtaactaaccatagcgtcttactgctccaccaagggtgccctggacatgctgaccaaggtgatggc

SEQ ID NO: 430 - DNA, 70 bp, Artificial Sequence
  Synthetic polynucleotide sequence
agggttcattgcaacacccatgacacagaaagggccacagaaagtggtggacaagattactgaaatgatc

SEQ ID NO: 431 - DNA, 70 bp, Artificial Sequence
  Synthetic polynucleotide sequence
ctcctgctgtttatctgtgttggaagaaatgtgctcactctgttactcttcattgcaagagcgtttattt

SEQ ID NO: 432 - DNA, 70 bp, Artificial Sequence
  Synthetic polynucleotide sequence
tgaacctggcacctgcctgtagtacttcatccatggaactgcatcttcctgatcgccgatctccttctgg

SEQ ID NO: 433 - DNA, 70 bp, Artificial Sequence
  Synthetic polynucleotide sequence
tcctaatgggatgatgcgagtacgagggatccgtgttggccgcggtggtcagggtgaataagccggtgtg

SEQ ID NO: 434 - DNA, 70 bp, Artificial Sequence
  Synthetic polynucleotide sequence
ccatatccaaaatacatagcaaatccaatcaacatccagactccacattgggcccaggtctcagtggtca

SEQ ID NO: 435 - DNA, 70 bp, Artificial Sequence
  Synthetic polynucleotide sequence
acatttggaggagttaatgggatctctcttcacctccttctcggcctgttcttacgcatggagccacgag

SEQ ID NO: 436 - DNA, 70 bp, Artificial Sequence
  Synthetic polynucleotide sequence
ttcctggaggaaacctatttggaattataatcctattctattgtgccatcattggtggtaaacttttggg

SEQ ID NO: 437 - DNA, 70 bp, Artificial Sequence
  Synthetic polynucleotide sequence
gctagataaggctccaagtataaaaagagcattaaagatatgattctggaacgtgaacagtgccaggcac

SEQ ID NO: 438 - DNA, 70 bp, Artificial Sequence
  Synthetic polynucleotide sequence
cttttttgtctgtggttttatttcttctgtatctgataagacagttttagtttcttcatgtgcagtatta

SEQ ID NO: 439 - DNA, 70 bp, Artificial Sequence
  Synthetic polynucleotide sequence
ggtactgatgctacaataaattggagatgcaagtataaaatgtgtagatggcaaataaataattctcatt

SEQ ID NO: 440 - DNA, 70 bp, Artificial Sequence
  Synthetic polynucleotide sequence
gctggtgttcctcaccttgtcagtggtggtgatgaagtttctcctggcggcgctggtcctgtctctcatt

SEQ ID NO: 441 - DNA, 70 bp, Artificial Sequence
  Synthetic polynucleotide sequence
aacttctttcttcaattaaaaaaaagtaccccttgccccagccttgcttcaatgcaaaaccttatcaacc

SEQ ID NO: 442 - DNA, 70 bp, Artificial Sequence
  Synthetic polynucleotide sequence
ttttttttttttttgacaggtaaactttttaatgatgttctaatgttgacaactgcagttttattttgct

SEQ ID NO: 443 - DNA, 70 bp, Artificial Sequence
  Synthetic polynucleotide sequence
tttcttttaagttaaaaccagcaaaacacaccatcagaaatgtagtcaaaattcgtatcaatactgcaat

SEQ ID NO: 444 - DNA, 70 bp, Artificial Sequence
  Synthetic polynucleotide sequence
cgatgactggaaagtggaatggagacattttcgaaaggaacacatgatttgaataggattatgaaaaagg

SEQ ID NO: 445 - DNA, 70 bp, Artificial Sequence
  Synthetic polynucleotide sequence
ggagtcaaggtagttttctgtaaactgtacttgggggatcagtgagagaaccttctggcatggtccctgg

SEQ ID NO: 446 - DNA, 70 bp, Artificial Sequence
  Synthetic polynucleotide sequence
ccttcccaaatcagcttcaaagaggaagccttccacagcaataaataaaaattgtgcaaatgtcacaatg

SEQ ID NO: 447 - DNA, 70 bp, Artificial Sequence
  Synthetic polynucleotide sequence
tgccagtttgaggtgaagctgtcaaaagcaatataacctgccagtaagatgaggcctgagagtgtggtgg

SEQ ID NO: 448 - DNA, 70 bp, Artificial Sequence
  Synthetic polynucleotide sequence
caatgctctacctgccgactctcaatgccgatatcatcgacaacggtgtggcacaaggcgataccggcta

SEQ ID NO: 449 - DNA, 70 bp, Artificial Sequence
  Synthetic polynucleotide sequence
aaagacaatagaatgtaatatataattattggtttaggctcacagagagatattagaggaattaggtaag

SEQ ID NO: 450 - DNA, 70 bp, Artificial Sequence
  Synthetic polynucleotide sequence
ttgcagaaaaaaaactcagagactatgaaatacataaaccgatatatcaagttatttgaaaaatacctat

SEQ ID NO: 451 - DNA, 70 bp, Artificial Sequence
  Synthetic polynucleotide sequence
aggaagtctgcattatcacgaggagcttggaaaggaggtaacacactcaaggcaaatttcaagtaactca

SEQ ID NO: 452 - DNA, 70 bp, Artificial Sequence
  Synthetic polynucleotide sequence
atgaagctacatcagctctgggatactgaaagtggaaaaggttgtccaaggaaggcccggggacaaagcc

SEQ ID NO: 453 - DNA, 70 bp, Artificial Sequence
  Synthetic polynucleotide sequence
aattttgttttgctttcctgagttcttctctcacttccacagcattattccctgctatcacaaacacatc

SEQ ID NO: 454 - DNA, 70 bp, Artificial Sequence
  Synthetic polynucleotide sequence
tcgtatccacacatcgaaatataatattctcaccttcttgccaattaatttatttgaacagttccaaaga

SEQ ID NO: 455 - DNA, 70 bp, Artificial Sequence
  Synthetic polynucleotide sequence
tttttttttttggggaaacataatgtttttaatttcacccctcacaaagcccagttgaatgcttcataac

SEQ ID NO: 456 - DNA, 70 bp, Artificial Sequence
  Synthetic polynucleotide sequence
ctttgtagtagttcatacccactcagagttataatggcaaacaaacagaaagcattagtacaagcccctc

SEQ ID NO: 457 - DNA, 70 bp, Artificial Sequence
  Synthetic polynucleotide sequence
tttttctattactagagaactccaggacattaagaatttcaaaagcgattttttcacccacggcttctac

SEQ ID NO: 458 - DNA, 70 bp, Artificial Sequence
  Synthetic polynucleotide sequence
gtaaaagccctgttccaatagggcctgactgggtcttgttaagagatacacagcttagaaatgctcagtg

SEQ ID NO: 459 - DNA, 70 bp, Artificial Sequence
  Synthetic polynucleotide sequence
ttttttaaagcttctagtacttttaggaaactcatgcagcaattttcttgcgatattctatcaataattc

SEQ ID NO: 460 - DNA, 70 bp, Artificial Sequence
  Synthetic polynucleotide sequence
aaacagcttctgtatcagtgcagggacatccttcctccaggcagcaggccttcaagcttcaagcctcagg

SEQ ID NO: 461 - DNA, 70 bp, Artificial Sequence
  Synthetic polynucleotide sequence
accacctgagtcaatttagtcacacaggactaaaatctaaatgagcaagatgttgttattagttagtatt

SEQ ID NO: 462 - DNA, 70 bp, Artificial Sequence
  Synthetic polynucleotide sequence
caaaagttcacatctcgtgtctagaacacaagatattcatattttcagacaggtaaccagtcggggagag

SEQ ID NO: 463 - DNA, 70 bp, Artificial Sequence
  Synthetic polynucleotide sequence
gcggaaggctccgccaaggcgcacgcagggcggggtgtaaggggagttcctgggactcccggtgcaccac

SEQ ID NO: 464 - DNA, 70 bp, Artificial Sequence
  Synthetic polynucleotide sequence
tgaaagaaagaaaagaaaaagagggaagagttaaggccagacagggacagggatggaaggagacagaggc

SEQ ID NO: 465 - DNA, 70 bp, Artificial Sequence
  Synthetic polynucleotide sequence
tgcccctccatccaaaactgccaagtgactcattgccttcccaacccttccagaggctttctgtgaaagt

SEQ ID NO: 466 - DNA, 70 bp, Artificial Sequence
  Synthetic polynucleotide sequence
gtccagtacaaacagagggtcaaccacgatccccacattgaagccctcacgcaccaaccgggtcttcatc

SEQ ID NO: 467 - DNA, 70 bp, Artificial Sequence
  Synthetic polynucleotide sequence
caaaaacaggtggatcagctccagccacatttattacaaaatagggaccgcagttttggtataaaaaaaa

SEQ ID NO: 468 - DNA, 70 bp, Artificial Sequence
  Synthetic polynucleotide sequence
atttcctcacaatcttcaatcaaattaaagttggattatatcaagtggctgtccattcagtttctttctg

SEQ ID NO: 469 - DNA, 70 bp, Artificial Sequence
  Synthetic polynucleotide sequence
gacggtgagtcaagggtgggacccctgctctatcaactggtttcctacccgcccaggccccgcccccaac

SEQ ID NO: 470 - DNA, 70 bp, Artificial Sequence
  Synthetic polynucleotide sequence
tggtggaccaacaagattgggttgagctgaaaattgccaaggatacataggggcgtgggttgagattcct

SEQ ID NO: 471 - DNA, 70 bp, Artificial Sequence
  Synthetic polynucleotide sequence
ttgggaaaagttcttaagttctgaaacgccgcggatcaatgtctttatggcagtgcctacaatatacacc

SEQ ID NO: 472 - DNA, 70 bp, Artificial Sequence
  Synthetic polynucleotide sequence
aaagttcttgtgtttgaccacatccttcatggcaccaagaatagtctcttcagtaatcgaatctaacaat

SEQ ID NO: 473 - DNA, 70 bp, Artificial Sequence
  Synthetic polynucleotide sequence
tgtcgttgaaaaccatctggccatagaacaacccaacacacaccttcctgagacgaagccttccccatga

SEQ ID NO: 474 - DNA, 70 bp, Artificial Sequence
  Synthetic polynucleotide sequence
aggaggacatcaaggccttaaacgccaaaatgaccaatattgagaaacagctctctgagatactcaggat

SEQ ID NO: 475 - DNA, 70 bp, Artificial Sequence
  Synthetic polynucleotide sequence
agacatcaccatcctccagcaaaaactactacaggaggcctatgagacacgtgaagatatcatcaggctg

SEQ ID NO: 476 - DNA, 70 bp, Artificial Sequence
  Synthetic polynucleotide sequence
gcgtggacatcgccaagagtgagatcttcaatggggacgtggagtggaagtcctgtgacctggaggaggt

SEQ ID NO: 477 - DNA, 70 bp, Artificial Sequence
  Synthetic polynucleotide sequence
ccagttcatttgctgttgctgttcctgctgcaaaaccaagaagccagactatcccccgatccctactttt

SEQ ID NO: 478 - DNA, 70 bp, Artificial Sequence
  Synthetic polynucleotide sequence
gcagataatgaaaagacttataaagcggtatgttttgaaagcacaagtagacaaagaaaatgatgaagtt

SEQ ID NO: 479 - DNA, 70 bp, Artificial Sequence
  Synthetic polynucleotide sequence
ggaatttagtcaaaagatatgtggctgctatgataagaaattccaaaacacatgagggacttacagaaga

SEQ ID NO: 480 - DNA, 70 bp, Artificial Sequence
  Synthetic polynucleotide sequence
aaaaacaggttgggcacaataaacaaccaagtataaggagctcagaagatttccatctaaatagtttcaa

SEQ ID NO: 481 - DNA, 70 bp, Artificial Sequence
  Synthetic polynucleotide sequence
gaaaacttgctgaagtgcggcatggaggtgtacaaaggctacatggatgacccgaggaacacggacaatg

SEQ ID NO: 482 - DNA, 70 bp, Artificial Sequence
  Synthetic polynucleotide sequence
gaagaaggaggtactctgcctactcccttcaatgtcatcccgagccccaagtctctctggtacctgatca

SEQ ID NO: 483 - DNA, 70 bp, Artificial Sequence
  Synthetic polynucleotide sequence
tgctaaaactgaagaaggcctgaccgaagagaactttaaggaactaaagcaagacatttctagtttccgc

SEQ ID NO: 484 - DNA, 70 bp, Artificial Sequence
  Synthetic polynucleotide sequence
tagctactatgactattttattgcttcctgtgaaatcacattctgtatttttctttttgtcttcacaaca

SEQ ID NO: 485 - DNA, 70 bp, Artificial Sequence
  Synthetic polynucleotide sequence
ggccaccttcaccaagtttgacagagatgggaatcgtattctggatgagaaggaacaggaaaaaatgcga

SEQ ID NO: 486 - DNA, 70 bp, Artificial Sequence
  Synthetic polynucleotide sequence
tgggtcttccagatgctatcgcgacagaacaattacaccgtcaacaacaagagaaatggagtggccaaag

SEQ ID NO: 487 - DNA, 70 bp, Artificial Sequence
  Synthetic polynucleotide sequence
gggaccaaagcaccaatatcaccctgagcaggaagggaattgtcaagctcaacatctacttccaagaatt

SEQ ID NO: 488 - DNA, 70 bp, Artificial Sequence
  Synthetic polynucleotide sequence
cacttgggaccaaggccggcaagtaaacaaaaagctcaacaagacagacttggccaaactcttgatattc

SEQ ID NO: 489 - DNA, 70 bp, Artificial Sequence
  Synthetic polynucleotide sequence
ggttaagaacaacatctggtatcccaaatttaatttcagcaagaggaatatccttcccaacatcaccact

SEQ ID NO: 490 - DNA, 70 bp, Artificial Sequence
  Synthetic polynucleotide sequence
gctcaagagtatttctgagagactgtctgtcctcaagggtgccaaacccgatgtctccaatggacaacca

SEQ ID NO: 491 - DNA, 70 bp, Artificial Sequence
  Synthetic polynucleotide sequence
tctgattgggcacttcttcaccatctgcatggccttcttggttctcagcttagctaagtccatcgtgttg

SEQ ID NO: 492 - DNA, 70 bp, Artificial Sequence
  Synthetic polynucleotide sequence
cactcttttcatcaagaacagcatcagctttccacgcttcaaggtcaacaggcgcaacctggtggaggag

SEQ ID NO: 493 - DNA, 70 bp, Artificial Sequence
  Synthetic polynucleotide sequence
tgaacaaaaacaaggtctacagccataagaaatttgacaagatggtggacactcctgcctccgagcctgc

SEQ ID NO: 494 - DNA, 70 bp, Artificial Sequence
  Synthetic polynucleotide sequence
aaaatggaaaatggcagtgagtaccgcaccctcctgaaggcttttggcatccgcttcgacgtgctggtat

SEQ ID NO: 495 - DNA, 70 bp, Artificial Sequence
  Synthetic polynucleotide sequence
tgagaataagactgttgttgtcaccacaattttggaatctccgtatgttatgatgaagaaaaatcatgaa

SEQ ID NO: 496 - DNA, 70 bp, Artificial Sequence
  Synthetic polynucleotide sequence
agctccaggtcacagaagatgtgaattcaagtatcaagacatttttttgtcccaatgatacctacaatga

SEQ ID NO: 497 - DNA, 70 bp, Artificial Sequence
  Synthetic polynucleotide sequence
cattgacatcgtggtggaaaacctggctggcctcaagtacagggtcatcaagggcaatatcgacaagttc

SEQ ID NO: 498 - DNA, 70 bp, Artificial Sequence
  Synthetic polynucleotide sequence
agaccaccttgcaccaggcacaaaacctctttaagctgttgaaccttcagtccctcttcgtgacatcgcg

SEQ ID NO: 499 - DNA, 70 bp, Artificial Sequence
  Synthetic polynucleotide sequence
tgaatactactgctctcatcccaaaggaagccaactctgaggaagtctttttgtttaaaccagaaaacat

SEQ ID NO: 500 - DNA, 70 bp, Artificial Sequence
  Synthetic polynucleotide sequence
aaaccagaaaatatctcagaagaaaatgcaacccacatatttattgccattaaaagtatagataaaagca

SEQ ID NO: 501 - DNA, 70 bp, Artificial Sequence
  Synthetic polynucleotide sequence
tgtgacacatcatactttaagcaggaaaaagagagcagacaagaaagagaatggaacaaaattattataa

SEQ ID NO: 502 - DNA, 70 bp, Artificial Sequence
  Synthetic polynucleotide sequence
atcaaaagaacctgttgctgatgaagaagaggaagacagtgatgatgatgttgaacctattactgaattt

SEQ ID NO: 503 - DNA, 70 bp, Artificial Sequence
  Synthetic polynucleotide sequence
ccgggagatagtgatgaagtacatccattataagctgtcgcagaggggctacgagtgggatgcgggagat

SEQ ID NO: 504 - DNA, 70 bp, Artificial Sequence
  Synthetic polynucleotide sequence
tggacgggtccggggagcagcccagaggcggggtttcatccaggatcgagcagggcgaatggggggggag

SEQ ID NO: 505 - DNA, 70 bp, Artificial Sequence
  Synthetic polynucleotide sequence
gaacgtgctggtggttctgggtgtggttctgttgggccagtttgtggtacgaagattcttcaaatcatga

SEQ ID NO: 506 - DNA, 70 bp, Artificial Sequence
  Synthetic polynucleotide sequence
ccttctttgtctttggggctgcactgtgtgctgagagtgtcaacaaggagatggaaccactggtgggaca

SEQ ID NO: 507 - DNA, 70 bp, Artificial Sequence
  Synthetic polynucleotide sequence
cattaactggggaagaattgtaaccatatttgcatttgaaggtattctcatcaagaaacttctacgacag

SEQ ID NO: 508 - DNA, 70 bp, Artificial Sequence
  Synthetic polynucleotide sequence
ggctcagggcggctgggatggcttttgtcacttcttcaggaccccctttccactggctttttggagaaaa

SEQ ID NO: 509 - DNA, 70 bp, Artificial Sequence
  Synthetic polynucleotide sequence
ggcgcgggaaagttgaactaataaagtttgtacgagttcagtggaggagaccgcaagttgagtggaggag

SEQ ID NO: 510 - DNA, 70 bp, Artificial Sequence
  Synthetic polynucleotide sequence
ctacgaccagactgaggacatcagggatgttcttagaagtttcatggacggtttcaccacacttaaggag

SEQ ID NO: 511 - DNA, 70 bp, Artificial Sequence
  Synthetic polynucleotide sequence
atggactgatgtcctcaagtgtgtggtcagcacagaccctggcctccgctcccactggctggtggctgca

SEQ ID NO: 512 - DNA, 70 bp, Artificial Sequence
  Synthetic polynucleotide sequence
gcagcagacaagggcaagaaaaatgctggaaatgcagaagatccccacacagaaacccagcagccagaag

SEQ ID NO: 513 - DNA, 70 bp, Artificial Sequence
  Synthetic polynucleotide sequence
ttaattgggaaaaagaacagtccacaggaagaggttgaactaaagaagttgaaacatttggagaagtctg

SEQ ID NO: 514 - DNA, 70 bp, Artificial Sequence
  Synthetic polynucleotide sequence
caaaattgcaatcgacaatctagagaaagcagaacttcttcagggaggagatctcttaaggcaaaggaaa

SEQ ID NO: 515 - DNA, 70 bp, Artificial Sequence
  Synthetic polynucleotide sequence
accagaatgcataaaacaagttgatcaagaacttaatggaaaacaagatgaaccgaaaaatgaacagtaa

SEQ ID NO: 516 - DNA, 70 bp, Artificial Sequence
  Synthetic polynucleotide sequence
tgaggaagatgatattgaaagaaggaaagaagttgaaagcatcttgaagaaaaactcagattggatatgg

SEQ ID NO: 517 - DNA, 70 bp, Artificial Sequence
  Synthetic polynucleotide sequence
agcacagcgctatggccgcgagctccggaggatgagtgacgagtttgtggactcctttaagaagggactt

SEQ ID NO: 518 - DNA, 70 bp, Artificial Sequence
  Synthetic polynucleotide sequence
tggccctgggggaaaattcttcaatcccttcagcaataatatggcctcccaacaaaacacagacaacctg

SEQ ID NO: 519 - DNA, 70 bp, Artificial Sequence
  Synthetic polynucleotide sequence
cacttgaagcagattgagataaagaagttcaagtacggtattgaagagcatggtaaggtgaaaatgcgag

SEQ ID NO: 520 - DNA, 70 bp, Artificial Sequence
  Synthetic polynucleotide sequence
cactcataaaatatttggaaaagaacttaatggtaaccagttaatggaaaaaagagaaactgaaggcaaa

SEQ ID NO: 521 - DNA, 70 bp, Artificial Sequence
  Synthetic polynucleotide sequence
tgagaagaagaggaagttcatcaagggggagataaagagtgaatttaaggacatcgaggagatcaaaacc

SEQ ID NO: 522 - DNA, 70 bp, Artificial Sequence
  Synthetic polynucleotide sequence
cccctgccccaacatcgtggactgctacattgcccgacctaccgagaagaaaatcttcacctacttcatg

SEQ ID NO: 523 - DNA, 70 bp, Artificial Sequence
  Synthetic polynucleotide sequence
cagatccatgtcccaatatagtggactgcttcatctccaagccctcagagaagaacattttcaccctctt

SEQ ID NO: 524 - DNA, 70 bp, Artificial Sequence
  Synthetic polynucleotide sequence
accccctcagaatggccaaaaacccccaagtcgtcccagcagctctgcttctaagaagcagtatgtatag

SEQ ID NO: 525 - DNA, 70 bp, Artificial Sequence
  Synthetic polynucleotide sequence
tgctgtctcctccatccagaaagccaagggctatcagctcctagaagaagagaaaatcgtttcccactat

SEQ ID NO: 526 - DNA, 70 bp, Artificial Sequence
  Synthetic polynucleotide sequence
tctacattatccaagtggtgttccgaaatgccctggaaattgggttcctggttggccaatattttctcta

SEQ ID NO: 527 - DNA, 70 bp, Artificial Sequence
  Synthetic polynucleotide sequence
tgtgggattaaccctgctcggtcctttggctccgcggtgatcacacacaacttcagcaaccactggattt

SEQ ID NO: 528 - DNA, 70 bp, Artificial Sequence
  Synthetic polynucleotide sequence
gggatccattacaccggctgctctatgaatcctgcccgctccctggctccagctgtcgtcactggcaaat

SEQ ID NO: 529 - DNA, 70 bp, Artificial Sequence
  Synthetic polynucleotide sequence
aatagtttttgggctgtattatgatgcaatctggcactttgccgacaaccagctttttgtttcgggcccc

SEQ ID NO: 530 - DNA, 70 bp, Artificial Sequence
  Synthetic polynucleotide sequence
tggaaatcttaccgctggtcatggtctcctggttgagttgataatcacatttcaattggtgtttactatc

SEQ ID NO: 531 - DNA, 70 bp, Artificial Sequence
  Synthetic polynucleotide sequence
gtccattggcctgtctgtcaccctgggccaccttgtcggaatctacttcactggctgctccatgaaccca

SEQ ID NO: 532 - DNA, 70 bp, Artificial Sequence
  Synthetic polynucleotide sequence
tctgctttatggggtcatgccgggagacatccgagagacccttgggatcaacgtggtccggaacagtgtc

SEQ ID NO: 533 - DNA, 70 bp, Artificial Sequence
  Synthetic polynucleotide sequence
tccgtggcccatatggttctaaataaaaaatatgggagctaccttggtgtcaacttgggttttggcttcg

SEQ ID NO: 534 - DNA, 70 bp, Artificial Sequence
  Synthetic polynucleotide sequence
gcttgttggactgctcattaggtgcttcattggagatgggaagacccgcctcatcctgaaggctcggtga

SEQ ID NO: 535 - DNA, 70 bp, Artificial Sequence
  Synthetic polynucleotide sequence
tgactcagtctttaaggcagaacaatctgaggacaaaccagagaaatatgaactcagtgtcatcatgtag

SEQ ID NO: 536 - DNA, 70 bp, Artificial Sequence
  Synthetic polynucleotide sequence
ttgtcaaggtttgaaactgacatttgatactaccttctcaccaaacacaggaaagaaaagtggtaaaatc

SEQ ID NO: 537 - DNA, 70 bp, Artificial Sequence
  Synthetic polynucleotide sequence
aaagaagagtgggaaattgaaggcctcctataaacgggattgttttagtgttggcagtaatgttgatata

SEQ ID NO: 538 - DNA, 70 bp, Artificial Sequence
  Synthetic polynucleotide sequence
tgatattggtggtcagatgggattgttcattggtgctagtatccttacaatactagagctctttgattat

SEQ ID NO: 539 - DNA, 70 bp, Artificial Sequence
  Synthetic polynucleotide sequence
aagttcaacaaatctgagcaatacataggggagaacatcctggtgctggacattttctttgaagtcctca

SEQ ID NO: 540 - DNA, 70 bp, Artificial Sequence
  Synthetic polynucleotide sequence
gaagggctgggcagccatcgaacccaagttccccacctcagcctgggccccagacctcccacccctccct

SEQ ID NO: 541 - DNA, 70 bp, Artificial Sequence
  Synthetic polynucleotide sequence
ccgccctgacatcctggacatgcctagcctgcacgtagcttttccgtcttcaccccaaataaagtcctaa

SEQ ID NO: 542 - DNA, 70 bp, Artificial Sequence
  Synthetic polynucleotide sequence
tcccagaaggtggacttggcactgaaacagctgggacacatccgcgagtacgaacagcgcctgaaagtgc

SEQ ID NO: 543 - DNA, 70 bp, Artificial Sequence
  Synthetic polynucleotide sequence
taggcaaatgaaagccatcgtgtctagaactcaaatctttgaactaatagaaatctctgatataagctgg

SEQ ID NO: 544 - DNA, 70 bp, Artificial Sequence
  Synthetic polynucleotide sequence
aactctggtggcaacaagtgtcaacagtgtaacaggcattcgcatcgaggatctgccaacttcagaaagc

SEQ ID NO: 545 - DNA, 70 bp, Artificial Sequence
  Synthetic polynucleotide sequence
tggcaggctttcctgtcctggacgccaatgacctagaagataaaaacagtcctttctactatgactggca

SEQ ID NO: 546 - DNA, 70 bp, Artificial Sequence
  Synthetic polynucleotide sequence
gatgaaagacagacagactctaaaaaagaagaaactatttccccaagtttaaataaaacagatgtgatac

SEQ ID NO: 547 - DNA, 70 bp, Artificial Sequence
  Synthetic polynucleotide sequence
gacatctactatcgaggtcagacagccctgcacatcgccattgagcgtcgctgcaaacactacgtggaac

SEQ ID NO: 548 - DNA, 70 bp, Artificial Sequence
  Synthetic polynucleotide sequence
agacgctacctcttgaagcaaaaagttaaaaaggtatcaagtatatacaagaaagacaaaggcaaagaat

SEQ ID NO: 549 - DNA, 70 bp, Artificial Sequence
  Synthetic polynucleotide sequence
aatgtgaaaagcttatggaggggaacaatacagagatcagatggaagaacgtgaagatcaactttgacaa

SEQ ID NO: 550 - DNA, 70 bp, Artificial Sequence
  Synthetic polynucleotide sequence
tatagacgttaccgcttaaggcaaaatgtcaaaaatatatcaagtatatacataaaagatggagacagag

SEQ ID NO: 551 - DNA, 70 bp, Artificial Sequence
  Synthetic polynucleotide sequence
ttcaaaatcatcctatgaaccaatagcaaccactctccgatggaagcaagaagacatttcagccactgtc

SEQ ID NO: 552 - DNA, 70 bp, Artificial Sequence
  Synthetic polynucleotide sequence
cctactatgccgactactcgcccacgcgccgctccattcactcgctgtgcaccagccactatctcgacct

SEQ ID NO: 553 - DNA, 70 bp, Artificial Sequence
  Synthetic polynucleotide sequence
ccatcataaccataattccataggaaagcaagttcccacctcaacaaatgccaatctcaataatgccaat

SEQ ID NO: 554 - DNA, 70 bp, Artificial Sequence
  Synthetic polynucleotide sequence
actgtctttgtgctggaggctgtgctgaagctggtggcatttggtctgaggcgcttcttcaaggaccgat

SEQ ID NO: 555 - DNA, 70 bp, Artificial Sequence
  Synthetic polynucleotide sequence
agaagactttggacttgctggtaccaccccataagcctgatgagatgacagtggggaaggtttatgcagc

SEQ ID NO: 556 - DNA, 70 bp, Artificial Sequence
  Synthetic polynucleotide sequence
tttggctttttgaactatttccgagacacctggaatatctttgacttcatcaccgtgattggcagtatca

SEQ ID NO: 557 - DNA, 70 bp, Artificial Sequence
  Synthetic polynucleotide sequence
tccaagaattactaaattgtactgacattatttttacacatatttttatcctggagatggtactaaaatg

SEQ ID NO: 558 - DNA, 70 bp, Artificial Sequence
  Synthetic polynucleotide sequence
atgtggcaccaactttgcatactactacttcatcagcttctacatgctctgtgccttcctggtcatcaac

SEQ ID NO: 559 - DNA, 70 bp, Artificial Sequence
  Synthetic polynucleotide sequence
tccaaaaagccttttttagaaagccaaaagttatagaaatccatgaaggcaataagatagacagctgcat

SEQ ID NO: 560 - DNA, 70 bp, Artificial Sequence
  Synthetic polynucleotide sequence
tttcagcaaccatcattcaacgtgcttataaaaattaccgcttgaggcgaaatgacaaaaatacatcaga

SEQ ID NO: 561 - DNA, 70 bp, Artificial Sequence
  Synthetic polynucleotide sequence
aatgcctgactcagacagcgaggcagtttatgagttcacacaggatgcccagcacagcgacctccgggac

SEQ ID NO: 562 - DNA, 70 bp, Artificial Sequence
  Synthetic polynucleotide sequence
ttacatggagatccaggagggtgtaaataacagtaatgaggactttagagaggaaaacttgaaaacagcc

SEQ ID NO: 563 - DNA, 70 bp, Artificial Sequence
  Synthetic polynucleotide sequence
tgccacctgcaccacgaacaataatcccaactcttgtgtcaacatcaaaaagatattcaccgatgtttaa

SEQ ID NO: 564 - DNA, 70 bp, Artificial Sequence
  Synthetic polynucleotide sequence
aattgaaagaatcctcaacagtgtaggctcaagaatgggcagcacagactctcttaataagaccaatggt

SEQ ID NO: 565 - DNA, 70 bp, Artificial Sequence
  Synthetic polynucleotide sequence
gagcaacgtggacttgcggaggtccctttatgccctctgcctggacaccagccgggaaacagatttgtga

SEQ ID NO: 566 - DNA, 70 bp, Artificial Sequence
  Synthetic polynucleotide sequence
cgataaggaatactactttgatcggaatccctccttgttcagatatgttttgaatttttattacacgggg

SEQ ID NO: 567 - DNA, 70 bp, Artificial Sequence
  Synthetic polynucleotide sequence
tattgcatggtttaccatggagtaccttttgcgattcttatcctcaccaaataaatggaagttcttcaaa

SEQ ID NO: 568 - DNA, 70 bp, Artificial Sequence
  Synthetic polynucleotide sequence
gagaatatgggtaagaaagacaaagtacaagataaccacttgtctcctaacaaatggaaatggacaaaga

SEQ ID NO: 569 - DNA, 70 bp, Artificial Sequence
  Synthetic polynucleotide sequence
ctaaaggacttttacgatatctacgaagttgctgctttgaagtggaaggccaagaaaaacagagagcact

SEQ ID NO: 570 - DNA, 70 bp, Artificial Sequence
  Synthetic polynucleotide sequence
gttaaactaaactgtgaacaaccttatgtgactacagcaataataagcatcccaacacctccagtaacca

SEQ ID NO: 571 - DNA, 70 bp, Artificial Sequence
  Synthetic polynucleotide sequence
ccgattggcaaagagtggtaccaccaatgccttcctgcagtacaagcagaatgggggccttgaggacagc

SEQ ID NO: 572 - DNA, 70 bp, Artificial Sequence
  Synthetic polynucleotide sequence
gaaagcagacgacggactgagaccaaactgcaaaacatcccagatcaccacagccatcatcagcatcccc

SEQ ID NO: 573 - DNA, 70 bp, Artificial Sequence
  Synthetic polynucleotide sequence
ggccttcatgtgtatattatcgggaattcttgtcttggccttgcctattgctattattaacgatcgcttc

SEQ ID NO: 574 - DNA, 70 bp, Artificial Sequence
  Synthetic polynucleotide sequence
taagaactctggcatcctgactcgagagtgtggcaatgaatttgcttatttttactttgtttccttcatc

SEQ ID NO: 575 - DNA, 70 bp, Artificial Sequence
  Synthetic polynucleotide sequence
gagctgaaaagagagatcaccaaggaaagtgcctgaaagggattattcccacgcctccaaaggatcctgg

SEQ ID NO: 576 - DNA, 70 bp, Artificial Sequence
  Synthetic polynucleotide sequence
gctggcccaggaagaaattttagaaattaacagagcaggtaggaaacctctcagaggcatgtcgatctga

SEQ ID NO: 577 - DNA, 70 bp, Artificial Sequence
  Synthetic polynucleotide sequence
cctcaagttcttcaagaatgccctaaaccttattgacctcatgtccatcgtccccttttacatcactctg

SEQ ID NO: 578 - DNA, 70 bp, Artificial Sequence
  Synthetic polynucleotide sequence
tcaggagggacagtctgcagatctagagagccaggcccccagtgagcctccacaccctcagatgtattaa

SEQ ID NO: 579 - DNA, 70 bp, Artificial Sequence
  Synthetic polynucleotide sequence
atatgtgactaccattttgtcacgcatcaatctggtgttcattgtgctatttactggagagtgtgtactg

SEQ ID NO: 580 - DNA, 70 bp, Artificial Sequence
  Synthetic polynucleotide sequence
gtacaggtaccgccgccgggcacctgccaccaagcaactgtttcattttttattttccatttgttcttaa

SEQ ID NO: 581 - DNA, 70 bp, Artificial Sequence
  Synthetic polynucleotide sequence
tataacaaagaggcaattaaagggaggattgacttacctataaaacaagacatgattattgacaaactaa

SEQ ID NO: 582 - DNA, 70 bp, Artificial Sequence
  Synthetic polynucleotide sequence
gggcaagtacaggaccatcagccagatcccacagttcacgctcaacttcgtggagttcaacttggagaag

SEQ ID NO: 583 - DNA, 70 bp, Artificial Sequence
  Synthetic polynucleotide sequence
agaaaaaagaaaagaagagcaagtcagatgataaaaacgaaaataaaaacgacccagagaagaaaaagaa

SEQ ID NO: 584 - DNA, 70 bp, Artificial Sequence
  Synthetic polynucleotide sequence
agactaatccagctgaacatacccaatgctctccctctatgaacgcagaggaaaactcccgcatctccat

SEQ ID NO: 585 - DNA, 70 bp, Artificial Sequence
  Synthetic polynucleotide sequence
catttctacacggtgatcaaagtgagaatgagctaccaggattggggggcaaggaagataggagagccaa

SEQ ID NO: 586 - DNA, 70 bp, Artificial Sequence
  Synthetic polynucleotide sequence
tgctcacagcaaggaattaatcacagcttggtacataggatttttggttcttattttttcgtctttcctt

SEQ ID NO: 587 - DNA, 70 bp, Artificial Sequence
  Synthetic polynucleotide sequence
aatacctcctctacgagaagaatgaggtgaaggcgcgagaccgggagtggaagaagtatgaattccatta

SEQ ID NO: 588 - DNA, 70 bp, Artificial Sequence
  Synthetic polynucleotide sequence
cgtctaccacgtcttcatatttttgctggtcttcagctgcctggtgctgtctgtgctgtccactatccag

SEQ ID NO: 589 - DNA, 70 bp, Artificial Sequence
  Synthetic polynucleotide sequence
tctttggggagcctctgaacctgtatgcaaggcctggcaagtcgaacggggatgtgcgggccctcaccta

SEQ ID NO: 590 - DNA, 70 bp, Artificial Sequence
  Synthetic polynucleotide sequence
aatttatttcactacatttgtgttctttatgttcttcattcttttgaatatgtttttggctatcatcaat

SEQ ID NO: 591 - DNA, 70 bp, Artificial Sequence
  Synthetic polynucleotide sequence
ctggggttcccacattctccttcccttcaggatccgccacaaacagacactttttgcttccttaaagtag

SEQ ID NO: 592 - DNA, 70 bp, Artificial Sequence
  Synthetic polynucleotide sequence
accttattgctgctctgccctttgacctgctttacatcttcaacatcaccgtgacctcgctggtgcacct

SEQ ID NO: 593 - DNA, 70 bp, Artificial Sequence
  Synthetic polynucleotide sequence
gtctgtggtggtaaagaaaaaaaagttcaagctggacaaagacaatggggtgactcctggagagaagatg

SEQ ID NO: 594 - DNA, 70 bp, Artificial Sequence
  Synthetic polynucleotide sequence
tcaactaccaggagaatgagatcatccagcagattgtgcagcatgaccgggagatggcccactgcgcgca

SEQ ID NO: 595 - DNA, 70 bp, Artificial Sequence
  Synthetic polynucleotide sequence
ccgtcttgtccgttcactcccagccagcagataccagctacatcctgcagcttcccaaagatgcacctca

SEQ ID NO: 596 - DNA, 70 bp, Artificial Sequence
  Synthetic polynucleotide sequence
agttaccagctgatatgcgtcagaagatacatgattactatgaacacagataccaaggcaaaatctttga

SEQ ID NO: 597 - DNA, 70 bp, Artificial Sequence
  Synthetic polynucleotide sequence
aggtactttaggatttttcatcgcaagtgatgccaaagaagttaaaagggcatttttttactgcaaggcc

SEQ ID NO: 598 - DNA, 70 bp, Artificial Sequence
  Synthetic polynucleotide sequence
ggggcgccacgctccgccatcagctcggtgtccacgggtctgcacagcctgtccaccttccgcggcctca

SEQ ID NO: 599 - DNA, 70 bp, Artificial Sequence
  Synthetic polynucleotide sequence
acaaccaggagaacgccatcatccaggagatcgtcaagtacgaccgcgagatggtgcagcaggccgagct

SEQ ID NO: 600 - DNA, 70 bp, Artificial Sequence
  Synthetic polynucleotide sequence
tgaatttccgtaccacattcgtgtccaagtcgggccaggtggtgtttgccccaaagtccatttgcctcca

SEQ ID NO: 601 - DNA, 70 bp, Artificial Sequence
  Synthetic polynucleotide sequence
atgcctgcatctactttgccatttccaagttcattggttttgggacagactcctgggtctacccaaacat

SEQ ID NO: 602 - DNA, 70 bp, Artificial Sequence
  Synthetic polynucleotide sequence
tgtttgagtcaggcggctccaagaagtgcatccaggttggcggggagttctacactcccagcaagttcga

SEQ ID NO: 603 - DNA, 70 bp, Artificial Sequence
  Synthetic polynucleotide sequence
aaatgaagataaacaaaaagaaaatgaagataaacaaaaagaaaatgaagataaaggaaaagaaaatgaa

SEQ ID NO: 604 - DNA, 70 bp, Artificial Sequence
  Synthetic polynucleotide sequence
ctccccgccttggatcgtcttcaacgtattgtctgatactttcttcctactggatctggtgctcaacttc

SEQ ID NO: 605 - DNA, 70 bp, Artificial Sequence
  Synthetic polynucleotide sequence
cacaccttttatgaaatttatcattcatggagcatcatatttcacatttctgctgttgcttaatctatac

SEQ ID NO: 606 - DNA, 70 bp, Artificial Sequence
  Synthetic polynucleotide sequence
agcacaacctggccaattacatgtttttcctgatgtatttgataaacaaggatgagacagaacacacggg

SEQ ID NO: 607 - DNA, 70 bp, Artificial Sequence
  Synthetic polynucleotide sequence
ctgcagtttgtcagaggcggggacatcattacggacaaaaaggacatgcgaaataactacctgaagtctc

SEQ ID NO: 608 - DNA, 70 bp, Artificial Sequence
  Synthetic polynucleotide sequence
tctgagaggttcgacatctccgaggtcaacaacaagtctgagtgcgagagcctcatgcacacaggccagg

SEQ ID NO: 609 - DNA, 70 bp, Artificial Sequence
  Synthetic polynucleotide sequence
acccttccaacatgcgcttcatgcagttccgcgccaaggacagctactctctggctctttccaaactaga

SEQ ID NO: 610 - DNA, 70 bp, Artificial Sequence
  Synthetic polynucleotide sequence
ccacgtgttcatctggatctcgttcatagacagctactttgaaatcctcttcctgttccaggccctgctc

SEQ ID NO: 611 - DNA, 70 bp, Artificial Sequence
  Synthetic polynucleotide sequence
tctgggagtctcctctccttctagctgccaaagataatgatgtccaggccctgaacaagttgctcaagta

SEQ ID NO: 612 - DNA, 70 bp, Artificial Sequence
  Synthetic polynucleotide sequence
caagaaataccaaatgctgataaatctttagaaatggaaatattaaagcagaaataccggctgaaggatc

SEQ ID NO: 613 - DNA, 70 bp, Artificial Sequence
  Synthetic polynucleotide sequence
gttttggaattaacaacttttaatccagatataaatctgttctgtagcatttcggtcatatttgaagtct

SEQ ID NO: 614 - DNA, 70 bp, Artificial Sequence
  Synthetic polynucleotide sequence
ccgtcgatgaagaccctgtttgtggacagctacagtgagatgcttttctttctgcagtcactgttcatgc

SEQ ID NO: 615 - DNA, 70 bp, Artificial Sequence
  Synthetic polynucleotide sequence
ctcttacaagatcgaggagatctcaccaagcacattaaaaaacagcctcttcccatcgcctattagctcc

SEQ ID NO: 616 - DNA, 70 bp, Artificial Sequence
  Synthetic polynucleotide sequence
aaatctcatagctttatgttttccccctcaaggagctattatgccaactttggggtgcctgtaaaaacag

SEQ ID NO: 617 - DNA, 70 bp, Artificial Sequence
  Synthetic polynucleotide sequence
aggaaaaatctttctgatcttttacggccttgttgggtgttccagcaccatcttgttcttcaacctcttc

SEQ ID NO: 618 - DNA, 70 bp, Artificial Sequence
  Synthetic polynucleotide sequence
cttctgccacgtgcacaagctggagaggtgcgcccgcgacaacctgggcttttcgcccccctcgagcccg

SEQ ID NO: 619 - DNA, 70 bp, Artificial Sequence
  Synthetic polynucleotide sequence
tgtgaactccatgagctgaaaaaattcagaaaaatgttctatgtgaagaaggacaaggacgaggatcagg

SEQ ID NO: 620 - DNA, 70 bp, Artificial Sequence
  Synthetic polynucleotide sequence
tgaaaacaaaatctgagaatggattggaatttacaagctcaggctcagccaacactgagaccaccaaagt

SEQ ID NO: 621 - DNA, 70 bp, Artificial Sequence
  Synthetic polynucleotide sequence
ttttggtcaaggctgcccagcagctacgcttcgtgcgccagtggtccgtctttggcaagacattatgccg

SEQ ID NO: 622 - DNA, 70 bp, Artificial Sequence
  Synthetic polynucleotide sequence
tttagaaaatacaacaataataatggagatgagattattccaactaatactctggaagagatcatgctag

SEQ ID NO: 623 - DNA, 70 bp, Artificial Sequence
  Synthetic polynucleotide sequence
agaatcagtgctcaggctcagaaatcctggatagaaagagcattttataaaagagaatgtgtccacatca

SEQ ID NO: 624 - DNA, 70 bp, Artificial Sequence
  Synthetic polynucleotide sequence
agtcatatatactcttatggaaaaggactcttatcccaggttcctcaaatcacatatttacttaaatctt

SEQ ID NO: 625 - DNA, 70 bp, Artificial Sequence
  Synthetic polynucleotide sequence
aaaggaagctccaaaagagataaacatagattttcaaaccaaaactctgattgcccagaatatacaagaa

SEQ ID NO: 626 - DNA, 70 bp, Artificial Sequence
  Synthetic polynucleotide sequence
tggcttgtgaggacttcaagaaggtcaagtcacagtccaagatggcatccaaggccaagaagatctttgc

SEQ ID NO: 627 - DNA, 70 bp, Artificial Sequence
  Synthetic polynucleotide sequence
tgaagagtacaagaaaatcaaatcaccatctaaactaagtcccaaggccaaaaagatctataatgaattc

SEQ ID NO: 628 - DNA, 70 bp, Artificial Sequence
  Synthetic polynucleotide sequence
ttacaagaagatcaagtcccctgccaagatggctgagaaggcaaagcaaatttatgaagaattcattcaa

SEQ ID NO: 629 - DNA, 70 bp, Artificial Sequence
  Synthetic polynucleotide sequence
caaccaatcaggaaaacaacaaaagaggacatccggaaacagataacatttttgaacgcacagatcgaca

SEQ ID NO: 630 - DNA, 70 bp, Artificial Sequence
  Synthetic polynucleotide sequence
atagacatcggttaaaaatgtcaaaagtcgctgacagtctactaagttacacggaacagtatttagaata

SEQ ID NO: 631 - DNA, 70 bp, Artificial Sequence
  Synthetic polynucleotide sequence
gtgtagtgatttcacagctattcttccagacaaacccaaccgcgctctcaagagattatcgacagaagaa

SEQ ID NO: 632 - DNA, 70 bp, Artificial Sequence
  Synthetic polynucleotide sequence
tgaagtcaaggtaaaccagaaacaaacagtcgttgctgtcaaaaaagagatcatgtattaccaacaggcc

SEQ ID NO: 633 - DNA, 70 bp, Artificial Sequence
  Synthetic polynucleotide sequence
attttaagaaaatgcaagataagacgcagatgcaggaaaaggcaaaggagatctacatgacctttctgtc

SEQ ID NO: 634 - DNA, 70 bp, Artificial Sequence
  Synthetic polynucleotide sequence
ccaagaagaacatccgaaaacgggggaccctggtggattatgagaaggactgctatgaccggctacacaa

SEQ ID NO: 635 - DNA, 70 bp, Artificial Sequence
  Synthetic polynucleotide sequence
ggagaggaaagaacactaggcaagtctaattctattaaaataaaaggagaaaatggaaaaaatgctaggg

SEQ ID NO: 636 - DNA, 70 bp, Artificial Sequence
  Synthetic polynucleotide sequence
ctatatgcatatggaaagggattcctaccccagatttctaaagtcagaaatgtaccaaaaacttttgaaa

SEQ ID NO: 637 - DNA, 70 bp, Artificial Sequence
  Synthetic polynucleotide sequence
tccttgaactccaccaccgactcagccctctgacagctacccaacagtccaggacagctgcatggcaccc

SEQ ID NO: 638 - DNA, 70 bp, Artificial Sequence
  Synthetic polynucleotide sequence
tgggagtactggcaagttcgagtggggcagtaaacacagcaaagagaatagaaacttctcagaagatgtg

SEQ ID NO: 639 - DNA, 70 bp, Artificial Sequence
  Synthetic polynucleotide sequence
agacttaaagaaggagcagaacaaaaaagtaattgaagaaaaggctaggatgatatatgaagattacatt

SEQ ID NO: 640 - DNA, 70 bp, Artificial Sequence
  Synthetic polynucleotide sequence
caaccagcatgtggtagacgagaaggcgaggctcatctacgaggactacgtatccatcctgtcccccaag

SEQ ID NO: 641 - DNA, 70 bp, Artificial Sequence
  Synthetic polynucleotide sequence
ctatccaccaagagcttccccttcttcaactctatgaacgtcaaccccctgtcatcacagagcatgtttt

SEQ ID NO: 642 - DNA, 70 bp, Artificial Sequence
  Synthetic polynucleotide sequence
aactgaaaaaggaagctaataaaaacattattgaagagaaagcaaggataatctatgaagactacatttc

SEQ ID NO: 643 - DNA, 70 bp, Artificial Sequence
  Synthetic polynucleotide sequence
atcctcgacaccaagtcttgcctcaagaacaaaaccaaggagaacgtggacgtgcccctggtcatctgcg

SEQ ID NO: 644 - DNA, 70 bp, Artificial Sequence
  Synthetic polynucleotide sequence
atagtatggaaaatatggaacttatgaagttaacaccagaaaaggtacagaactggaacagtgaaattct

SEQ ID NO: 645 - DNA, 70 bp, Artificial Sequence
  Synthetic polynucleotide sequence
tttatggaatgatcctggaatccaggaatgctatgatagacgacgagaatatcaattatctgactctacc

SEQ ID NO: 646 - DNA, 70 bp, Artificial Sequence
  Synthetic polynucleotide sequence
ttaatgggtttaatggagacagtgagaaggcaaccaaagtgcaggacatcaaaaacaacctgaaagaggc

SEQ ID NO: 647 - DNA, 70 bp, Artificial Sequence
  Synthetic polynucleotide sequence
ccagtttgtcttcgacgcggtgacagacgtcatcatacagaacaatctcaagtacattggcctttgctga

SEQ ID NO: 648 - DNA, 70 bp, Artificial Sequence
  Synthetic polynucleotide sequence
ctgctttgagaacgtgacatccatcatgtttctcgtcgccctcagcgaatacgaccaagtcctggtggag

SEQ ID NO: 649 - DNA, 70 bp, Artificial Sequence
  Synthetic polynucleotide sequence
agcaagatatcctgctggctaggaaagccaccaagggaattgtggagcatgacttcgttattaagaagat

SEQ ID NO: 650 - DNA, 70 bp, Artificial Sequence
  Synthetic polynucleotide sequence
cttaaacaagacagacttgcttgaggagaaggtgcaaattgtgagcatcaaagactatttcctagaattt

SEQ ID NO: 651 - DNA, 70 bp, Artificial Sequence
  Synthetic polynucleotide sequence
caagaaggatcttttggaagagaaaatcatgtactctcatctaattagctatttcccagaatacacagga

SEQ ID NO: 652 - DNA, 70 bp, Artificial Sequence
  Synthetic polynucleotide sequence
tgtttgggactatcctggaactaccctggttcaaaagcacatccgtcatcctctttctcaacaaaaccga

SEQ ID NO: 653 - DNA, 70 bp, Artificial Sequence
  Synthetic polynucleotide sequence
tggttattcagaagaggagtgtaaacaatacaaagcagtggtctacagtaacaccatccagtcaattatt

SEQ ID NO: 654 - DNA, 70 bp, Artificial Sequence
  Synthetic polynucleotide sequence
tgaggcagccagctacatccagagtaagtttgaggacctgaataagcgcaaagacaccaaggagatctac

SEQ ID NO: 655 - DNA, 70 bp, Artificial Sequence
  Synthetic polynucleotide sequence
taaacaatataaagtagttgtctacagcaatactatacagtccatcattgcaatcataagagccatggga

SEQ ID NO: 656 - DNA, 70 bp, Artificial Sequence
  Synthetic polynucleotide sequence
ctacatccaagcacaatttgaaagcaaaaaccgctcacccaacaaagaaatatattgtcacatgacttgt

SEQ ID NO: 657 - DNA, 70 bp, Artificial Sequence
  Synthetic polynucleotide sequence
tacggtctacttgatgaattcctggccttttggggatgtgctgtgcaagatagtaatttccattgattac

SEQ ID NO: 658 - DNA, 70 bp, Artificial Sequence
  Synthetic polynucleotide sequence
tagattgtacactaacattctctcatccaacctggtactgggaaaacctgctgaagatctgtgttttcat

SEQ ID NO: 659 - DNA, 70 bp, Artificial Sequence
  Synthetic polynucleotide sequence
catctgcatcttcctcttctccttcatcgtccccgtgctcgtcatctctgtctgctacagcctcatgatc

SEQ ID NO: 660 - DNA, 70 bp, Artificial Sequence
  Synthetic polynucleotide sequence
tgatcaacatctgtatctgggtcctggcctcaggcgttggcgtgcccatcatggtcatggctgtgacccg

SEQ ID NO: 661 - DNA, 70 bp, Artificial Sequence
  Synthetic polynucleotide sequence
caccatcaaccccatgtgctacgcactctgcaacaaagccttccgggacacctttcgcctgctgctgctt

SEQ ID NO: 662 - DNA, 70 bp, Artificial Sequence
  Synthetic polynucleotide sequence
tgaaaacacagtttccacttccctgggccattccaaagatgagaactctaagcaaacatgcatcagaatt

SEQ ID NO: 663 - DNA, 70 bp, Artificial Sequence
  Synthetic polynucleotide sequence
atgcctgtcaccattatgactattttatactggaggatctataaggaaactgaaaagcgtaccaaagagc

SEQ ID NO: 664 - DNA, 70 bp, Artificial Sequence
  Synthetic polynucleotide sequence
cctccagatggtccaagatccagattgtgacgaagcagacaggcaatgagtgtgtgacagccattgagat

SEQ ID NO: 665 - DNA, 70 bp, Artificial Sequence
  Synthetic polynucleotide sequence
gaagaattcagtgctgaagagactgaggaaacttttgtgaaagctgaaactgaaaaaagtgactatgaca

SEQ ID NO: 666 - DNA, 70 bp, Artificial Sequence
  Synthetic polynucleotide sequence
tttaagatgtccttcaaaagagaaactaaagtcctgaagactctgtcggtgatcatgggtgtgtttgtgt

SEQ ID NO: 667 - DNA, 70 bp, Artificial Sequence
  Synthetic polynucleotide sequence
atggtgaccctgttgctgagtctgtctgggagctggactgcgagggggagatttctttagacaaaataac

SEQ ID NO: 668 - DNA, 70 bp, Artificial Sequence
  Synthetic polynucleotide sequence
agaaagctctcccagcagaaggagaagaaagccactcagatgctcgccattgttctcggcgtgttcatca

SEQ ID NO: 669 - DNA, 70 bp, Artificial Sequence
  Synthetic polynucleotide sequence
aatctatgtggtgctgaaacaaaggagacggaaaaggatcctcactcgacagaacagtcagtgcaacagt

SEQ ID NO: 670 - DNA, 70 bp, Artificial Sequence
  Synthetic polynucleotide sequence
cgtcatctacactgtcttcaacgccgagttccgcaacgtcttccgcaaggccctgcgtgcctgctgctga

SEQ ID NO: 671 - DNA, 70 bp, Artificial Sequence
  Synthetic polynucleotide sequence
tttactcaacatttggagctttctacatcccactggcattgattttgatcctttactacaaaatatatag

SEQ ID NO: 672 - DNA, 70 bp, Artificial Sequence
  Synthetic polynucleotide sequence
ataatcaattggctgggctactccaactctctgcttaaccccgtcatttacgcatacttcaacaaggact

SEQ ID NO: 673 - DNA, 70 bp, Artificial Sequence
  Synthetic polynucleotide sequence
actccctcatcaatccaataatctacactgtgtttaatgaagagtttcggcaagcttttcagaaaattgt

SEQ ID NO: 674 - DNA, 70 bp, Artificial Sequence
  Synthetic polynucleotide sequence
aactccctcatcaaccccataatctataccatgtccaatgaggactttaaacaagcattccataaactga

SEQ ID NO: 675 - DNA, 70 bp, Artificial Sequence
  Synthetic polynucleotide sequence
caccatttactccacgctgggtgcgttttatatccccttgactttgatactgattctctattaccggatt

SEQ ID NO: 676 - DNA, 70 bp, Artificial Sequence
  Synthetic polynucleotide sequence
tgtgtcatttttcattcccttaaccatcatggtgatcacctactttctaactatcaagtcactccagaaa

SEQ ID NO: 677 - DNA, 70 bp, Artificial Sequence
  Synthetic polynucleotide sequence
gtgtttttcctctttttgcttatgtggtgtcccttctttattacaaatataactttagttttatgtggat

SEQ ID NO: 678 - DNA, 70 bp, Artificial Sequence
  Synthetic polynucleotide sequence
cattaccaatattctgtctgttctttgtgagaagtcctgtaaccaaaagctcatggaaaagcttctgaat

SEQ ID NO: 679 - DNA, 70 bp, Artificial Sequence
  Synthetic polynucleotide sequence
aagtctccaaatatcccgtacgtgtatattcggcatcaaggcgaagttcagaactacaagccccttcagg

SEQ ID NO: 680 - DNA, 70 bp, Artificial Sequence
  Synthetic polynucleotide sequence
tccatgctatattggatgtggatgcagagaatcaaatattaaagacaagtgtatggtaccaagaggtctg

SEQ ID NO: 681 - DNA, 70 bp, Artificial Sequence
  Synthetic polynucleotide sequence
aggctggaataacattggcataattgatttgatagaaaagaggaagttcaaccagaactctaactctacg

SEQ ID NO: 682 - DNA, 70 bp, Artificial Sequence
  Synthetic polynucleotide sequence
acatggctgggttactgtaacagcaccatgaaccccatcatctacccactcttcatgcgggacttcaagc

SEQ ID NO: 683 - DNA, 70 bp, Artificial Sequence
  Synthetic polynucleotide sequence
tgatgataaggtgtgcttgatcagccaggactttggctatacgatttactctaccgcagtggcattttat

SEQ ID NO: 684 - DNA, 70 bp, Artificial Sequence
  Synthetic polynucleotide sequence
gaggtcaaattccagcatgataacttcacagaggaagcaaaagatatttgcaggctcttcttggctaaga

SEQ ID NO: 685 - DNA, 70 bp, Artificial Sequence
  Synthetic polynucleotide sequence
tgcctgtgaagactttaagaaaacgaaaaatgcagacaaaattgcttccaaagccaagatgatttattct

SEQ ID NO: 686 - DNA, 70 bp, Artificial Sequence
  Synthetic polynucleotide sequence
gaggatgcggagaagaaccagaaaatcatgcagtggatcattgagggggaaaaggagatcagcaggcacc

SEQ ID NO: 687 - DNA, 70 bp, Artificial Sequence
  Synthetic polynucleotide sequence
ttccagaaattcattgagagcgataagttcacacggttttgccagtggaagaatgtggagctcaacatcc

SEQ ID NO: 688 - DNA, 70 bp, Artificial Sequence
  Synthetic polynucleotide sequence
aatgtatgcaatgaaatgcttagataagaagaggatcaaaatgaaacaaggagaaacattagctttaaat

SEQ ID NO: 689 - DNA, 70 bp, Artificial Sequence
  Synthetic polynucleotide sequence
atgaagttactccagatgaaaaactgggagagaaagggaaggaaattatgaccaagtacctcaccccaaa

SEQ ID NO: 690 - DNA, 70 bp, Artificial Sequence
  Synthetic polynucleotide sequence
aggatgttctggacattgaacagttctctacggtcaagggcgtggagctggagcctaccgaccaggactt

SEQ ID NO: 691 - DNA, 70 bp, Artificial Sequence
  Synthetic polynucleotide sequence
taaacaaactgcgagaactaaatgagaaacttgaatataaaaggcaagctctaaattctattcaaaatgc

SEQ ID NO: 692 - DNA, 70 bp, Artificial Sequence
  Synthetic polynucleotide sequence
aagatttcaagaaaagcaagggacctcaacaaattcaccttaaagcaaaagcaatatatgagaaatttat

SEQ ID NO: 693 - DNA, 70 bp, Artificial Sequence
  Synthetic polynucleotide sequence
tttaaagagcagctcagcaaaaagggaaattataggtattacttcaaaaaagcaagcgatgagtttgcct

SEQ ID NO: 694 - DNA, 70 bp, Artificial Sequence
  Synthetic polynucleotide sequence
gcaaaaagctacaaaaaaaaagaataaagaagaggaaaggtgaagctatggctctaaatgagaaaagaat

SEQ ID NO: 695 - DNA, 70 bp, Artificial Sequence
  Synthetic polynucleotide sequence
caagaagcggctgaagaagaggaagggctaccagggtgctatggtggagaagaagattctgatgaaagta

SEQ ID NO: 696 - DNA, 70 bp, Artificial Sequence
  Synthetic polynucleotide sequence
actaatgaaaagtatagaacaagatgcagtgaatacttttaccaaatatatatctccagatgctgctaaa

SEQ ID NO: 697 - DNA, 70 bp, Artificial Sequence
  Synthetic polynucleotide sequence
ttttagagagaagacaagaatataataagcagaaaaaaaaattggcagtcctagaagacgaaaaatctgg

SEQ ID NO: 698 - DNA, 70 bp, Artificial Sequence
  Synthetic polynucleotide sequence
ccagtattggtattatacactaagcgatgatgaatcttttcttcttgaaattaggcagactcttcaaaac

SEQ ID NO: 699 - DNA, 70 bp, Artificial Sequence
  Synthetic polynucleotide sequence
aggttgagcccagtgacaccatcgagaatgtcaaggcaaagatccaagataaggaaggcatccctcctga

SEQ ID NO: 700 - DNA, 70 bp, Artificial Sequence
  Synthetic polynucleotide sequence
ttgctgaacttgatacattaagtgaagagtcatacaaagacagcacgctaataatgcaattactgagaga

SEQ ID NO: 701 - DNA, 70 bp, Artificial Sequence
  Synthetic polynucleotide sequence
gttgtaggatatgcccttgactataatgaatacttcagggatttgaatcatgtttgtgtcattagtgaaa

SEQ ID NO: 702 - DNA, 70 bp, Artificial Sequence
  Synthetic polynucleotide sequence
gcattgccctcaacgaccactttgtcaagctcatttcctggtatgacaacgaatttggctacagcaacag

SEQ ID NO: 703 - DNA, 70 bp, Artificial Sequence
  Synthetic polynucleotide sequence
aacctacactaaccttaaccgccttattagccagattgtgtcctccatcactgcttccctgagatttgat

SEQ ID NO: 704 - DNA, 70 bp, Artificial Sequence
  Synthetic polynucleotide sequence
tggagctgtggtcaccgctatgatgtgtaggaggaagagctcaggtggaaaaggagggagctgctctcag

SEQ ID NO: 705 - DNA, 70 bp, Artificial Sequence
  Synthetic polynucleotide sequence
atgacccagatcatgtttgagaccttcaacaccccagccatgtacgttgctatccaggctgtgctatccc

SEQ ID NO: 706 - DNA, 70 bp, Artificial Sequence
  Synthetic polynucleotide sequence
gaagaagaaacagctcatgaggctacggaaacaggccgagaagaacgtggagaagaaaattgacaaatac

SEQ ID NO: 707 - DNA, 70 bp, Artificial Sequence
  Synthetic polynucleotide sequence
atgagggcaagatgaagctggattacatcctgggcctgaagatagaggatttcttagagagacgcctgca

SEQ ID NO: 708 - DNA, 70 bp, Artificial Sequence
  Synthetic polynucleotide sequence
ggtggtgaacacggtgggctcagagaatccggatgaagccgcttttttatactaagttggcattataaaa

SEQ ID NO: 709 - DNA, 70 bp, Artificial Sequence
  Synthetic polynucleotide sequence
tttatgctctgaggctttattgcttaattttgctaattctttgccttgcctgtatgatttattggatgtt

SEQ ID NO: 710 - DNA, 70 bp, Artificial Sequence
  Synthetic polynucleotide sequence
caaatatgtatccgctcatgagacaataaccctgataaatgcttcaataatattgaaaaaggaagagtat

SEQ ID NO: 711 - DNA, 70 bp, Artificial Sequence
  Synthetic polynucleotide sequence
cctggaaaggctggaggagaaccattacaacacctatatatccaagaagcatgcagagaagaattggttt

SEQ ID NO: 712 - DNA, 70 bp, Artificial Sequence
  Synthetic polynucleotide sequence
tataaatgtgaatttaatcaattcctttcatagttttataattctctggcagttccttatgatagagttt

SEQ ID NO: 713 - DNA, 70 bp, Artificial Sequence
  Synthetic polynucleotide sequence
cttgaagactgctggagttaataccaccgacaaagagatggaggtgcttcacttaagaaatgtctccttt

SEQ ID NO: 714 - DNA, 70 bp, Artificial Sequence
  Synthetic polynucleotide sequence
accctgacacaagaagttcttgttcttcaggagatgattctgttttttctccagaccccatgccttacga

SEQ ID NO: 715 - DNA, 70 bp, Artificial Sequence
  Synthetic polynucleotide sequence
atgccacatgtcagtgtttgaaaggatttgctggggatggaaaactatgttctgatatagatgaatgtga

SEQ ID NO: 716 - DNA, 70 bp, Artificial Sequence
  Synthetic polynucleotide sequence
agataagtgatggagatgtgataatttcaggaaacaaaaatttgtgctatgcaaatacaataaactggaa

SEQ ID NO: 717 - DNA, 70 bp, Artificial Sequence
  Synthetic polynucleotide sequence
tggccatcaaagtgttgagggaaaacacatcccccaaagccaacaaagaaatcttagacgaagcatacgt

SEQ ID NO: 718 - DNA, 70 bp, Artificial Sequence
  Synthetic polynucleotide sequence
cgaatgctatttttgtttcatttaatattacaattattctgtcttcattggagctttggatagatgaaaa

SEQ ID NO: 719 - DNA, 70 bp, Artificial Sequence
  Synthetic polynucleotide sequence
tgctacaagatgaatacaaaaggaaataaatttggatactgcaaaaacaaggaaaacagatttcttccct

SEQ ID NO: 720 - DNA, 70 bp, Artificial Sequence
  Synthetic polynucleotide sequence
aggacgttgccaggacttacacgtttacagatccagcaactgctctgcccagtgccacaaccatggggtg

SEQ ID NO: 721 - DNA, 70 bp, Artificial Sequence
  Synthetic polynucleotide sequence
tggcaaactacttggatagtatgtatattatgttaaatattcgaattgtgctagttggactggagatttg

SEQ ID NO: 722 - DNA, 70 bp, Artificial Sequence
  Synthetic polynucleotide sequence
cactctgtagtattagaaatataagccaagttcttgagaagaagagaaacaactgttttgttgaatctgg

SEQ ID NO: 723 - DNA, 70 bp, Artificial Sequence
  Synthetic polynucleotide sequence
aggtccgccggggccaccctacagtgcacagtgaaaccaagtatgtggagctaattgtgatcaacgacca

SEQ ID NO: 724 - DNA, 70 bp, Artificial Sequence
  Synthetic polynucleotide sequence
gcttgcaggcacaaagtgtgcagatggaaaaatctgcctgaatcgtcaatgtcaaaatattagtgtcttt

SEQ ID NO: 725 - DNA, 70 bp, Artificial Sequence
  Synthetic polynucleotide sequence
gcccttggagccccaggtccttcaggacgatctcccaattagcctcaaaaaggtgcttcagaccagtctg

SEQ ID NO: 726 - DNA, 70 bp, Artificial Sequence
  Synthetic polynucleotide sequence
aagagatctacagacttcaacacatgtagaaacactactaactttttcagctttgaaaaggcattttaaa

SEQ ID NO: 727 - DNA, 70 bp, Artificial Sequence
  Synthetic polynucleotide sequence
atctttaaaagaaatgaaataagtaaatcatgtaacagagagaatgcagagtataatcgtaattcatccg

SEQ ID NO: 728 - DNA, 70 bp, Artificial Sequence
  Synthetic polynucleotide sequence
tatgaaaagacgtgtaataaccatgatatacaatgtaaagagatttttggccaagatgcaaggagtgcat

SEQ ID NO: 729 - DNA, 70 bp, Artificial Sequence
  Synthetic polynucleotide sequence
taaaaaattcacttataagtatacttggcctagcctatgttgcaggaatatgtcgtccacctattgattg

SEQ ID NO: 730 - DNA, 70 bp, Artificial Sequence
  Synthetic polynucleotide sequence
tatcctcgtaatgtagaagaagaaaccaaatacattgaactgatgattgtgaatgatcaccttatgttta

SEQ ID NO: 731 - DNA, 70 bp, Artificial Sequence
  Synthetic polynucleotide sequence
agaaagaaatcacactgccttcaagactcatatattacatcaaccaagactcggaaagcccttatcacgt

SEQ ID NO: 732 - DNA, 70 bp, Artificial Sequence
  Synthetic polynucleotide sequence
agcatgcactcttcaagtataaccctgatgaaaagaattatgacagcacctgtgggatggatggtgtgtt

SEQ ID NO: 733 - DNA, 70 bp, Artificial Sequence
  Synthetic polynucleotide sequence
gttattgttaatatagtggattccattttggatgtcattggtgttaaggtgttattatttggtttggaga

SEQ ID NO: 734 - DNA, 70 bp, Artificial Sequence
  Synthetic polynucleotide sequence
ggaagaatctaaagcaaacattgaaagtaaacgacccaaagcaaagagtgtcaagaaacaaaaaaagtaa

SEQ ID NO: 735 - DNA, 70 bp, Artificial Sequence
  Synthetic polynucleotide sequence
gcctctgccagcagtctcgcctgacccccaagcagatcaagtccagatgccaagatcctgcctctggtga

SEQ ID NO: 736 - DNA, 70 bp, Artificial Sequence
  Synthetic polynucleotide sequence
atgtactggcgaagaatatttggtaaagcagggcatcgatctctcaccctggggcttgtggaagaatcac

SEQ ID NO: 737 - DNA, 70 bp, Artificial Sequence
  Synthetic polynucleotide sequence
tgatacaagtgaagagtgagctgcccctggatccgctgccagtccccactgaggaaggaaaccccctcct

SEQ ID NO: 738 - DNA, 70 bp, Artificial Sequence
  Synthetic polynucleotide sequence
gcagcccctgctggccgtacagttcaccaatcttaccatggacactgaaattcgcatagagtgtaaggcg

SEQ ID NO: 739 - DNA, 70 bp, Artificial Sequence
  Synthetic polynucleotide sequence
ggcattatacagcggcctctttgcctatggaggatggaattacttgaatttcgtcacagaggaaatgatc

SEQ ID NO: 740 - DNA, 70 bp, Artificial Sequence
  Synthetic polynucleotide sequence
cgagtcactgcctaataaatatagcactaaagtaggagacaaaggaactcagctctctggtggccagaaa

SEQ ID NO: 741 - DNA, 70 bp, Artificial Sequence
  Synthetic polynucleotide sequence
gggccagccactgccagacacggtgcaggacctggagagcaggaaagggaaacagacgcgacagcaacaa

SEQ ID NO: 742 - DNA, 70 bp, Artificial Sequence
  Synthetic polynucleotide sequence
tagagactaatgctgacatcattaaaaatgaaaatgaagacaaacaaaaagaagaggtcaagaaggaaaa

SEQ ID NO: 743 - DNA, 70 bp, Artificial Sequence
  Synthetic polynucleotide sequence
tacagggattggcttcgtcatcactctgactggagtccctgcgtattatctctttattatatgggacaag

SEQ ID NO: 744 - DNA, 70 bp, Artificial Sequence
  Synthetic polynucleotide sequence
aaagaaaaacagactggaaaagagtaacccatatttcatgtcaggggccaattcacagaaacagatgtga

SEQ ID NO: 745 - DNA, 69 bp, Artificial Sequence
  Synthetic polynucleotide sequence
gaccggatctctgggtcacgttgattctcacagctcattagttggctactgtcctcaggaagatgcctt

SEQ ID NO: 746 - DNA, 69 bp, Artificial Sequence
  Synthetic polynucleotide sequence
aatcacttttttctatgatggatatgaaaaattcaaggcagtatgcacagaatggacgagtgcagccca

SEQ ID NO: 747 - DNA, 69 bp, Artificial Sequence
  Synthetic polynucleotide sequence
gaggcatttgactaagccaacctcctctcacagcctctgtatctctgcaggccatactggttccattgt

SEQ ID NO: 748 - DNA, 70 bp, Artificial Sequence
  Synthetic polynucleotide sequence
cccaggtattcattttgacctaatttcacctcaagtggagaatcgctgaccttgaaccagcgcccttcga

SEQ ID NO: 749 - DNA, 69 bp, Artificial Sequence
  Synthetic polynucleotide sequence
tggtgcttcccacggaggagttttggcagccagacttctggaggaattggttgtatagaagatcctagt

SEQ ID NO: 750 - DNA, 69 bp, Artificial Sequence
  Synthetic polynucleotide sequence
ggattgctggatggaaaccctggaataggctacttgatggctctcaagaccttagaaccccagaaccat

SEQ ID NO: 751 - DNA, 69 bp, Artificial Sequence
  Synthetic polynucleotide sequence
cagattctgcatttgcgatgttactagcagcagaagtcagattgtagaggtcctggcggctgattctag

SEQ ID NO: 752 - DNA, 69 bp, Artificial Sequence
  Synthetic polynucleotide sequence
gtagttccaaaaacccatctaaatttcttgagttcctgaattttgaacaggattacctggagcctggag

SEQ ID NO: 753 - DNA, 69 bp, Artificial Sequence
  Synthetic polynucleotide sequence
ttttctggtggatttaatgctgactcactggtacaaacagctgttgaagctcagagctggaggtgagct

SEQ ID NO: 754 - DNA, 70 bp, Artificial Sequence
  Synthetic polynucleotide sequence
ccttattcccaaatgtctctatccttttgactggagcatcttctgcacaaccttgggagcccatccaagg

SEQ ID NO: 755 - DNA, 21 bp, Artificial Sequence
  Synthetic polynucleotide sequence
aaaggagctcaaatgagtgga

SEQ ID NO: 756 - DNA, 21 bp, Artificial Sequence
  Synthetic polynucleotide sequence
aagtggagaatcgctgacctt

SEQ ID NO: 757 - DNA, 21 bp, Artificial Sequence
  Synthetic polynucleotide sequence
aacagttttctcgatggcctg

SEQ ID NO: 758 - DNA, 21 bp, Artificial Sequence
  Synthetic polynucleotide sequence
aagcgaagcagtggttcaggt

(19) United States
(12) Patent Application Publication
(10) Pub. No.: US 2005/0208512 A1
(43) Pub. Date: Sep. 22, 2005

(54) Determining the chemosensitivity of cells to cytotoxic agents

(75) Inventors: Wolfgang Sadee, Columbus, OH (US); Ying Huang, Newton, MA (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 10/957,432

(22) Filed: Oct. 1, 2004

Related U.S. Application Data
(60) Provisional application No. 60/508,260, filed on Oct. 1, 2003.

(51) Int. Cl. C12Q 1/68; C12M 1/34

(52) U.S. Cl. 435/6; 435/287.2

(57) ABSTRACT

Gene expression analysis systems are provided for identifying the chemosensitivity gene profile of a cancer cell, the analysis systems comprising a plurality of polynucleotide probes, wherein each of said polynucleotide probes comprises a polynucleotide sequence that is complementary to a target region of a gene that encodes a protein associated with transport of molecules into and out of cells and that is a marker for the sensitivity or resistance of cancer cells to cytotoxic agents.

### PRIORITY CLAIM

This application claims priority to U.S. Provisional Patent Application 60/508,260, filed Oct. 1, 2003, which is incorporated herein by reference, in its entirety.

### STATEMENT ON FEDERALLY FUNDED RESEARCH

The present invention was made with support from National Institutes of Health Grant NOs. GM61390, GM43102 and GM99004. The United States Government has certain rights in the invention.

### BACKGROUND

Membrane transporters, ion exchangers, and ion channels are proteins involved in drug uptake and secretion by cells, and influence, if not determine, cellular drug targeting. Thus, these factors are expected to play a critical role in chemosensitivity. Membrane transporters, ion exchangers and ion channels are encoded by numerous gene families, together comprising 4.1% of genes in the human genome. Collectively, these proteins are believed to provide nutrients to cells across lipid bilayer membranes, provide the means for transporting amino acids, dipeptides, monosaccharides, monocarboxylic acids, organic cations, phosphates, nucleosides, and water-soluble vitamins, remove unwanted materials from the cell, and establish the electrochemical gradient across cellular membranes, among other functions. Their physiological relevance is underscored by the discovery of numerous disorders that are caused by mutations in membrane transporter genes.

Transporters are thought to play a key role in drug entry into cells and expulsion from tissues endowed with efflux pumps. The electrochemical gradient across membranes is also germane to drug partitioning into and out of cells and cell organelles, such as mitochondria. Drug absorption appears to occur predominantly via passive transcellular and paracellular transport mechanisms. However, recent studies indicate that carrier-mediated drug transport may play a more important role than previously thought. For a majority of drugs it remains unknown that transporters play a role in their absorption and targeting in the body.

Transporter proteins have been shown to have some involvement in the efficacy of cancer therapies. Use of cytotoxic agents is an important mode of treatment for many forms of cancer. However, only a limited proportion of cancer patients respond favorably to most chemotherapeutic drugs, and drug efficacy varies widely among these patients. Treatment according to standard drug protocols can result in the selection of more resistant and aggressive cancer cells. Previous studies have revealed several genetic factors that influence the chemosensitivity of cancer cells, including genes involved in drug uptake and secretion, drug metabolism, DNA repair and apoptosis. But due to the lack of predictability regarding the genetic bases for development of drug resistance, there are few clear options for treatment. Thus, cancer patients are often treated according to a standard regimen without any consideration of individual differences in chemosensitivity. This approach commonly leads to the development of resistance of the patient's cancer during treatment, and often results in treatment failure.

What are lacking are tools for predicting the likelihood that a particular cancer will be responsive to a chemotherapy regimen, and in particular, identifying agents to which the cancer will be sensitive or resistant. Also lacking are tools for profiling genetic factors influencing sensitivity and resistance of cancers to therapeutic agents. Such tools, and the resulting gene expression profiles, would be predictive of treatment response of a cancer to a particular drug, and would allow for increased predictability regarding chemosensitivity or chemoresistance of cancers to enable the design of optimal treatment regimens for patients. Such tools would likewise enable the identification of new drugs that modulate expression of genes that affect chemosensitivity, particularly new agents that alter expression of these genes to overcome drug resistance or enhance chemosensitivity.

### SUMMARY OF THE INVENTION

In one aspect, the present invention provides a gene expression analysis system, for example, arrays, for identifying the chemosensitivity gene profile of a cancer cell, the analysis system comprising a plurality of polynucleotide probes, wherein each of said polynucleotide probes comprises a polynucleotide sequence that is complementary to a target region of a gene that encodes a protein associated with transport of molecules into and out of cells and that is a marker for the sensitivity or resistance of cancer cells to cytotoxic agents. In one embodiment, the plurality of polynucleotide probes comprises at least two or more probes, each of which comprises a polynucleotide sequence that is complementary to a target region of a chemosensitivity gene listed in one of FIG. 9 and FIG. 10, or in one of Tables 1-6. Provided in FIG. 8 are examples of polynucleotide probes that are complementary to and hybridize with target regions of chemosensitivity genes. The present invention also provides arrays comprising a plurality of oligonucleotide probes designed to be complementary to and hybridize under stringent conditions with a gene listed in one of FIG. 9 and FIG. 10, or in one of Tables 1-6. The present invention also provides arrays comprising a plurality of oligonucleotides, wherein: a) the oligonucleotides are chosen from the nucleic acid sequences listed in FIG. 8, and wherein the array comprises 10 or more of said oligonucleotides; or b) the oligonucleotides comprise nucleotide probes designed to be complementary to, or hybridize under stringent conditions with, 10 or more chemosensitivity genes listed in listed in one of FIG. 9 and FIG. 10, or in one of Tables 1-6. In some embodiments, the oligonucleotides comprise nucleotide probes designed to be complementary to, or hybridize under stringent conditions with target regions of 10, 20, 30, 40, 50, 60, 70, 80, 90, 100, 200, or more chemosensitivity genes listed in one of FIG. 9 and FIG. 10, or in one of Tables 1-6.

In another aspect, the present invention provides methods for detecting the chemosensitivity gene expression profile for a cancer cell. The chemosensitivity gene expression profile reflects the expression levels of a plurality of target polynucleotides in a sample, wherein the target polynucleotides encode gene products that are markers for cancer cell chemosensitivity. In one embodiment, the method comprises contacting a polynucleotide sample obtained from cells of the specific cancer of interest to polynucleotide probes to detect and measure the amount of target polynucleotides in the sample. The measured levels of expression of target polynucleotides provides an expression profile for the cancer cells that is compared to the drug-gene correlations listed in FIG. 9.

Expression in the cancer cells of a gene that has a positive correlation (r>0) with a drug indicates that the cancer cells would be sensitive to the drug. Expression in the cancer cells of a gene that has a negative correlation (r<0) with a drug indicates that the cancer cells would be resistant to the drug. The chemosensitivity expression profile can be used, for example: (a) in the prediction of the chemosensitivity of a particular cancer cell or cell type to a therapeutic agent; (b) in the choice of drug therapy for a patient in need of the same; (c) in the identification of targets for altering the chemosensitivity of a cancer; and (d) in the identification of novel agents for modulating the chemosensitivity of a cancer.

In another aspect the present invention provides new methods for identifying and characterizing new agents that modulate the chemosensitivity of a cancer by altering the expression of one or more transporter genes, which are markers for cancer cell chemosensitivity. The method comprises treating a sample of cells from the cancer with a test agent, obtaining polynucleotide samples from untreated cancer cells and the treated cancer cells, and contacting the polynucleotide samples to polynucleotide probes to detect and measure the amount of target polynucleotides in the sample and thereby obtain an expression profile of genes, such as genes that are involved in cellular transport, which are markers for chemosensitivity. The method further comprises comparing the transporter gene expression profiles of the control and treated cells to determine whether the agent altered the expression of any of the genes correlated with chemosensitivity or chemoresistance to various drugs.

### BRIEF DESCRIPTION OF THE DRAWINGS

FIG. 1 shows expression of transporter gene families correlating with potencies of drugs that are chemically similar to the respective natural substrates. Panel a. Nucleoside transporters positively correlate with nucleoside analogs. A-TGdR: alpha-2′-deoxythioguanosine (NSC 71851); azacytidine (NSC 102816); B-TGdR: beta-2′-deoxythioguanosine (NSC 71261); thioguanine (NSC 752); AraC, cytarabine (NSC 63878), 5FU, fluorouracil (NSC 19893); 6 MP: 6-mercaptopurine (NSC 755); IdA: inosine-glycodialdehyde (NSC 118994); gemcitabine (NSC 613327). Panel b. Folate transporters positively correlate with folate analogs. Aminopterin (NSC 132483); aminopterin-d: aminopterin derivative (NSC 134033); an-antifol (NSC 623017 and NSC 633713); BAF: Baker's-soluble-antifolate (NSC 139105); methotrexate (NSC 740); methotrexate-d: methotrexate derivative (NSC 174121); trimetrexate (NSC 352122). Panel c. Amino acid transporters correlate with amino acid analogs, L-asparaginase (NSC 109229); acivicin (NSC 163501); L-alanosine (NSC 153353); PALA: N-phosphonoacetyl-L-aspartic-acid (NSC 224131). The color code represents the bootstrap P value and reflects the sign of the correlation coefficient.

FIG. 2 shows sorted correlation coefficients between ABCB1 expression and cytotoxic potency of 119 drugs in the NCI60 panel. Known ABCB1-MDR1 substrates such as bisantrene and doxorubicin show strong negative correlations with ABCB1 expression (chemoresistance). CPT derivatives show no significant correlation, indicating that they are not MDR1 substrates. A correlation coefficient of −0.3 is the approximate cutoff for statistical significance, but for each correlation, we additionally compute a bootstrap P value to assess significance. FIG. 3 shows validation of novel gene-drug relationships by siRNA. Human cancer cells were transfected with siRNA targeted against ABCB1- (●) or ABCB-5 (●) or mock-siRNA (∘). After 24 hours, cells were exposed to various concentrations of drug for 4 days and cell growth measured with the SRB assay. Results were expressed as percentage of control cells with no drug treatment (means ±SD from 6 replicates). (Upper Panel) Enhanced chemosensitivity of NCI/ADR-RES cells to Paclitaxel and GA by siRNA-targeting of ABCB1. The ABCB1 siRNA had no effect on potency to 5FU; (Lower Panel) Enhanced chemosensitivity of SK-MEL-28 cells to CPT, 10-OH and 5FU by siRNA-targeting of ABCB5. There is no effect on potency to mitoxathone by siRNA targeting ABCB5. FIG. 4 shows hierarchical cluster analysis of the NCI-60 cell lines based on expression profiles of 57 genes with greatest variance across the cell lines (filtered by SD≧0.39). Data from 62 hybridizations were used, one for each cell line, plus duplicate analysis of TK-10 and MCF7/ADR-RES. BR: breast cancer; CNS: CNS cancer; CO: colon cancer; LC: lung cancer; LE: leukemia; ME: melanoma; OV: ovarian cancer; PR: prostate cancer; RE: renal cancer; UK: unknown origin.

FIG. 5 shows comparison of ATP1B1 mRNA levels by real-time quantitative RT-PCR, cDNA microarray and long oligo microarray, plotted as abundance (log2) of the ATP1B1 transcript relative to its abundance in the reference pool of 12 cell lines. The RT-PCR data are normalized to β-actin. Cell lines tested are: 1, SR; 2, SK-MEL-28; 3, SW-620; 4, ACHN; 5, HL-60; 6, SN12C; 7, T-47D; 8, SF-295; 9, COLO205; 10, 786-0; 11, K562; 12, OVCAR-5.

FIG. 6 shows the dependence of the log ratio M on overall spot intensity A based on statistical analyses that were carried out using the statistical software package R (found on the internet at the website url r-project.org). The plot of M=log₂R/G vs. A=log₂\sqrt{R*G}.

FIG. 7 shows box plots of the log ratios for each of 60 slides analyzed according as described in connection with FIG. 6, for multiple slide normalization.

FIG. 8 shows a listing of oligonucloeotide probes according to the present disclosure.

FIG. 9 shows gene-drug correlations according to the present disclosure.

FIG. 10 shows ABC transporter gene-drug correlations according to the present disclosure.

### DETAILED DESCRIPTION OF THE INVENTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to that this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The disclosure of all patents, patent applications (and any patents that issue thereon, as well as any corresponding published foreign patent applications), GenBank and other accession numbers and associated data, and publications mentioned throughout this description are hereby incorporated by reference herein. It is expressly not admitted, however, that any of the documents incorporated by reference herein teach or disclose the present invention.

The present invention may be understood more readily by reference to the following detailed description of the embodiments of the invention and the Examples included herein. However, before the present methods, compounds and compositions are disclosed and described, it is to be understood that this invention is not limited to specific methods, specific nucleic acids, specific polypeptides, specific cell types, specific host cells or specific conditions, etc., as such may, of course, vary, and the numerous modifications and variations therein will be apparent to those skilled in the art. It is also to be understood that the terminology used herein is for the purpose of describing specific embodiments only and is not intended to be limiting.

Definitions

“Transporter genes” refers to genes that produce gene products, such as proteins, that direct the transport of chemical agents into and out of cells, and comprise amino acids having sequences that comprise conserved protein motifs or domains that were identified by sequence analysis, for example, by employing Hidden Markov Models (HMMs; Krogh et al. (1994) J. Mol. Biol. 235:1501-1531; Collin et al. (1993) Protein Sci. 2:305-314), BLAST (Basic Local Alignment Search Tool; Altschul (1993) J. Mol. Evol. 36:290-300; and Altschul et al. (1990) J. Mol. Biol. 215:403-410) or other analytical tools. Transporter genes may be naturally-occurring, recombinant or variant transporter genes that encode proteins that include membrane transporters, ion exchangers, ion channel proteins, and ATPases. Transporter genes also encode other proteins, including proteins that facilitate or control the movement of chemicals into and out of cells, and recombinant and variant forms thereof that share at least 50% amino acid sequence identity with naturally occurring transporter proteins, or functional domains or portions thereof. “Transporter(s)” are proteins that are encoded by transporter genes.

“Chemosensitivity” refers to the propensity of a cell to be affected by a cytotoxic agent, wherein a cell may range from sensitive to resistant to such an agent. The expression of a chemosensitivity gene, either alone or in combination with other factors or gene expression products, can be a marker for or indicator of chemosensitivity.

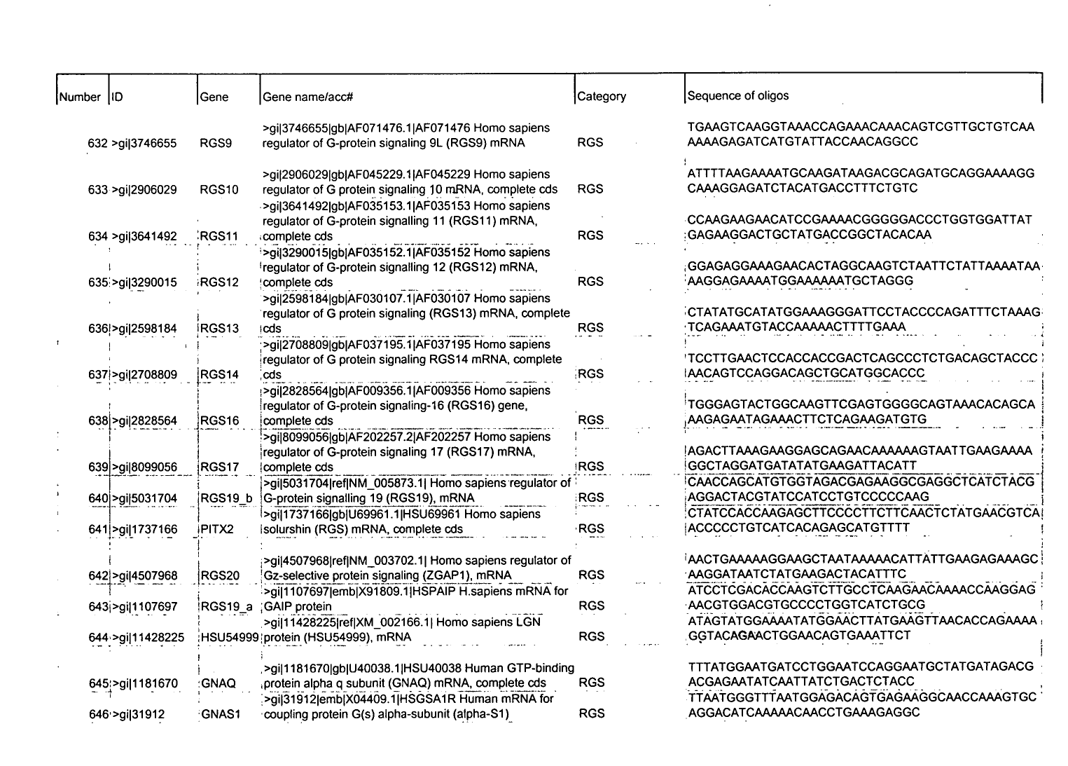

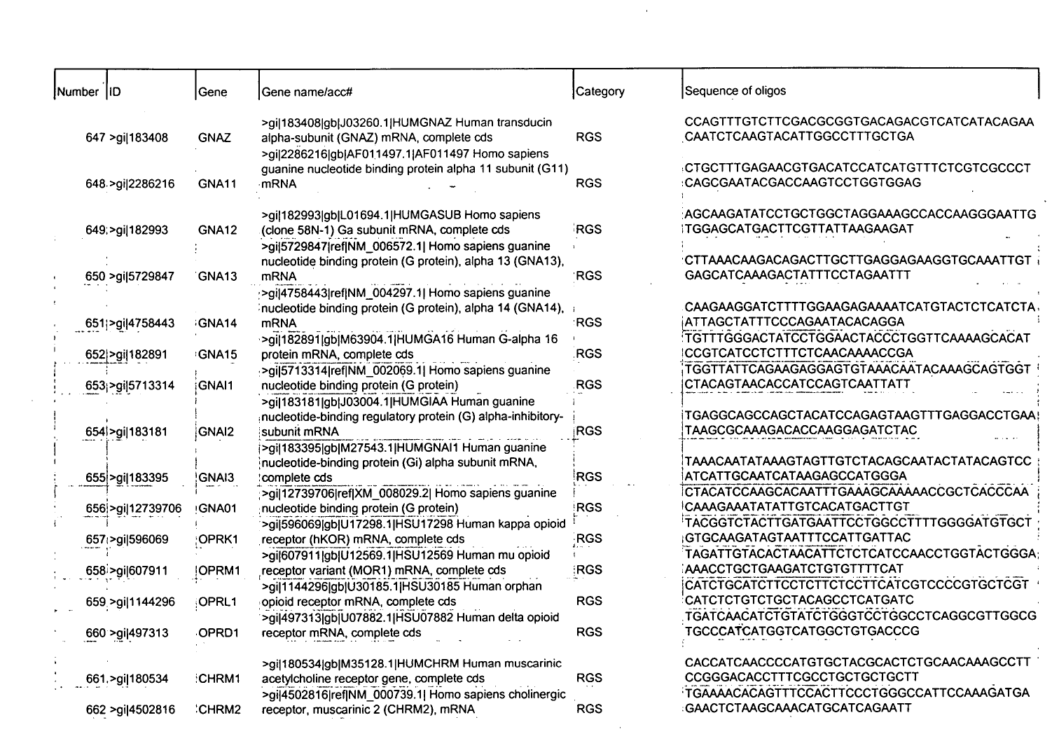

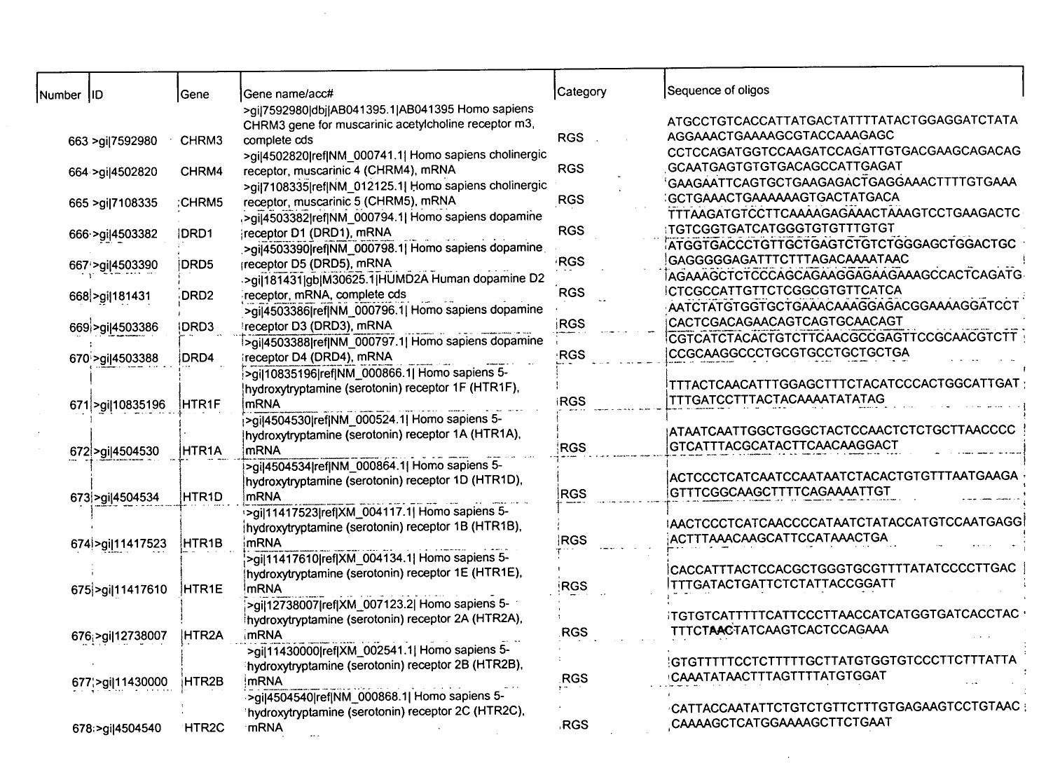

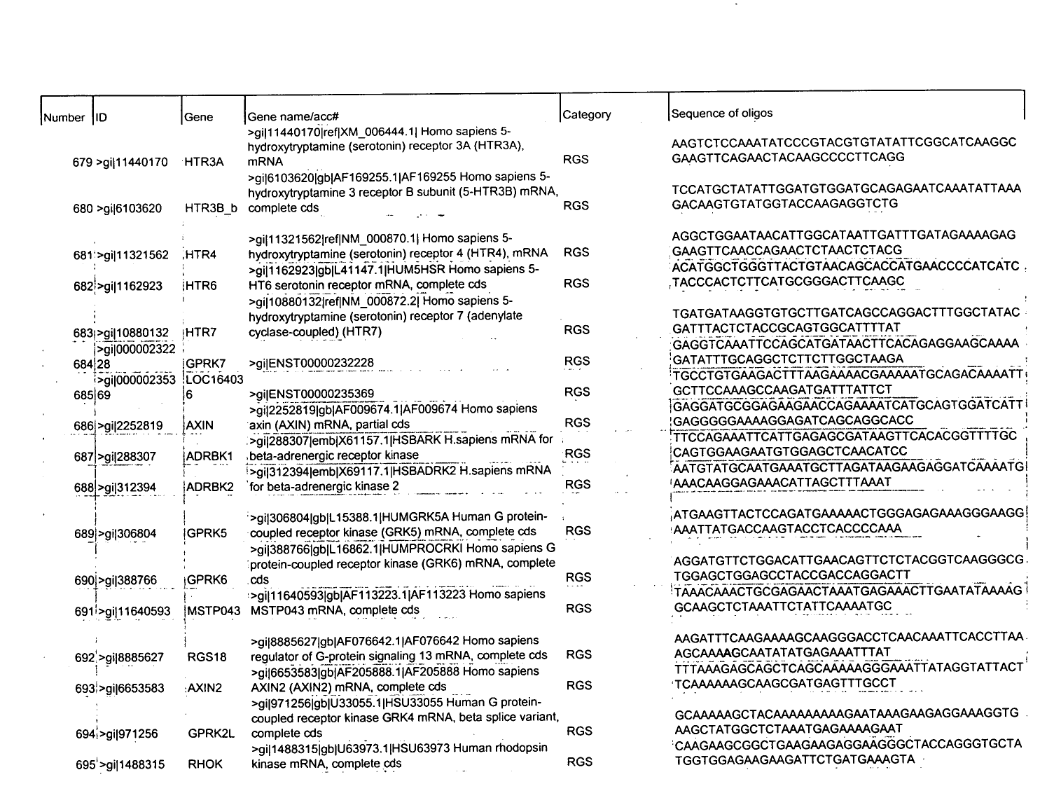

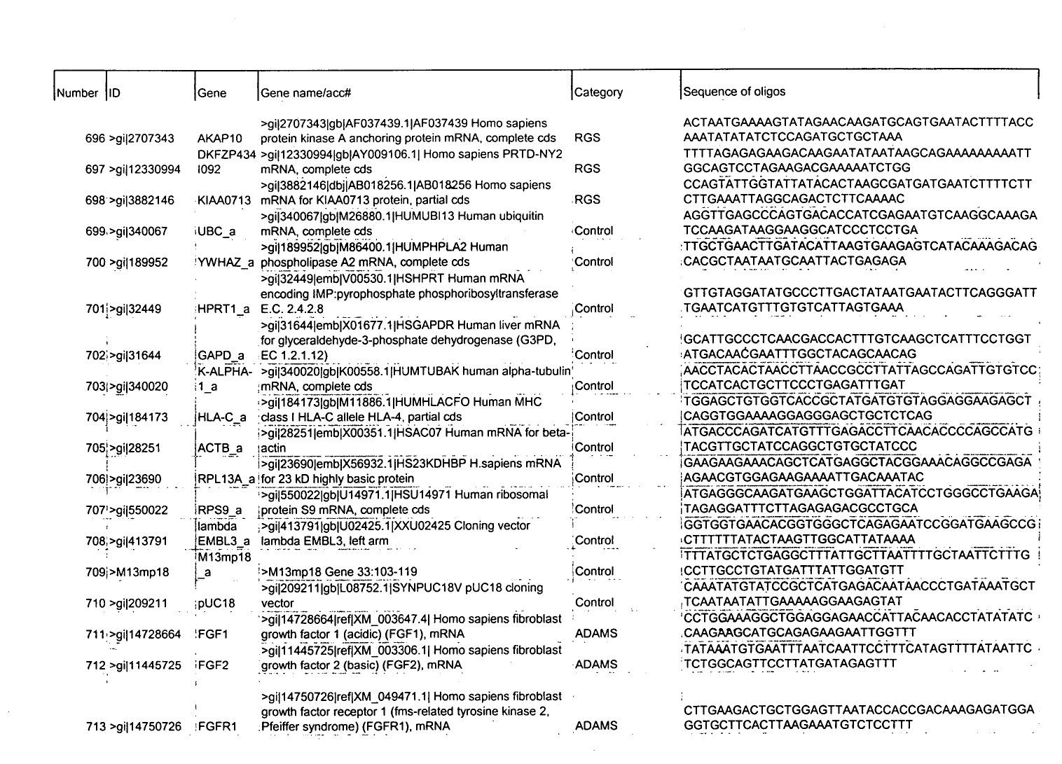

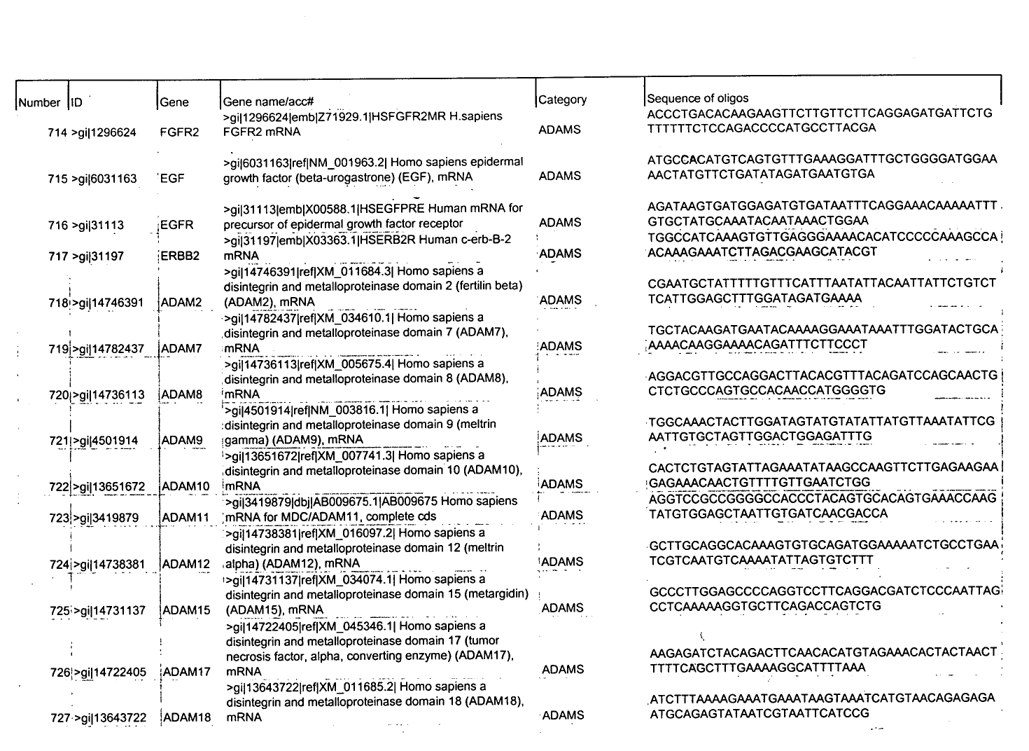

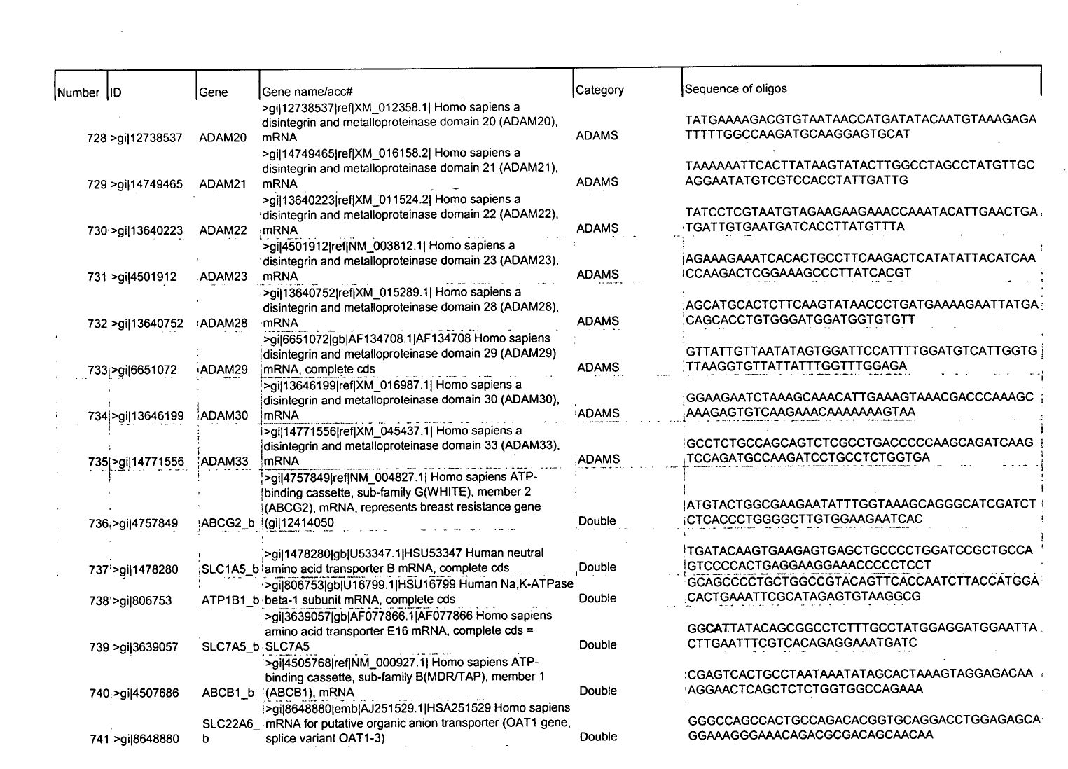

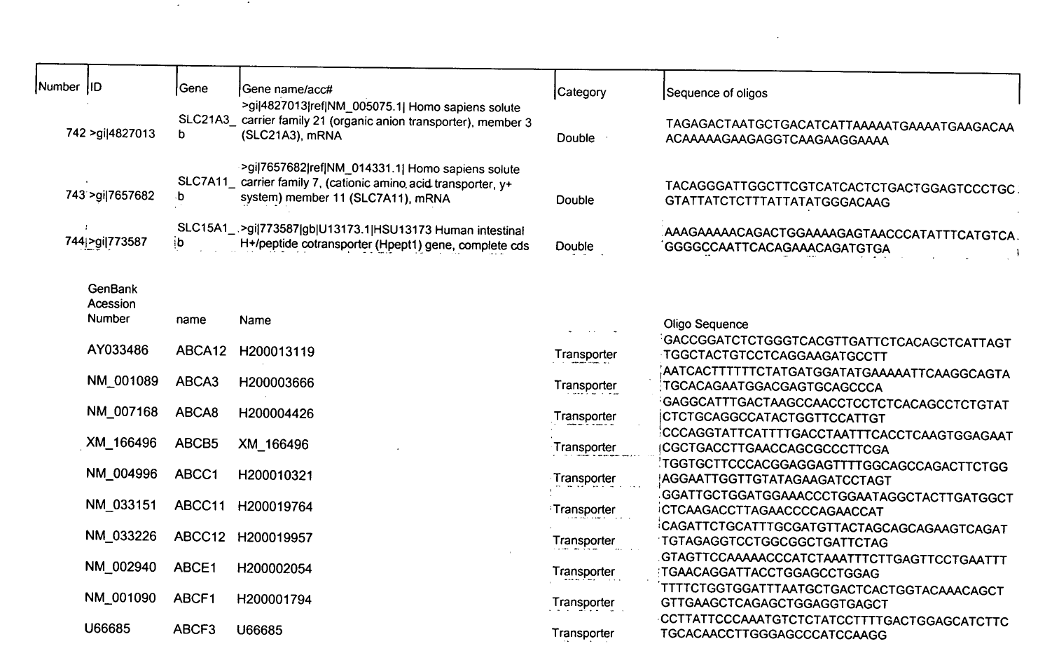

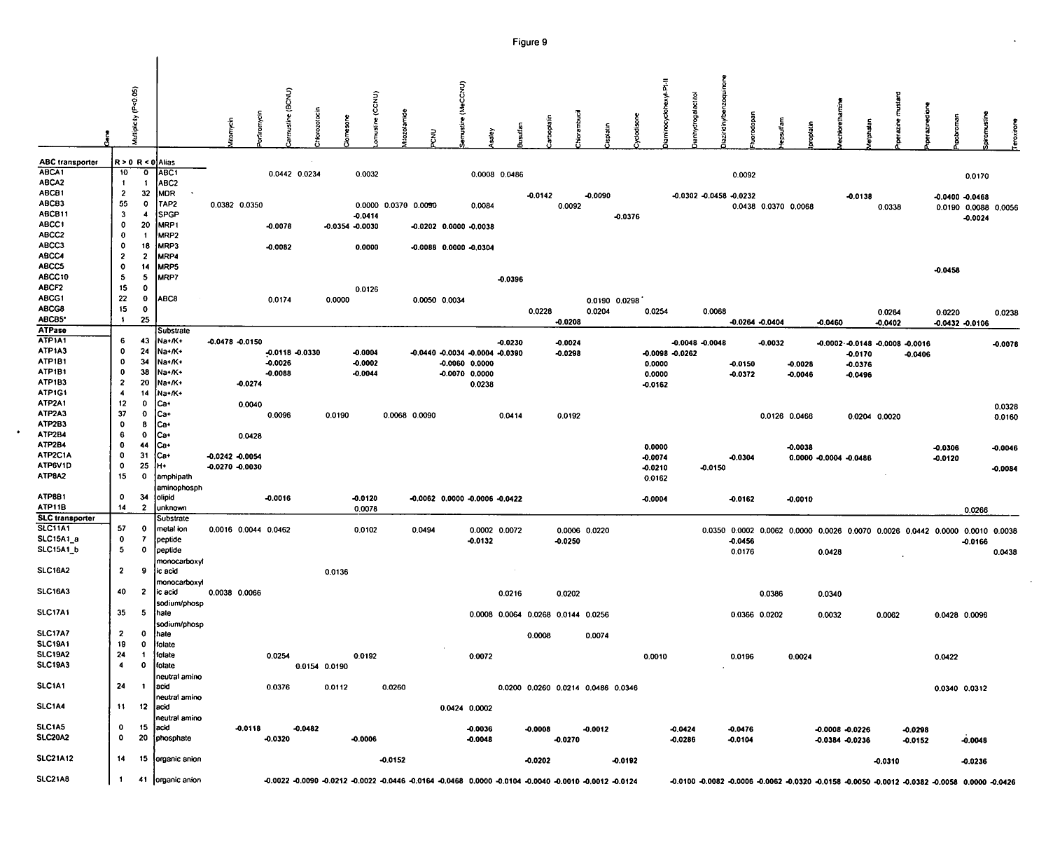

“Chemosensitivity gene” refers to a gene whose protein product influences the chemosensitivity of a cell to one or more cytotoxic agents. According to the instant invention, along a scale that is a continuum, relatively high expression of a given gene in drug-sensitive cell lines is considered a positive correlation, and high expression in drug resistant cells is considered a negative correlation. Thus, negative correlation indicates that a chemosensitivity gene is associated with resistance of a cancer cell to a drug, whereas positive correlation indicates that a chemosensitivity gene is associated with sensitivity of a cancer cell to a drug. Chemosensitivity genes may themselves render cells more sensitive or more resistant to the effects of one or more cytotoxic agents, or may be associated with other factors that directly influence chemosensitivity. That is to say, some chemosensitivity genes may or may not directly participate in rendering a cell sensitive or resistant to a drug, but expression of such genes may be related to the expression of other factors which may influence chemosensitivity. Expression of a chemosensitivity gene can be correlated with the sensitivity of a cell or cell type to an agent, wherein a negative correlation may indicate that the gene affects cellular resistance to the drug, and a positive correlation may indicate that the gene affects cellular sensitivity to a drug. According to the instant disclosure, chemosensitivity genes have been identified among known and putative transporter genes. FIG. 8 lists these genes, along with specific oligonucleotide probes for the genes. FIG. 8 also lists the accession numbers for the known genes, whereby the full sequences of the genes may be referenced, and which are expressly incorporated herein by reference thereto as of the filing of this application for patent.

“Array” or “microarray” refers to an arrangement of hybridizable array elements, such as polynucleotides, which in some embodiments may be on a substrate. The arrangement of polynucleotides may be ordered. In some embodiments, the array elements are arranged so that there are at least ten or more different array elements, and in other embodiments at least 100 or more array elements. Furthermore, the hybridization signal from each of the array elements may be individually distinguishable. In one embodiment, the array elements comprise nucleic acid molecules. In some embodiments, the array comprises probes to tow or more chemosensitivity genes, and in other embodiments the array comprises probes to 10, 15, 20, 25, 30, 35, 40, 45, 50, 55, 60, 65, 70, 75, 80, 85, 90, 95, 100, 150, 200, 250 or more chemosensitivity genes. In some embodiments, the array comprises probes to genes that encode products other than chemosensitivity proteins. In some embodiments, the array comprises probes to 5, 10, 20, 30, 40, 50, 60, 70, 80, 90, 100, or more genes that encode products other than chemosensitivity proteins.

“Gene,” when used herein, broadly refers to any region or segment of DNA associated with a biological molecule or function. Thus, genes include coding sequence, and may further include regulatory regions or segments required for their expression. Genes may also include non-expressed DNA segments that, for example, form recognition sequences for other proteins. Genes can be obtained from a variety of sources, including cloning from a source of interest or synthesizing from known or predicted sequence information, and may include sequences encoding desired parameters.

“Hybridization complex” refers to a complex between two nucleic acid molecules by virtue of the formation of hydrogen bonds between purines and pyrimidines.

“Identical” or percent “identity,” when used herein in the context of two or more nucleic acid or polypeptide sequences, refer to two or more sequences or subsequences that may be the same or have a specified percentage of amino acid residues or nucleotides that are the same, when compared and aligned for maximum correspondence. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

“Isolated,” when used herein in the context of a nucleic acid or protein, denotes that the nucleic acid or protein is essentially free of other cellular components with that it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant molecular species present in a preparation is substantially purified. An isolated gene is separated from open reading frames that flank the gene and encode a protein other than the gene of interest.

“Marker,” as used herein in reference to a chemosensitivity gene, means an indicator of chemosensitivity. A marker may either directly or indirectly influence the chemosensitivity of a cell to a cytotoxic agent, or it may be associated with other factors that influence chemosensitivity.

“Naturally-occurring” and “wild-type,” are used herein to describe something that can be found in nature as distinct from being artificially produced by man. For example, a polypeptide or polynucleotide sequence that is present in an organism (including viruses) that can be isolated from a source in nature and that has not been intentionally modified by man in the laboratory is naturally-occurring. In particular, “wild-type” is used herein to refer to the naturally-occurring or native forms of transporter proteins and their encoding nucleic acid sequences. Therefore, in the context of this application, ‘wild-type’ includes naturally occurring variant forms for transporter genes, either representing splice variants or genetic variants between individuals, which may require different probes for selective detection.

“Nucleic acid,” when used herein, refers to deoxyribonucleotides or ribonucleotides, nucleotides, oligonucleotides, polynucleotide polymers and fragments thereof in either single- or double-stranded form. A nucleic acid may be of natural or synthetic origin, double-stranded or single-stranded, and separate from or combined with carbohydrate, lipids, protein, other nucleic acids, or other materials, and may perform a particular activity such as transformation or form a useful composition such as a peptide nucleic acid (PNA). Unless specifically limited, the term encompasses nucleic acids containing known analogues of natural nucleotides that have similar binding properties as the reference nucleic acid and may be metabolized in a manner similar to naturally-occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19: 5081; Ohtsuka et al. (1985) J. Biol. Chem. 260: 2605-2608; Cassol et al. (1992); Rossolini et al. (1994) Mol. Cell. Probes 8: 91-98). The term nucleic acid is used interchangeably with gene, cDNA, and mRNA encoded by a gene.

An “Oligonucleotide” or “oligo” is a nucleic acid and is substantially equivalent to the terms amplimer, primer, oligomer, element, target, and probe, and may be either double or single stranded.

“Plurality” refers to a group of at least two or more members.

“Polynucleotide” refers to nucleic acid having a length from 25 to 3,500 nucleotides.

“Probe” or “Polynucleotide Probe” refers to a nucleic acid capable of hybridizing under stringent conditions with a target region of a target sequence to form a polynucleotide probe/target complex. Probes comprise polynucleotides that are 15 consecutive nucleotides in length. Probes may be 15, 16, 17, 18 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 6, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 polynucleotides in length. In some embodiments, probes are 70 nucleotides in length. Probes may be less than 100% complimentary to a target region, and may comprise sequence alterations in the form of one or more deletions, insertions, or substitutions, as compared to probes that are 100% complementary to a target region.

“Purified,” when used herein in the context of nucleic acids or proteins, denotes that a nucleic acid or protein gives rise to essentially one band in an electrophoretic gel. Particularly, it means that the nucleic acid or protein is at least 50, 55, 60, 65, 70, 75, 80, 85, 90, 91, 92, 93, 94, 95, 96, 97, 98, 99 or 100% pure with respect to the presence of any other nucleic acid or protein species.

“Sample” refers to an isolated sample of material, such as material obtained from an organism, containing nucleic acid molecules. A sample may comprise a bodily fluid; a cell; an extract from a cell, chromosome, organelle, or membrane isolated from a cell; genomic DNA, RNA, or cDNA in solution or bound to a substrate; or a biological tissue or biopsy thereof. A sample may be obtained from any bodily fluid (blood, urine, saliva, phlegm, gastric juices, etc.), cultured cells, biopsies, or other tissue preparations.

“Stringent hybridization conditions” and “stringent hybridization wash conditions” in the context of nucleic acid hybridization experiments such as Southern and northern hybridizations are sequence dependent, and are different under different environmental parameters. Nucleic acids having longer sequences hybridize specifically at higher temperatures. An extensive guide to the hybridization of nucleic acids is found in Tijssen (1993) Laboratory Techniques in Biochemistry and Molecular Biology—Hybridization with Nucleic Acid Probes part I chapter 2 “Overview of principles of hybridization and the strategy of nucleic acid probe assays,” Elsevier, N.Y. Generally, highly stringent hybridization and wash conditions are selected to be 5° C. lower than the thermal melting point (Tₘ) for the specific sequence at a defined ionic strength and pH. Typically, under “stringent conditions” a probe will hybridize to its target subsequence, but to no other sequences. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids that have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for 15 minutes. An example of stringent wash conditions is a 0.2×SSC wash at 65° C. for 15 minutes (see, Sambrook, infra., for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., 10 to 50 nucleotides), stringent conditions typically involve salt concentrations of less than 1.0 M Na ion, typically 0.01 to 1.0 M Na ion concentration (or other salts) at pH 7.0 to 8.3, and the temperature is typically at least 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially similar if the polypeptides that they encode are substantially similar. This occurs, e.g., when a copy of a nucleic acid is created using the maximum codon degeneracy permitted by the genetic code.

“Substrate” refers to a support, such as a rigid or semi-rigid support, to which nucleic acid molecules or proteins are applied or bound, and includes membranes, filters, chips, slides, wafers, fibers, magnetic or nonmagnetic beads, gels, capillaries or other tubing, plates, polymers, and microparticles, and other types of supports, which may have a variety of surface forms including wells, trenches, pins, channels and pores.

“Target polynucleotide,” as used herein, refers to a nucleic acid to which a polynucleotide probe can hybridize by base pairing and that comprises all or a fragment of a gene that encodes a protein that is a marker for chemosensitivity in cancer cells. In some instances, the sequences of target and probes may be 100% complementary (no mismatches) when aligned. In other instances, there may be up to a 10% mismatch. Target polynucleotides represent a subset of all of the polynucleotides in a sample that encode the expression products of all transcribed and expressed genes in the cell or tissue from which the polynucleotide sample is prepared. The gene products of target polynucleotides are markers for chemosensitivity of cancer cells; some may directly influence chemosensitivity by mediating drug transport. Alternatively, they may direct or influence cancer cell characteristics that indirectly confer or influence sensitivity or resistance. For example, these proteins may function by establishing or maintaining the electrochemical gradient, or providing necessary nutrients for cancer cells. Or they may be less directly involved and are expressed in conjunction with other factors that directly influence chemosensitivity.

“Target Region” means a stretch of consecutive nucleotides comprising all or a portion of a target sequence such as a gene or an oligonucleotide encoding a protein that is a marker for chemosensitivity. Target regions may be 15, 16, 17, 18 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 6, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200 or more polynucleotides in length. In some embodiments, target regions are 70 nucleotides in length, and lack secondary structure. Target regions may be identified using computer software programs such as OLIGO 4.06 software (National Biosciences, Plymouth Minn.), LASERGENE software (DNASTAR, Madison Wis.), MACDNASIS (Hitachi Software Engineering Co., San Francisco, Calif.) and the like.

Polynucleotide Probes

The polynucleotide probes can be genomic DNA or cDNA or mRNA, or any RNA-like or DNA-like material, such as peptide nucleic acids, branched DNAs and the like. The polynucleotide probes can be sense or antisense polynucleotide probes. Where target polynucleotides are double stranded, the probes may be either sense or antisense strands. Where the target polynucleotides are single stranded, the nucleotide probes are complementary single strands.

The polynucleotide probes can be prepared by a variety of synthetic or enzymatic schemes that are well known in the art. The probes can be synthesized, in whole or in part, using chemical methods well known in the art Caruthers et al. (1980) Nucleic Acids Res. Symp. Ser. 215-233). Alternatively, the probes can be generated, in whole or in part, enzymatically.

Nucleotide analogues can be incorporated into the polynucleotide probes by methods well known in the art. The only requirement is that the incorporated nucleotide analogues must serve to base pair with target polynucleotide sequences. For example, certain guanine nucleotides can be substituted with hypoxanthine that base pairs with cytosine residues. However, these base pairs are less stable than those between guanine and cytosine. Alternatively, adenine nucleotides can be substituted with 2,6-diaminopurine that can form stronger base pairs than those between adenine and thymidine. Additionally, the polynucleotide probes can include nucleotides that have been derivatized chemically or enzymatically. Typical chemical modifications include derivatization with acyl, alkyl, aryl or amino groups.

The polynucleotide probes may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as P³², P³³ or S³⁵, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

The polynucleotide probes can be immobilized on a substrate. Preferred substrates are any suitable rigid or semi-rigid support, including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Target Polynucleotides

In order to conduct sample analysis, a sample containing polynucleotides that will be assessed for the presence of target polynucleotides are obtained. The samples can be any sample containing target polynucleotides and obtained from any bodily fluid (blood, urine, saliva, phlegm, gastric juices, etc.), cultured cells, biopsies, or other tissue preparations.

DNA or RNA can be isolated from the sample according to any of a number of methods well known to those of skill in the art. For example, methods of purification of nucleic acids are described in Tijssen (1993) Laboratory Techniques in Biochemistry and Molecular Biology: Hybridization With Nucleic Acid Probes, Part I. Theory and Nucleic Acid Preparation, Elsevier, New York N.Y. In one case, total RNA is isolated using the TRIZOL reagent (Life Technologies, Gaithersburg Md.), and mRNA is isolated using oligo d(T) column chromatography or glass beads. Alternatively, when polynucleotide samples are derived from an mRNA, the polynucleotides can be a cDNA reverse transcribed from an mRNA, an RNA transcribed from that cDNA, a DNA amplified from that cDNA, an RNA transcribed from the amplified DNA, and the like. When the polynucleotide is derived from DNA, the polynucleotide can be DNA amplified from DNA or RNA reverse transcribed from DNA.

Suitable methods for measuring the relative amounts of the target polynucleotide transcripts in samples of polynucleotides are Northern blots, RT-PCR, or real-time PCR, or RNase protection assays. Fore ease in measuring the transcripts for target polynucleotides, it is preferred that arrays as described above be used.

The target polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as P³², P³³ or S³⁵, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

Hybridization Complexes

Hybridization causes a denatured polynucleotide probe and a denatured complementary target polynucleotide to form a stable duplex through base pairing. Hybridization methods are well known to those skilled in the art (See, e.g., Ausubel (1997; Short Protocols in Molecular Biology, John Wiley & Sons, New York N.Y., units 2.8-2.11, 3.18-3.19 and 4-6-4.9). Conditions can be selected for hybridization where exactly complementary target and polynucleotide probe can hybridize, i.e., each base pair must interact with its complementary base pair. Alternatively, conditions can be selected where target and polynucleotide probes have mismatches but are still able to hybridize. Suitable conditions can be selected, for example, by varying the concentrations of salt in the prehybridization, hybridization and wash solutions, or by varying the hybridization and wash temperatures. With some membranes, the temperature can be decreased by adding formamide to the prehybridization and hybridization solutions.

Hybridization conditions are based on the melting temperature (Tₘ) the nucleic acid binding complex or probe, as described in Berger and Kimmel (1987) Guide to Molecular Cloning Techniques, Methods in Enzymology, vol 152, Academic Press. The term “stringent conditions, as used herein, is the “stringency” that occurs within a range from Tm-5 (5° below the melting temperature of the probe) to 20° C. below Tm. As used herein “highly stringent” conditions employ at least 0.2×SSC buffer and at least 65° C. As recognized in the art, stringency conditions can be attained by varying a number of factors such as the length and nature, i.e., DNA or RNA, of the probe; the length and nature of the target sequence, the concentration of the salts and other components, such as formamide, dextran sulfate, and polyethylene glycol, of the hybridization solution. All of these factors may be varied to generate conditions of stringency that are equivalent to the conditions listed above.

Hybridization can be performed at low stringency with buffers, such as 6.times.SSPE with 0.005% Triton X-100 at 37.degree. C., which permits hybridization between target and polynucleotide probes that contain some mismatches to form target polynucleotide/probe complexes. Subsequent washes are performed at higher stringency with buffers, such as 0.5.times.SSPE with 0.005% Triton X-100 at 50.degree. C., to retain hybridization of only those target/probe complexes that contain exactly complementary sequences. Alternatively, hybridization can be performed with buffers, such as 5.times.SSC/0.2% SDS at 60.degree. C. and washes are performed in 2.times.SSC/0.2% SDS and then in 0.1.times.SSC. Background signals can be reduced by the use of detergent, such as sodium dodecyl sulfate, Sarcosyl or Triton X-100, or a blocking agent, such as salmon sperm DNA.

Array Construction

The nucleic acid sequences can be used in the construction of arrays, for example, microarrays. Methods for construction of microarrays, and the use of such microarrays, are known in the art, examples of which can be found in U.S. Pat. Nos. 5,445,934, 5,744,305, 5,700,637, and 5,945,334, the entire disclosure of each of which is hereby incorporated by reference. Microarrays can be arrays of nucleic acid probes, arrays of peptide or oligopeptide probes, or arrays of chimeric probes—peptide nucleic acid (PNA) probes. Those of skill in the art will recognize the uses of the collected information.

One particular example, the in situ synthesized oligonucleotide Affymetrix GeneChip system, is widely used in many research applications with rigorous quality control standards. (Rouse R. and Hardiman G., “Microarray technology—an intellectual property retrospective,” Pharmacogenomics 5:623-632 (2003).). Currently the Affymetrix GeneChip uses eleven 25-oligomer probe pair sets containing both a perfect match and a single nucleotide mismatch for each gene sequence to be identified on the array. Using a light-directed chemical synthesis process (photolithography technology), highly dense glass oligo probe array sets (>1,000,000 25-oligomer probes) can be constructed in a ˜3×3-cm plastic cartridge that serves as the hybridization chamber. The ribonucleic acid to be hybridized is isolated, amplified, fragmented, labeled with a fluorescent reporter group, and stained with fluorescent dye after incubation. Light is emitted from the fluorescent reporter group only when it is bound to the probe. The intensity of the light emitted from the perfect match oligoprobe, as compared to the single base pair mismatched oligoprobe, is detected in a scanner, which in turn is analyzed by bioinformatics software (http://www.affymetrix.com). The GeneChip system provides a standard platform for array fabrication and data analysis, which permits data comparisons among different experiments and laboratories.

Microarrays according to the invention can be used for a variety of purposes, as further described herein, including but not limited to, screening for the resistance or susceptibility of a cancer to a drug based on the genetic expression profile of the cancer.

Chemosensitivity Gene Expression Analysis System

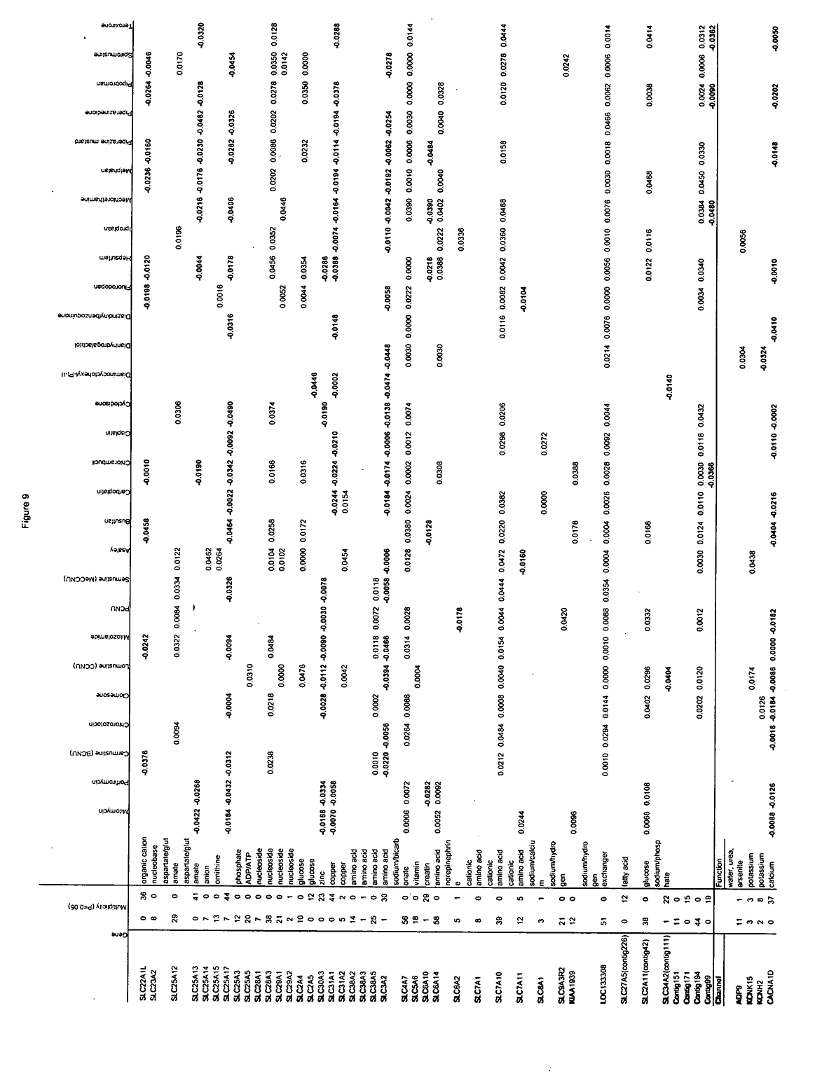

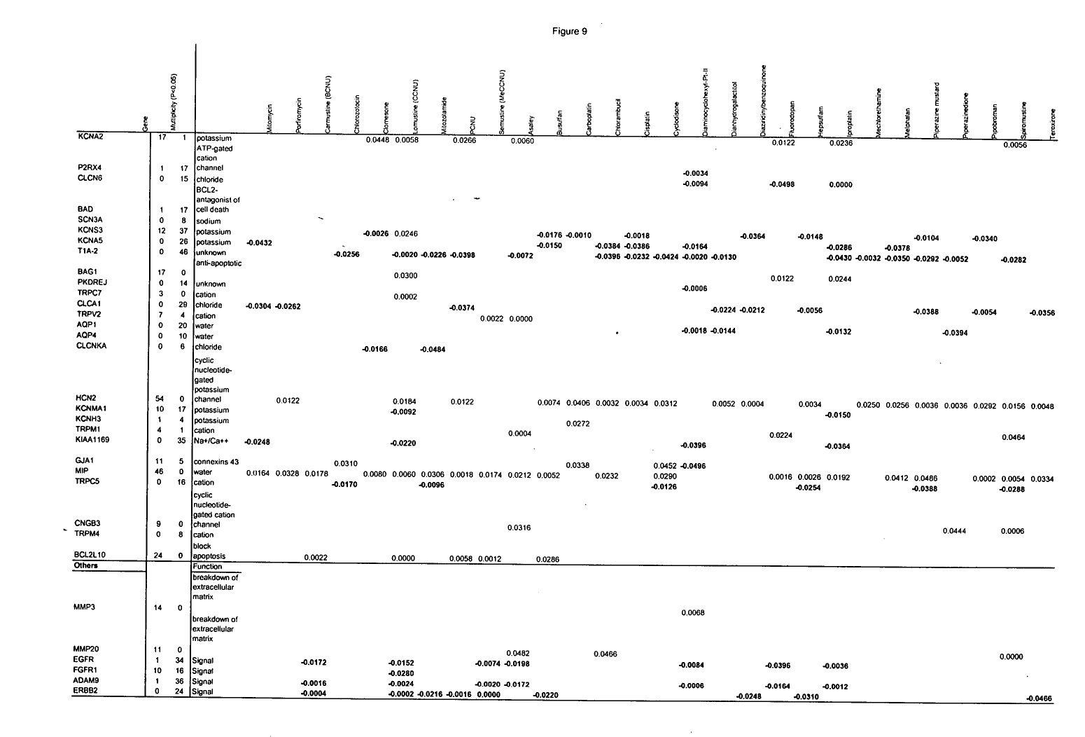

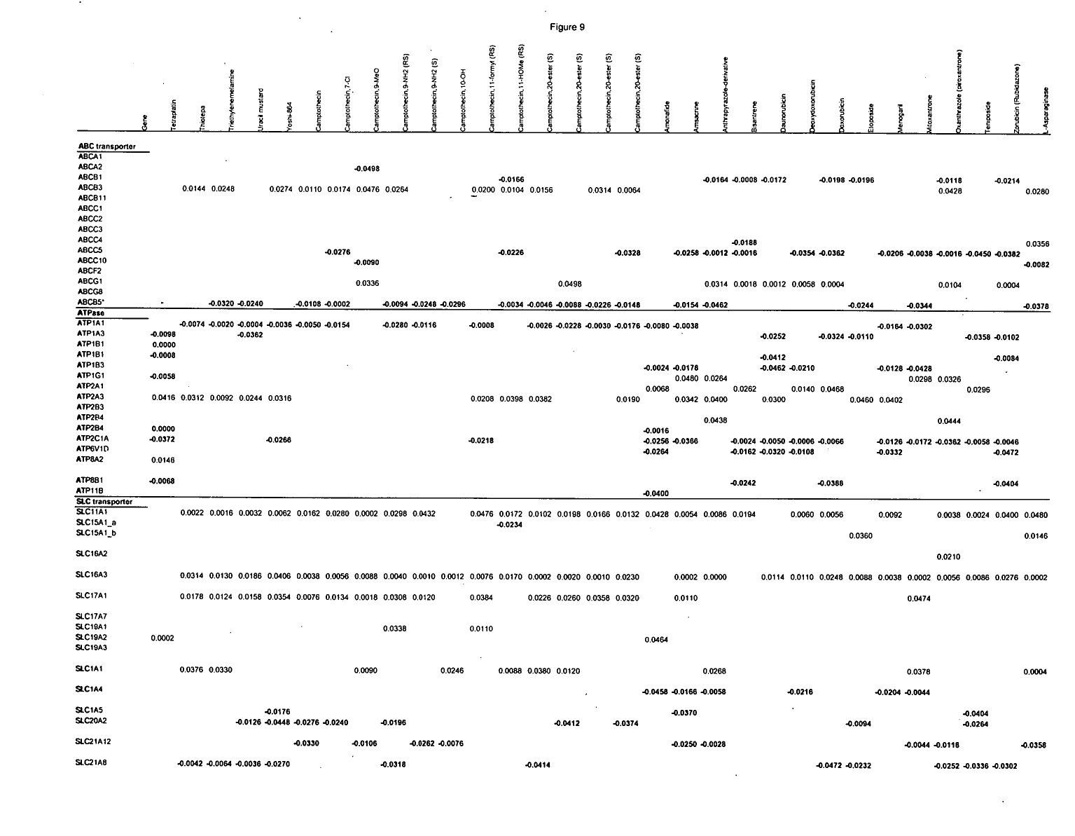

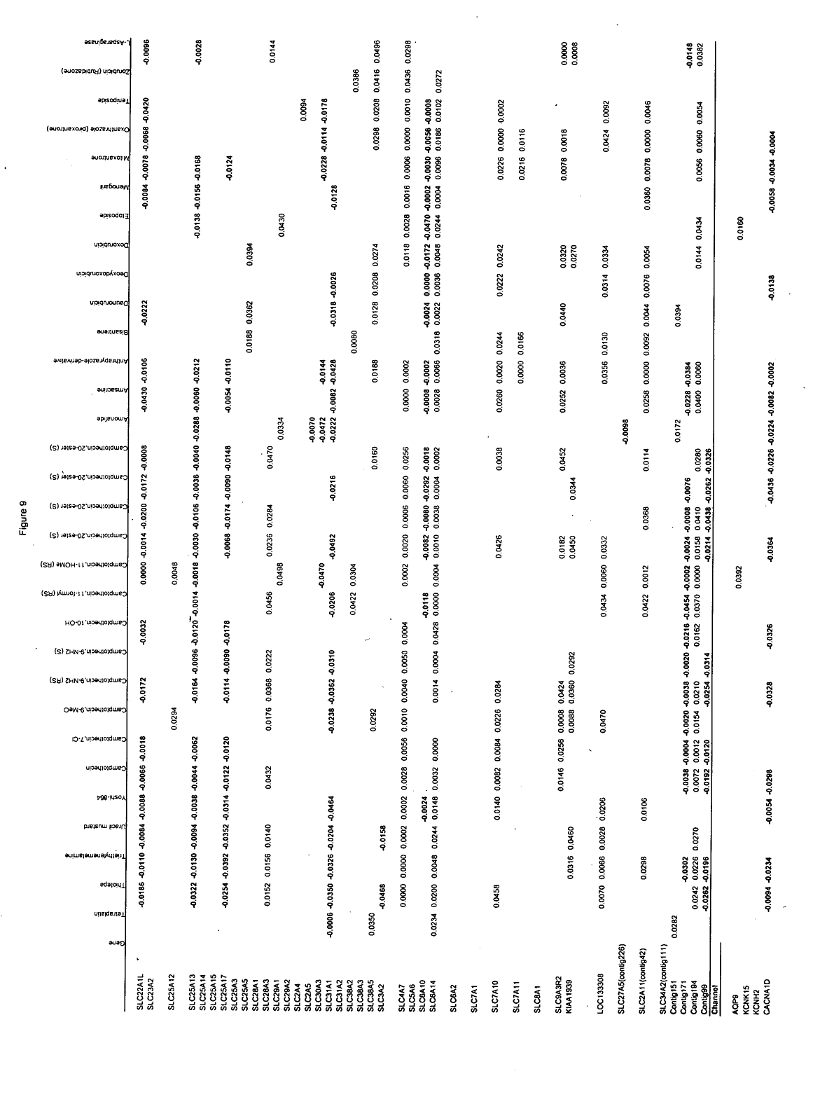

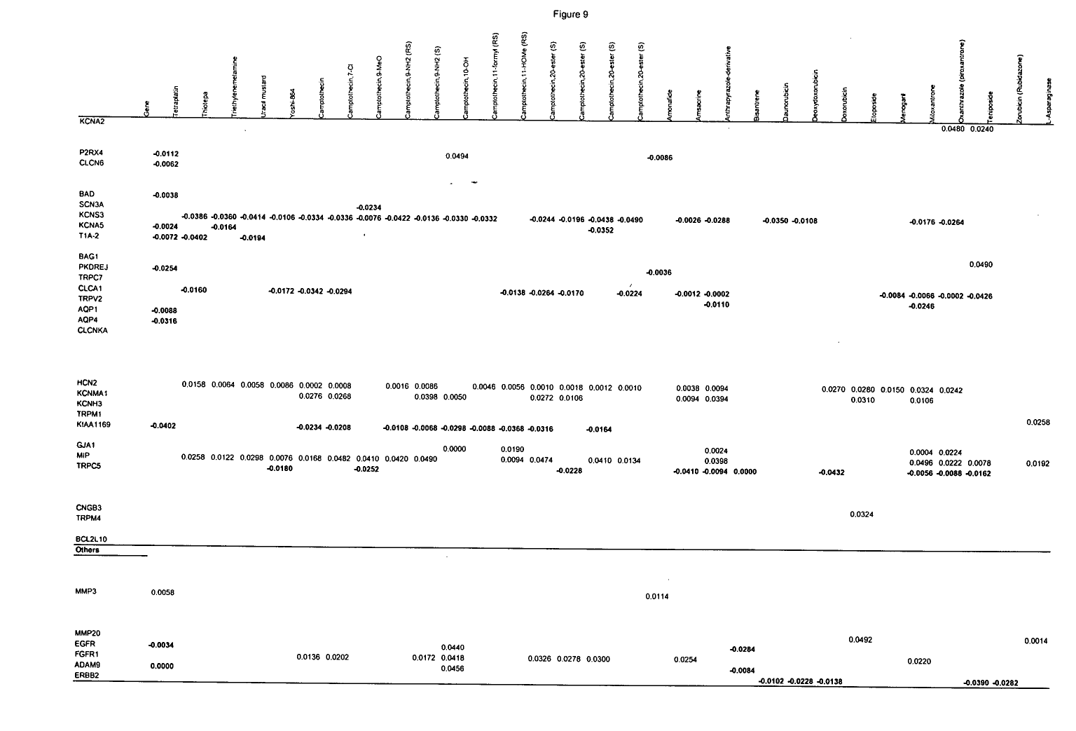

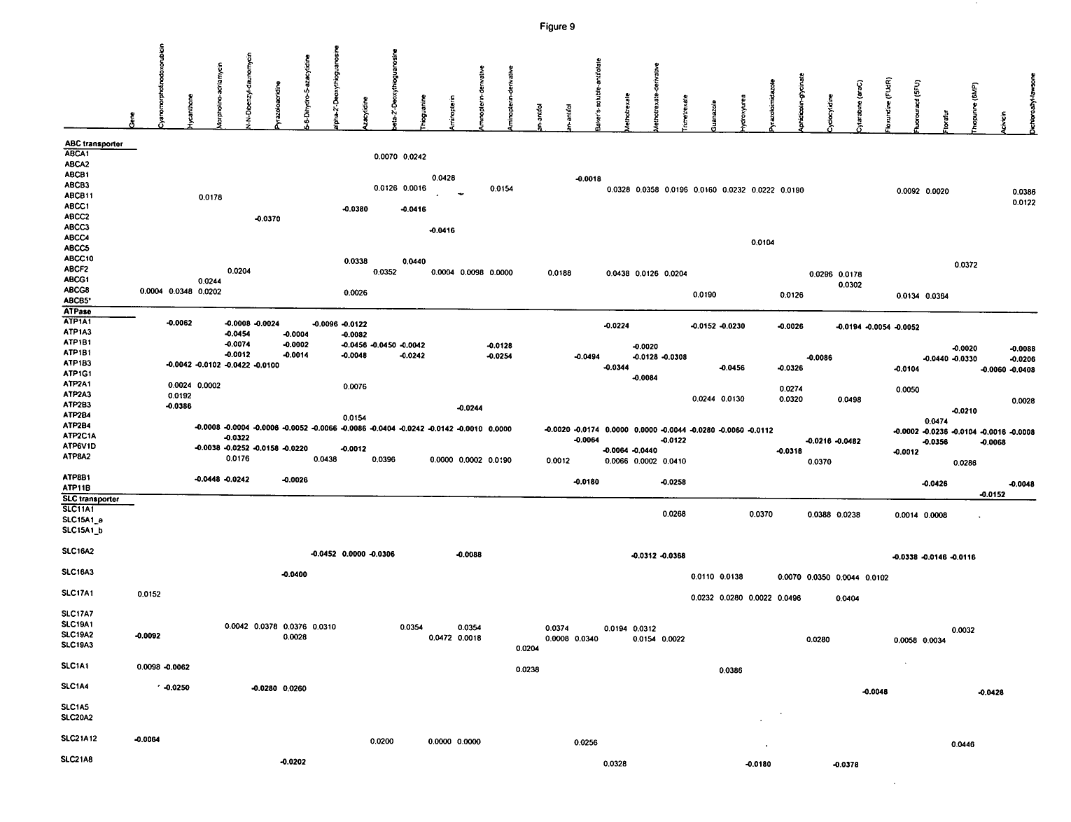

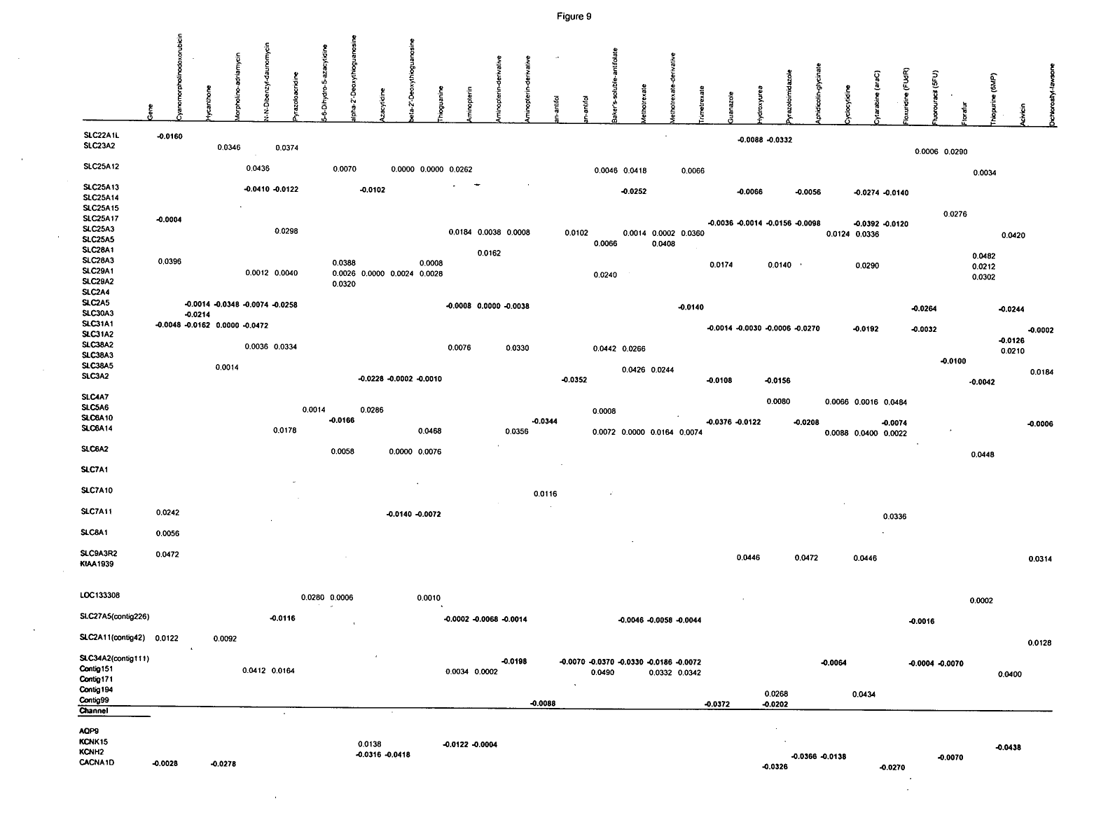

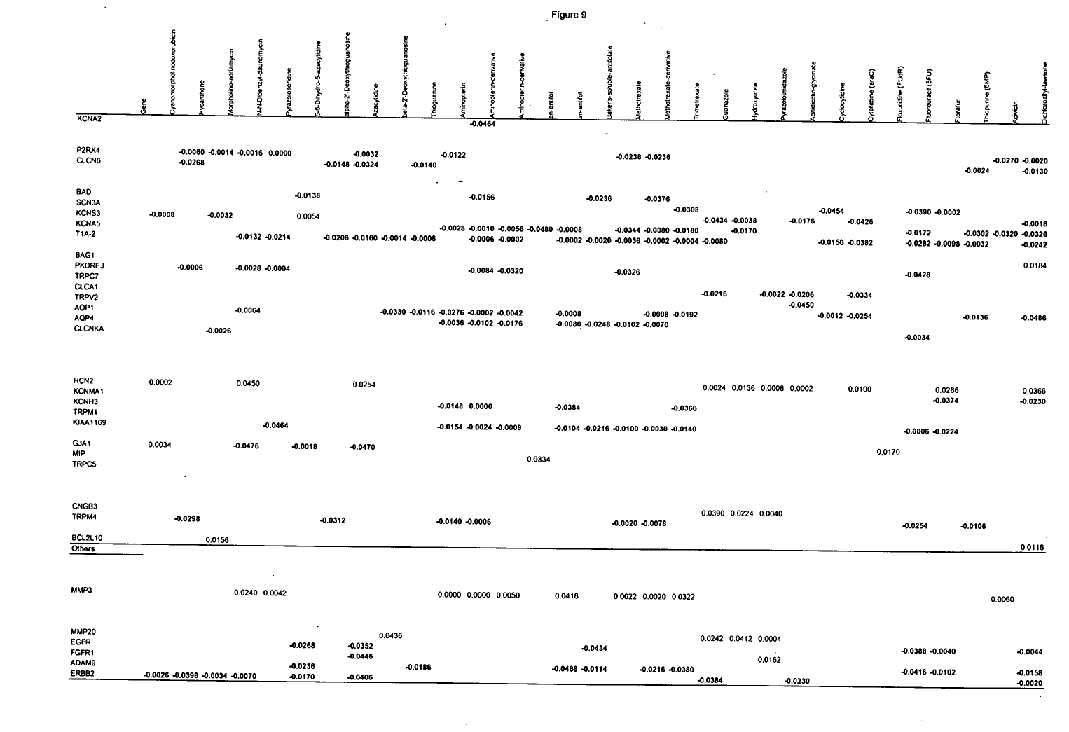

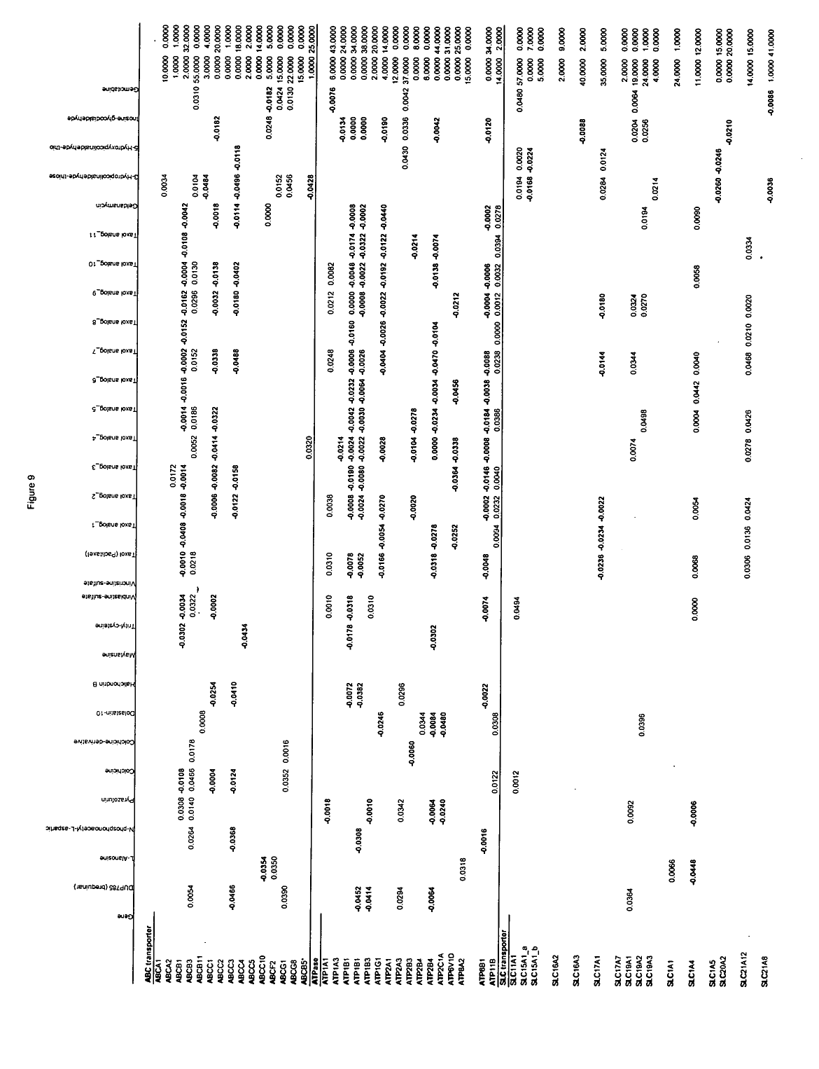

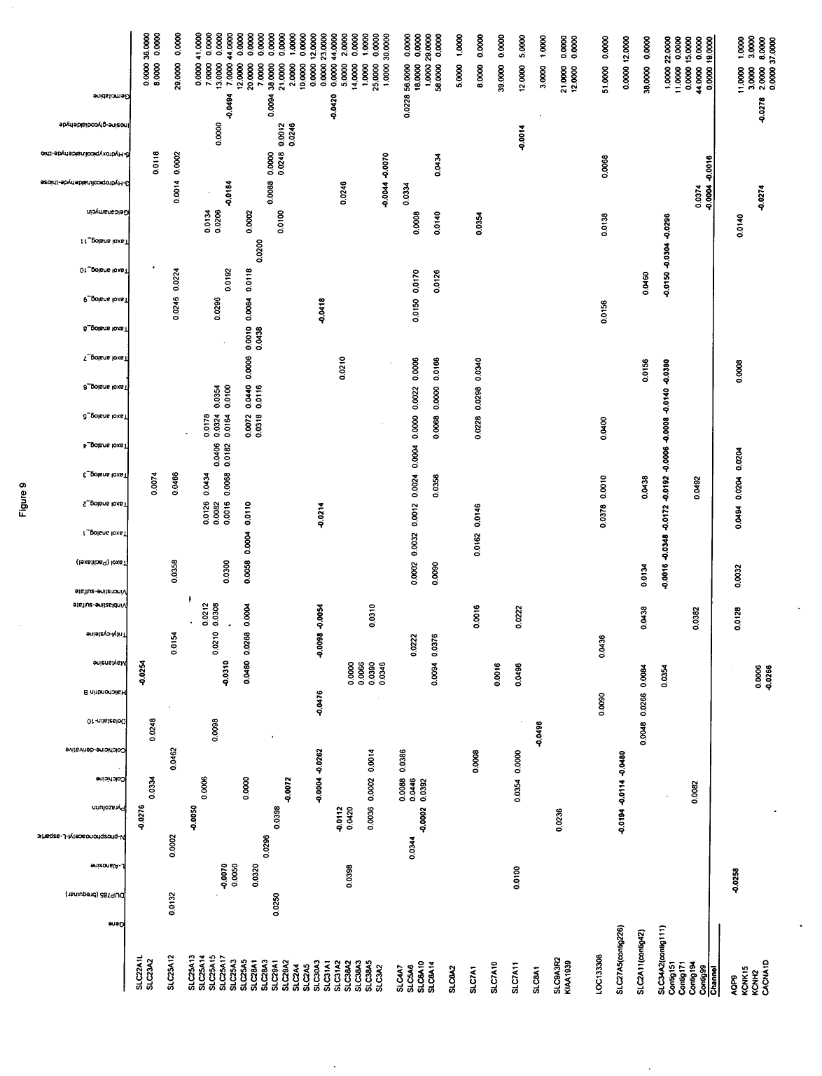

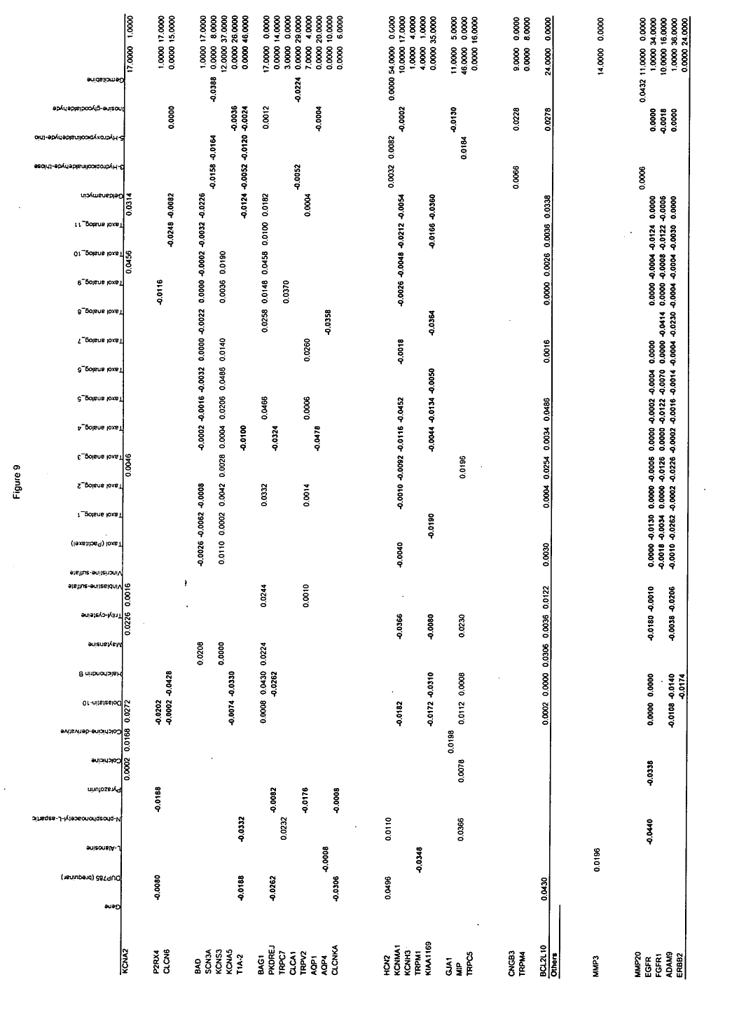

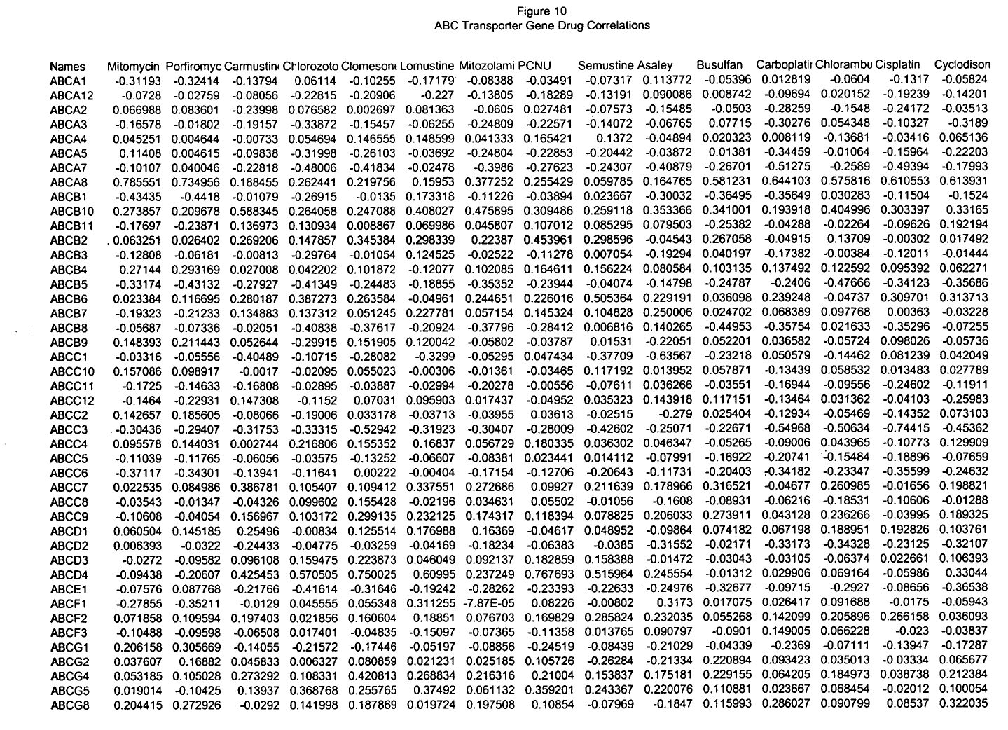

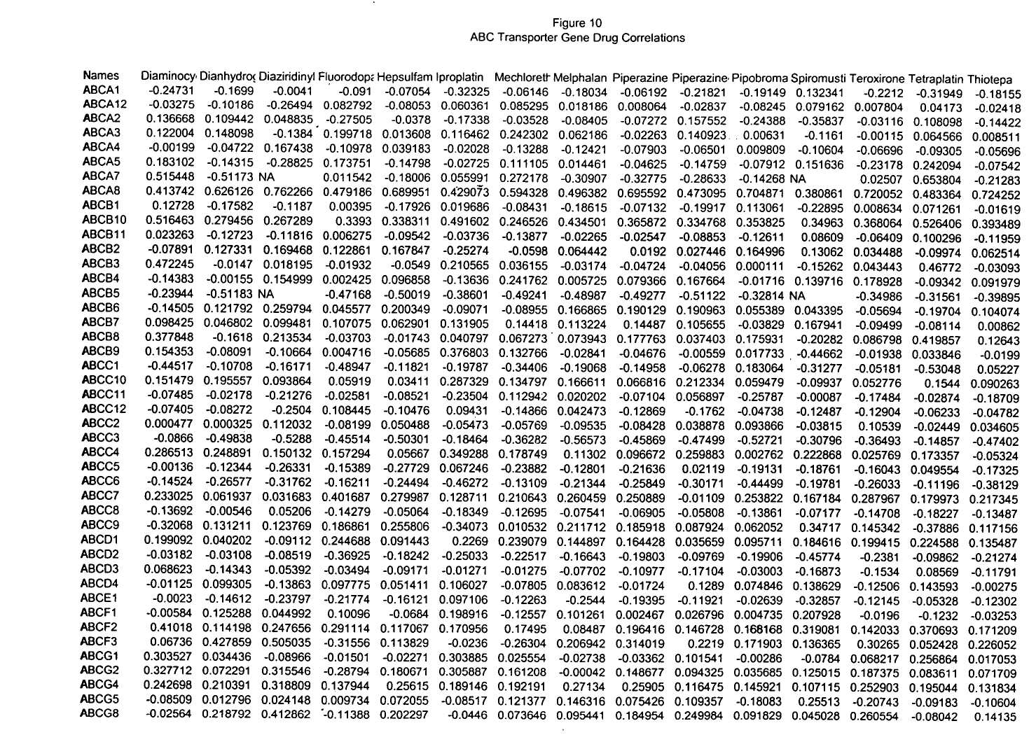

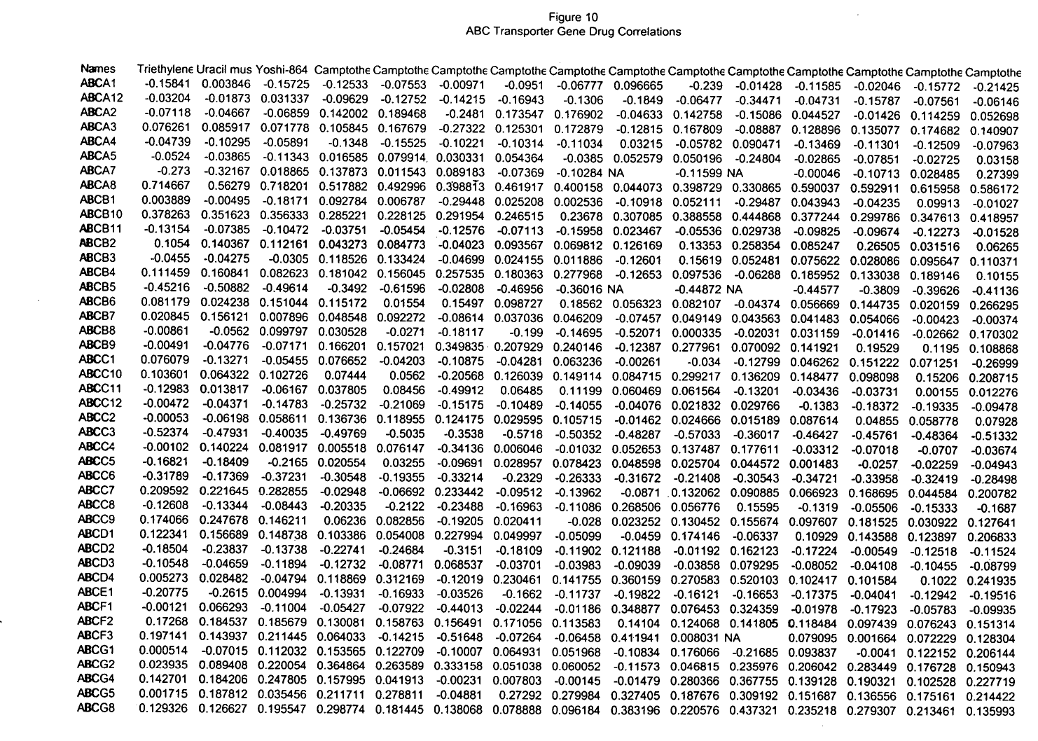

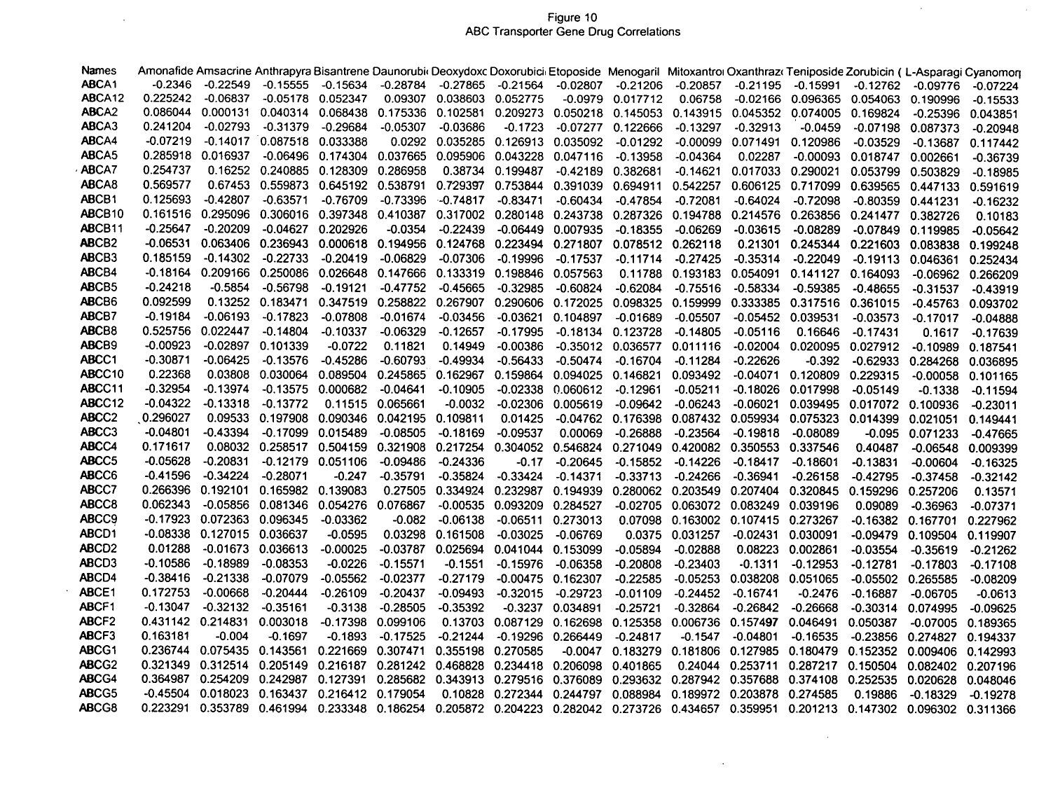

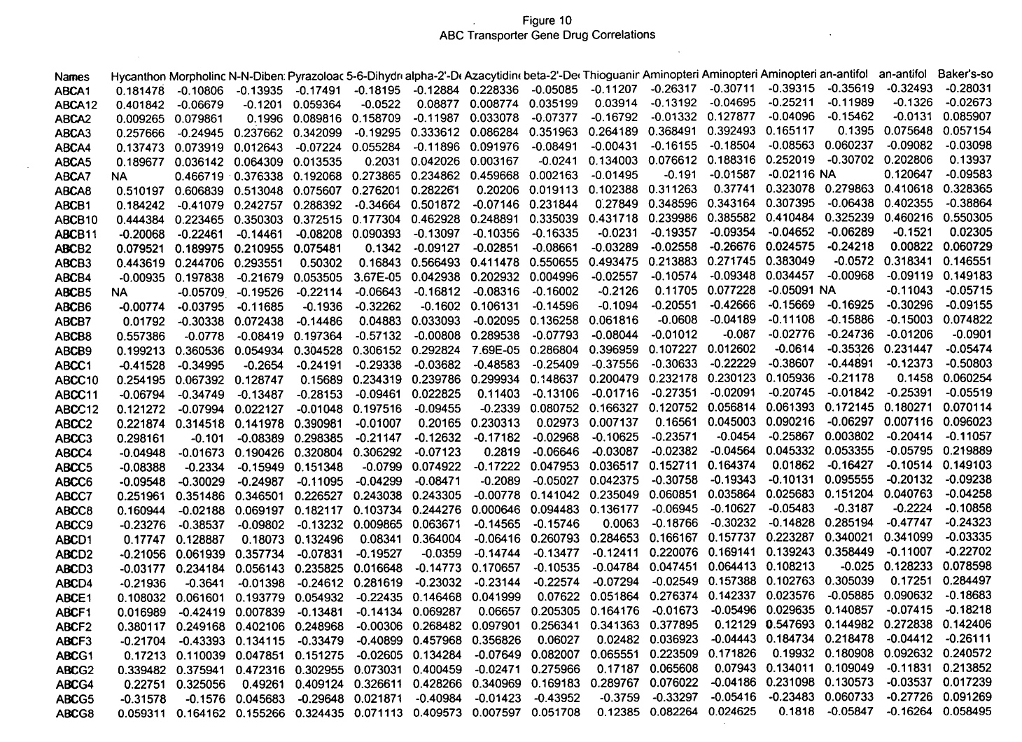

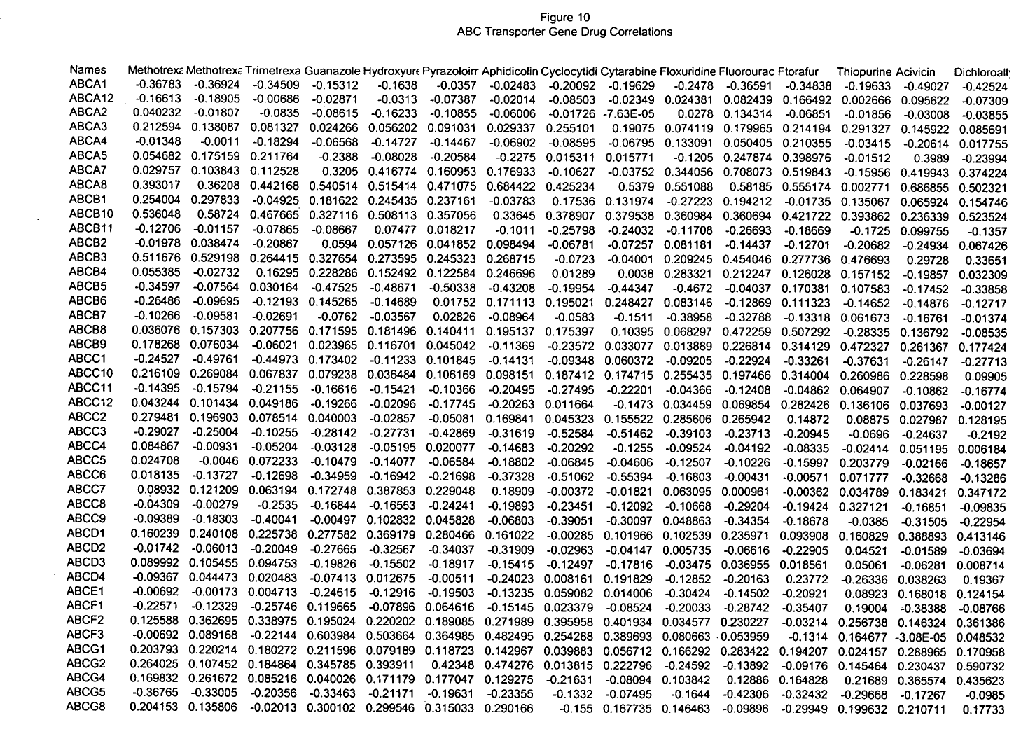

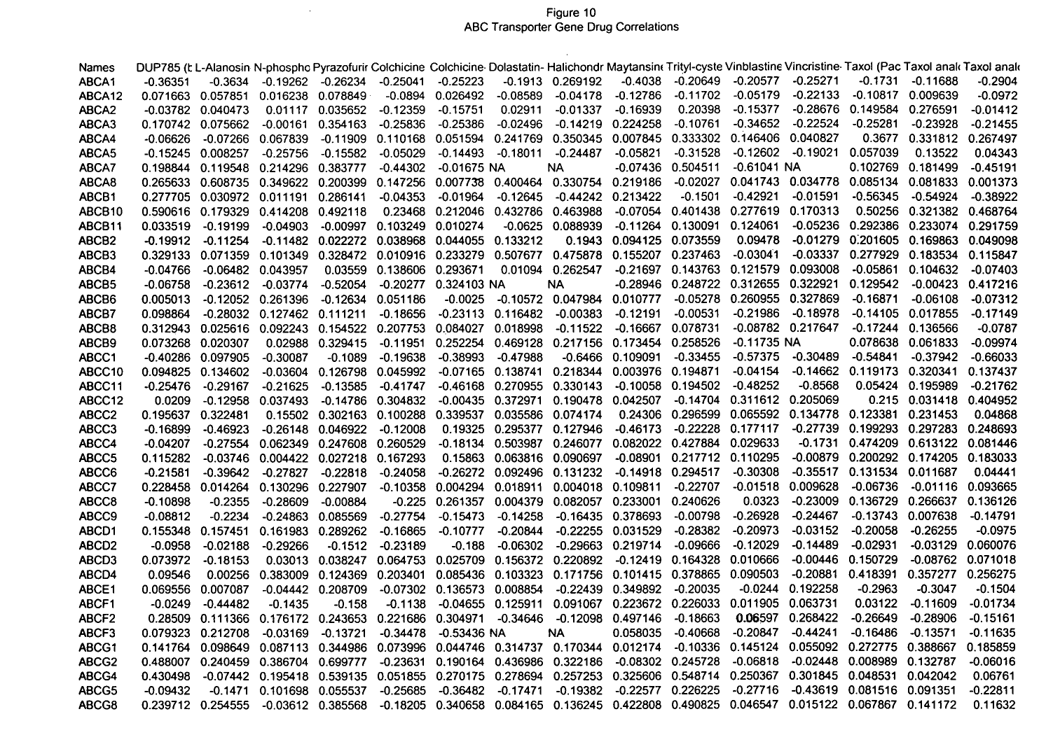

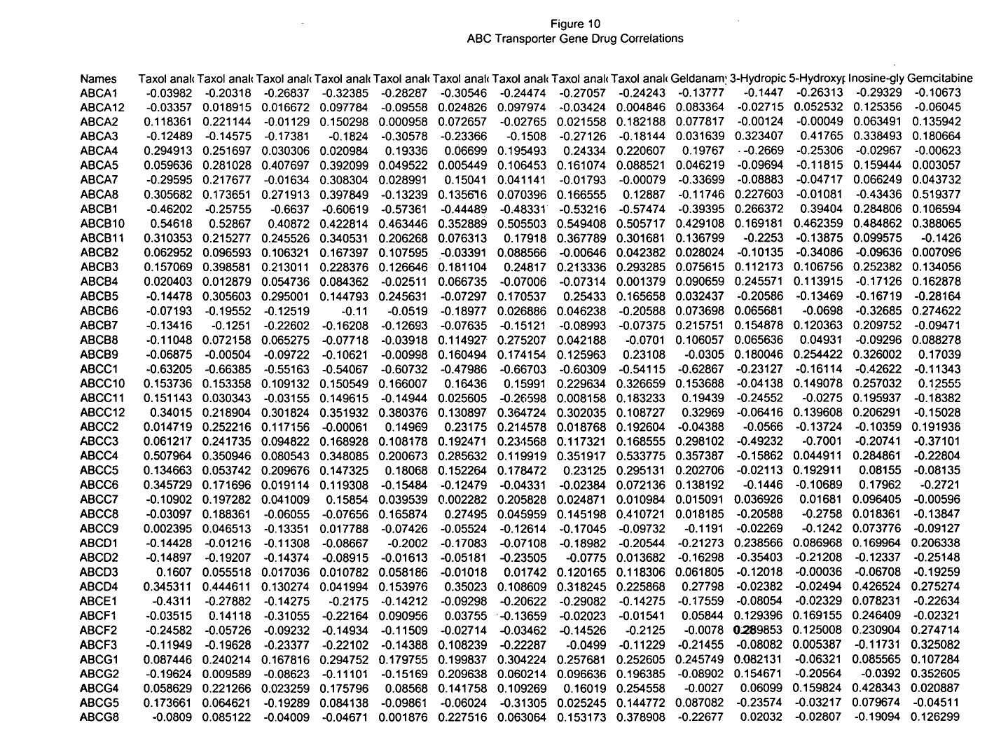

In one aspect, the present invention provides a chemosensitivity gene expression analysis system comprising a plurality of polynucleotide probes, wherein each of said polynucleotide probes comprises a nucleic acid sequence that is complimentary under strict hybridization conditions to at least a portion of a gene that encodes a protein that is a marker for the sensitivity of cancer cells to cytotoxic agents, as presented in FIG. 9 and FIG. 10, and in TABLES 1-6. In some embodiments, polynucleotides probes are provided on an array. Examples of probes are presented in FIG. 8.

When the polynucleotide probes are employed as hybridizable array elements in an array, the array elements are organized in an ordered fashion so that each element is present at a specified location on the substrate. Because the array elements are at specified locations on the substrate, the hybridization patterns and intensities (which together create a unique expression profile) can be interpreted in terms of expression levels of particular genes and can be correlated with a particular disease or condition or treatment.

The gene expression analysis system, in some embodiments in the form of an array, can be used for gene expression analysis of target polynucleotides that represent the expression products of cells of interest, particularly cancer cells. The array can also be used in the prediction of the responsiveness of a patient to a therapeutic agent, such as the response of a cancer patient to a chemotherapeutic agent. Further, as described below, the array can be employed to investigate the profile of a cancer cell in terms of its likely sensitivity or resistance to chemotherapeutic agents. Furthermore, as described below, the array can be employed to characterize a therapeutic agent's chemosensitivity profile for use in treating various cancers. The array can also be used to identify new agents, as described below, which can modulate the chemosensitivity of a cancer cell to one or more therapeutic agents by altering the expression of genes that are markers for and influence chemosensitivity.

The gene expression analysis system can be used to purify a subpopulation of mRNAs, cDNAs, genomic fragments and the like, in a sample. Typically, samples will include target polynucleotides and other non-target nucleic acids that may undesirably affect the hybridization background. Therefore, it may be advantageous to remove these non-target nucleic acids from the sample. One method for removing the non-target nucleic acids is by contacting the polynucleotide sample with the array, hybridizing the target polynucleotides contained therein with immobilized polynucleotide probes under hybridizing conditions. The non-target nucleic acids that do not hybridize to the polynucleotide probes are then washed away, and thereafter, the immobilized target polynucleotide probes can be released in the form of purified target polynucleotides.

Examples of the types of molecules that may be used as probes are cDNA molecules, oligonucleotides that contain 15, 16, 17, 18 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 5, 6, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more nucleotides, and other gene probes that comprise nucleobases including synthetic gene probes such as, for example, peptide nucleic acids. At least some of said polynucleotide probes comprise a polynucleotide sequence that is complementary to a target region of a gene that encodes a protein associated with transport of molecules into and out of cells and that is a marker for the sensitivity or resistance of cancer cells to cytotoxic agents. In one embodiment, the plurality of polynucleotide probes comprises at least two or more probes, each of which comprises a polynucleotide sequence that is complementary to a target region of a chemosensitivity gene listed in one of FIG. 9 and FIG. 10, or in one of Tables 1-6. Provided in FIG. 8 are examples of polynucleotides probes that are complementary to and hybridize with target regions of chemosensitivity genes, as well as several control probes that do not hybridize with chemosensitivity genes. The chemosensitivity gene probes include those oligos indicated as “transporter,” “channel,” “conting;” control probes are indicated as “control,” “ADAMS,” “RGS,” or “double.”

In some embodiments, the probes are attached to a solid support such as for example a glass substrate. Among the probes are molecules that hybridize under stringent conditions with transcripts of the newly-identified chemosensitivity transporters shown in FIG. 9 and FIG. 10, and in TABLES 1-6. The array comprises two or more probes, each of which probes are specific for and hybridize to a transcripts one of the chemosensitivity transporters.

The present invention also provides arrays comprising a plurality of oligonucleotides, wherein: a) the oligonucleotides are chosen from the nucleic acid sequences listed in FIG. 9, and wherein the array comprises 10 or more of said oligonucleotides; or b) the oligonucleotides comprise nucleotide probes designed to be complementary to, or hybridize under stringent conditions with, 10 or more chemosensitivity genes listed in listed in one of FIG. 9 and FIG. 10, or in one of Tables 1-6. In some embodiments, the oligonucleotides comprise nucleotide probes designed to be complementary to, or hybridize under stringent conditions with target regions of 10, 20, 30, 40, 50, 60, 70, 80, 90, 100, 200, or more chemosensitivity genes listed in one of FIG. 9 and FIG. 10, or in one of Tables 1-6.

In another aspect, the present invention provides a physical embodiment of the expression profile for a cancer cell of proteins that transport molecules into and out of cells and that are markers for the sensitivity of cancer cells to cytotoxic agents. The expression profile comprises the polynucleotide probes of the invention. The expression profile also includes a plurality of detectable complexes, in some embodiments in the form of a gene expression analysis system, and in some embodiments in the form of an array. Each complex is formed by hybridization of one or more polynucleotide probes to one or more complementary target polynucleotides in a sample. The polynucleotide probes are hybridized to a complementary target polynucleotide forming target/probe complexes. A complex is detected by incorporating at least one labeling moiety in the complex. Labeling moieties are described herein and are well known in the art.

In another embodiment, the chemosensitivity expression profile comprises a printed report that shows the expression of the analysis of an array. The printed report may be in the form of a developed or digital film of the hybridized and developed gene expression analysis system. The printed report may also be a manually or computer generated numerical analysis of the developed gene expression analysis system. The printed report may optionally contain gene-drug correlation information. The expression profiles provide “snapshots” that can show unique expression patterns that are characteristic of susceptibility or resistance of a cell to one or more cytotoxic chemotherapeutic agents.

The chemosensitivity expression profile can be used, as further described below: (a) in the prediction of the chemosensitivity of a particular cancer cell or cell type to a therapeutic agent; (b) in the choice of drug therapy for a patient in need of the same; (c) in the identification of targets for altering the chemosensitivity of a cancer; and (d) in the identification of novel agents for modulating the chemosensitivity of a cancer.

Methods of Predicting Response to Therapeutic Agents

In another aspect, the present invention provides a method of predicting the response of a specific cancer, and more particularly a cancer in a patient, to treatment with a therapeutic agent. The method comprises contacting a polynucleotide sample obtained from the cells of the specific cancer to polynucleotide probes to measure the levels of expression of one or, in some embodiments, a plurality of target polynucleotides. The expression levels of the target polynucleotides are then used to provide an expression profile for the cancer cells that is then compared to the drug-gene correlations, such as those listed in FIG. 9 and FIG. 10, and in Tables 1-6, wherein a positive correlation between a drug and a gene expressed in the cancer cells indicates that the cancer cells would be sensitive to the drug, and wherein a negative correlation between a drug and a gene expressed in the cancer cells indicates that the cancer cells would be resistant to the drug.

Methods of Identifying New Therapeutic Agents

The present invention provides novel methods for identifying and characterizing new agents that modulate the chemosensitivity of a cancer by altering the expression of one or more transporter genes. The method comprises treating a sample of cells from the cancer with an agent, and thereafter determining any change in expression of genes, such as transporter protein genes, which are markers for chemosensitivity. This is done by obtaining polynucleotide samples from untreated cancer cells and the treated cancer cells, and contacting the polynucleotide samples to polynucleotide probes to determine the levels of target polynucleotides to obtain transporter gene chemosensitivity expression profiles. In some embodiments, the measurement is made using an array or micro array as described above that comprises one or more probes, examples of which are presented in FIG. 9 and FIG. 10, and in Tables 1-6. The method further comprises comparing the transporter gene expression profiles of the control and treated cells to determine whether the agent alters the expression of any of the chemosensitive or chemoresistant genes. In some embodiments, separate cultures of cells are exposed to different dosages of the candidate agent. The effectiveness of the agent's ability to alter chemosensitivity can be tested using standard assays that use, for example, the one or more of the NCI60 cancer cell lines. The agent is tested by conducting assays in that sample cancer cells are co treated with the newly identified agent along with a previously known therapeutic agent. The choice of previously known therapeutic agent is determined based upon the gene-drug correlation between the gene or genes whose expression is affected by the new agent. The present invention further provides novel methods for identifying and characterizing new agents that modulate the chemosensitivity of a cancer by altering the activity of one or more transporter genes. The method comprises treating a sample of cells from the cancer with an agent, which is capable of inhibiting the activity of a transporter protein implicated in chemosensitivity by correlation analysis between gene expression and drug potency in multiple cancer cell lines. For example, an inhibitor of an efflux pump will increase the potency of an anticancer drug if the efflux pump is highly expressed. This permits one to search either for inhibitors of the chemosensitivity gene or to test whether an anticancer agent is subject to transport by the chemosensitivity gene product.

Any cell line that is capable of being maintained in culture may be used in the method. In some embodiments, the cell line is a human cell line, such as, for example, any one of the cells from the NCI60 cell lines. According to one approach, RNA is extracted from such cells, converted to cDNA and applied to arrays to that probes have been applied, as described above.

### EXAMPLES

The invention may be better understood by reference to the following examples, which serve to illustrate but not to limit the present invention.

### Example 1

### Identification of Chemosensitivity Gene Drug Correlations

Gene-Drug Correlations:Gene expression profiles of membrane transporters and channels were compared with potency of 119 drugs in the NCI60 panel of cancer cells shown in Table A.

TABLE A
NCI60 Cancer Cell Lines (12 reference pool lines)

| Colon | Renal | Ovarian | Melanoma | CNS | Leukemia | Breast | Lung |
|---|---|---|---|---|---|---|---|
| βCOLO205 | 786-0 | IGROV1 | βLOXIMVI | SF-268 | CCRF-CEM | βMCF7 | A549/ |
| HCC-2998 | A498 | βOVCAR-3 | MALME-3M | SF-295 | βHL-60(TB) | NCI/ADR- | ATCC |
| HCT-116 | ACHN | βOVCAR-4 | M14 | SF-539 | βK-562 | RES | EKVX |
| HCT-15 | βCAKI-1 | VCAR-5 | SK-MEL-2 | βSNB-19 | MOLT-4 | MDA-MB-231/ | HOP-62 |
| HT29 | RXF393 | VCAR-8 | SK-MEL-28 | SNB-75 | RPMI-8226 | ATCC | HOP-92 |
| KM12 | SN12C | SK-OV-3 | SK-MEL-5 | U251 | SR | βHS578T | βNCI-H226 |
| SW-620 | TK-10 | | UACC-257 | Prostate | | MDA-MB-435 | NCI-H23 |
| | UO-31 | | UACC-62 | βPC-3 | | MDA-N | NCI-H322M |
| | | | | DU-145 | | BT-549 | NCI-H460 |
| | | | | | | T-47 | NCI-H522 |

Gene expression and chemo-sensitivity were analyzed and chemosensitivity genes were identified by employing correlation analysis according to the method of Scherf et al. that combined genome-wide expression profiling with drug activity data to identify putative gene-drug relationships. The Scherf study generated a number of testable hypotheses, but several problems remained unresolved.

TABLE 1
Select transporter genes showing correlations with chemosensitivity

| | Multiplicity | | | | | |
|---|---|---|---|---|---|---|
| | P < 0.001 | | P < 0.05 | | | |
| Gene | r > 0 | r < 0 | r > 0 | r < 0 | Substrate | Representative drug |
| SLC transporters | | | | | | |
| SLC23A2 | 1 | 0 | 8 | 0 | nucleobase | [5FU] |
| SLC28A1 | 0 | 0 | 7 | 0 | nucleoside | [Aminopterin][6MP] |
| SLC28A3 | 2 | 0 | 38 | 0 | nucleoside | [Thioguanine][Cytarabine (araC)][Gemcitabine] |
| SLC29A1 | 2 | 0 | 21 | 0 | nucleoside | [CCNU][Azacytidine][Thioguanine] |
| SLC29A2 | 0 | 0 | 2 | 1 | nucleoside | [alpha-2′-Deoxythioguanosine][Inosine-glycodialdehyde] |
| SLC19A1 | 0 | 0 | 19 | 0 | folate | [6MP][Gemcitabine] |
| SLC19A2 | 2 | 0 | 24 | 1 | folate | [Tetraplatin][lproplatin][an-antifol][Trimetrexate] |
| SLC19A3 | 0 | 0 | 4 | 0 | folate | [an-antifol] |
| SLC1A1 | 1 | 0 | 24 | 1 | amino acid | [L-Asparaginase][L-Alanosine] |
| SLC1A4 | 3 | 1 | 11 | 12 | amino acid | [Asaley][Taxol analog][Acivicin][L-Alanosine] |
| SLC3A1 | 0 | 0 | 4 | 0 | amino acid | [L-Asparaginase] |
| SLC7A2 | 0 | 0 | 13 | 0 | amino acid | [L-Alanosine] |
| SLC7A3 | 0 | 0 | 12 | 0 | amino acid | [L-Asparaginase] |
| SLC7A8 | 0 | 0 | 0 | 14 | amino acid | [N-phosphonoacetyl-L-aspartic-acid] |
| SLC7A9 | 0 | 0 | 3 | 1 | amino acid | [Acivicin] |
| SLC7A11 | 2 | 0 | 12 | 5 | amino acid | [Anthrapyrazole][Colchicine][L-Alanosine] |
| SLC15A1 | 0 | 0 | 5 | 0 | peptide | [Fluorodopan][Teroxirone][Etoposide][L-Asparaginase] |
| SLC25A12 | 4 | 0 | 29 | 0 | aspartate glutamate | [Thioguanine][N-phosphonoacetyl-L-aspartic-acid] |
| SLC25A13 | 0 | 0 | 0 | 41 | aspartate glutamate | [L-Asparaginase][CPT][Hepsulfam] |
| SLC38A2 | 1 | 0 | 14 | 0 | amino acid | [Maytansine][Acivicin][L-Alanosine] |
| SLC38A5 | 2 | 0 | 25 | 0 | amino acid | [Clomesone][Colchicine][L-Asparaginase] |
| SLC2A5 | 0 | 2 | 0 | 12 | glucose | [Aminopterin][Aminopterin] |
| SLC2A11 | 2 | 0 | 38 | 0 | glucose | [Anthrapyrazole][Oxanthrazole] |
| SLC9A3R2 | 2 | 0 | 21 | 0 | sodium/hydrogen | [CPT, 9-MeO][L-Asparaginase] |
| LOC133308 | 7 | 0 | 51 | 0 | sodium/hydrogen | [CCNU][6MP][Doxorubicin][Taxol analog] |
| SLC4A7 | 18 | 0 | 56 | 0 | sodium bicarbonate | [Mitomycin][Spiromustine][CPT, 10-OH][Mitoxantrone] |
| ABC transporters | | | | | Alias | |
| ABCB1 | 0 | 3 | 2 | 32 | MDR | [Bisantrene][Taxol analog] |
| ABCB5 | 0 | 1 | 1 | 25 | | [CPT, 7-Cl] |
| ABCC3 | 0 | 2 | 0 | 18 | MRP3 | [Vincristine][Methotrexate-derivative] |
| Ion pump | | | | | Substrate | |
| ATP1A1 | 0 | 5 | 6 | 43 | sodium/potassium | [Uracil mustard][CPT, 11-formyl (RS)] |
| ATP1A3 | 0 | 3 | 0 | 24 | sodium/potassium | [CCNU][Daunorubicin][5-6-Dihydro-5-azacytidine] |
| ATP1B1 | 0 | 10 | 0 | 34 | sodium/potassium | [CCNU][Tetraplatin][Inosine-glycodialdehyde] |
| ATP1B3 | 0 | 0 | 2 | 20 | sodium/potassium | [Daunorubicin][5FU] |
| ATP1G1 | 0 | 0 | 4 | 14 | sodium/potassium | [Tetraplatin][Taxol analog] |
| ATP2A1 | 1 | 0 | 12 | 0 | calcium | [Morpholino-adriamycin][Doxorubicin][5FU] |
| ATP2A3 | 0 | 0 | 37 | 0 | calcium | [BCNU][Gemcitabine] |
| ATP2B3 | 0 | 0 | 0 | 8 | calcium | [Colchicine-derivative][Taxol analog] |
| ATP2B4 | 0 | 11 | 0 | 44 | calcium | [Tetraplatin][Methotrexate][5FU][Taxol analog] |
| ATP2C1A | 0 | 3 | 0 | 31 | calcium | [Iproplatin][Mechlorethamine][Deoxydoxorubicin] |
| ATP6V1D | 0 | 0 | 0 | 25 | proton | [Daunorubicin][Methotrexate][Taxol analog] |
| Ion channel | | | | | | |
| AQP1 | 0 | 4 | 0 | 20 | water | [Aminopterin][an-antifol][Methotrexate] |
| AQP4 | 0 | 1 | 0 | 10 | water | [L-Alanosine] |
| AQP9 | 1 | 0 | 11 | 1 | water, urea, arsenite | [Taxol analog] |
| MIP | 2 | 0 | 46 | 0 | water | [Pipobroman][Halichondrin B] |
| CACNA1D | 0 | 4 | 0 | 37 | calcium | [Mitozolamide][Cyclodisone][Deoxydoxorubicin] |

Referring to Table 1, activities of the underlined drugs have negative correlations with expression of the corresponding genes. Others drugs have positive correlations. For each gene, multiplicity, the number of drugs with positive or negative correlation values (r) is shown with two cut-off point, P<0.05 and P<0.001. Only selected genes are shown.

As described herein, Applicant examined gene expression using in one embodiment a custom-designed 70mer oligonucleotide array, which is in principle more specific than cDNA array and more suitable for studying closely related genes. Numerous putative and confirmed gene-drug pairs emerged from an analysis of the 70-mer oligo.

The present work surveyed large number of transporter genes thought to play a pervasive role in drug sensitivity. Moreover, genes encoding ATPases and ion channels were surveyed since they play an important role in establishing and maintaining the electrochemical gradient across the membrane, which is important for drug transport and cell viability. Alteration in drug accumulation within the cells of target tissues ranks as a common resistance mechanisms occurring in tumor cells. By focusing on these genes, significant gene-drug correlations allow prediction of the sensitivity of cancer cells to particular drugs and also allow for the generation of hypotheses that are readily testable using classical transporter assays. The array probes were assembled to include genes from each transporter subfamily by searching existing databases, including EST collections (Brown et al.). For example, we included at least 40 of the 48 known human ABC transporter genes.

To determine the relationship between gene expression of all the 732 probes and cytotoxic drug potency generated for the same 60 cancer cell lines, Applicant calculated Pearson correlation coefficients for each gene-drug pair. Positively correlating genes are likely to reflect chemosensitivity whereas genes with negative correlations suggest chemoresistance. The validity of this approach is supported by significant correlations obtained for gene-pairs that reflect previously published transporter-substrate interactions. This analysis yielded 732 correlation coefficients for each of the selected 119 drugs. Of the 87,108 gene-drug correlations, 2.5% were either above 0.262 or below −0.267. Therefore, as a first approximation, 0.3 was selected as a threshold for potentially significant correlations.

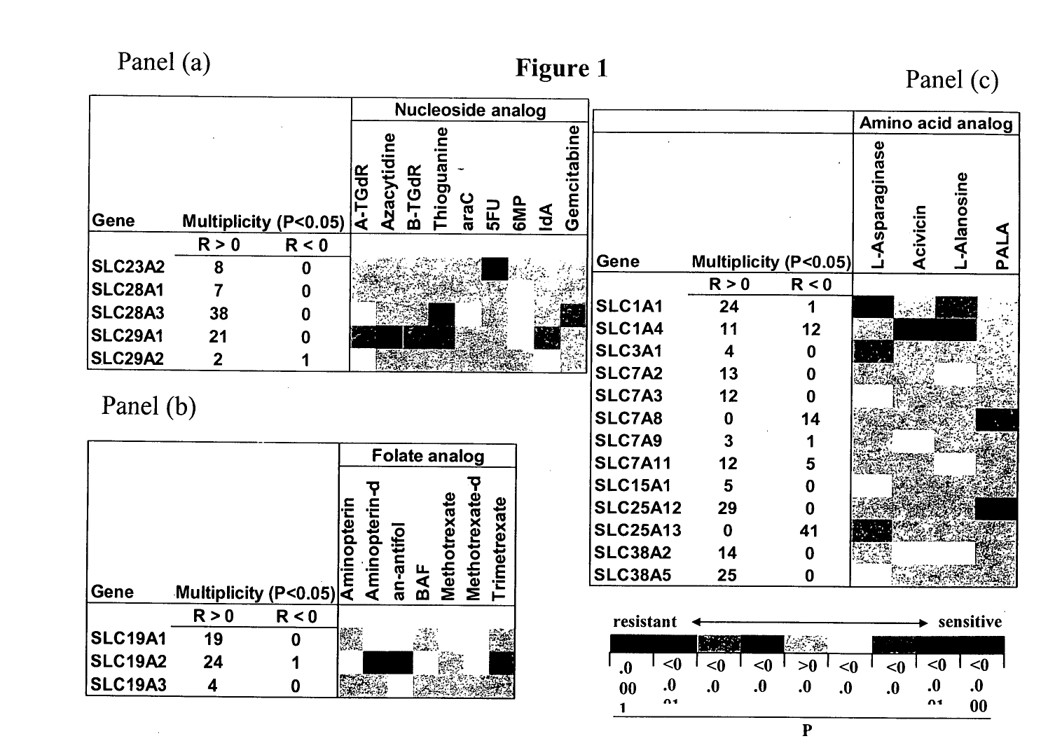

To assess statistical significance, for each gene-drug correlation, Applicant computed an unadjusted bootstrap P value. Novel candidate genes involved in chemosensitivity were selected when bootstrap P value of the correlation was less than 0.001. For transporter genes previously implicated in chemosensitivity, Applicant used P<0.05 as the cut-off. Using these criteria, Applicant identified 177 (0.2%) gene-drug pairs showing positive correlations, 210 pairs (0.2%) showing negative correlations, involving 145 genes linked to at least one drug. Applicant used a more relaxed stringency (P<0.05) to identify potential substrates in the 119-drug panel. Table 1 and FIG. 1 show select genes and representative substrates. For each gene listed, the number of correlated drugs (multiplicity) is shown for both cut-off points, P<0.001 and P<0.05 (Table 1, and Tables 4 to 6). The criteria for assessing validity of a candidate gene include concordance (Pearson correlation coefficient r>0.3) in at least one comparison between the oligo-probe array with other expression datasets for the NCI60, where available. In accordance with these findings, new model systems useful for predicting the chemosensitivity or resistance of a cancer are provided.

Selection of Genes

Hidden Markov Models (HMMs) of transporter and ion channel genes were selected by searching the Pfam Database 6.1 (this information can be found on the internet at URL //pfam.wust1.edu/) with keywords and seed sequences chosen from known transporter and channel families (this information can be found on the internet at URL //www-biology.ucsd.edu/˜msaier/transport/toc.html). HMMs were run against the Genpept database using hmmsearch/hmmer-2.1.1-intel-linux (this information can be found on the internet at URL //hmmer.wust1.edu/). Only hits with a probability of 0.0001 or lower were selected. Using the multiple alignment program ClustalW, redundant accession numbers were filtered out. In addition, new putative transporter and channel gene sequences were collected. An automated search method was applied that uses converged PSI-Blast against the human EST database for identification of new gene candidates (14). Resulting contig sequences representing two or more overlapping ESTs were used for the array. Since this work was done before the release of the human sequence, contigs identified in our search were then run against the human genome database, and annotated genes matching the contigs were identified. Several contigs did not match any annotated genes, and therefore, might represent yet uncharacterized genes. Identity of these putative genes will be studied separately. Housekeeping genes and negative controls were the same as in the Atlas 1.2 Human Array by Clontech.

Design of Oligomers

Coding region sequences only were used for the design of the oligomers. To select the 70mers, an algorithm was applied that takes the following four criteria into account: uniqueness, internal palindrome structure (reverse Smith-Waterman algorithm is used to detect palindrome sequence), melting temperature ^{TM} and localization of the 70mer probe within the gene sequence (15). For the design of the 70mers, a TM of 70° C., an internal palindrome structure value of 100 and a uniqueness cutoff of 15 bp were chosen. All oligomers were designed to be located as close toward the 3′ end as possible.

### Example 2

### Solute Carriers (SLCs) and Chemosensitivity

Solute carriers encode the transportome for amino acids, peptides, sugars, monocarboxylic acid, organic cations, phosphates, nucleosides and water-soluble vitamins. Table 1 and FIG. 1 summarize the results for select SLC genes that showed significant Pearson correlation for at least one drug. Several identified transporters have previously been implicated in drug transport or they transport natural substrates similar in structure to correlated drugs. Thus, nucleobase transporters (SLC23A2) and nucleoside transporters of both ENT (equilibrative) and CNT (concentrative) families showed positive correlations with a number of drug analogues (FIG. 1a), as expected for transporters that facilitate drug entry into cells. For example, SLC29A1 (equilibrative nucleoside transporter 1, ENT1) positively correlated with azacytidine (FIG. 1a) and ENT2 with alpha-2′-deoxythioguanosine, consistent with the notion that these transporters are essential for nucleoside drug uptake. These results indicate that individual nucleoside transporters play a significant role in chemosensitivity; moreover, significant correlations identify putative substrates among the compounds tested at the NCI.

FIG. 1b reveals that SLC19A1, A2 and A3, members of the reduced folate carrier protein family, positively correlated with folate analogs, such as aminopterin and trimetrexate. This result is consistent with previous findings and extends the spectrum of putative substrates. Therefore, impaired transport of folate drugs is a potential mode of drug resistance. Again the correlation analysis indicates which drugs are likely substrates for individual folate transporters.

Amino acid transporters had received less attention as drug carriers. FIG. 1c depicts several amino acid transporters that correlate with amino acid analogs, a finding not previously noted. For example, SLC38A2 (or ATA2), a member of the amino acid transport system A, positively correlated with acivicin and L-alanosine, amino acid analog drugs. SLC25A12, encoding a calcium-stimulated aspartate/glutamate carrier protein (Aralar1) located at the mitochondrial inner membrane, showed positive correlation with N-phosphonoacetyl-L-aspartic-acid. In contrast, SLC25A13, encoding Citrin, another calcium-stimulated aspartate/glutamate transporter in mitochondria homologous to Aralar 1, showed negative correlation to L-asparaginase (−0.55), possibly by providing aspartate precursor to the cells. Moreover, the correlation coefficient was −0.96 (confidence interval −1.00 to −0.87) for the six leukemic lines, targets of L-asparaginase treatment. This parallels previous results with the NCI60 implicating ASNS (asparagine synthetase) as a resistance gene, particularly in leukemic cells. Both SLC25A13 and ASNS play a role in urea and arginine synthesis and are located in chromosome 7q21.3 with a distance of less than 100 kb. Possible co-expression or chromosomal amplification involving these two genes should be considered in future studies.

Several SLC genes correlated with multiple drugs of different structure (Table 4). This may not reflect a transporter-substrate relationship, but rather alternative functions of the transporter. Select nutrient transporters (glucose, amino acids, organic anions, peptides) may be upregulated, satisfying the increased energy need of cancer cells. Thus, glucose transporters could affect drug potency by serving as drug carriers or modulating cellular drug toxicity. Shown in Table 4, expression of several glucose transporters (e.g., SLC2A5) is positively or negatively correlated with numerous drugs. Since highly significant correlations are included, these results provide the rationale for further analysis of underlying mechanisms, including a relationship between glucose metabolism and apoptosis.

Intracellular pH has been shown to affect cellular response to anticancer drugs. Two SLC ion exchangers function as pH regulators in tumor cells, a bicarbonate transporter and a sodium-proton exchanger. Among members of Na⁺/H⁺ exchanger (NHE) family, SLC9A3R2 showed positive association with multiple drugs, conferring chemo-sensitivity (Table 4). Moreover, an EST encoding a hypothetical protein LOC133308 with Na⁺/H⁺ exchanger motif positively correlated with several drugs. Among the bicarbonate transporters, SLC4A 7 positively correlated with 56 drugs. Therefore, genes affecting pH have pervasive effects on multiple drugs.

### Example 3

### ABC Transporters and Chemoresistance

Among 40 genes tested that encode ABC transporters, 11 showed negative correlations (Table 2). Nine of these genes had been previously implicated in drug resistance. Only four genes showed highly significant negative correlations (P<0.001). Expression data obtained by other methods validated results for three of these genes (ABCB1, ABCC3, and ABCB5—a putative novel resistance gene) (Table 2).

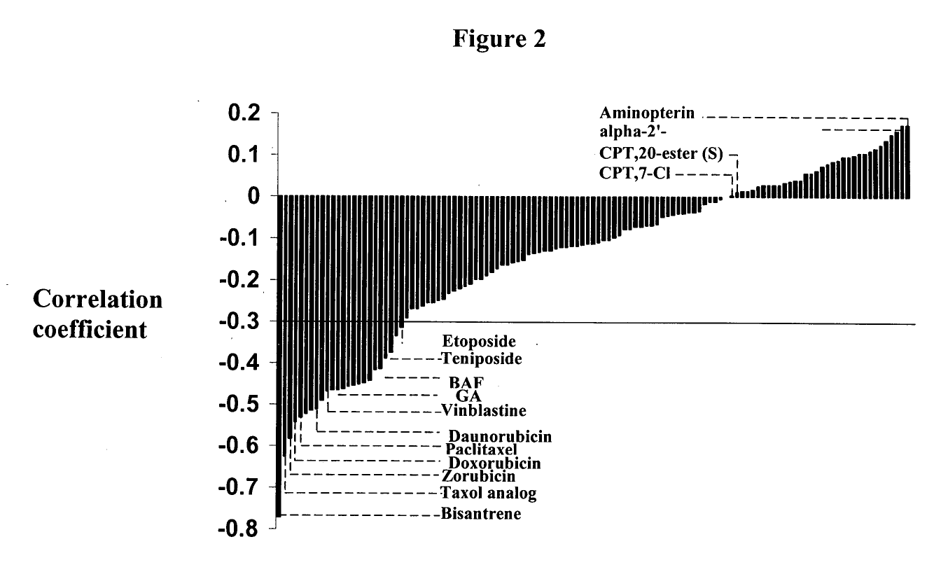

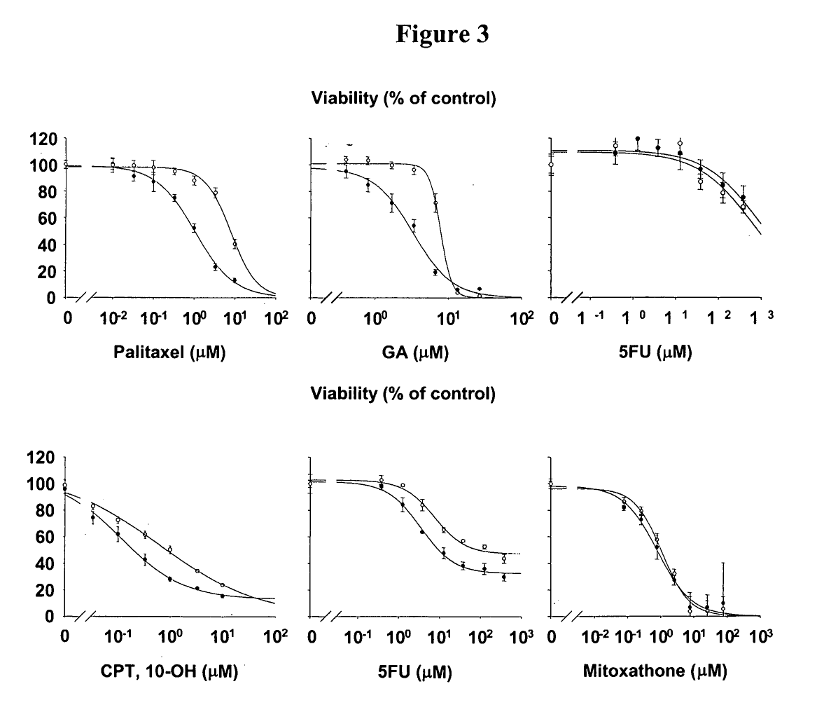

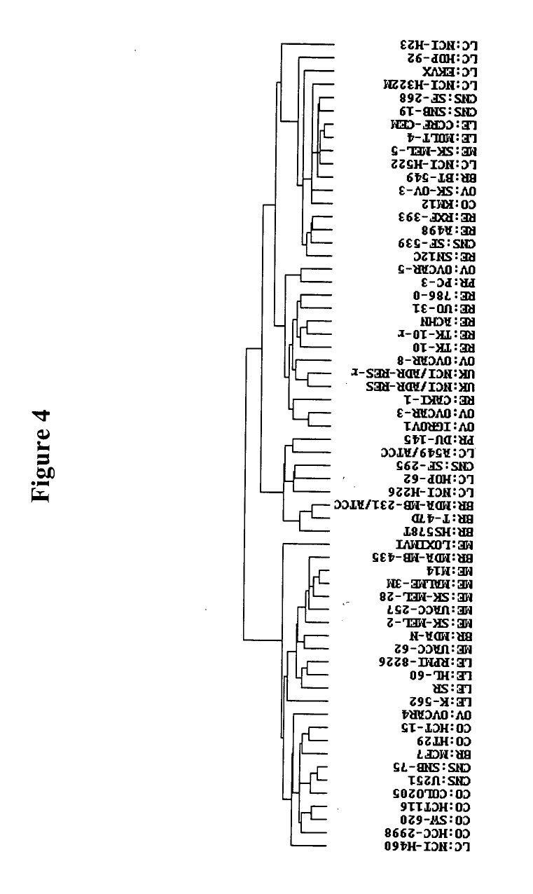

Expression levels of ABCB1 (or MDR1, Pgp) significantly correlated with potency of many drugs (Table 1). A plot of the ordered ABCB1 correlation coefficients for all 119 drugs revealed a clear separation between known ABCB1 substrates and non-substrates (FIG. 2). Using the dual criteria of P<0.05 and r<−0.3, we identified all known substrates of ABCB1 plus geldanamycin (GA) (NSC 330500) and Baker's-soluble-antifol (BAF) (NSC 139105) (Table 2). These results were validated by silencing of ABCB1 gene expression using RNA interference (RNAi) (see below). ABCC3, encoding multidrug resistance-associated protein 3 (MRP3), also showed significant negative correlation with a methotrexate-derivative, which is consistent with the observation that overexpression of ABCC3 resulted in high-level resistance to methotrexate. ABCB5 (P<0.001 for CPT, 7-Cl) showed strong negative correlation with CPT, 7-Cl. ABCB5 is selectively expressed in melanoma cells, suggesting a tissue-specific role in chemoresistance (see RNAi validation).

The known chemoresistance genes ABCA2, ABCB2, ABCB11, ABCC1, ABCC2, ABCC4, and ABCC5 were negatively associated with several drugs (P<0.05) (Table 2). However, the suggested drug substrates differed from those reported before, and the relatively low correlations argue against a significant role in the NCI60 panel. Moreover, measured expression of these genes did not correlate well with results obtained by real-time RT-PCR (Table 1 and Gottesman et al. unpublished data). This may be related to insufficient sensitivity of the 70-mer oligo array. Further validation is needed before chemoresistance can be inferred or excluded.

TABLE 2
Drugs showing significant negative correlation with ABCB1.

| | | | r (70-mer | |
|---|---|---|---|---|
| Drug | NSC No. | P value | array) | r (cDNA array) |
| Taxol analog_7 | 666608 | 0.000 | −0.62 | −0.50 |
| Taxol analog_10 | 673187 | 0.000 | −0.52 | −0.42 |
| Bisantrene | 337766 | 0.001 | −0.77 | −0.49 |
| Taxol (Paclitaxel) | 125973 | 0.001 | −0.53 | −0.54 |
| Taxol analog_3 | 658831 | 0.001 | −0.45 | −0.42 |
| Taxol analog_5 | 664402 | 0.001 | −0.49 | −0.40 |
| Taxol analog_6 | 664404 | 0.002 | −0.51 | −0.39 |
| Baker's-soluble- | 139105 | 0.002 | −0.39 | −0.30 |
| antifol | | | | |
| Taxol analog_2 | 656178 | 0.002 | −0.45 | −0.43 |
| Vinblastine-sulfate | 49842 | 0.003 | −0.47 | −0.33 |
| Geldanamycin | 330500 | 0.004 | −0.46 | −0.48 |
| Taxol analog_11 | 673188 | 0.011 | −0.47 | −0.48 |
| Oxanthrazole | 349174 | 0.012 | −0.46 | −0.07 |
| Taxol analog_8 | 671867 | 0.015 | −0.42 | −0.42 |
| Taxol analog_9 | 671870 | 0.016 | −0.44 | −0.49 |
| Anthrapyrazole- | 355644 | 0.016 | −0.45 | −0.12 |
| derivative | | | | |
| Daunorubicin | 82151 | 0.017 | −0.51 | −0.23 |
| Etoposide | 141540 | 0.020 | −0.31 | −0.09 |
| Doxorubicin | 123127 | 0.020 | −0.54 | −0.27 |
| Zorubicin | 164011 | 0.021 | −0.58 | −0.31 |
| Taxol analog_1 | 600222 | 0.041 | −0.46 | −0.53 |
| 5,6-Dihydro- | 264880 | 0.463 | −0.16 | −0.30 |
| 5-azacytidine | | | | |

Referring to Table 2, the results from both 70-mer oligo arrays and cDNA arrays (http://discover.nci.nih.gov) are shown. P values were calculated for the 70-mer oligo array data only. The listed drugs fulfill both criteria for the 70-mer oligo array data: P<0.05 and r<−0.3; the criteria for cDNA array data are: r<−0.3 only. We identified 19 putative ABCB1 substrates, all but two are known substrates among the 119 drugs. The two remaining drugs were validated as substrates by siRNA. Note that the cDNA array failed to identify several substrates and yielded r=−0.3 for 5,6-dihydro-5-azacytidine, which is not a substrate for ABCB1.

The results from both 70-mer oligo arrays and cDNA arrays (http://discover.nci.nih.gov) are shown. P values were calculated for the 70-mer oligo array data only. The listed drugs fulfill both criteria for the 70-mer oligo array data: P<0.05 and r<−0.3; the criteria for cDNA array data are: r<−0.3 only. We identified 19 putative ABCB1 substrates, all but two are known substrates among the 119 drugs. The two remaining drugs were validated as substrates by siRNA. Note that the cDNA array failed to identify several substrates and yielded r=−0.3 for 5,6-dihydro-5-azacytidine, which is not a substrate for ABCB1.

### Example 4

### Ion Pumps (ATPases), Channels and Chemosensitivity

To identify ion pumps associated with drug activity, we investigated ATPases that maintain cellular electrical gradients (Table 1 and Table 3). Genes encoding ATP1A1, 1A3, 1B1, 1B3 and 1G1—isoforms of the α, β and γ subunits of Na⁺/K⁺-ATPase—showed negative correlations with a number of drugs. Na⁺/K⁺-ATPase, responsible for maintaining electro-chemical gradients, plays a role in cell proliferation and appears to serve as resistance factor. Moreover, genes encoding subunits of the calcium pumps, ATP2A1, A3, B3, B4, and C1A showed either positive or negative correlation with drugs. Opposite effects may be due to the mechanism of action and charge of the chemotherapeutic agent. Calcium content, release, and transfer from the endoplasmic reticulum to mitochondria appears to play a key role in apoptosis, thus implicating calcium flux as an important factor in drug toxicity. In addition, ATP6V1D, encoding a subunit of vacuolar H⁺-ATPase, which mediates acidification of intracellular organelles, negatively correlated with 25 drugs, which is consistent with previous observations that vacuolar ATPase-mediated pH regulation is a factor in anticancer drug resistance. Our combined results indicate that sodium/potassium, calcium, and proton ATPases modulate chemoresistance.

TABLE 3
Enhanced chemosensitivity by siRNAs-targeting of ABCB1 or ABCB5

| | | siRNA | | IC50 (μM) | | |
|---|---|---|---|---|---|---|
| Gene | Cell line | downregulation (%) | Drug | Mock | siRNA | Fold reversal |
| ABCB1 | NCI/ADR-RES | 74 | Palitaxel | 8.01 | 1.06 | 7.57 |
| | | | Bisantrene | >100 | 23.7 | >4.22 |
| | | | Geldanamycin | 6.84 ± 1.08 | 3.60 ± 0.60* | 1.95 ± 0.45 |
| | | | Baker's antifol | 420 ± 120 | 131 ± 36.7* | 3.23 ± 0.45 |
| | | | 5FU | 900 | 1000 | 0.90 |
| | HCT-15 | 68 | Palitaxel | 0.31 | 0.18 | 1.72 |
| | | | Bisantrene | 7.63 | 5.47 | 1.39 |
| | | | Geldanamycin | 9.91 ± 3.96 | 8.20 ± 3.88* | 1.26 ± 0.14 |
| | | | Baker's antifol | 48.2 ± 5.18 | 33.7 ± 6.23* | 1.47 ± 0.29 |
| ABCB5 | SK-MEL-28 | 80 | CPT,10-OH | 0.48 ± 0.12 | 0.14 ± 0.02* | 3.62 ± 1.35 |
| | | | 5FU | 12.4 ± 4.60 | 6.43 ± 2.67* | 1.96 ± 0.16 |
| | | | Camptothecin | 0.13 | 0.06 | 2.17 |
| | | | Mitoxathone | 1.08 | 0.79 | 1.37 |

Human cancer cells were transfected with ABCB1, ABCB5 or mock siRNA. Drug activity was measured with the SRB assay. IC₅₀ is the concentration that produced 50% inhibition of cell growth compared to controls. Results represent the mean of two or mean±SD of at least three independent experiments.

Table 1 also lists genes encoding channels. AQP1 and AQP4, encoding water channel proteins, negatively correlated with folate and amino-acid drugs. Both AQP1 and AQP4 are highly expressed in brain tumors and carcinomas, but are undetectable in normal epithelial cells. On the other hand, AQP9 and MIP, aquaporins involved in transport of water, urea, and glycerol, positively correlated with several drugs. These gene products either mediate drug transport directly or are representative of tumor characteristics that indirectly confer sensitivity or resistance.

Ion channels modulate electrochemical gradients generated by ion pumps andion exchangers. Maintenance of a strong electrochemical gradient is not only vital to the cell, but also affects subcellular drug equilibration and transport. Thus, K⁺ and Cl⁻ leak currents tend to polarize cells, whereas Ca²⁺ and Na⁺ channels depolarize cells, with expected opposite effects on drug equilibration in and out of the cell, or cell organelles. However, Ca²⁺ flux is also important in apoptotic signaling, so that the net effect on drug potency is difficult to predict. In this study, CACNA1D, encoding the alpha 1D subunit of the L-type calcium channel, showed negative correlation with several drugs, including deoxydoxorubicin (Table 1). Interestingly, L-type calcium channel antagonists block ABCB1 and thereby are thought to overcome drug resistance. It remains to be seen whether blocking CACNA1D could have contributed to this effect. Moreover, several genes encoding subunits of sodium, chloride, potassium and other cation channels correlated with drug activity, confirming that ion channels modulate drug response, possibly by affecting the cell's resting potential, or providing key metal ion cofactors. It will be important to understand the role of ion channels in the cell's reaction to toxic stimuli, as loss of ADP-ATP gradients during the course of toxic reactions directly alters electrochemical gradients.

### Example 5

### siRNA-Induced Silencing of ABCB1 and ABCB5 Expression: Validating Gene-Drug Correlations

Negative correlations between ABCB1 and GA and BAF suggested that these drugs are substrates of ABCB1. To validate this new finding, we used a chemically synthesized siRNA duplex targeting ABCB1 in NCI/ADR-RES and HCT-15 cells, which express high level of ABCB1. Real-time RT-PCR demonstrated that 40 hours after treatment, siRNA substantially reduced ABCB1 mRNA levels (Table 3).

We next compared growth-inhibitory IC₅₀ values of siRNA-treated to that of mock-treated cells using a sulforhodamine B (SRB) cell proliferation assay. Sensitivity of NCI/ADR-RES to paclitaxel, bisantrene, GA, and BAF was increased 2.4 to 7.6-fold by ABCB1 siRNA transfection (Table 3 and FIG. 3). Sensitivity to 5FU, a non-Pgp substrate, was unaffected by siRNA silencing (data not shown). Therefore, application of RNAi gene silencing supports the hypothesis that GA and BAF are ABCB 1 substrates.

To identify suitable ABCB5 domains for siRNA-mediated gene silencing, siRNA duplexes against three target domains were synthesized and transfected into SK-MEL-28 cells. Real time RT-PCR demonstrated that siRNA-ABCB5_{—}957 was most effective in down-regulating ABCB5 (data not shown). siRNA-ABCB5_{—}957 transfected SK-MEL-28 cells were significantly (2-3 fold) more sensitive to camptothecin, the camptothecin analog CPT, 10-OH, and 5FU, as compared to control cells transfected with mock siRNA (FIG. 3, Table 3). In contrast, no change in potency was observed for mitoxathone (FIG. 3) and AMSA (data not shown). These results support the hypothesis that ABCB5 represents a novel chemoresistance gene. Whether the chemoresistance conferred by ABCB5 expression is due to increased drug efflux, or other mechanisms, remains to be determined. Since ABCB5 is selectively expressed in melanoma cells and two breast cancer cell lines of suspected melanoma origin in the NCI-60, it may serve as an important resistance factor in the treatment of melanoma.

### Example 6

### Methods

Oligonucleotide microarrays. A spotted 70-mer oligonucleotide microarray was developed to measure transporter and channel gene expression as described. Each probe was printed 4 times per array to enhance precision of the measurements.

Array hybridization. Total RNA was extracted from cell cultures maintained at the National Cancer Institute under conditions and passage numbers close to those used in a previous cDNA array study. Expression of each gene was assessed by the ratio of expression level in the sample against a pooled control sample from 12 diverse cell lines of the NCI-60. 12.5 μg total RNA was used for cDNA synthesis and then labeled with Cy5 or Cy3 (control) by amino-allyl coupling. The protocol is available at http://derisilab.ucsf.edu/pdfs/amino-allyl-protocol.pdf. In brief, samples from test cells were labeled with Cy5, and the pooled RNA control was labeled with Cy3. The samples were then mixed, and the labeled cDNA was resuspended in 20 μL HEPES buffer (25 mM, pH 7.0) containing 1 μL of tRNA, 1.5 μL of polyA⁺ 0.45 μL of 10% SDS. The mixture was hybridized to the slides for 16 h at 65° C. Slides were washed, dried and scanned in an Affymetrix 428 scanner to detect Cy3 and Cy5 fluorescence.

Spot filtering. Background subtraction and calculation of medians of pixel measurements per spot was carried out using GenePix Software 3.0 (Foster City, Calif.). Spots were filtered out if they had both red and green intensity less than 250 units after subtraction of the background, or if they were flagged for any visual reason.

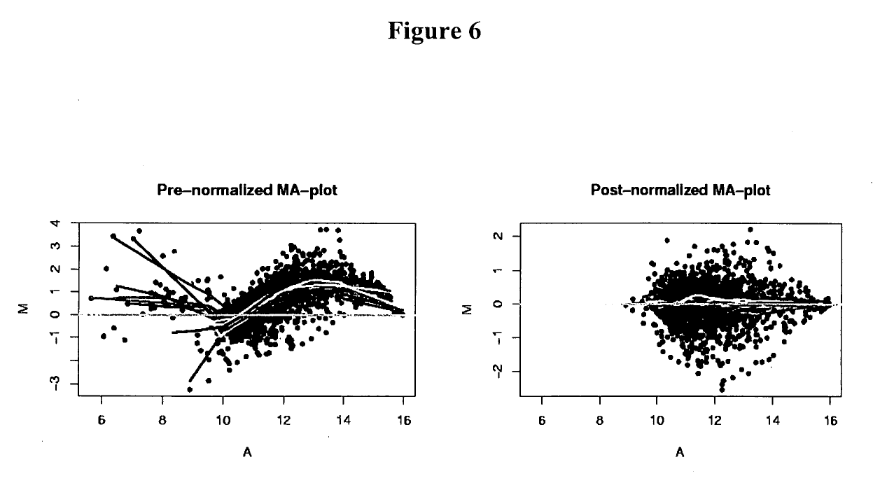

Normalization. Most statistical analyses were carried out using the statistical software package R (found on the internet at the website url r-project.org). The plot of M=log₂R/G vs. A=log₂\sqrt{R*G} (FIG. 6) shows dependence of the log ratio M on overall spot intensity A. Therefore, an intensity-dependent normalization method was preferred over a global method. To correct intensity- and dye-bias we used location and scale normalization methods, which are based on robust, locally linear fits, implemented in the SMA R package. This method is based on transformations:

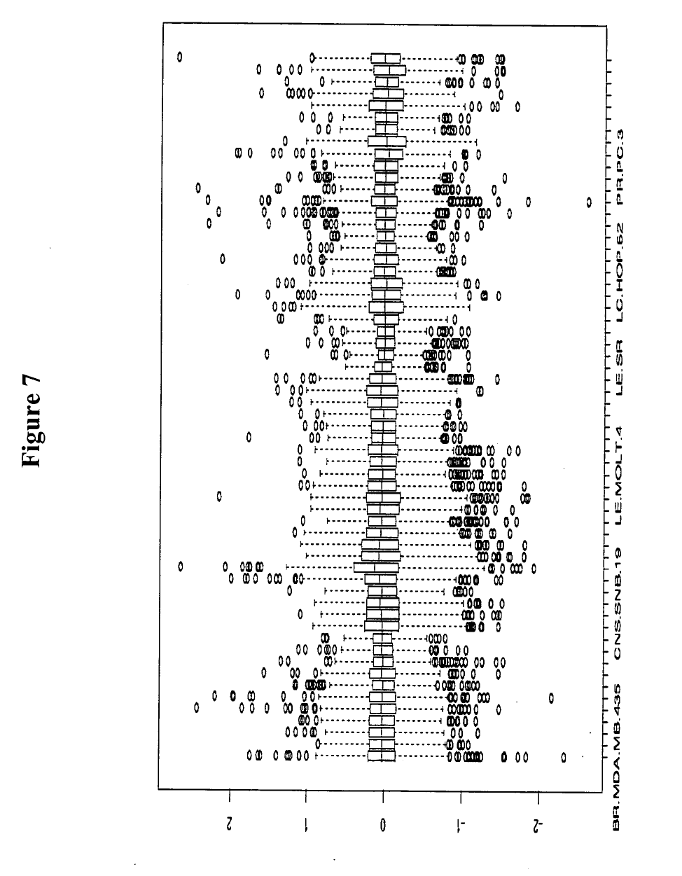

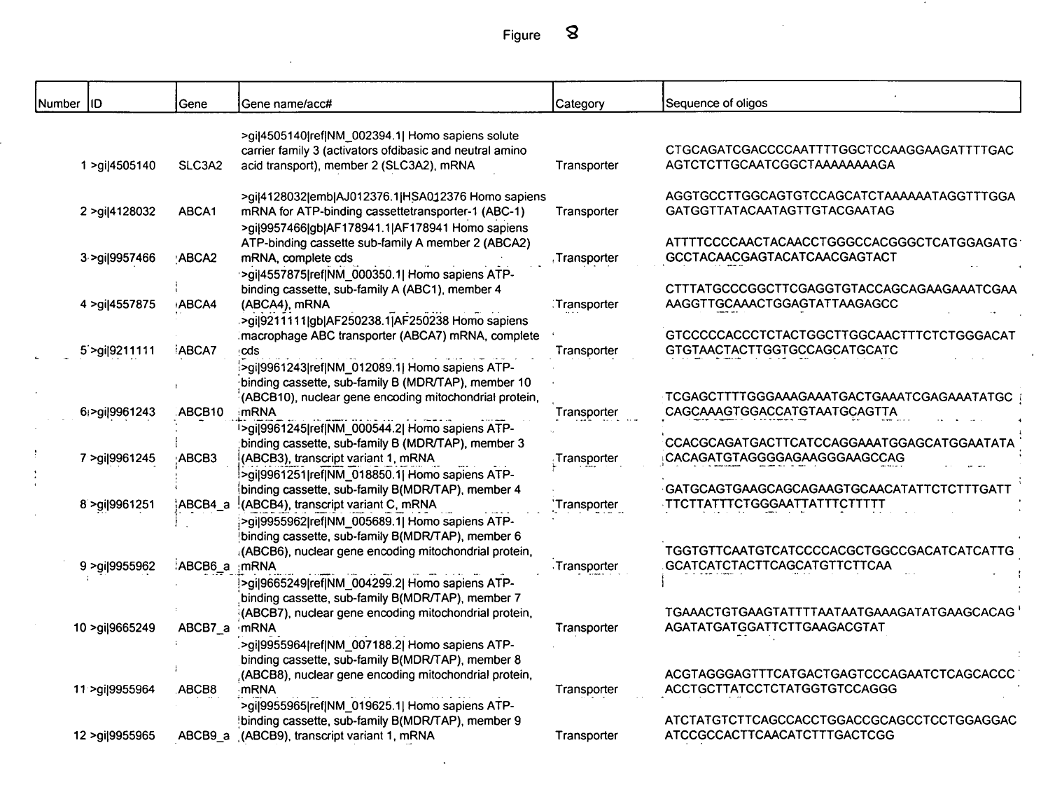

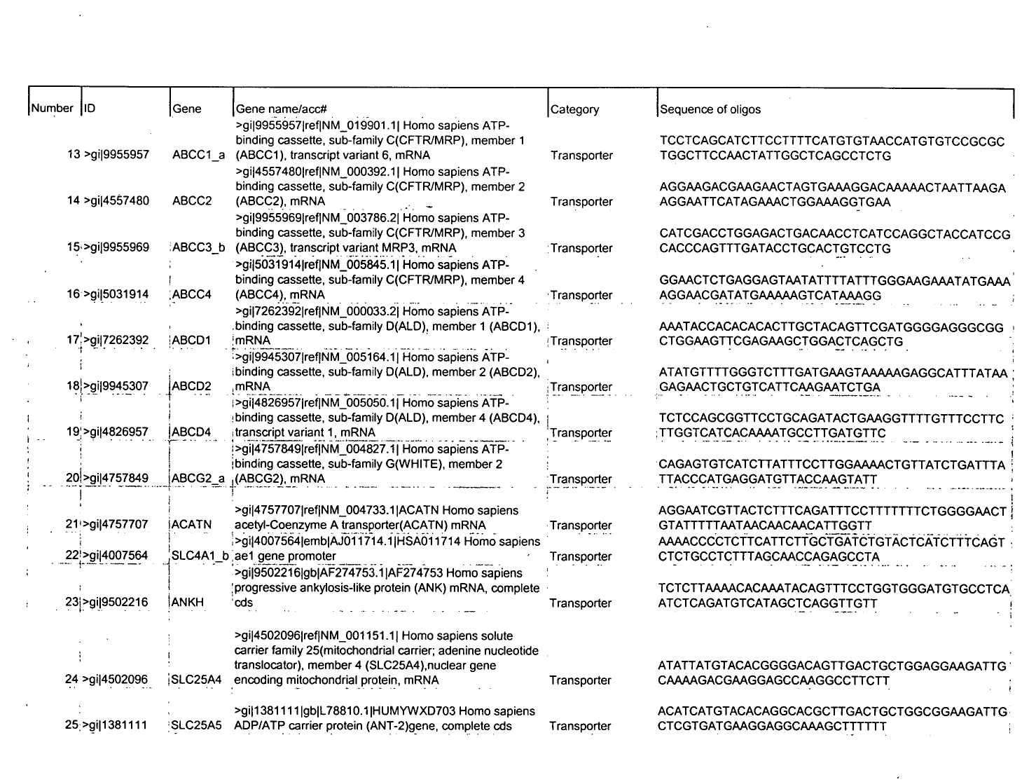

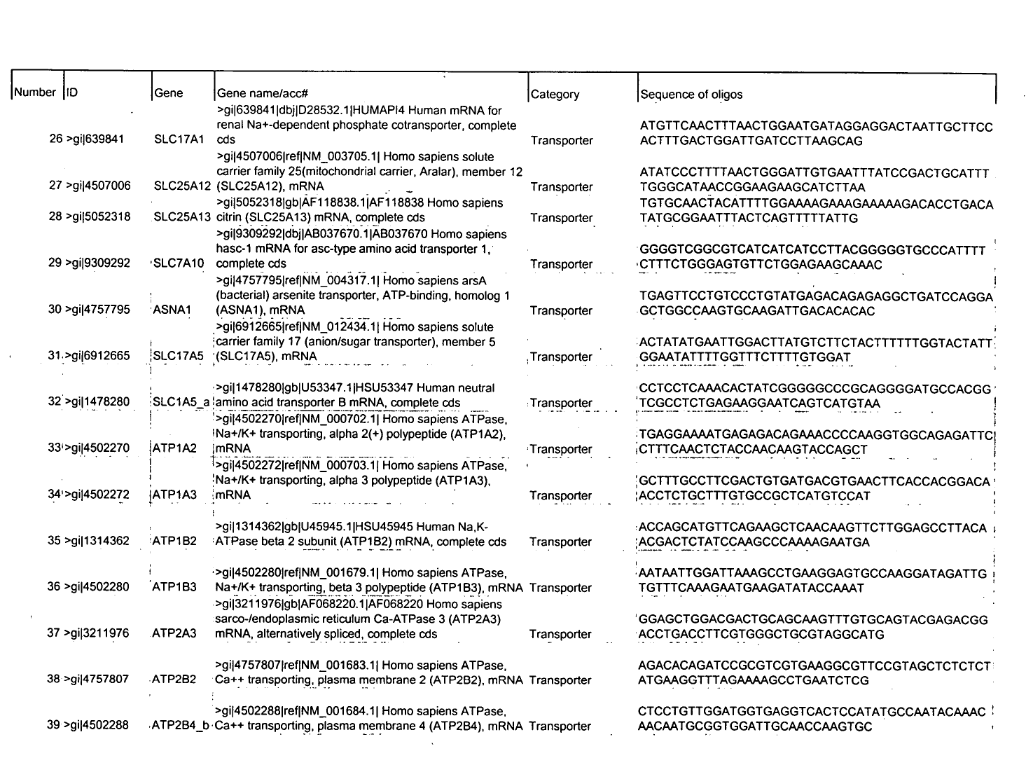

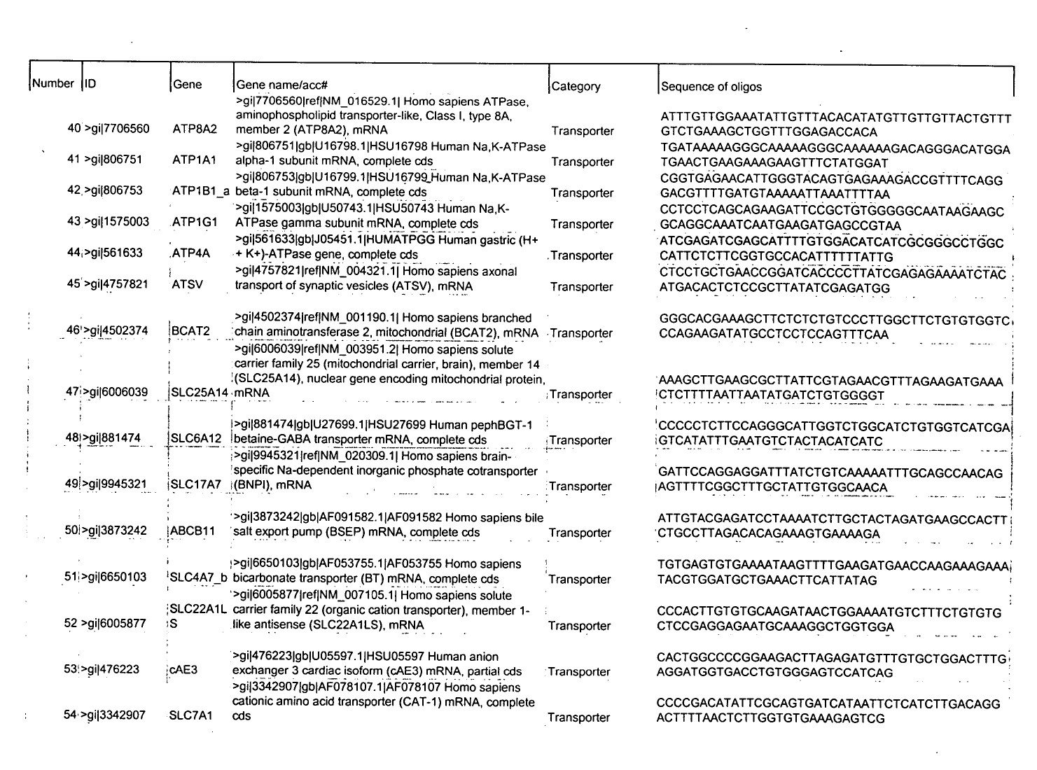

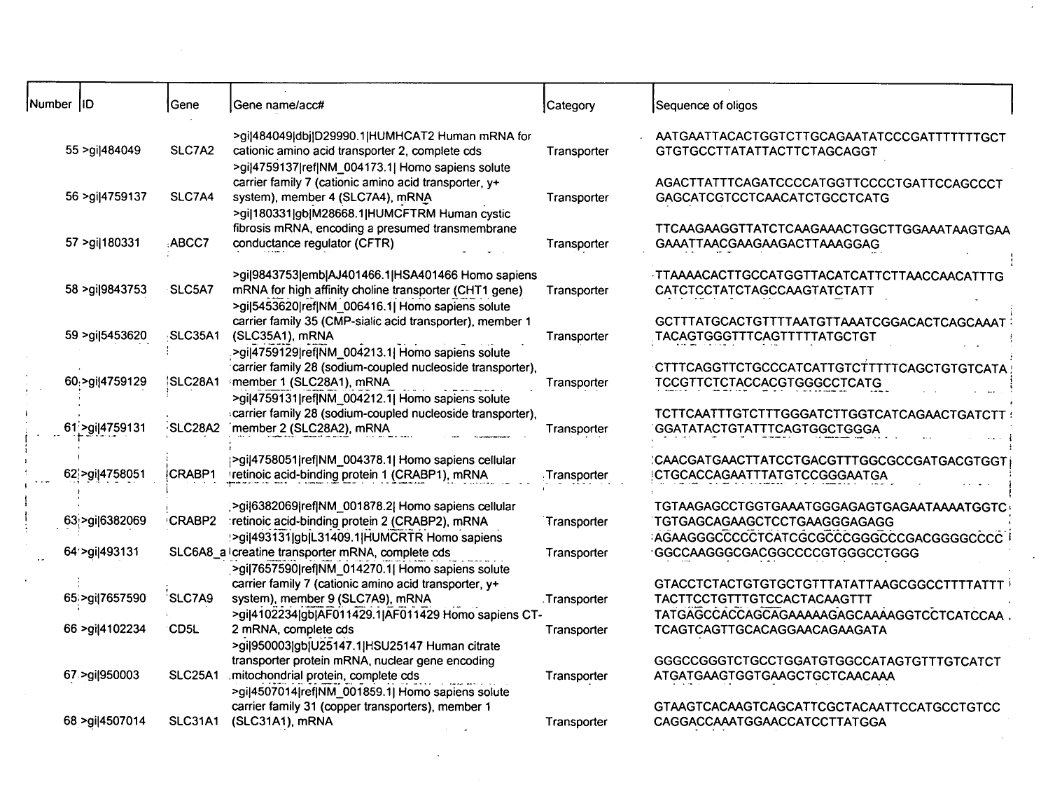

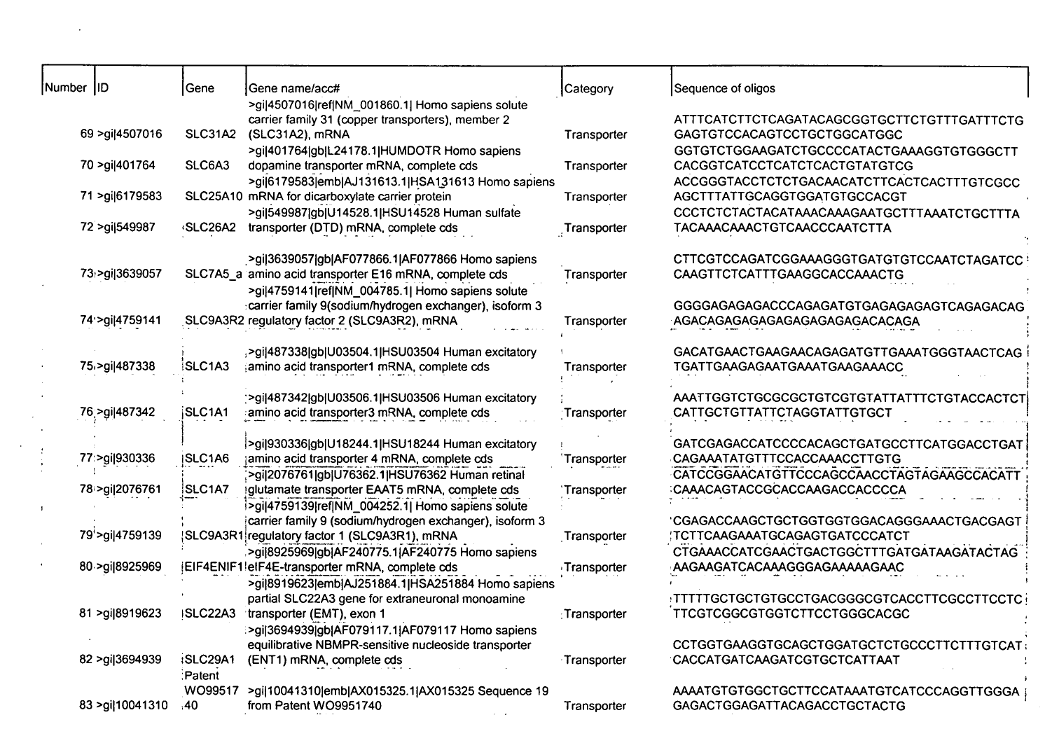

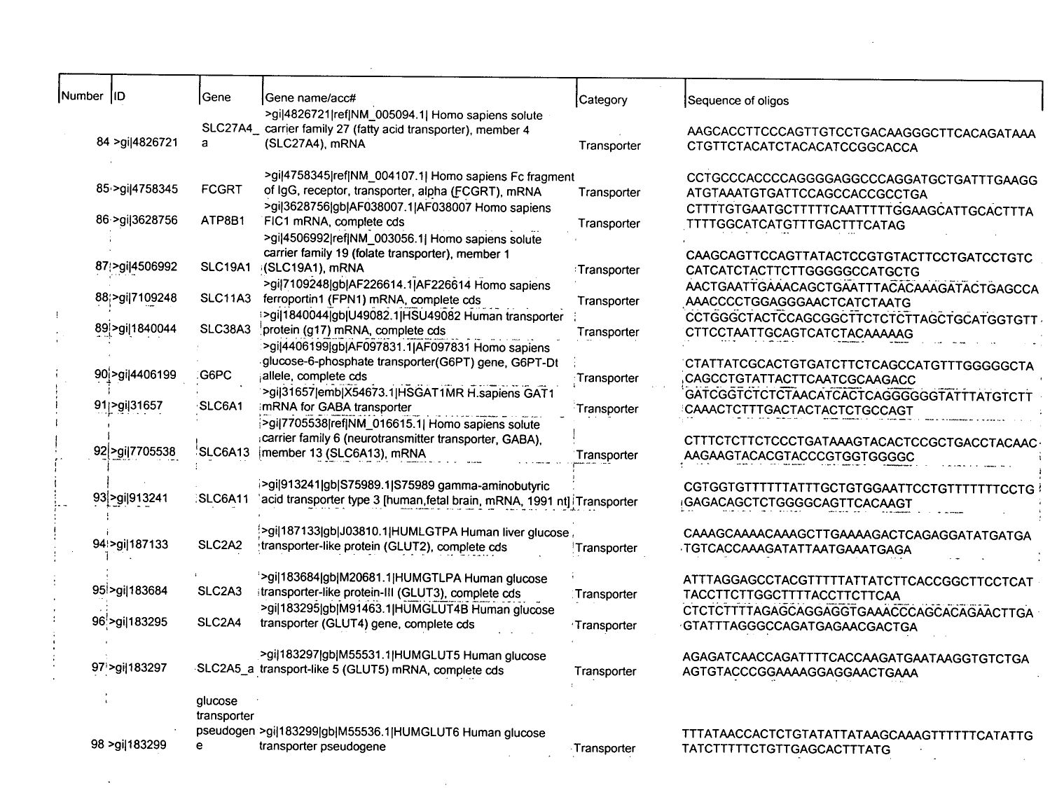

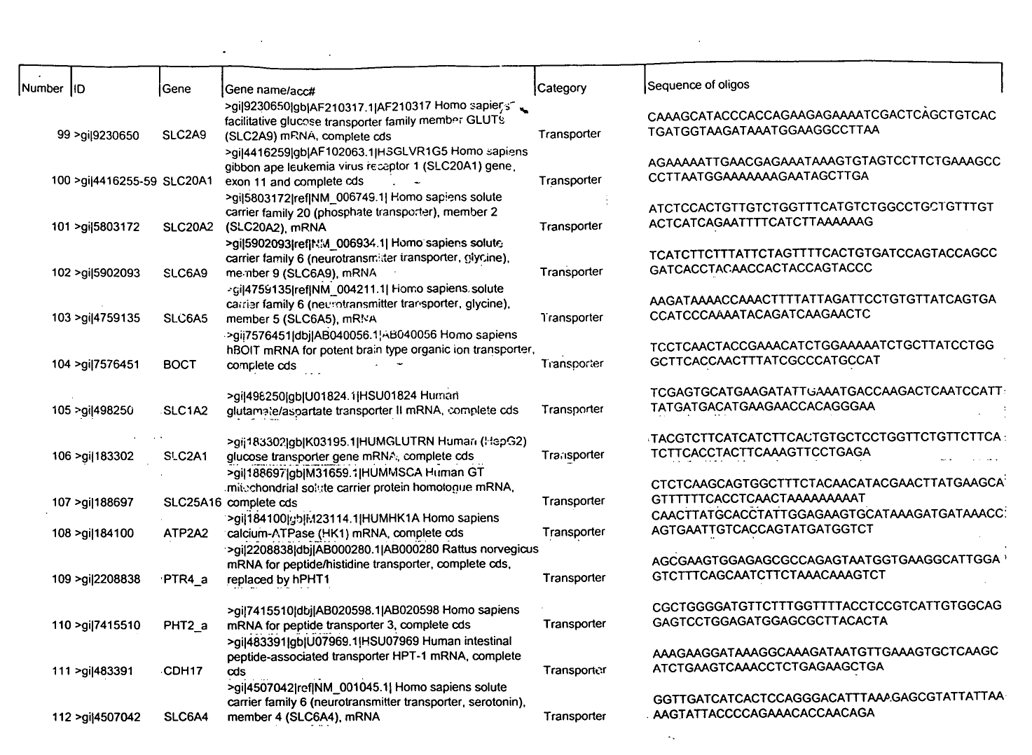

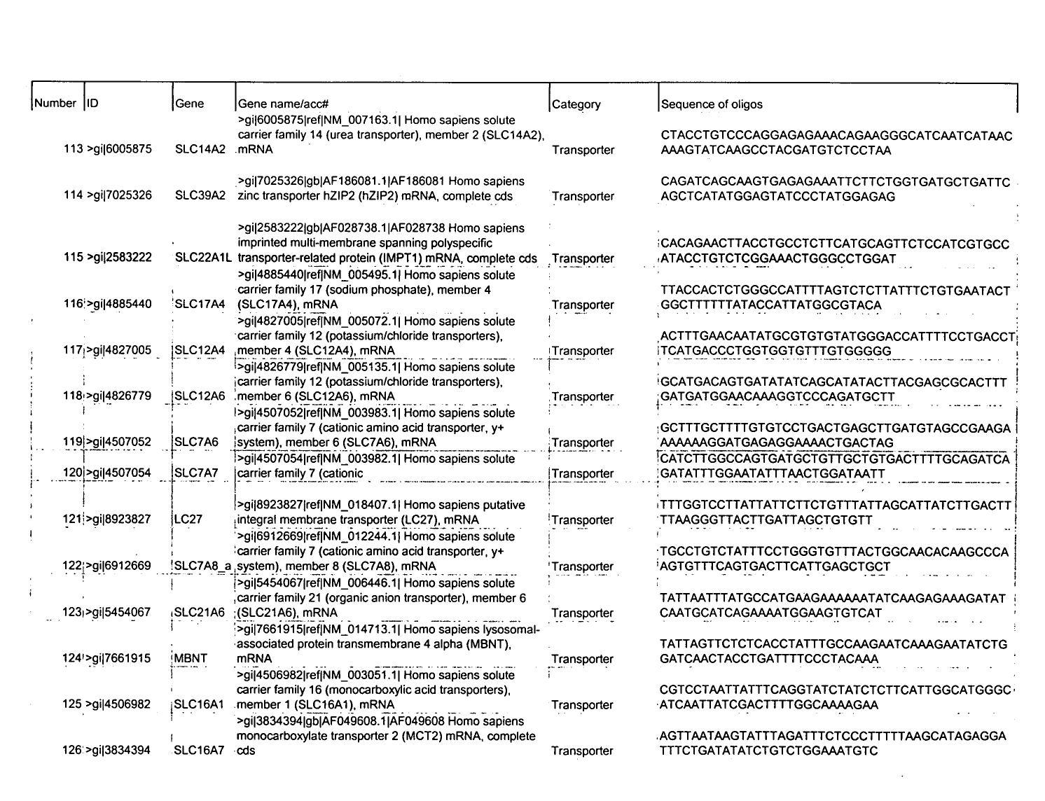

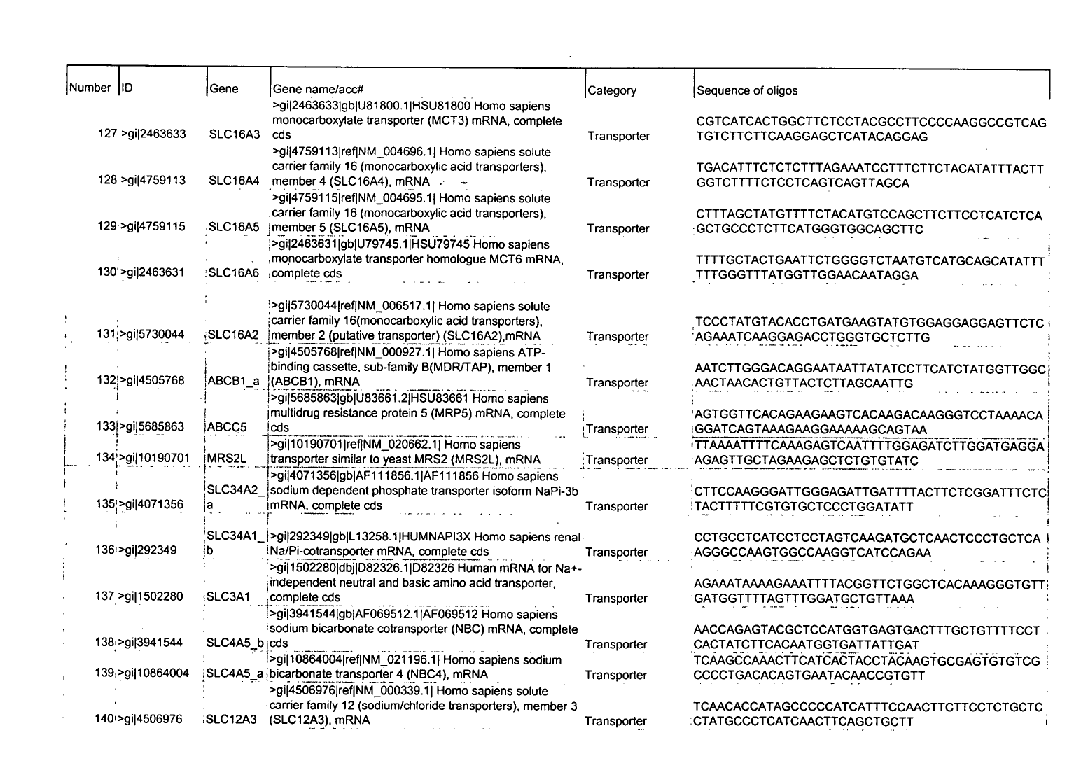

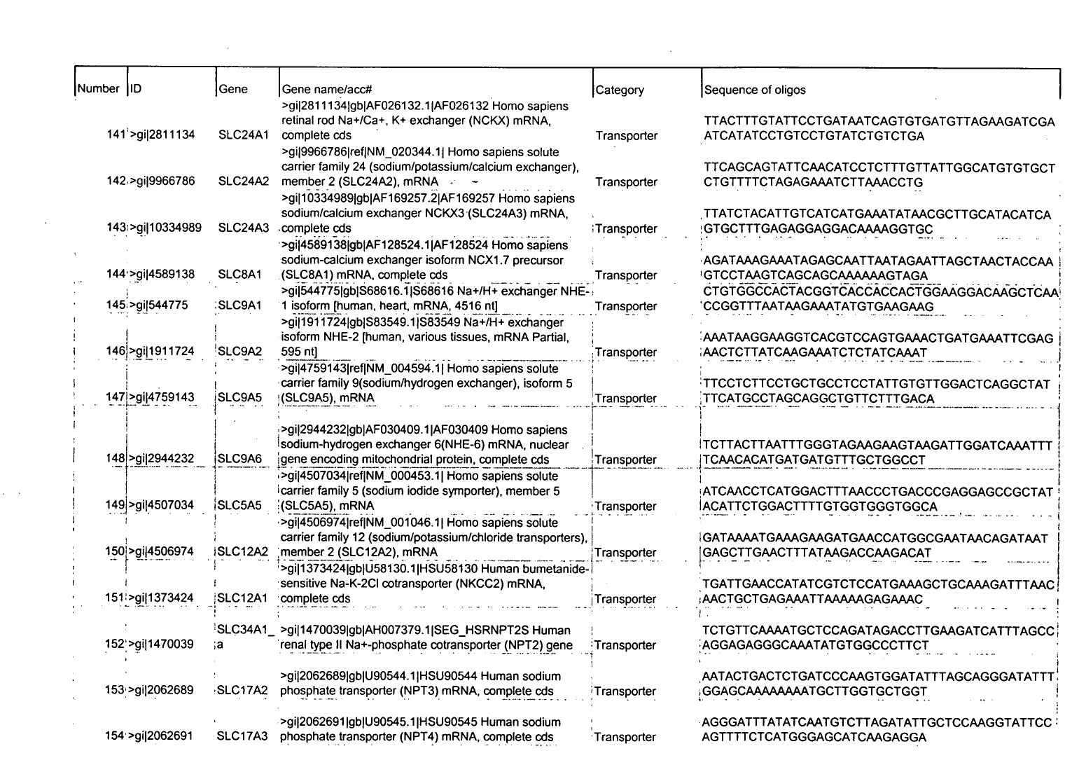

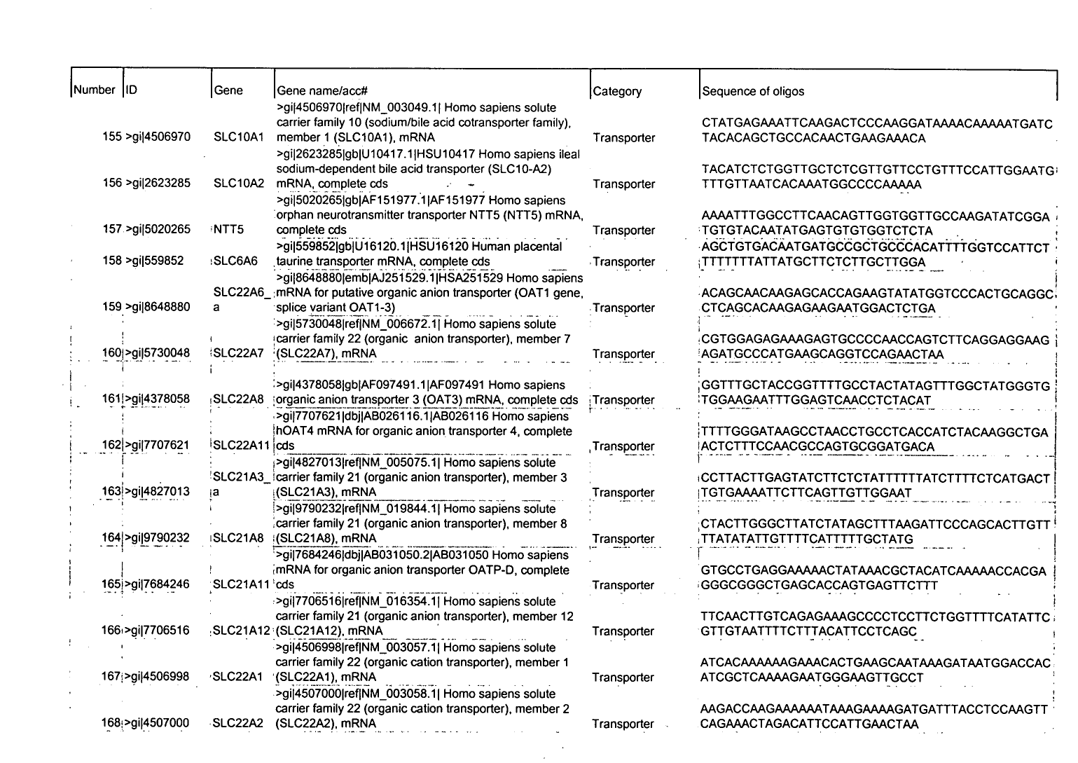

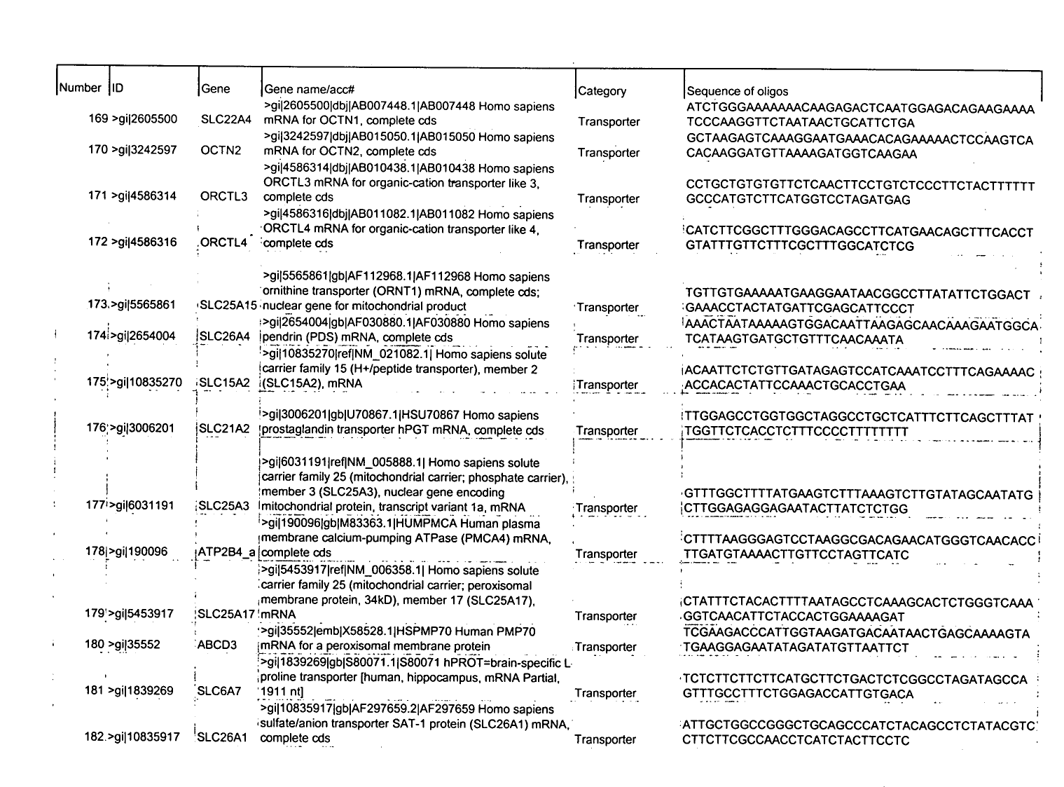

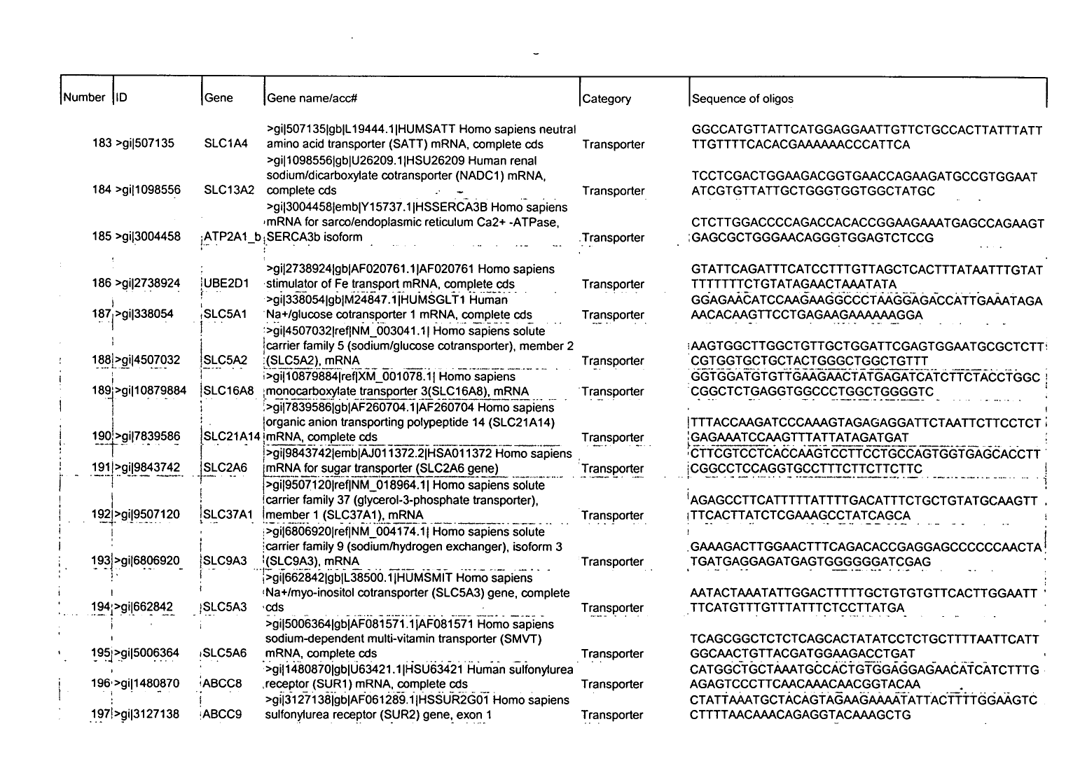

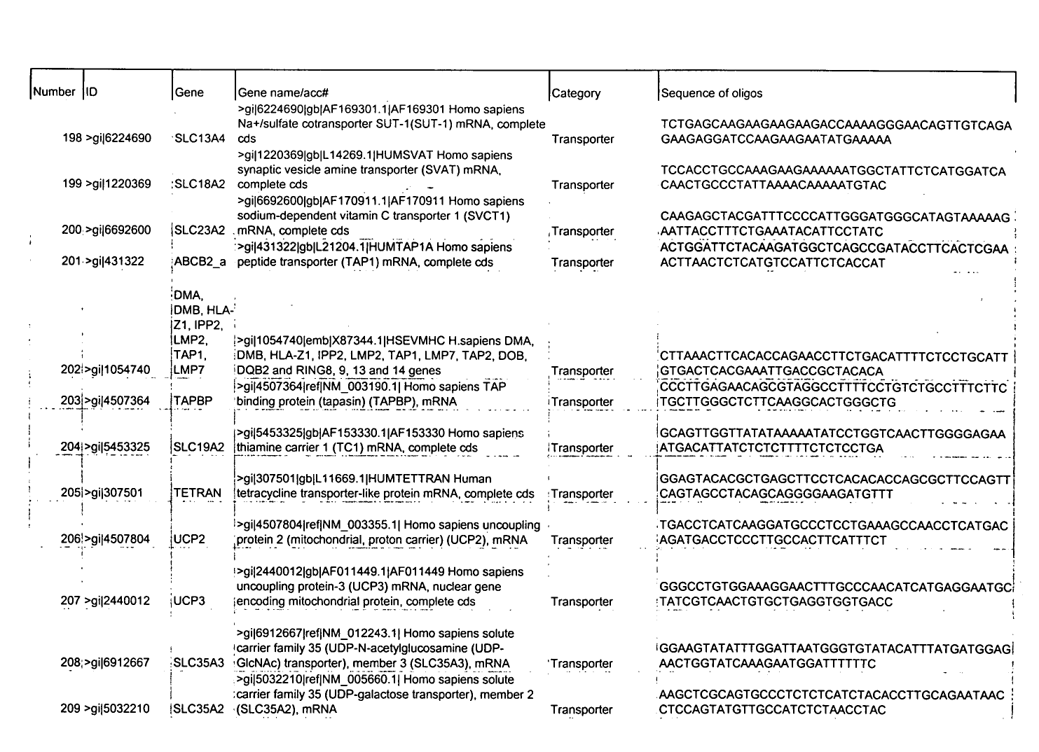

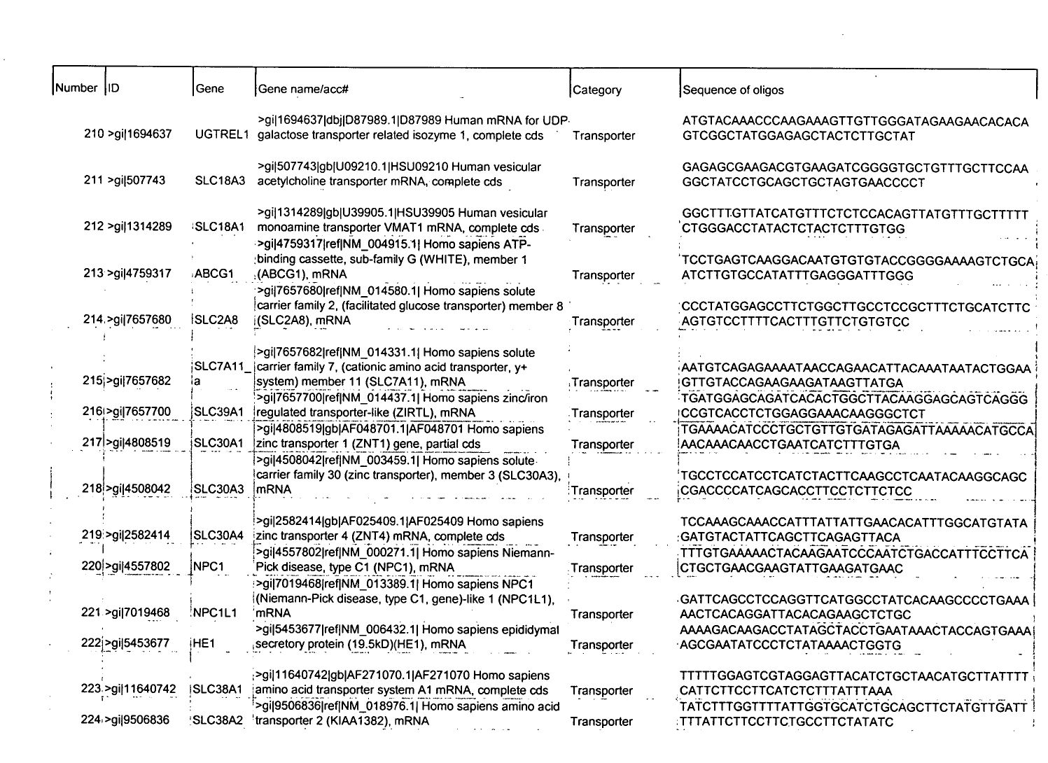

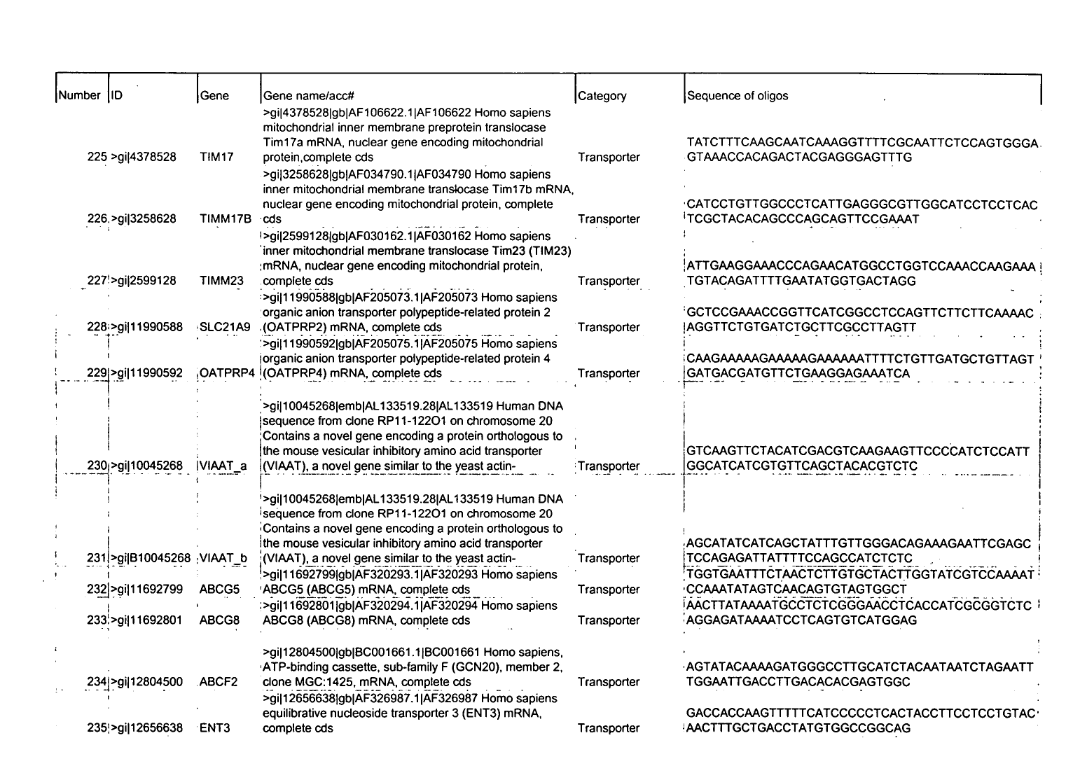

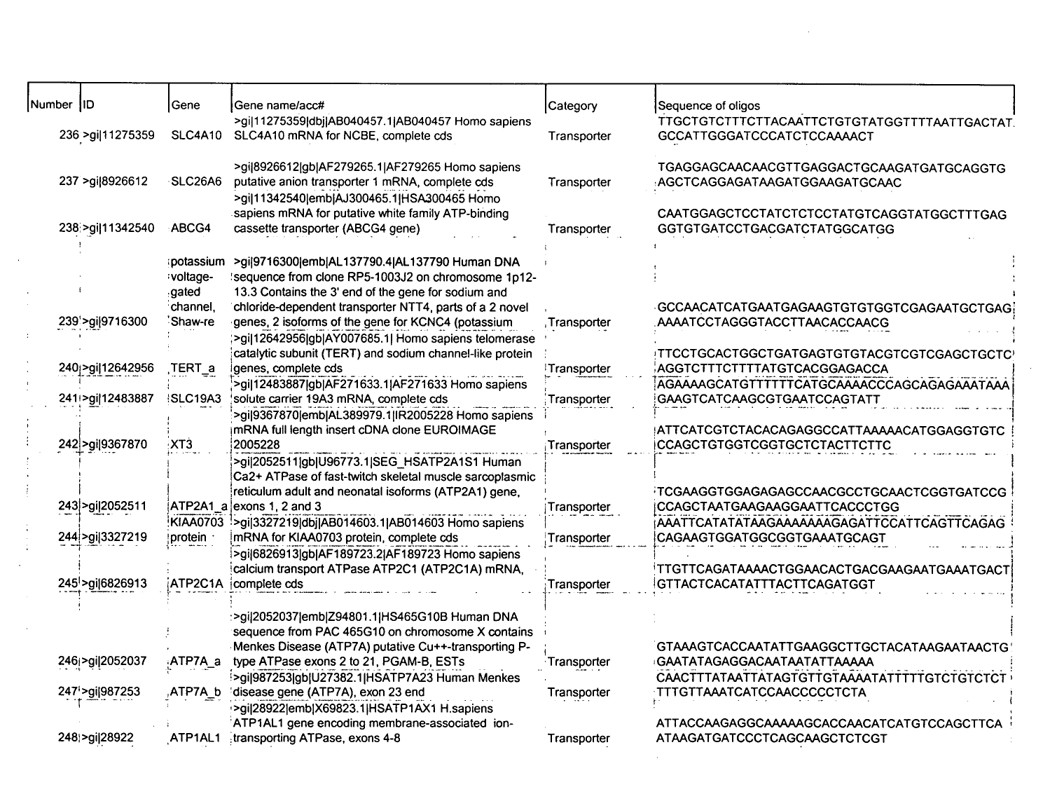

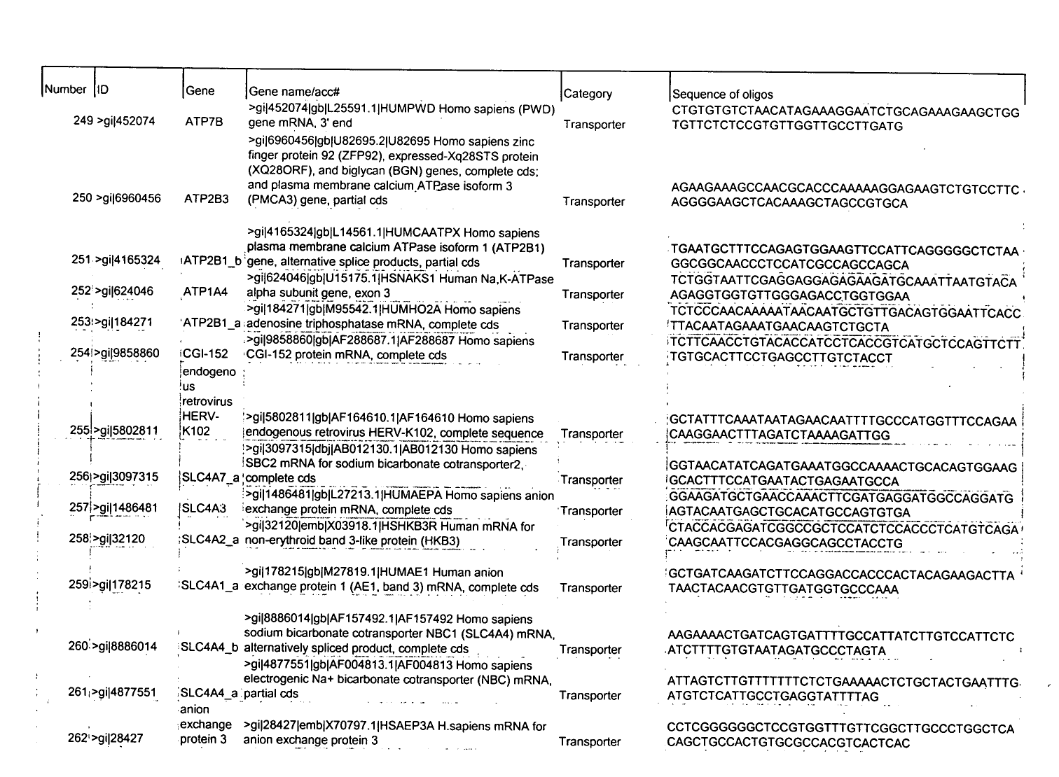

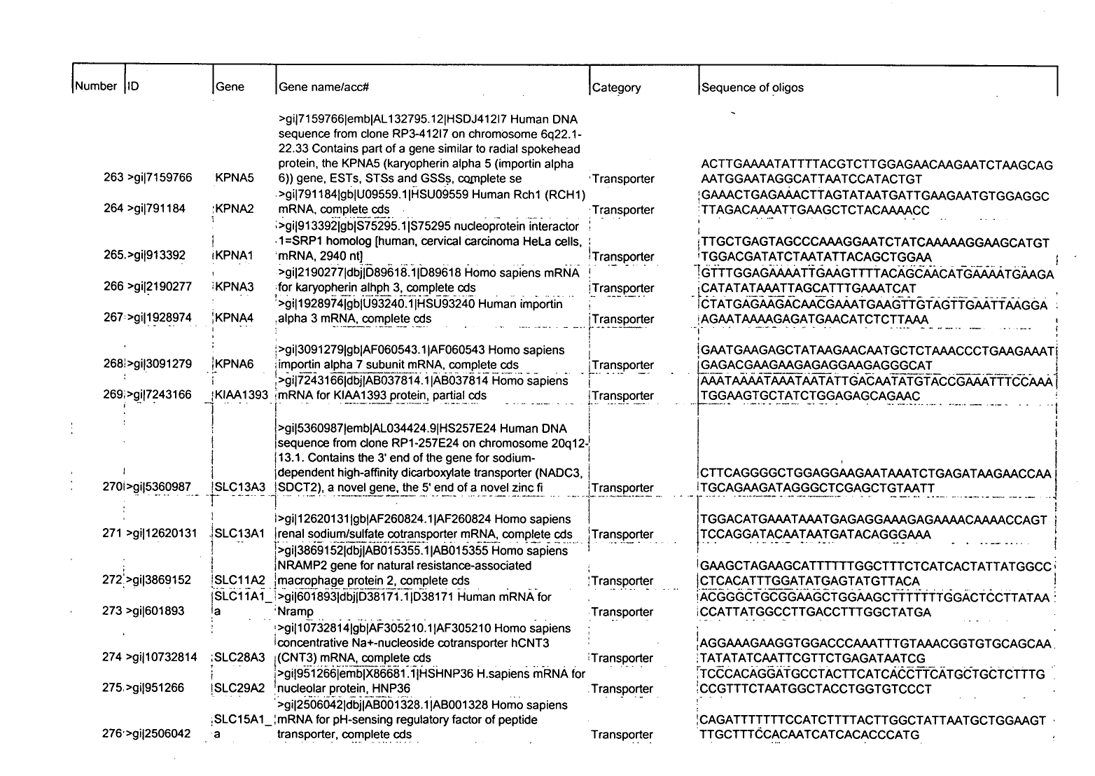

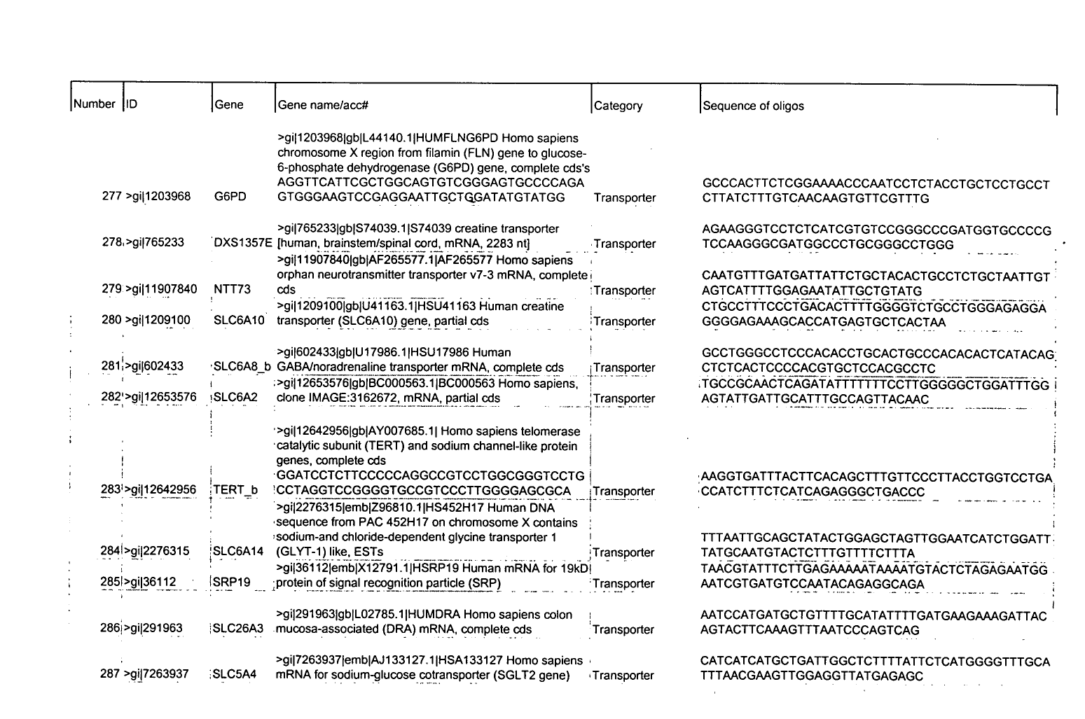

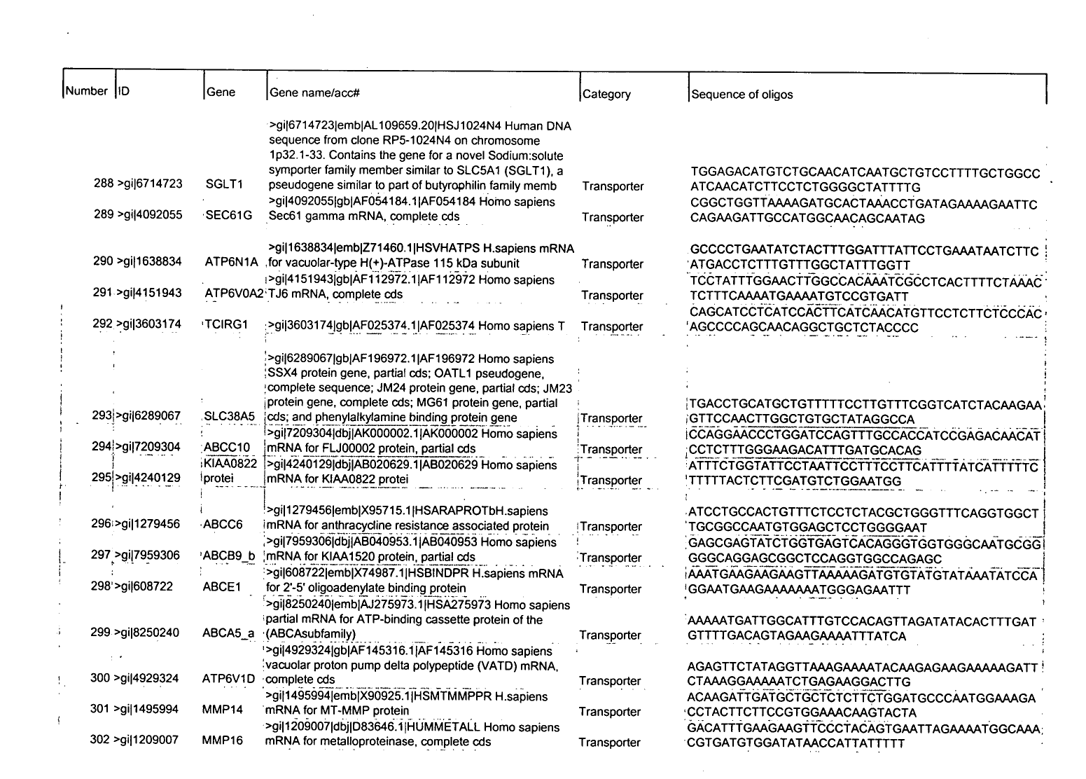

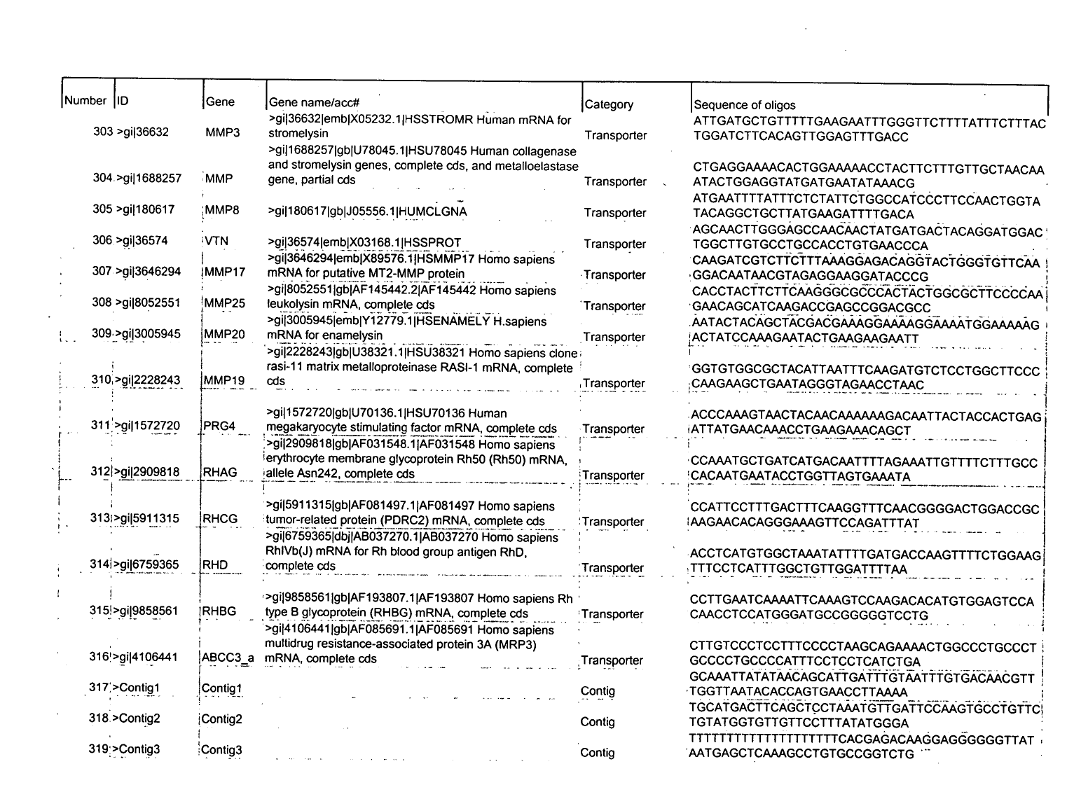

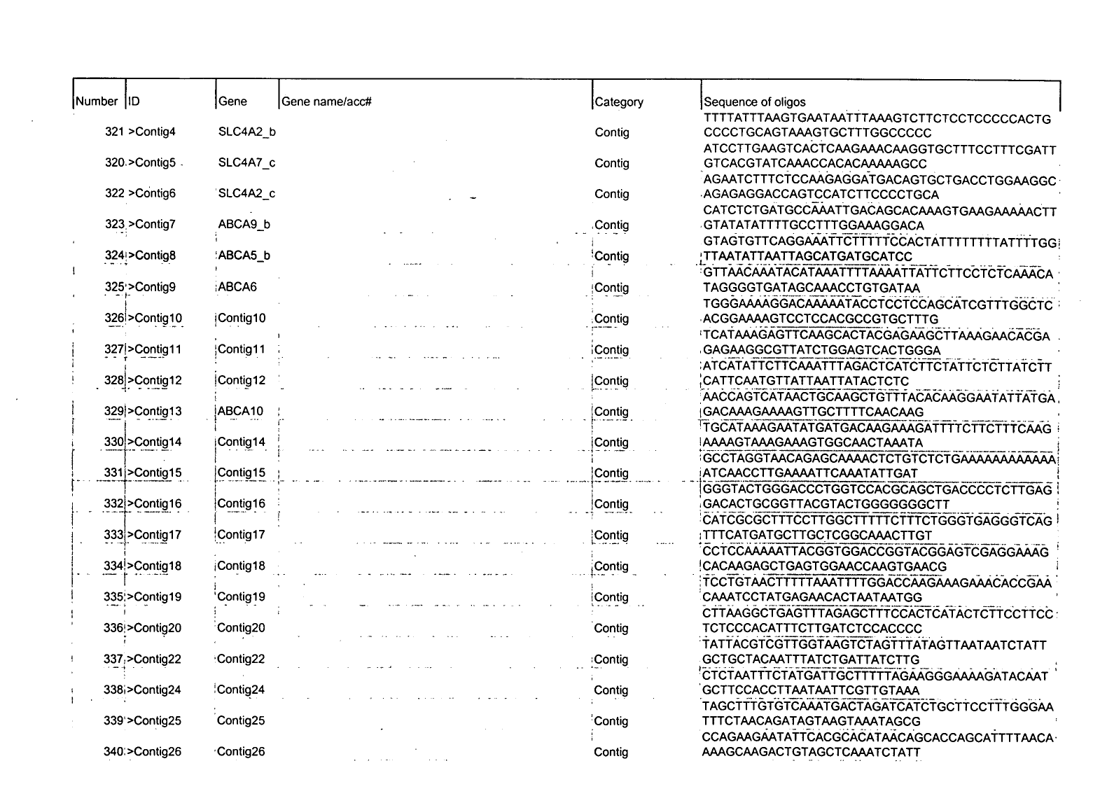

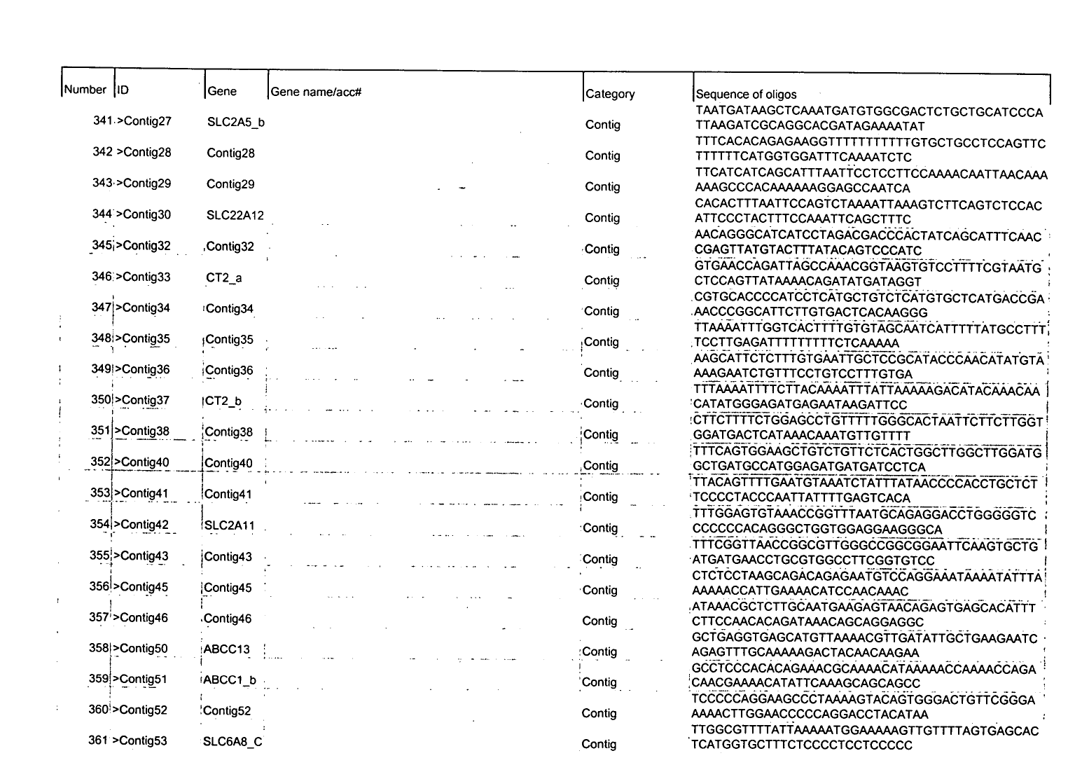

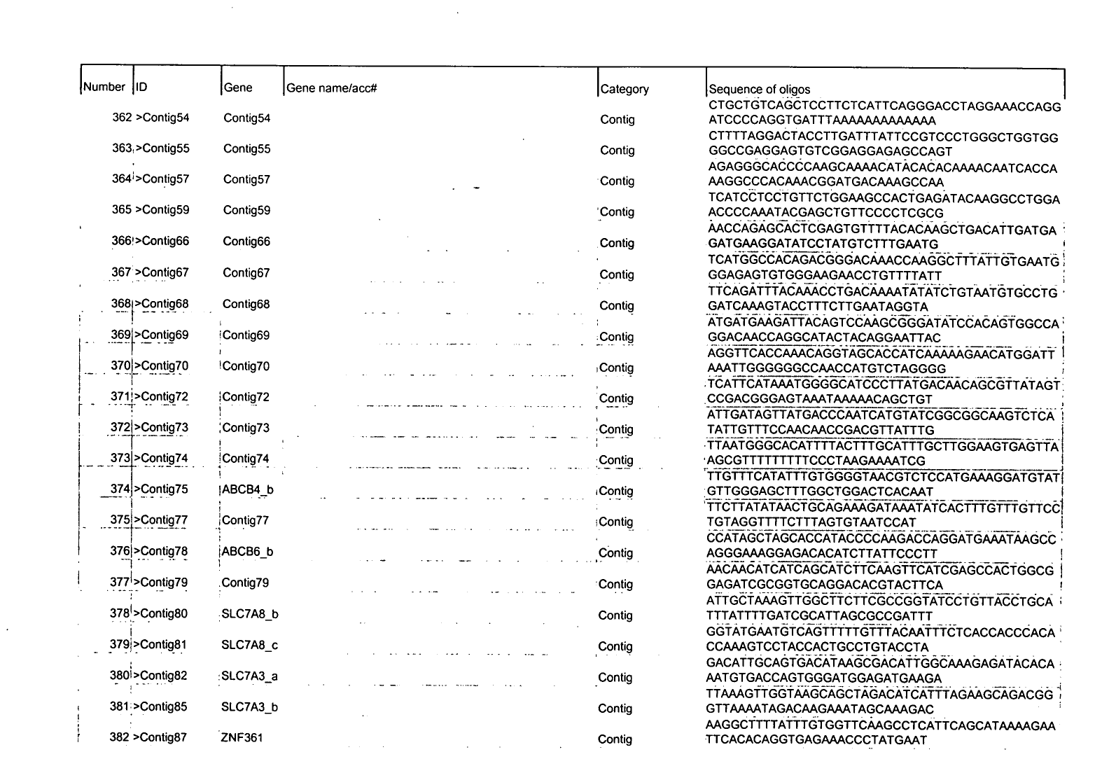

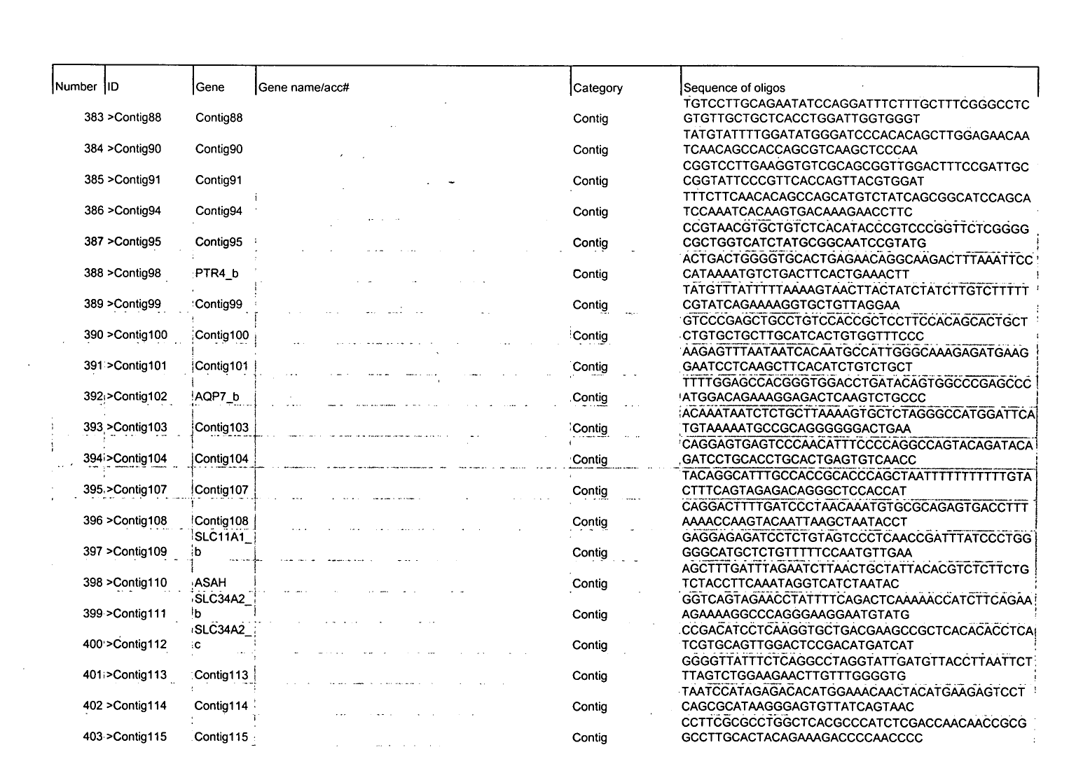

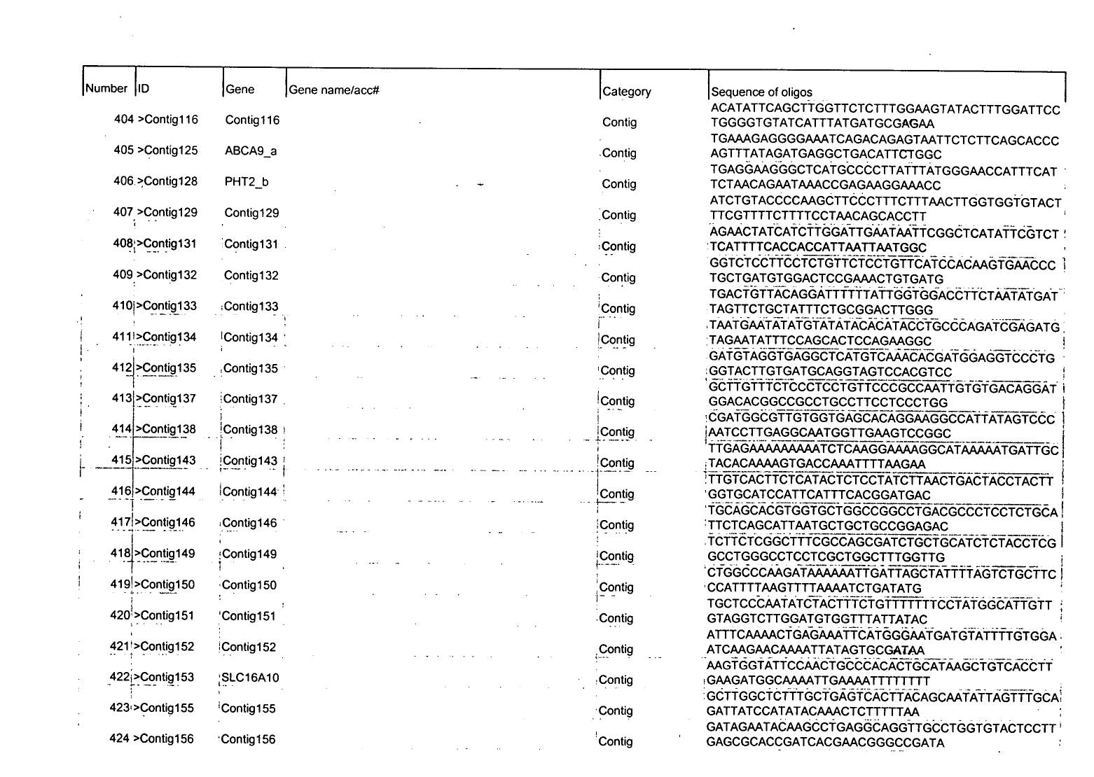

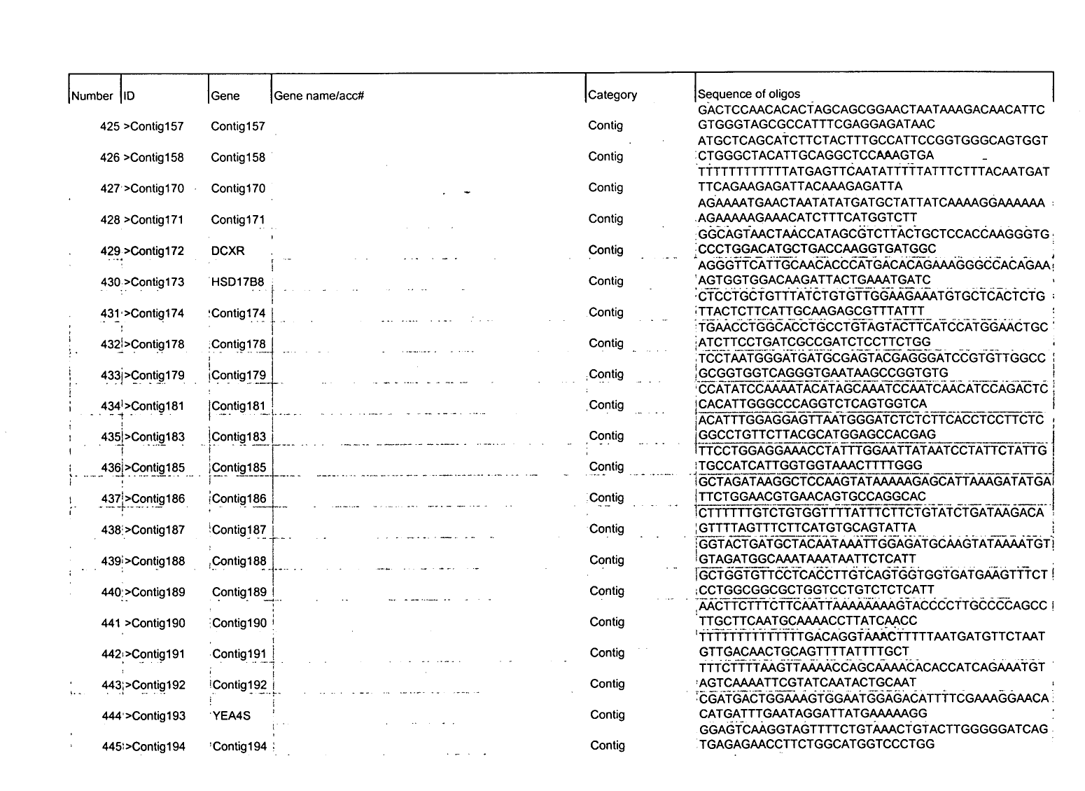

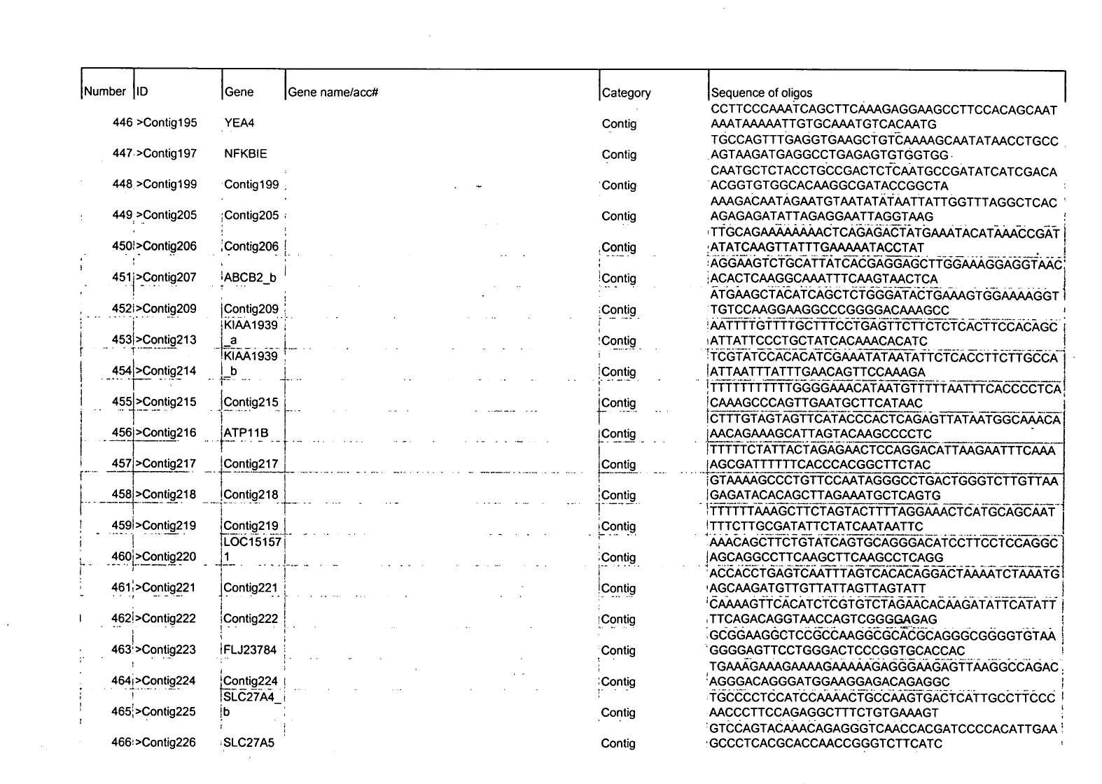

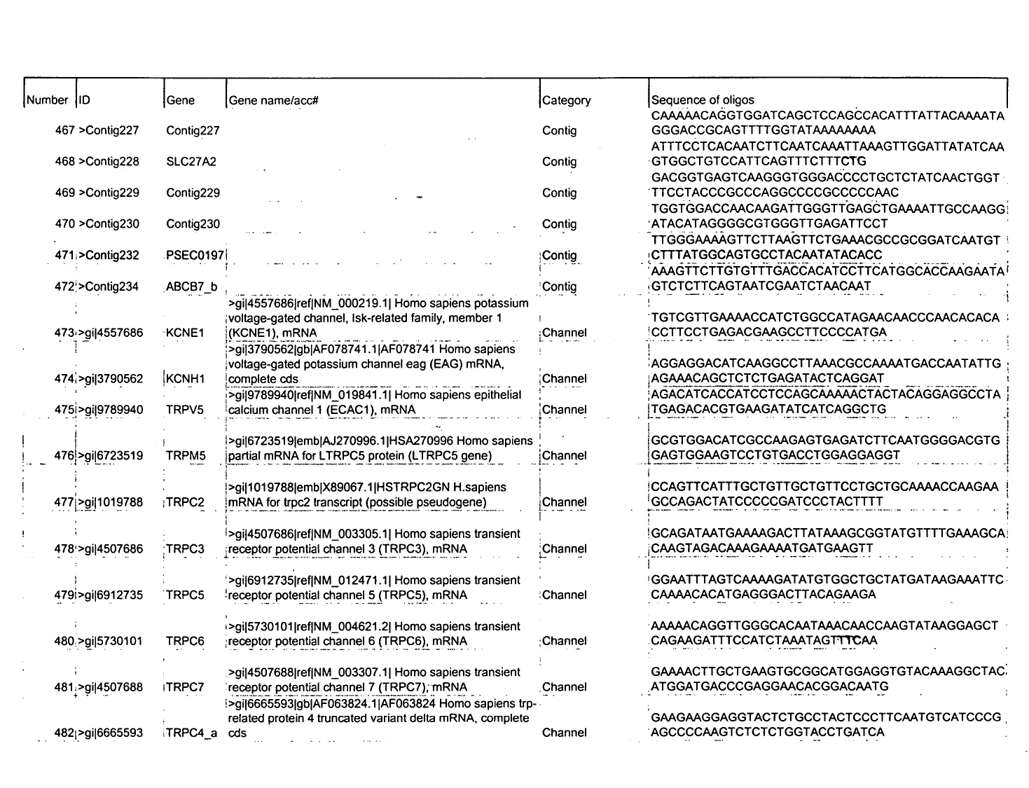

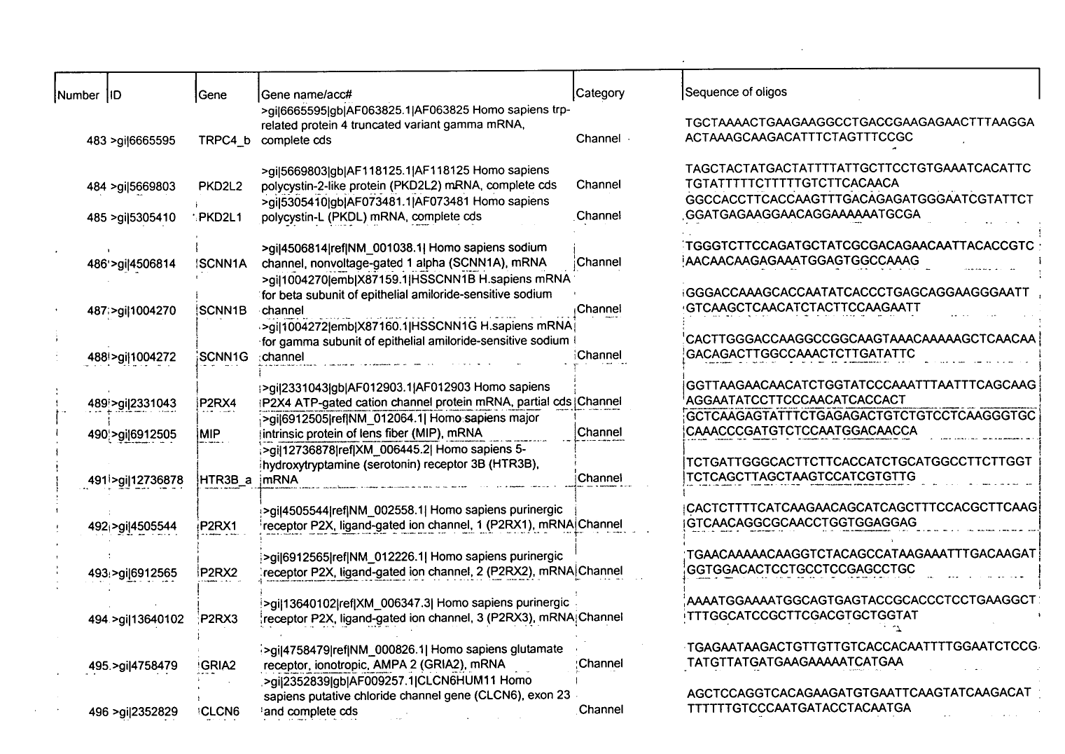

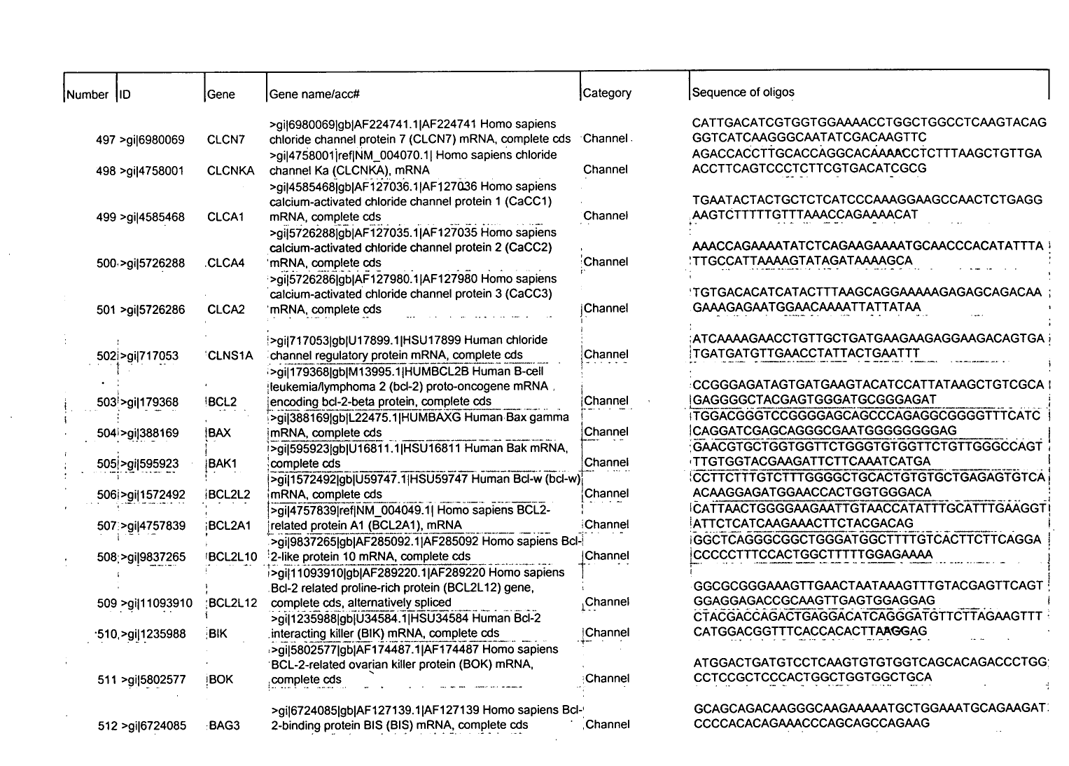

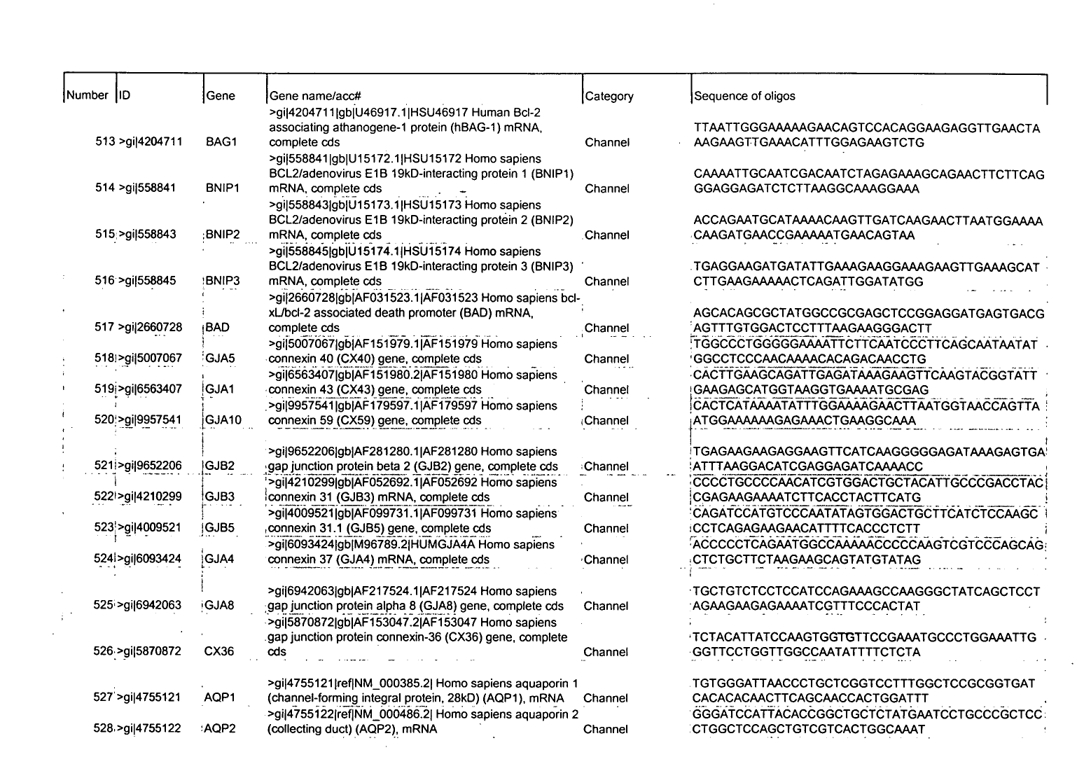

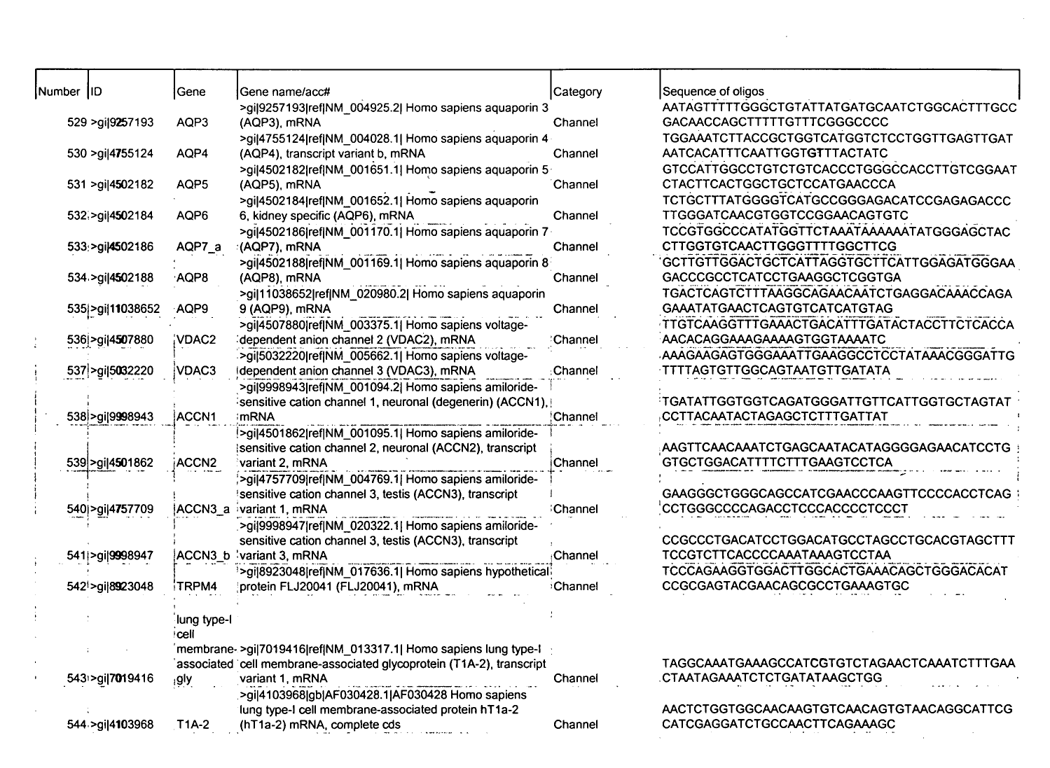

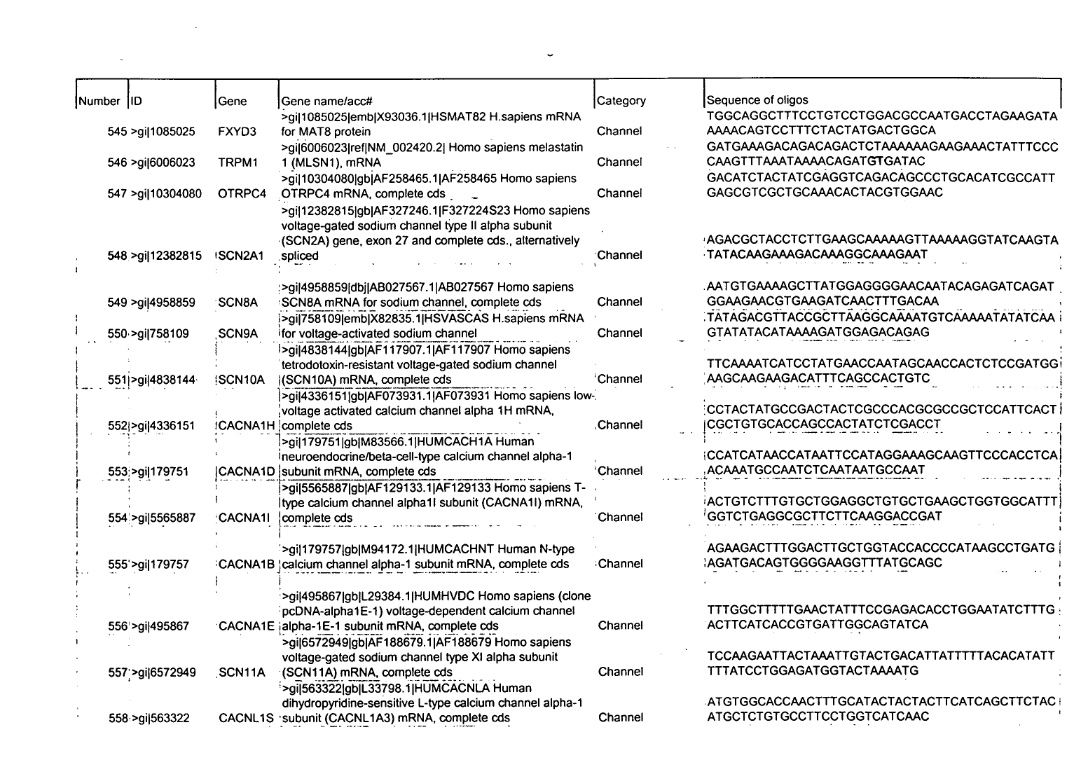

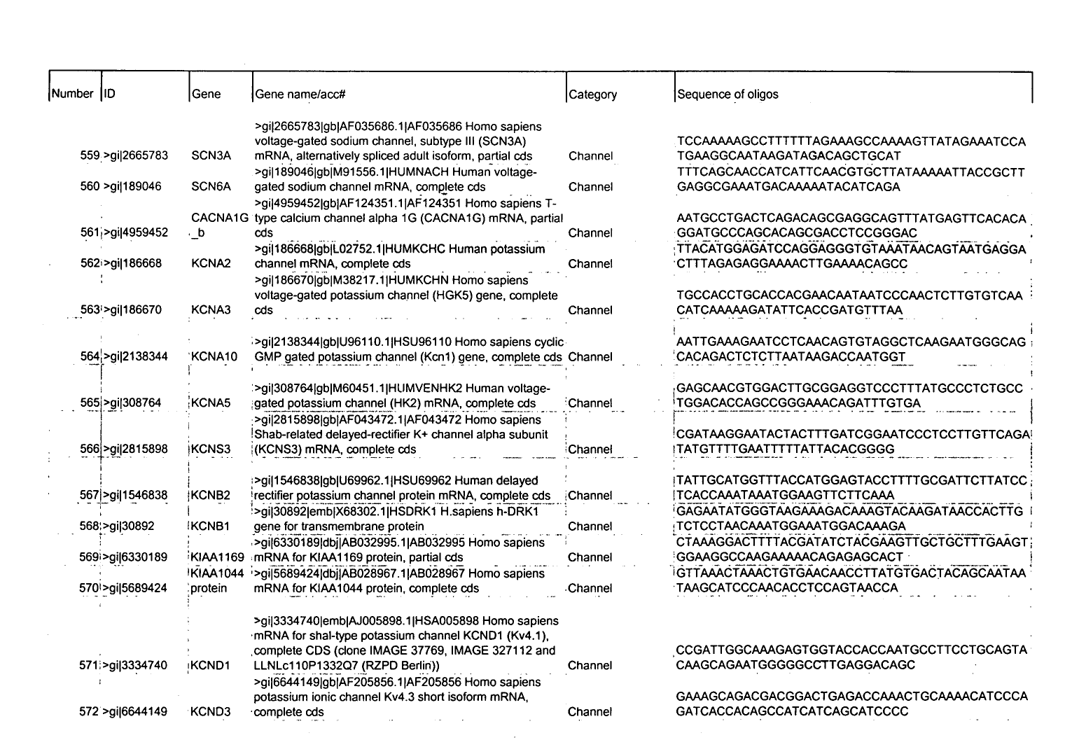

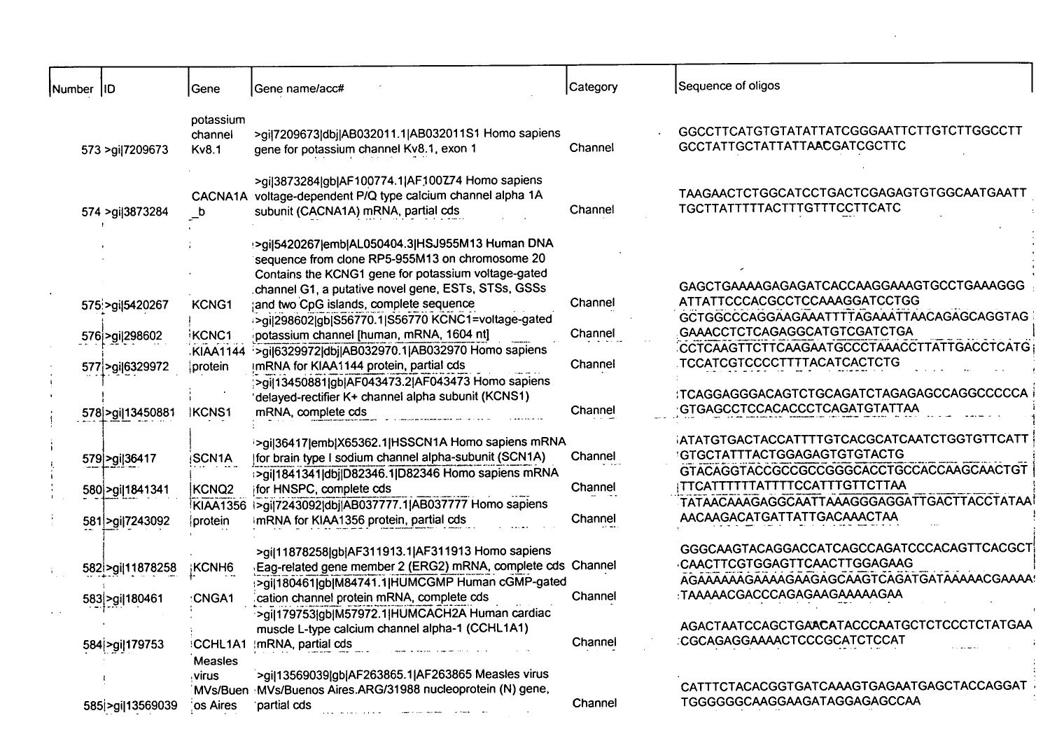

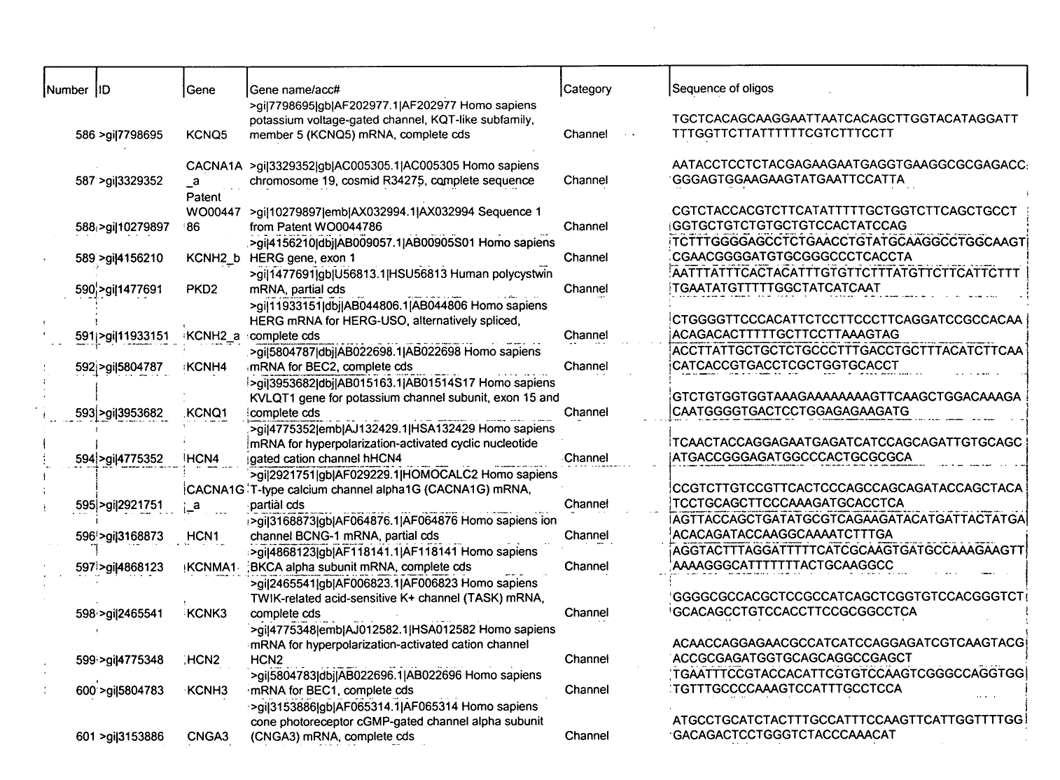

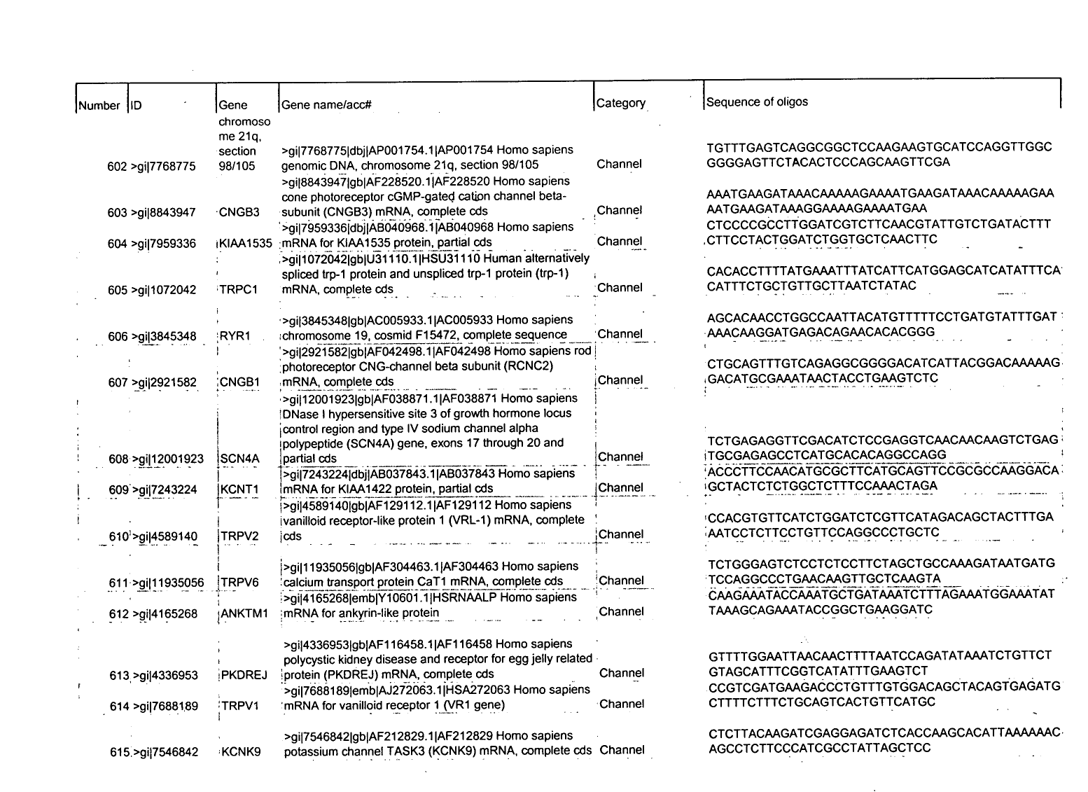

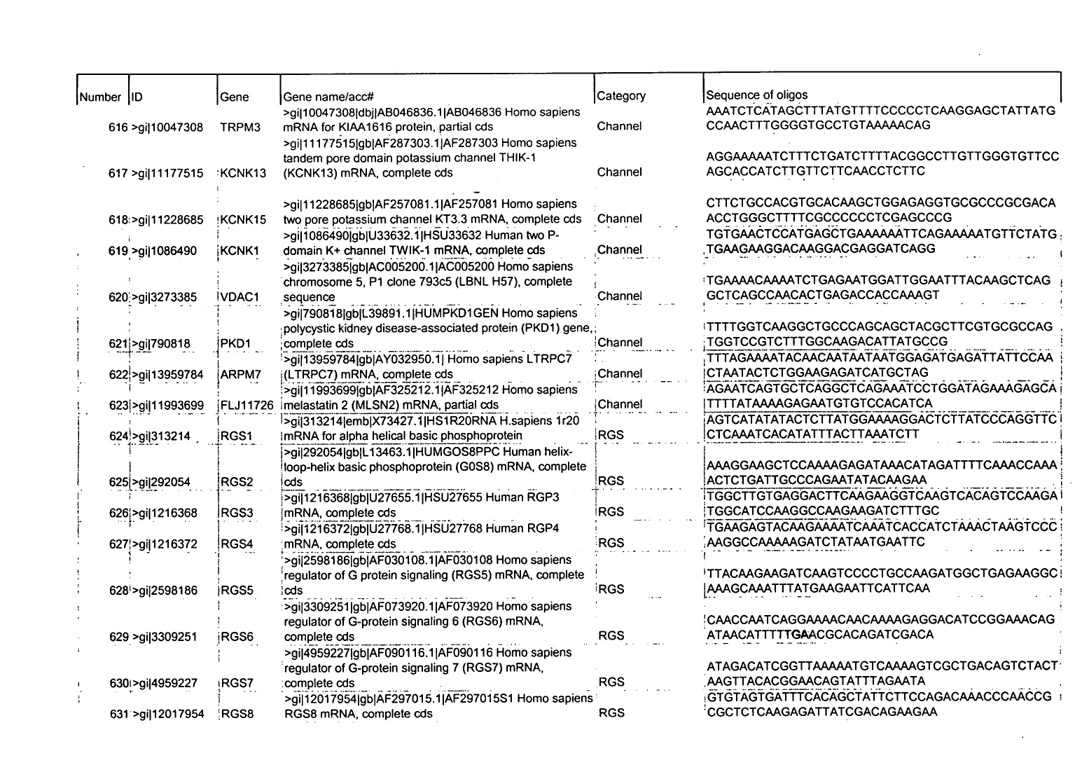

R/G→log₂R/G−cⱼ(A)=log₂R/kⱼ(A)*G→(1/aⱼ)*log₂R/kⱼ(A)*G, where cⱼ(A) is the Lowess fit of the M vs. A plot for spots on the j^{th} grid of each slide, and aⱼ is the scale factor for the j^{th} grid (to obtain equal variances along individual slides). After performing these transformations, the gene expression level of each probe was set to be the median of the four copies of that probe. The box plots of the log ratios for each of the 60 slides are centered close to zero with similar spreads, and on average, 10 outliers per slide (FIG. 7). In this situation we decided not to adjust for scale normalization between slides, as the noise introduced by scale normalization of different slides may be more detrimental than a small difference in scale. This approach resulted in high concordance with cDNA array data.

Correlation analysis between gene expression and drug activity. Growth inhibition data (GI₅₀ values for 60 human tumor cell lines) were those obtained by the Developmental Therapeutics Program (found on the internet at the website url dtp.nci.nih.gov). Values were expressed as potencies by using the negative log of the molar concentration calculated in the NCI screen. We focused on 118 drugs for which the mechanism of action is largely understood, plus the clinically used drug gemcitabine. Pearson correlation coefficients were calculated for assessment of gene-drug relationships. Confidence intervals and unadjusted p-values were obtained using Efron's bootstrap resampling method, with 10,000 bootstrap samples for each gene-drug comparison. To reduce the number of false positive correlations among 87,000 comparisons, we controlled for false discovery rate (FDR) as described. However, because of the computational limitations introduced by the bootstrapping technique, using 10,000 samplings yielded only bootstrap estimators with a resolution of 0.0001. To control FDR at the level 0.05, criteria would have to be too stringent, i.e. only P value=0 was regarded as significant. Therefore an arbitrary cut-off of 0.001 was used for the unadjusted bootstrap P values. This is expected to detect more “true” gene-drug associations, at the expense of increasing the number of false positive ones, to be validated by other means.

RNAi-mediated downregulation of gene expression. SiRNA duplexes for ABCB1 were chemically synthesized by QIAGEN Inc. (Valencia, Calif.). The target sequence is 5′-AAG CGA AGC AGT GGT TCA GGT-3′, beginning from nt 2113 of the ABCB1 mRNA sequence NM_{—}000927, as recommended (found on the internet at the website url www1.qiagen.com/products/genesilencing/cancersirnaset.aspx). Chemically synthesized mock siRNA (fluorescein labeled, non-silencing) was also purchased from QIAGEN. SiRNA duplexes for ABCB5 were synthesized by Silencer siRNA construction kit (Ambion, Austin, Tex.). The three target sequences are
5′-AAAGGAGCTCAAATGAGTGGA-3′ (ABCB5_{—}772),
5′-AAGTGGAGAATCGCTGACCTT-3′ (ABCB5_{—}957), and
5′-AACAGTTTTCTCGATGGCCTG-3′ (ABCB5_{—}1141), which are located at nt 772, 957 and 1141 of the ABCB5 mRNA sequence XM_{—}291215, respectively.

Cell lines, obtained from Division of Cancer Treatment and Diagnosis at NCI, were cultured in RPMI 1640 containing 10% heat-inactivated fetal calf serum in a 5% CO₂ incubator at 37° C. Transfection was performed with TransMessenger Transfection Reagent (QIAGEN). To down-regulate ABCB1 or ABCB5, cancer cells were transfected with 0.3 or 0.6 μM siRNA. For RNA extraction, cells were harvested 48 hours after transfection. To measure cytotoxic drug potency, cells grown in 6-well plates were subcultured into 96-well plates 24 hours after transfection.

Cytotoxicity assay. 5FU, Camptothecin, and Mitoxathone were obtained from Sigma. The other compounds were from Developmental Therapeutics Program at NCI. Drug potency was tested using a proliferation assay with sulforhodamine B (SRB), a protein-binding reagent 37 In each experiment, 3000-5000 cells per well were seeded in 96-well plates and incubated for 24 hours. Anticancer drugs were added in a dilution series in 6 replicated wells. After 4 days, incubation was terminated by replacing the medium with 100 μl 10% trichloroacetic acid (Sigma, St. Louis, Mo.) in 1×PBS, followed by incubation at 4° C. for at least 1 hour. Subsequently, the plates were washed with water and air-dried. The plates were stained with 100 μl 0.4% SRB (Sigma) in 1% acetic acid for 30 min at room temperature. Unbound dye was washed off with 1% acetic acid. After air-drying and re-solubilization of the protein-bound dye in 10 mM Tris-HCl (pH 8.0), absorbance was read by a micro-plate reader at 570 nm. To determine the IC₅₀ values, the absorbance of control cells without drug was set at 1. Dose-response curves were plotted using SigmaPlot software (RockWare, Golden, Colo.). Each experiment was performed independently at least three times.

Real-time quantitative RT-PCR. Total RNA was prepared by using the RNeasy Mini Kit (Qiagen), following the manufacturer's protocol. The integrity of the RNA was assessed by denaturing agarose gel electrophoresis (visual presence of sharp 28S and 18S bands) and by spectrophotometry. One microgram of total RNA was incubated with DNase I, and reverse transcribed with oligo dT with Superscript II RT-PCR (Life Technologies). One microliter of RT product was amplified by primer pairs specific for selected genes. Primers were designed with Primer Express software (Applied Biosystems), and ACTB (beta-actin) was used as a normalizing control. Relative gene expression was measured with the GeneAmp 7000 Sequence Detection system (Applied Biosystems, Foster City, Calif.). Conditions and primer sequences are available on request.

### Example 7

### Hierachical Clustering of NCI-60 According to Transportome Gene Expression

Hierarchical clustering by gene expression was used to group the 60 cell lines. This is an important validation step, as cells with similar origin should cluster together, as shown previously with other array results. Generally, cells of similar tissue origin tended clustered together (FIG. 4), with some notable differences from that based on expression of 1,376 genes reported by Scherf et al. MDA-MB-435 and its Erb/B2 transfectant MDA-N were clustered together, and both cell lines clustered with melanomas since they express genes characteristic of melanoma cells. These cell lines express high levels of ABCB5, a novel resistance gene proposed in this study. Overall, cell clustering support the validity of the array results. Failure of some cell lines to cluster with their tissue of origin is probably due to the different gene panel used. Genes relevant to chemosensitivity may not reflect fully the physiology of the cell, so that clustering on the basis of chemosensitivity does not reflect the tissue of origin. Cell lines NCI/ADR-RES and TK-10 tested in duplicate, using independent labeling and hybridizations, clustered together, supporting reproducibility of the analysis.

### Example 8

### Comparing Gene Expression Data Obtained with the 70-mer Oligo Array To Multiple Expression Datasets Using Other Arrays or Methods

To validate the microarray results, mRNA expression data obtained with the 70-mer arrays were compared to those obtained with cDNA, Affymetrix HG-6800 and Affymetrix U133A arrays (unpublished data). 137, 235 and 476 genes were commonly represented between the 70-mer and cDNA, HG-6800 and U133A arrays, respectively. The mean Pearson Correlation coefficients between the 70-mer oligo and cDNA arrays for all the 60 cell lines was 0.43±0.14 (p<0.05). This indicates that for a majority of common genes, these two arrays yielded similar results. Gene-by-gene analysis revealed that correlations strongly depend on the relative expression level (hybridization intensity). The higher the expression the greater the correlation coefficient (Anderle et al., to be published).

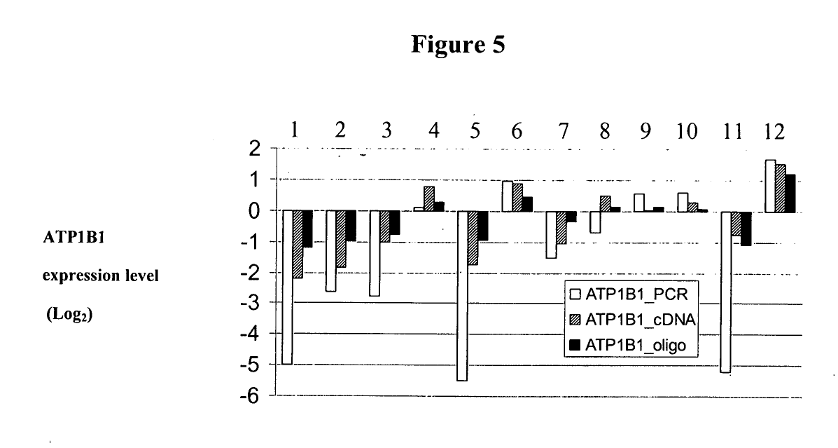

To validate the array data further, we determined ATP1B1 expression by real-time RT-PCR and compared the result with those from our 70-mer oligo and cDNA arrays. The RT-PCR experiment agreed well the array results (FIG. 5).

We also compared gene expressions between the 70-mer oligo array and RT-PCR results for 40 ABC transporters genes (Gottesman et al., accompanying report). However, the majority of these genes were poorly expressed and close to the limit of detection of the 70-mer array in a majority of cell lines. Therefore, we used all expression data for comparison, or only the top and bottom 40% or 20% values for each gene expression data set. Under these three conditions, 22%, 63%, and 78% of the genes showed significant correlations (P<0.05) between the 70-mer array and RT-PCR data, indicating that the different assays yielded comparable results.

TABLE 4
SLC transporters showing significant correlations with drugs.

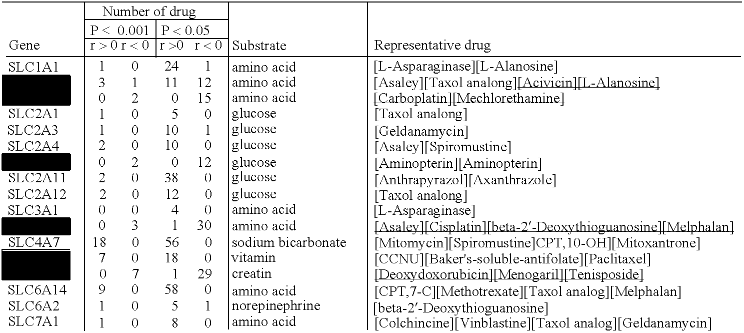

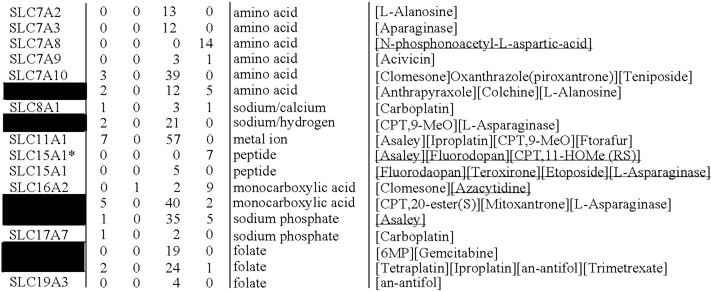

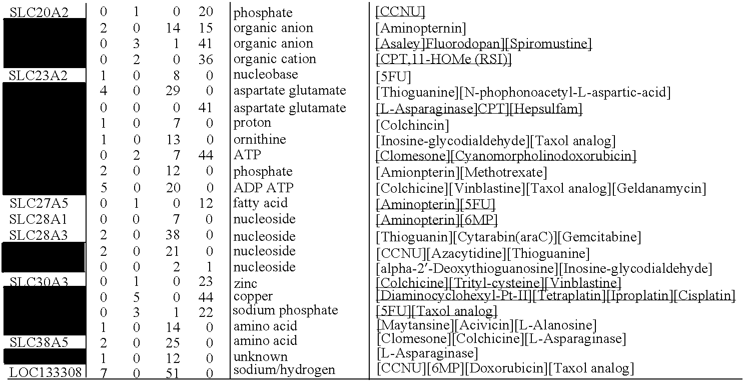

Activities of the underlined drugs have negative correlations with expression of the corresponding genes. Shadowed genes have concordant expression patterns in at least one comparison between results obtained with 70-mer arrays, cDNA arrays, Affymetrix arrays, and RT-PCR. For each gene, the number of drugs with positive or negative correlation values ® is shown, with P<0.05 and P<0.001. *: The pH-sensing regulatory splice variant of SLC15A1.

TABLE 5
ABC transporters that show significant correlations with drugs.

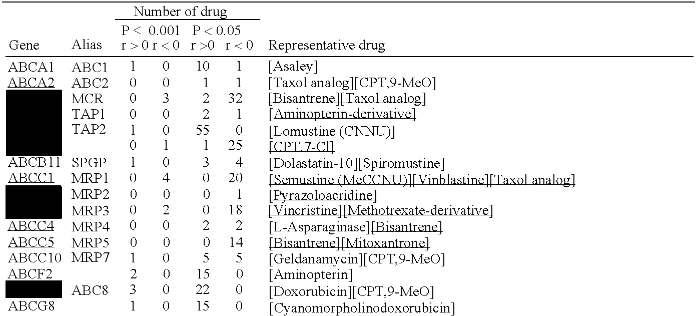

Known drug resistance genes are included if P<0.05; for all others, P<0.001 was taken as the criterion for inclusion. Underlined genes are those previously reported to be involved in chemo-resistance. Underlined drugs are those showing negative correlation with the corresponding genes. Shadowed genes are those showing concordant expression patterns in at least one comparisons between results obtained with 70-mer arrays, cDNA arrays, Affymetrix arrays, and RT-PCR. For each gene, the number of drugs with positive or negative correlation values ® is shown, with P<0.05 and P<0.001.

TABLE 6
Ion pumps and channels showing significant drug correlations.

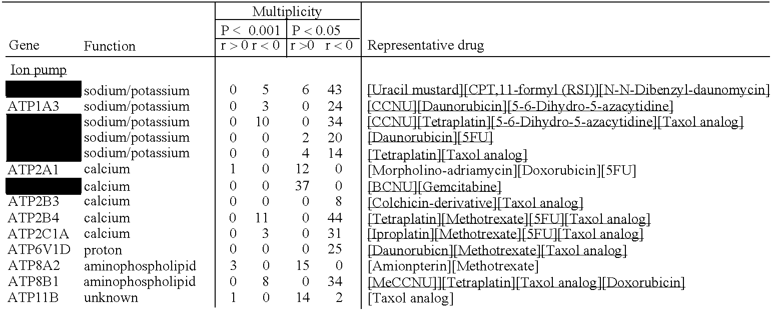

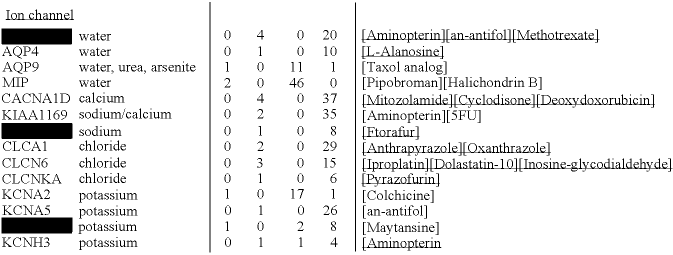

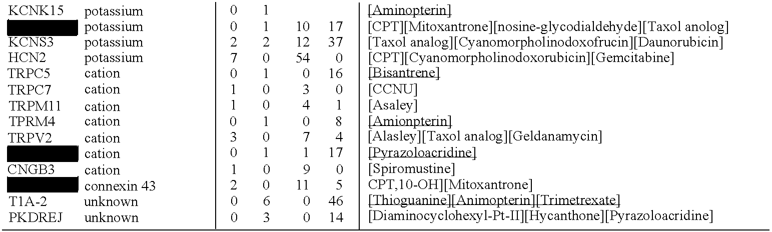

Underlined drugs correlate negatively with the corresponding genes. Shadowed genes have concordant expression patterns in at least one comparison between results obtained with 70-mer arrays, cDNA arrays, Affymetrix arrays, and RT-PCR. For each gene, the number of drugs with significantly positive or negative gene-drug correlation coefficients, r, (P<0.05 and P<0.001) is shown.

### Example 9

### Representation of all Significant Gene-Drug Correlations

We compiled all gene-drug correlations reaching a bootstrap p value of <0.001, and <0.05 for previously published substrate-transporter interactions (Tables 4 to 6). It is important to emphasize that there is some risk of false-positive relationships, and moreover, many significant gene-drug pairs will be missed. False negative results are particularly likely because significant correlation require not only that a cytotoxic drug be a substrate, but that this interaction also play a significant role in variability across the NCI60. Spurious significant correlations can also occur if genes are coordinately expressed, or the oligo probes lack specificity. These problems are addressed by correlating each gene with numerous chemicals that have been tested against the NCI60. Typically we use 120 test drugs, but for finding high correlations, we have used subsets of ˜1,500, ˜4,500 and more drugs. Once a significant drug-gene correlation is identified, chemical similarity searches can be used to find additional substrates. In results presented in Tables 4, 5 and 6, we highlight those genes that show concordance (Pearson correlation coefficient r>0.3) in at least one comparison with other expression studies, using different arrays or methods. Therefore, each listed correlation requires separate validation before it can be considered reliable.

### Example 10

### Methods for Cluster Analysis

Clustering of cell lines by gene expression profiles. Hierarchical clustering can be used to group cell lines in terms of their patterns of gene expression^{38, 41}. To obtain cell-cell cluster trees for 57 genes that showed robust patterns across the 60 cell lines (i.e., genes that passed the filter S.D.>=0.39), we used the programs “Cluster” and “TreeView” 42 with average linkage clustering and a correlation metric.

Comparison between the 70-mer oligo, cDNA, and Affymetrix arrays and RT-PCR studies. Gene expression profiles for the NCI-60 have been measured using cDNA arrays and Affymetrix oligonucleotide chips (HG-6800). Both sets are available on the internet at the website url discover.nci.nih.gov. For the cDNA arrays, each cell type was hybridized against a reference pool of mRNA from 12 highly diverse cell lines³⁸. The cDNA data were normalized using Gaussian-windowed moving-average fits without background subtraction⁴³ and log2 transformed³⁸. Average differences from the Affymetrix data were calculated using the Affymetrix GeneChip software, with spot intensity floored at 30 (i.e., all values lower than 30 were set to 30), then log2 transformed. To begin the comparison analysis, we used UniGene clustering and Genbank sequence information to identify genes common to the different types of arrays. For that purpose, we used parseUniGene, an early version of the program MatchMiner 44 (found on the internet at the website url discover.nci.nib.gov), with UniGene build 132 (February 2001). 137 genes were common to the 70-mer arrays and cDNA arrays, 235 genes were common to the 70-mer arrays and Affymetrix arrays, and 102 genes were common to all three array types. Pearson correlation coefficient served as an index of the concordance between expression levels of common genes for each cell line, and across the 60 cell lines for each gene. Correlation coefficients (r) of 0.3 were taken to indicate that the two arrays yield concordant results.

1. An array for determining the chemosensitivity of a cancer cell to a particular agent, comprising a plurality of polynucleotide probes designed to be complementary to and hybridize under stringent conditions with a target region of at least one gene listed in one of FIG. 15 or 16, wherein at least one of the polynucleotide probes is a control probe.

2. The array of claim 1, wherein the polynucleotide probes are immobilized on a substrate.

3. The array of claim 2, wherein the polynucleotide probes are between 10 and 80 nucleotides in length.

4. The array of claim 3 wherein the polynucleotide probes are 70 nucleotides in length.

5. The array of claim 4 wherein the polynucleotide probes are selected from the group consisting of oligonucleotides, cDNA molecules, and synthetic gene probes comprising nucleobases.

6. The array of claim 1, wherein one or more of the polynucleotide probes has a sequence corresponding to one or more of the oligonucleotide sequences listed in FIG. 8.

7. The array of claim 1, comprising at least 10 control probes and at least 10 polynucleotide probes designed to be complementary to and hybridize under stringent conditions with a target region of at least one gene listed in one of FIG. 15 or 16.

8. A method for detecting a chemosensitivity gene expression profile a cancer cell, comprising
hybridizing at least one target nucleic acid from a sample containing the cancer cell to an array of polynucleotide probes immobilized on a surface, said array comprising a plurality of polynucleotide probes, at least one of which is a control probe, and wherein at least one of said polynucleotide probes is complementary to a target region of at least one chemosensitivity gene listed in one of FIG. 15 or 16; and
quantifying the hybridization of said target nucleic acids to said array,
wherein the expression profile of the cell provides an indication of the likely chemosensitivity or chemoresistance of the cells to a variety of different cytotoxic agents.

9. The method of claim 8, wherein said array comprises mismatch control polynucleotide probes.

10. The method of claim 9, wherein said quantifying comprises calculating the difference in hybridization signal intensity between each of said polynucleotide probes and its corresponding mismatch control probe.

11. The method of claim 10, wherein said quantifying comprises calculating the average difference in hybridization signal intensity between each of said polynucleotide probes and its corresponding mismatch control probe for each gene.

12. The method of claim 8, wherein said plurality of polynucleotide probes is 100 or more.

13. The method of claim 8, wherein for each target region of at least one chemosensitivity gene, said array comprises at least 10 different polynucleotide probes complementary to a target region of each chemosensitivity gene.

14. The method of claim 8, wherein said oligonucleotides are from 15 to 100 nucleotides in length.

15. The method of claim 8, wherein said oligonucleotides are 70 nucleotides in length.

16. The method of claim 8, wherein said pool of target nucleic acids is a pool of mRNAs.

17. The method of claim 8, wherein said pool of target nucleic acids is a pool of RNAs in vitro transcribed from a pool of cDNAs.

18. The method of claim 8, wherein said pool of target nucleic acids is amplified from a biological sample by an in vivo or an in vitro method.

19. The method of claim 8, wherein said pool of target nucleic acids comprises fluorescently labeled nucleic acids.

20. The method of claim 8, wherein each different polynucleotide probe is localized in a predetermined region of said surface, the density of said different polynucleotide probes is greater than about 60 different polynucleotide probes per 1 cm².

21. The method of claim 8, comprising the step of comparing the pattern of chemosensitivity gene expression with gene-drug correlations shown in FIG. 9 to identify matches between the genes expressed in the cells and genes that correlate with chemosensitivity or chemoresistance.

22. A method for predicting the effect of a cytotoxic agent on a cancer cell obtained from a mammalian subject, comprising
hybridizing a sample containing target nucleic acids obtained from a cancer cell from a mammalian subject to an array of polynucleotide probes immobilized on a surface, said array comprising a plurality of different polynucleotide probes, at least one of which is a control probe, and wherein at least one of said polynucleotide probes is complementary to a target region of at least one chemosensitivity gene listed in one of FIG. 9 or 10; and
quantifying the hybridization of said nucleic acids to said array,
wherein the expression profile of the cells provides an indication of the chemosensitivity or chemoresistance of the cells to a variety of different cytotoxic agents.

23. The method of claim 22, comprising the step of comparing the pattern of chemosensitivity gene expression with the gene-drug correlations listed in FIG. 9 to identify matches between the genes expressed in the cells and genes that correlate with chemosensitivity or chemoresistance.

24. A method of identifying and characterizing an agent that modulates the expression or activity of one or more chemosensitivity genes, comprising:
exposing a culture of mammalian cells to said candidate agent;
determining the effect of the candidate agent on expression of one or more chemosensitivity genes listed in one of FIG. 15 or 16, or one of Tables 1-6.

25. The method of claim 24 wherein the effect of the candidate agent on transcription of chemosensitivity genes is determined by measuring the levels of transcripts of said chemosensitivity genes in said cells.

26. The method of claim 24 wherein the levels of transcripts are measured using an array that comprises polynucleotide probes that hybridize with at least 10 chemosensitivity gene transcripts, wherein not more than 100 polynucleotide probes are complementary to genes that do not influence chemosensitivity.

27. The method of claim 24 wherein the polynucleotide probes are oligonucleotides selected from the oligonucleotides listed in FIG. 8.

28. The method of claim 24 wherein the array comprises 10 or more of said oligonucleotides.

29. The method of claim 24 wherein the oligonucleotides comprise polynucleotide probes designed to be complementary to, or hybridize under stringent conditions with, 10 or more chemosensitivity genes listed in listed in one of FIG. 9 and FIG. 10, or in one of Tables 1-6.

30. The method of claim 24 wherein the oligonucleotides comprise nucleotide probes designed to be complementary to, or hybridize under stringent conditions with target regions of 10, 20, 30, 40, 50, 60, 70, 80, 90, 100, 200, or more chemosensitivity genes listed in listed in one of FIG. 9 and FIG. 10, or in one of Tables 1-6
